SEQUENCE LISTING (51 sequences)

SEQ ID NO: 1 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 2 - protein, 466 aa, Homo sapiens
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQ

SEQ ID NO: 3 - protein, 31 aa, Homo sapiens
YGNKKNNNQNNNVVEPEPEPEPEPEPEPVSE

SEQ ID NO: 4 - protein, 386 aa, Homo sapiens
MEDEMPKTLYVGNLSRDVTEALILQLFSQIGPCKNCKMIMDTAGNDPYCFVEFHEHRHAAAALAAMNGRKIMGKEVKVNWATTPSSQKKDTSSSTVVSTQRSQDHFHVFVGDLSPEITTEDIKAAFAPFGRISDARVVKDMATGKSKGYGFVSFFNKWDAENAIQQMGGQWLGGRQIRTNWATRKPPAPKSTYESNTKQLSYDEVVNQSSPSNCTVYCGGVTSGLTEQLMRQTFSPFGQIMEIRVFPDKGYSFVRFNSHESAAHAIVSVNGTTIEGHVVKCYWGKETLDMINPVQQQNQIGYPQPYGQWGQWYGNAQQIGQYMPNGWQVPAYGMYGQAWNQQGFNQTQSSAPWMGPNYGVQPPQGQNGSMLPNQPSGYRVAGYETQ

SEQ ID NO: 5 - protein, 695 aa, Homo sapiens
MEEKPGQPQPQHHHSHHHPHHHPQQQQQQPHHHHHYYFYNHSHNHHHHHHHQQPHQYLQHGAEGSPKAQPKPLKHEQKHTLQQHQETPKKKTGYGELNGNAGEREISLKNLSSDEATNPISRVLNGNQQVVDTSLKQTVKANTFGKAGIKTKNFIQKNSMDKKNGKSYENKSGENQSVDKSDTIPIPNGVVTNNSGYITNGYMGKGADNDGSGSESGYTTPKKRKARRNSAKGCENLNIVQDKIMQQETSVPTLKQGLETFKPDYSEQKGNRVDGSKPIWKYETGPGGTSRGKPAVGDMLRKSSDSKPGVSSKKFDDRPKGKHASAVASKEDSWTLFKPPPVFPVDNSSAKIVPKISYASKVKENLNKTIQNSSVSPTSSSSSSSSTGETQTQSSSRLSQVPMSALKSVTSANFSNGPVLAGTDGNVYPPGGQPLLTTAANTLTPISSGTDSVLQDMSLTSAAVEQIKTSLFIYPSNMQTMLLSTAQVDLPSQTDQQNLGDIFQNQWGLSFINEPSAGPETVTGKSSEHKVMEVTFQGEYPATLVSQGAEIIPSGTEHPVFPKAYELEKRTSPQVLGSILKSGTTSESGALSLEPSHIGDLQKADTSSQGALVFLSKDYEIESQNPLASPTNTLLGSAKEQRYQRGLERNDSWGSFDLRAAIVYHTKEMESIWNLQKQDPKRIITYNEAMDSPDQ

SEQ ID NO: 6 - protein, 621 aa, Homo sapiens
MAELTVEVRGSNGAFYKGFIKDVHEDSLTVVFENNWQPERQVPFNEVRLPPPPDIKKEISEGDEVEVYSRANDQEPCGWWLAKVRMMKGEFYVIEYAACDATYNEIVTFERLRPVNQNKTVKKNTFFKCTVDVPEDLREACANENAHKDFKKAVGACRIFYHPETTQLMILSASEATVKRVNILSDMHLRSIRTKLMLMSRNEEATKHLECTKQLAAAFHEEFVVREDLMGLAIGTHGSNIQQARKVPGVTAIELDEDTGTFRIYGESADAVKKARGFLEFVEDFIQVPRNLVGKVIGKNGKVIQEIVDKSGVVRVRIEGDNENKLPREDGMVPFVFVGTKESIGNVQVLLEYHIAYLKEVEQLRMERLQIDEQLRQIGSRSYSGRGRGRRGPNYTSGYGTNSELSNPSETESERKDELSDWSLAGEDDRDSRHQRDSRRRPGGRGRSVSGGRGRGGPRGGKSSISSVLKDPDSNPYSLLDNTESDQTADTDASESHHSTNRRRRSRRRRTDEDAVLMDGMTESDTASVNENGLVTVADYISRAESQSRQRNLPRETLAKNKKEMAKDVIEEHGPSEKAINGPTSASGDDISKLQRTPGEEKINTLKEENTQEAAVLNGVS

SEQ ID NO: 7 - protein, 526 aa, Homo sapiens
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 8 - protein, 326 aa, Homo sapiens
MSNGYEDHMAEDCRGDIGRTNLIVNYLPQNMTQDELRSLFSSIGEVESAKLIRDKVAGHSLGYGFVNYVTAKDAERAINTLNGLRLQSKTIKVSYARPSSEVIKDANLYISGLPRTMTQKDVEDMFSRFGRIINSRVLVDQTTGLSRGVAFIRFDKRSEAEEAITSFNGHKPPGSSEPITVKFAANPNQNKNVALLSQLYHSPARRFGGPVHHQAQRFRFSPMGVDHMSGLSGVNVPGNASSGWCIFIYNLGQDADEGILWQMFGPFGAVTNVKVIRDFNTNKCKGFGFVTMTNYEEAAMAIASLNGYRLGDKILQVSFKTNKSHK

SEQ ID NO: 9 - protein, 582 aa, Homo sapiens
MEALSRAGQEMSLAALKQHDPYITSIADLTGQVALYTFCPKANQWEKTDIEGTLFVYRRSASPYHGFTIVNRLNMHNLVEPVNKDLEFQLHEPFLLYRNASLSIYSIWFYDKNDCHRIAKLMADVVEEETRRSQQAARDKQSPSQANGCSDHRPIDILEMLSRAKDEYERNQMGDSNISSPGLQPSTQLSNLGSTETLEEMPSGSQDKSAPSGHKHLTVEELFGTSLPKEQPAVVGLDSEEMERLPGDASQKEPNSFLPFPFEQLGGAPQSETLGVPSAAHHSVQPEITTPVLITPASITQSNEKHAPTYTIPLSPVLSPTLPAEAPTAQVPPSLPRNSTMMQAVKTTPRQRSPLLNQPVPELSHASLIANQSPFRAPLNVTNTAGTSLPSVDLLQKLRLTPQHDQIQTQPLGKGAMVASFSPAAGQLATPESFIEPPSKTAAARVAASASLSNMVLAPLQSMQQNQDPEVFVQPKVLSSAIPVAGAPLVTATTTAVSSVLLAPSVFQQTVTRSSDLERKASSPSPLTIGTPESQRKPSIILSKSQLQDTLIHLIKNDSSFLSTLHEVYLQVLTKNKDNHNL

SEQ ID NO: 10 - protein, 142 aa, Homo sapiens
MGLKLNGRYISLILAVQIAYLVQAVRAAGKCDAVFKGFSDCLLKLGDSMANYPQGLDDKTNIKTVCTYWEDFHSCTVTALTDCQEGAKDMWDKLRKESKNLNIQGSLFELCGSGNGAAGSLLPAFPVLLVSLSAALATWLSF

SEQ ID NO: 11 - protein, 419 aa, Homo sapiens
MFALADINSFYASCEKVFRPDLRNEPVIVLSNNDGCVIARSPEAKALGIRMGQPWFQVRQMRLEKKIHVFSSNYALYHSMSQRVMAVLESLSPAVEPYSIDEMFIDLRGINHCISPEFFGHQLREQVKSWTGLTMGVGIAPTKTLAKSAQWATKQWPQFSGVVALTAENRNRILKLLGLQPVGEVWGVGHRLTEKLNALGINTALQLAQANTAFIRKNFSVILERTVRELNGESCISLEEAPPAKQQIVCSRSFGERITDKDAMHQAVVQYAERAAEKLRGERQYCRQVTTFVRTSPTLLQQCAVEKLSLPTQDSRDIIAAACRALNHVWREGYRYMKAGVMLADFTPSGIAQPGLFDEIQPRKNSEKLMKTLDELNQSGKGKVWFAGRGTAPEWQMKREMLSQCYTTKWRDIPLARLG

SEQ ID NO: 12 - protein, 238 aa, Homo sapiens
MLCCMRRTKQVEKNDDDQKIEQDGIKPEDKAHKAATKIQASFRGHITRKKLKGEKKDDVQAAEAEANKKDEAPVADGVEKKGEGTTTAEAAPATGSKPDEPGKAGETPSEEKKGEGDAATEQAAPQAPASSEEKAGSAETESATKASTDNSPSSKAEDAPAKEEPKQADVPAAVTAAAATTPAAEDAAAKATAQPPTETGESSQAEENIEAVDETKPKESARQDEGKEEEPEADQEHA

SEQ ID NO: 13 - protein, 480 aa, Homo sapiens
MSTLCPPPSPAVAKTEIALSGKSPLLAATFAYWDNILGPRVRHIWAPKTEQVLLSDGEITFLANHTLNGEILRNAESGAIDVKFFVLSEKGVIIVSLIFDGNWNGDRSTYGLSIILPQTELSFYLPLHRVCVDRLTHIIRKGRIWMHKERQENVQKIILEGTERMEDQGQSIIPMLTGEVIPVMELLSSMKSHSVPEEIDIADTVLNDDDIGDSCHEGFLLNAISSHLQTCGCSVVVGSSAEKVNKIVRTLCLFLTPAERKCSRLCEAESSFKYESGLFVQGLLKDSTGSFVLPFRQVMYAPYPTTHIDVDVNTVKQMPPCHEHIYNQRRYMRSELTAFWRATSEEDMAQDTIIYTDESFTPDLNIFQDVLHRDTLVKAFLDQVFQLKPGLSLRSTFLAQFLLVLHRKALTLIKYIEDDTQKGKKPFKSLRNLKIDLDLTAEGDLNIIMALAEKIKPGLHSFIFGRPFYTSVQERDVLMT

SEQ ID NO: 14 - protein, 169 aa, Mus musculus
MLASRALSLIGKRAISTSVCLRAHGSVVKSEDYAFPTYADRRDYPLPDVAHVTMLSASQKALKEKEKADWSSLSRDEKVQLYRIQFNESFAEMNRGTNEWKTVVGMAMFFIGFTALVLIWEKSYVYGPIPHTFDRDWVAMQTKRMLDMKANPIQGFSAKWDYDKNEWKK

SEQ ID NO: 15 - protein, 553 aa, Homo sapiens
MLSVRVAAAVVRALPRRAGLVSRNALGSSFIAARNFHASNTHLQKTGTAEMSSILEERILGADTSVDLEETGRVLSIGDGIARVHGLRNVQAEEMVEFSSGLKGMSLNLEPDNVGVVVFGNDKLIKEGDIVKRTGAIVDVPVGEELLGRVVDALGNAIDGKGPIGSKTRRRVGLKAPGIIPRISVREPMQTGIKAVDSLVPIGRGQRELIIGDRQTGKTSIAIDTIINQKRFNDGSDEKKKLYCIYVAIGQKRSTVAQLVKRLTDADAMKYTIVVSATASDAAPLQYLAPYSGCSMGEYFRDNGKHALIIYDDLSKQAVAYRQMSLLLRRPPGREAYPGDVFYLHSRLLERAAKMNDAFGGGSLTALPVIETQAGDVSAYIPTNVISITDGQIFLETELFYKGIRPAINVGLSVSRVGSAAQTRAMKQVAGTMKLELAQYREVAAFAQFGSDLDAATQQLLSRGVRLTELLKQGQYSPMAIEEQVAVIYAGVRGYLDKLEPSKITKFENAFLSHVVSQHQALLGTIRADGKISEQSDAKLKEIVTNFLAGFEA

SEQ ID NO: 16 - protein, 81 aa, Homo sapiens
MLRQIIGQAKKHPSLIPLFVFIGTGATGATLYLLRLALFNPDVCWDRNNPEPWNKLGPNDQYKFYSVNVDYSKLKKERPDF

SEQ ID NO: 17 - DNA, 21 bp, Homo sapiens
  source 1..21: mol_type = genomic DNA
ccaaggacaggagtgaatgac

SEQ ID NO: 18 - DNA, 20 bp, Homo sapiens
  source 1..20: mol_type = genomic DNA
gtatggaatcctgtggcatc

SEQ ID NO: 19 - DNA, 21 bp, Homo sapiens
  source 1..21: mol_type = genomic DNA
ctacagtctgtggcagtttca

SEQ ID NO: 20 - DNA, 30 bp, Homo sapiens
  source 1..30: mol_type = genomic DNA
aaactcaaaggacttggcggtactttatat

SEQ ID NO: 21 - DNA, 24 bp, Homo sapiens
  source 1..24: mol_type = genomic DNA
catttctacttcggtgtgccttcg

SEQ ID NO: 22 - DNA, 21 bp, Homo sapiens
  source 1..21: mol_type = genomic DNA
tgtcggatctgctgcccaaac

SEQ ID NO: 23 - DNA, 26 bp, Homo sapiens
  source 1..26: mol_type = genomic DNA
cgtatttattggagcagggggtactg

SEQ ID NO: 24 - DNA, 27 bp, Homo sapiens
  source 1..27: mol_type = genomic DNA
caatttccacctctgtgtgtgtacgag

SEQ ID NO: 25 - DNA, 23 bp, Homo sapiens
  source 1..23: mol_type = genomic DNA
agtcgtggcgtgcgtctaactga

SEQ ID NO: 26 - protein, 31 aa, Rattus norvegicus
EESEEEVEEPEENQQSPEVVYGNKKNNQNNN

SEQ ID NO: 27 - protein, 33 aa, Rattus norvegicus
DDSGTFYDQTVSNDLEEHLEEPYGNKKNNQNNN

SEQ ID NO: 28 - protein, 31 aa, Rattus norvegicus; identical to SEQ ID NO: 3

SEQ ID NO: 29 - protein, 21 aa, Homo sapiens
EPVAEPEPDPEPEPEEEPVSE

SEQ ID NO: 30 - DNA, 58 bp, Homo sapiens
  source 1..58: mol_type = genomic DNA
ttccagtaaataggcatggagttgcatggcttacactcggacctcgtcgacatgcatt

SEQ ID NO: 31 - DNA, 57 bp, Homo sapiens
  source 1..57: mol_type = genomic DNA
catagtgcttctgccacatgataacgagcttacactcggacctcgtcgacatgcatt

SEQ ID NO: 32 - DNA, 57 bp, Homo sapiens
  source 1..57: mol_type = genomic DNA
tgttcatctcagcaaagctctccttgaacttacactcggacctcgtcgacatgcatt

SEQ ID NO: 33 - DNA, 54 bp, Homo sapiens
  source 1..54: mol_type = genomic DNA
gcaaggggtggtcacgccgatccatattacactcggacctcgtcgacatgcatt

SEQ ID NO: 34 - DNA, 55 bp, Homo sapiens
  source 1..55: mol_type = genomic DNA
tggaaattgctcgcttgccaactaggcttacactcggacctcgtcgacatgcatt

SEQ ID NO: 35 - DNA, 54 bp, Homo sapiens
  source 1..54: mol_type = genomic DNA
aataccctggtagccaacattctgccttacactcggacctcgtcgacatgcatt

SEQ ID NO: 36 - DNA, 54 bp, Homo sapiens
  source 1..54: mol_type = genomic DNA
cagcatctctcacttcttccactcgtttacactcggacctcgtcgacatgcatt

SEQ ID NO: 37 - DNA, 57 bp, Homo sapiens
  source 1..57: mol_type = genomic DNA
ttttcgtagtcccacttggaggctaagccttacactcggacctcgtcgacatgcatt

SEQ ID NO: 38 - DNA, 54 bp, Homo sapiens
  source 1..54: mol_type = genomic DNA
ggatggggttcaccttcatgtccagcttacactcggacctcgtcgacatgcatt

SEQ ID NO: 39 - DNA, 54 bp, Homo sapiens
  source 1..54: mol_type = genomic DNA
ctgcttggccacccactctttgtcaattacactcggacctcgtcgacatgcatt

SEQ ID NO: 40 - DNA, 54 bp, Homo sapiens
  source 1..54: mol_type = genomic DNA
acaaccgtcttccactcgttcgagccttacactcggacctcgtcgacatgcatt

SEQ ID NO: 41 - DNA, 54 bp, Homo sapiens
  source 1..54: mol_type = genomic DNA
caggaggccttctccttctccttcaattacactcggacctcgtcgacatgcatt

SEQ ID NO: 42 - DNA, 54 bp, Homo sapiens
  source 1..54: mol_type = genomic DNA
ccttctggctggcagacaggtgcttgttacactcggacctcgtcgacatgcatt

SEQ ID NO: 43 - protein, 21 aa, Homo sapiens
AAGCACAGAGAAGAGGCAATC

SEQ ID NO: 44 - DNA, 20 bp, Homo sapiens
  source 1..20: mol_type = genomic DNA
aagcacttgcggtgcacgat

SEQ ID NO: 45 - DNA, 19 bp, Homo sapiens
  source 1..19: mol_type = genomic DNA
tggctgtttgctggattct

SEQ ID NO: 46 - DNA, 28 bp, Homo sapiens
  source 1..28: mol_type = genomic DNA
atacctttttagggtttgctgaagatgg

SEQ ID NO: 47 - DNA, 24 bp, Homo sapiens
  source 1..24: mol_type = genomic DNA
gatcagcgtaagtggggaaagcat

SEQ ID NO: 48 - DNA, 22 bp, Homo sapiens
  source 1..22: mol_type = genomic DNA
acgcacaccacgactcaagagc

SEQ ID NO: 49 - DNA, 24 bp, Homo sapiens
  source 1..24: mol_type = genomic DNA
ggctctgggttgttctttctgtcc

SEQ ID NO: 50 - DNA, 18 bp, Homo sapiens
  source 1..18: mol_type = genomic DNA
ggcaaggggtggtcacgc

SEQ ID NO: 51 - DNA, 25 bp, Homo sapiens
  source 1..25: mol_type = genomic DNA
ccttacacccgcatagataacagcc

(19) United States
(12) Patent Application Publication
(10) Pub. No.: US 2023/0190887 A1
(43) Pub. Date: Jun. 22, 2023

(54) TARGETING G3BP AGGREGATION TO PREVENT NEURODEGENERATION

(71) Applicants: University of South Carolina, Columbia, SC (US); Ramot at Tel-Aviv University Ltd., Tel Aviv (IL); Tel HaShomer Medical Research Infrastructure and Services Ltd., Ramat-Gan (IL)

(72) Inventors: Amar N. Kar, Lexington, SC (US); Pabitra Sahoo, West Columbia, SC (US); Jeffery Twiss, Columbia, SC (US); Sean McGill, Columbia, SC (US); Eran Perlson, Tel Aviv (IL); Topaz Altman, Tel Aviv (IL); Ariel Razvan Ionescu, Tel Aviv (IL); Tal Gradus-Pery, Tel Aviv (IL); Amir Dori, Herzliya (IL)

(73) Assignees: University of South Carolina, Columbia, SC (US); Ramot at Tel-Aviv University Ltd., Tel Aviv (IL); Tel HaShomer Medical Research Infrastructure and Services Ltd., Ramat-Gan (IL)

(21) Appl. No.: 17/989,817

(22) Filed: Nov. 18, 2022

Related U.S. Application Data
(63) Continuation-in-part of application No. 16/881,096, filed on May 22, 2020.
(60) Provisional application No. 62/876,852, filed on Jul. 22, 2019.

(51) Int. Cl. A61K 38/46; A61P 25/28

(52) U.S. Cl. CPC A61K 38/465; A61P 25/28

(57) ABSTRACT

Testing peptides in in vitro models of neurodegenerative disorders such as Parkinson's disease, Alzheimer's disease, Frontotemporal dementia, Amyotrophic lateral sclerosis, to evaluate systems and methods of treatment therefore.

This invention was made with government support under R01 NS04151596 awarded by the National Institutes of Health. The government has certain rights in the invention.

### BACKGROUND OF THE INVENTION

### 1) Field of the Invention

The present invention relates to testing peptides in in vitro models of neurodegenerative disorders such as Parkinson's disease, Alzheimer's disease, Frontotemporal dementia, Amyotrophic lateral sclerosis, to evaluate systems and methods of treatment.

### 2) Description of Related Art

Neurodegenerative disease is a common and growing cause of mortality and morbidity worldwide. Roughly about vie (5) million Americans suffer from Alzheimer's disease (AD); one (1) million from Parkinson's disease (PD); four hundred thousand (400,000) from multiple sclerosis (MS); thirty thousand (30,000) from Amyotrophic lateral sclerosis (ALS), and three thousand (3000) from Huntington's disease (HD).

In recent years, altered RNA processing has emerged as a key contributing factor in several neurodegenerative diseases. Many of the neurodegenerative diseases share features such as protein aggregates containing proteins such as Tau, alpha-synuclein and beta-amyloid, as well as aberrant cytosolic protein complexes consisting of stress granules (SG) associated RNA binding proteins (RBPs), such as TDP-43, FUS and TIA-1. Studies suggest that in a diseased neuron, both altered RBP function and increased cytoplasmic aggregation of RBPs together contribute to the disease progression. There has been limited success in developing potential therapeutic interventions that directly target formation of aberrant SG and cytoplasmic aggregates.

Stress granules are cytosolic inclusions of proteins that function in regulating translation of messenger RNAs during cellular stress, such as heat shock, oxygen deprivation, or oxidative damage. These structures sequester non-essential mRNAs and allow the cell to tailor the production of proteins that help in mitigating the stressful insults. Upon alleviation of stress, the stress granules usually dissipate, and this disassembly correlates with the resumption of widespread protein synthesis. RNA binding proteins such as, T cell intracellular antigen 1 (TIA-1) and RasGAP-associated endoribonuclease (G3BP) nucleate stress granules formation which is followed by recruitment of ribosomal subunits, translation initiations factors and other RBPs.

The potential importance of stress granules in neurodegenerative diseases is highlighted by the number of stress-granule associated-RBPs implicated in the neurodegenerative and neurological disorders such as Ataxin-2 (in spinocerebellar ataxia), survival motor neuron (SMN) (in spino-muscular atrophy), fragile X mental retardation protein (FMRP) (in fragile X syndrome), Tar-DNA binding protein 43 (TDP-43) (in amyotrophic lateral sclerosis (ALS) and fronto-temporal lobar degeneration (FTLD)), fused in sarcoma (FUS) (in ALS) and TIA-1 (in ALS and FTD). Neurodegeneration-associated mutations in the above-mentioned proteins show increased protein aggregation and elevated levels of stress granules with reduced stress granule dynamics.

Current therapeutic interventions for reducing the formation of toxic cytoplasmic aggregates and promoting disassembly of aberrant stress granules have focused on either activating molecular chaperones, e.g., activating heat shock response, to facilitate protein folding or express components of the heat shock response engineered to remove aggregated proteins, e.g. heat shock proteins (HSP 104), or enhance clearance of aggregated proteins via the ubiquitin proteasome system and autophagy. However, limited success has been achieved in determining the efficacy of these interventions in disease development and progression.

This suggested to the inventors of the current disclosure that the formation of pathologically persistence granules contribute significantly to the disease development in neurodegenerative diseases. Accordingly, it is an object of the present disclosure to provide modalities, and systems and methods for use thereof, which may foster the disassembly of stress granules for therapeutic potential and neuro-protection.

Citation or identification of any document in this application is not an admission that such a document is available as prior art to the present disclosure.

### SUMMARY OF THE INVENTION

The above objectives are accomplished according to the present invention by providing in a first embodiment, a method for correcting disrupted axonal and synaptic protein synthesis. The method may include introducing an effective amount of at least one compound to a subject to restore an amount of at least one binding protein or disassociating at least one condensate in the subject comprising the binding protein via introduction of at least one peptide to restore local protein synthesis events. Furhter, the at least one binding protein may comprise SEQ ID NO: 1. Still, the at least one compound may include doxycycline. Further yet, the at least one peptide may include SEQ ID NO: 3. Even further, the subject may have at least one nervouse system condition. Further yet, the at least one nervouse system condition may be amyotrophic lateral sclerosis. Even further, ceasing cytoplasmic mislocalization of the at least one binding protein or dissociating the at least one condensate comprising the binding protein reverses effects of amyotrophic lateral sclerosis. Again, the at least one condensate may include a pathological ribonucleoprotein. Moreover, presence of the pathological ribonucleoprotein may interfere with axonal and pre-synaptic protein synthesis. Again still yet, mislocalization the at least one binding protein may bind and sequester nuclear-encoded mitochondrial mRNAs and depletes a level of the nuclear-encoded mitochondrial mRNAs in at least one axon. Further again, dissociating the condensates may restore local translation and resolve binding protein derived toxicity in both axons and neuromuscular junctions.

In a further aspect, the disclosure provides a therapeutic treatment for at least one nervouse system condition. The treatment may include correcting disrupted axonal and synaptic protein synthesis in a subject via restoring localization via dox re-introduction of at least one binding protein or disassociating at least one condensate in the subject comprising the binding protein via introduction of at least one peptide and restoring local protein synthesis events in intra-muscular nerves and motor neurons. Further, the at least one binding protein may include SEQ ID NO: 1. Still yet, the treatment may include administering an effective amount of doxycycline to restore localization of the at least one binding protein. Even further, the at least one peptide may include SEQ ID NO: 3. Still further yet, the at least one nervouse system condition may be amyotrophic lateral sclerosis. Even further yet, ceasing cytoplasmic mislocalization of the at least one binding protein or dissociating the at least one condensate comprising the binding protein condensates reverses effects of amyotrophic lateral sclerosis. Moreover, the at least one condensate may be a pathological ribonucleoprotein. Even furhter, the presence of the pathological ribonucleoprotein may interfere with axonal and pre-synaptic protein synthesis. Again still, mislocalization of the at least one binding protein may bind and sequester nuclear-encoded mitochondrial mRNAs and deplete a level of the nuclear-encoded mitochondrial mRNAs in at least one axon. Further still again, dissociating the condensates may restore local translation and may resolve binding protein derived toxicity in both axons and neuromuscular junctions.

These and other aspects, objects, features, and advantages of the example embodiments will become apparent to those having ordinary skill in the art upon consideration of the following detailed description of example embodiments.

### BRIEF DESCRIPTION OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1A shows representative images for NF-labeled (white) 7 days in vitro (DIV7) E18 midbrain neurons under control conditions (i) or after treatments with 100 μM MPP+ (ii) or 100 μM MPP⁺ with 190-208 G3BP1 (iv) or control 168-189 (iii) peptides.

FIG. 1B shows quantification of neurite degeneration in midbrain neurons in control or 100 μM MPP⁺-treated neurons in the presence of 190-208 G3BP1 or control 168-189 peptides.

FIG. 2A shows representative images for NF-labeled (white) DIV7 E 18 cortical neurons under control conditions (i) or after treatment with 1 μM Aβ (ii) or 1 μM Aβ with 190-208 G3BP1 (iv) or control 168-189 (iii) peptides.

FIG. 2B shows quantification of neurite degeneration in midbrain neurons in control or 1 μM AB peptide-treated neurons in the presence of 190-208 G3BP1 or control 168-189 peptides.

FIG. 3A shows quantification of MPP+-induced degeneration of DIV7 E12.5 motor neuron neurites after exposure of cultures to 100 μM MPP+ and either 10 μM 190-208 G3BP1 or control 168-189 peptides.

FIG. 3B shows Representative images of G3BP1 (green) and NF-labeled (red) E12.5 motor neurons under control or after treatment with 100 μM MPP⁺ [scale bar=10 μm].

FIG. 3C shows distribution of sizes of endogenous G3BP1 aggregate per 100pm neurite from motor neurons cultures as treated in FIG. 2B.

FIG. 3D shows endogenous G3BP1 aggregate sizes per 100 μm neurite indicated as bins for motor neurons either untreated or treated with MPP+alone or with addition of 100 μM 190-208 G3BP1 or 168-189 control peptide.

FIG. 4A shows the size of endogenous G3BP1 aggregates per 100 μm of neurite shown as indicated bins from midbrain cultures under control conditions and after treatment with MPP⁺ alone or with 190-208 G3BP1 or 168-189 peptides.

FIG. 4B shows size distribution of endogenous G3BP1 aggregates per 100μm of neurite shown from cortical neurons treated with 10 μM Aβ peptide or with 100 μM 190-208 G3BP1 or 168-189 control peptide.

FIG. 5A shows representative images of HEK cells transfected with GFP-tagged wild-type or ALS/FTD-associated mutants (P362L, A381T, or E384K) TIA1.

FIG. 5B shows quantification of the percentage of cells with TIA1-GFP puncta greater than 2 μm² area in transfected cells relative TIA1-WT-GFP expressing cells.

FIG. 5C shows HEK cells expressing TIA1-GFP or ALS/FTD-associated mutants TIA1-P362L, TIA1-A381T, TIA1-E384K treated with sodium arsenite (0.5 mM) for 30 minutes followed by treatment with either 100 μM 190-208 G3BP1 or 168-189 control peptide.

FIG. 6A shows embryonic motor neurons isolated from E12.5 mouse embryos exposed to sodium arsenite (0.5 mM) for 30 minutes and then treated with 10 μM 190-208 or 168-198 peptides at DIV7.

FIG. 6B shows E12.5 motor neurons treated as described in FIG. 5A and immunostaining used to detect endogenous G3BP1 protein.

FIG. 6C shows DIV7 E12.5 motor neurons expressing TIA1-GFP or ALS/FTD-associated mutants TIA1-A381T, TIA1-E384K treated with sodium arsenite (0.5 mM) for 30 minutes followed.

FIG. 6D shows DIV7 E12.5 motor neurons expressing flag-tagged wildtype TDP43 or ALS-associated mutants TDP43-M337V and TDP43-Q331K treated with sodium arsenite (0.5 mM) for 30 minutes followed by treatment with either 10 μM 190-208 G3BP1 or 168-189 control peptide.

FIG. 7A shows immunofluorescence for G3BP1 signals in the cell body (asterisk) and axons (arrows) of a cultured DRG neuron.

FIG. 7B shows single optical planes for axons of naive DRG cultures co-labeled for indicated proteins.

FIG. 7C shows axonal G3BP1 higher colocalization coefficients for SG than PB proteins by Fisher's Z transformation

FIG. 7D shows that proximity ligation analyses (PLA) shows higher colocalization for G3BP1 and HuR than G3BP1 and DCP1A (G3BP1+HuR PLA=0.038±0.003 and G3BP1+DCP1A PLA=0.027±0.002 signals/m².

FIG. 7E shows confocal images for G3BP1 and TIA1 in naive and 7 d post-injured (‘regenerating’) sciatic nerve.

FIG. 7F shows upper image panels of each pair that show G3BP1 and TIA1 merged with NF signals in a single plane.

FIG. 7G shows quantification of G3BP 1 levels in axons of DRGs cultured from naive versus 7 d injury-conditioned animals

FIG. 7H shows G3BP 1 immunofluorescence in axons of DRGs cultured from naive versus 7 d injury-conditioned animals.

FIG. 8A shows representative images for axons of DRG neurons transfected with indicated G3BP 1 constructs versus eGFP.

FIG. 8B shows quantification of axonal aggregates for G3BP1-GFP, G3BP1^{S149A}-GFP, and G3BP1^{S149E}-GFP.

FIG. 8C shows FRAP analyses for neurons transfected with constructs as in FIG. 8A are shown as average normalized % recovery±standard error of the mean (SEM).

FIG. 8D shows exposure-matched confocal images for G3BP1P^{S149} and NF.

FIG. 8E shows quantifications of the signals shown in FIG. 7D as mean±SEM.

FIG. 8F shows distal axons of cultured DRGs immunostained with pan-G3BP 1 versus G3BP 1P^{S149} antibodies.

FIG. 8G shows quantification of the signals from FIG. 7F with a significant increase in the ratio of G3BP 1P^{S149} immunoreactivity to G3BP1 aggregates moving distally to the growth cone.

FIG. 9A shows images of FISH/IF for Nrn1 mRNA and G3BP1 protein shown for axons of naive and 7 d injury conditioned DRG neurons.

FIG. 9B shows quantification of colocalizations for Nrn1, Impβ1, and Gap43 mRNAs with G3BP 1 in axons of neurons cultured from naive or 7 d injury-conditioned animals shown as average Pearson's coefficient±SEM (N≥21 neurons over 3 repetitions.

FIG. 9C shows schematics of translation reporter constructs used in FIGS. 9D-H.

FIG. 9D shows representative FRAP image sequences for DRG neurons co-transfected with GFP^{MYR} 5′/3′nrn1 plus BFP or G3BP1-BFP.

FIG. 9E shows quantifications of FRAP assays from DRGs expressing GFP^{MYR} 5′/3′nrn1.

FIG. 9F shows quantifications of FRAP assays from DRGs expressing GFP^{MYR} 5′/3′impβ1.

FIG. 9G shows quantifications of FRAP assays from DRGs expressing mCh^{MYR} 5′/3′gap43.

FIG. 9H shows HEK293T cells transfected with GFP^{MYR} 5′/3′nrn1, GFP^{MYR} 5′/3′impβ1, and mCh^{MYR} 5′/3′gap43 show significant enrichment of GFP^{MYR} 5′/3′nrn1 and GFP^{MYR} 5′/3′impβ1 mRNAs coimmunoprecipitating with G3BP1 versus control.

FIG. 10A shows a schematic of G3BP1 domains.

FIG. 10B shows representative images for NF-labeled DRG neurons transfected with indicated constructs.

FIG. 10C shows the extent of axon regeneration at 7 d post sciatic nerve crush in adult rats transduced with AAV5 encoding G3BP1-BFP, G3BP1 B domain-BFP, G3BP1 D domain-BFP, or GFP control.

FIG. 10D shows animals transduced with AAV5 encoding G3BP1 B domain-BFP versus GFP were subjected to sciatic nerve crush and regeneration was assessed by muscle M response in tibialis anterior and gastrocnemius.

FIG. 10E shows quantitation of axon growth from DRGs (left) and cortical neurons (right) treated with cell-permeable 168-189 or 190-208 G3BP1 peptides.

FIG. 11A shows representative images for puromycin (Puro) incorporation in DRG neurons transfected with the indicated constructs.

FIG. 11B shows significant increase in axonal puromycin signals in the G3BP1 B domain-expressing neurons with no significant change in the cell body puromycin incorporation.

FIG. 11C shows that G3BP1 depleted DRG cultures similarly show increased puromycin incorporation in axons with no significant change in cell body puromycin incorporation.

FIG. 11D shows quantitation of endogenous axonal NRN1, IMPβ1, and GAP43 protein levels in DRG cultures transfected with GFP, G3BP1-GFP, and G3BP1 B domain-GFP.

FIG. 11E shows RTddPCR for axonal mRNAs co-precipitating with G3BP1-GFP in DRG neurons.

FIG. 12A shows representative images for puromycin incorporation in axons of control, 168-189 peptide and 190-208 peptide-treated DRG cultures are shown

FIG. 12B shows quantitation of puromycin incorporation into distal DRG axons under these conditions shows a significant increase in axonal protein synthesis for the 190-208 peptide-treated cultures compared to control and 168-189 peptide exposure.

FIG. 12C shows FRAP analyses for DRGs for GFP^{MYR} 5′/3′nrn1, GFP^{MYR} 5′/3′impβ1 and GFP^{MYR} 5′/3′gap43 in axons of DRGs expressing BFP or G3BP1-BFP±10 μM 190-208 G3BP1 peptide.

FIG. 12D shows representative images of G3BP1-mCh in DRG axons under control conditions and after treatment with 190-208 G3BP1 or 168-189 peptides for 15 min.

FIG. 12E shows density of G3BP1-mCh aggregates along 100 μm length axons from DRG cultures treated as in FIG. 12D.

FIG. 12F shows the size of G3BP1-mCh aggregates from DRG cultures treated as in FIG. 12D.

FIG. 13A shows representative confocal images for G3BP1 (magenta), FMRP (green), FXR (red) and neurofilament (blue) immunoreactivity along axons for E18 cortical neuron cultures (7 DIV)±1 μM Aβ oligomer for 6 hours.

FIG. 13B shows the size distribution for aggregates of G3BP1 (i), FMRP (ii) and FXR (iii) along control vs. Aβ oligomer treated axons as in FIG. 13A.

FIG. 13C shows confocal images for G3BP1 (magenta), FUS-TLS (green), TDP43 (red) and neurofilament (blue) immunoreactivity along axons for cortical neurons treated as in FIG. 13A.

FIG. 13D shows the size distribution for TDP43 (i), FUS-TLS (ii), TIA1 (iii) aggregates along control vs. Aβ oligomer-treated axons as in FIG. 13C.

FIG. 13E shows quantification for colocalization of FMRP, FXR, TDP43, FUS-TLS and TIA1 with G3BP1. in axons of E18 cortical. neurons treated as in 13A shown as average Pearson's coefficient±SEM.

FIG. 13F shows the overall levels of these proteins based. in exposure matched images (N≥100 aggregates over three repetitions and *p≤0.01, **p≤0.005, ***p≤0.001 for entire population distributions by Fishers exact test for B and D; N≥25 neurons over 3 repetitions and *p≤0.01, **p≤0.005, *** p≤0.001 by one-way ANOVA with Tukey HSD post-hoc for FIGS. 13E-13F).

FIG. 14A shows confocal images for G3BP1 (magenta), FMRP (green), FXR (red) and neurofilament (blue) immunoreactivity along axons for E18 midbrain neuron cultures (7 DIV) ±100 μM MPP+ for 6 hours.

FIG. 14B shows the size distribution for aggregates of G3BP1 (i), FMRP (ii) and. FXR (iii) along control vs, MPP⁺-treated axons.

FIG. 14C shows representative confocal images for G3BP1 (magenta), FUS-TLS (green), TDP43 (red) and. neurofilament (blue) immunoreactivity along axons for midbrain neurons treated as in FIG. 13A.

FIG. 14D shows the size distribution for TDP43 (i), FUS-TLS (ii), TIA1 (iii) aggregates along control vs. MPP⁺-treated axons as in FIG. 14C.

FIG. 14E shows quantifications for colocalization of FMRP, FXR, TDP43, FUS-TLS and TIA4 with G3BP1 in axons of E18 midbrain neurons treated as in A are shown as average Pearson's coefficient±SEM.

FIG. 14F shows overall levels of these proteins based in exposure matched images (N≥100 aggregates over three repetitions and *p≤0.01, **p≤0.005, ***p≤0.001 for entire population distributions by Fishers exact test for B and D; N≥25 neurons over 3 repetitions and *p≤0.01, **p≤0.005, ***p≤0,001 by one-way ANOVA with Tukey HSD post-hoc for FIGS. 13E-13F).

FIG. 15A shows confocal images for G3BP1 (magenta), FMRP (green), FXR (red) and neurofilament (blue) immunoreactivity along axons for control and 1 μM Aβ oligomer-treated E18 cortical neurons (7 DIV)±cell permeable G3BP1 190-208 peptide.

FIG. 15B shows the size distribution for aggregates of G3BP1 (i), FMRP (ii) and FXR (iii) along control vs. Aβ-treated axons and Aβ treated+G3BP1 190-208 or cell permeable peptide with scrambled sequence.

FIG. 15C confocal images for G3BP1 (magenta), FUS-TLS (green), TDP43 (red) and neurofilament (blue) immunoreactivity along axons for cortical neurons treated as in A.

FIG. 15D shows the size distribution for TDP43 (i), FUS-TLS (ii), TIA1 (iii) aggregates along axons of control vs. Aβ-treated and Aβ treated+G3BP1 190-208 or cell permeable peptide with scrambled sequence.

FIG. 15E quantifications for colocalization of FMRP, FXR, TDP43, FUS-TLS and TIA1 with G3BP1 in axons of E18 cortical neurons treated as in C are shown as average Pearson's coefficient±SEM (N≥120 aggregates over three repetitions and *p≤0.01, **p≤0.005, ***p≤0.001. for entire population distributions by Fishers exact test for B and D; N≥25 neurons over 3 repetitions and *p≤0.01, **p≤0.005, ***p≤0.001 by one-way ANOVA with Tukey HSD post-hoc for E).

FIG. 16A shows schematic of the microfluidic culture set up. E18 cortical neurons were plated into the cell-body compartment (blue); after 7 DIV axons from these neurons extend through the microchannels into the axon compartment (pink).

FIG. 16B shows representative montage images of axonal compartment of DIV7 E18 cortical neurons stained with neurofilament for control and Aβ oligomer (1 μM for 16 hours)±190-208 G3BP1 peptide (1 μM).

FIG. 16C shows quantitation of degeneration indices for cultures from B (N=3 repetitions and *p≤0.01, **p≤0.005 by one-way ANOVA with Tukey HSD post-hoc).

FIG. 17 shows at a, c Immunofluorescence images and b, d Quantification of non-ALS and ALS patient intra-muscular nerves TDP-43 (b) or phosphorylated TDP-43 (pTDP-43) (d) signal. within NM-positive axons, normalized to NFH intensity; e, f images (e) and quantification (f) of pTDP-43 in axons (NFH) of C9ORF72 and control. iPS-MN; g Western-blots for TDP-43, human-specific-TDP-43 (hTDP-43) and pTDP-43 in TDPΔNLS and control mice sciatic nerve axoplasm (SN axoplasm); h-k Images (h, j) and quantification (i, k) of TDP-43 (h) or pTDP-43 (j) intensity in ChAT-positive sciatic nerve axons of TDPΔNLS^{ChAT::tdTomato} and control^{ChAT::tdTomato} mice; l-m images of TDP-43 (l) or pTDP-43 (m) in NMJs before, two-weeks (l) and four-weeks (m) after dox retraction. from TDPΔNLS mice diet; and o, p Western-blots (o) and quantification (p) of hTDP-43, TDP-43 and tERK (left), and pTDP-43 and tubulin (right) in protein lysates of TDPΔNLS or control MNs/axons isolated from radial MFCs. n=3 repeats.

FIG. 18 shows at a-c Images (a), colocalization profiles (b) and quantification (c) of TDP-43-G3BP1-SytoRNA colocalization in control or ΔNLS axons; d-f Images (d), colocalization profiles (e) and quantification (f) of pTDP-43-G3BP1-SytoRNA colocalization in control or C9ORF72 iPS-MN; and g-i Images (g), colocalization profiles (h) and quantification (i) of pTDP-43-G3BP1 colocalization in non-ALS or ALS patient intra-muscular nerves.

FIG. 19 shows at a images and b quantification of OPP puncta density C9ORF72 or control iPS-MN axons; c Images and d-f quantification of NFH, pTDP-43 and G3BP1 and their colocalization in C9ORF72 iPS-MN axons either treated or not with G3BP1 peptide; quantifications of G3BP1 particle density (d), G3BP1-pTDP-43 coloc-particle density (e) and G3BP1-pTDP-43 coloc-particle size (f) from C9ORF72 and control iPS-MN axons, either treated or not with. G3BP1 peptide; g Images and h quantification of OPP puncta density from C9ORF72 and control iPS-MN axons either treated or not with G3BP1 peptide; i Images and j quantification of OPP puncta density in control or SLS MN axons; k Images and l quantification of OPP puncta density in in vitro NMJs from control or ΔNLS co-cultures; m Images and n quantification of OPP-labeled sciatic nerve sections from TDPΔNLS^{ChAT::tdTomato} or control^{ChAT::tdTomato} mice; and o Images and p quantification of pre-synaptic OPP in NMJs from TDPΔNLS^{ChAT::tdTomato} or control^{ChAT::tdTomato} mice.

FIG. 20 shows at a Volcano-plot of sciatic nerve axoplasm proteome analysis from TDPΔNLS and control mice; b Venn-diagram of all Mitocarta proteins that were up-regulated. (upper panel) or down-regulated (lower panel); c GO analyses categories; d-g Images and quantification of Cox4i (d, e) and ATP 5A1 (f, g) levels in ChAT-positive axons within TDPΔNLS^{ChAT::tdTomato} and control sciatic nerve cross-sections; h Western-blot and quantification of Cox4i in isolated axons from TDPΔNLS or control VINs cultures; control sciatic axoplasm (i) or pure axons of TDPΔNLS or control MNs cultured in radial MFC (j); k. l TDP-43-RIP of somata (upper panel) and pure axons from radial MFC (lower panel) in C9ORF72 and control iPS-MNs, immunoblotted for TDP-43 and. tubulin as a loading control; m-o Images (in) and quantification (n, o) of Cox4il mRNA-pTDP-43 colocalization (n) and Cox4il mRNA-pTDP-43-G3BP1. colocalization (o) in C9ORF72 and control iPS-MN axons; p Immunoblot. for Cox4 following OPP pull-down (upper panel) and Coomassie staining (total protein) of input lysates (lower panel) from. TDPΔNLS and. control sciatic axoplasms labeled with OPP.

FIG. 21. shows at a Images and. b quantification of TMRE signal in HB9::GFP MN axons untreated (control) or treated with anisomycin (aniso), cycloheximide (CHX) or NaAsO₂; c Images and d quantification of OPP and mitochondria colocalization in TDPΔNLS or control MN axons; e images and f quantification of mitochondria density within sciatic nerve longitudinal sections of TDPΔNLS^{Thy1::MitoDendra} and control mice labeled also for NFH and Tau; g Images and h quantification of pre-synaptic mitochondrial volume within NMJs of TDPΔNLS^{Thy1::MitoDendra} and control^{Thy1::MitoDendra} mice; i Images and j quantification of pre-synaptic TMRE signal intensity within in vitro NMJs from TDPΔNLS^{Thy1::MitoDendra}, control^{Thy1::MitoDendra} co-cultures and TDPΔNLS^{Thy1::MitoDendra} co-cultures treated with G3B1 peptides in NMJ compartment.

FIG. 22 shows at a Schematic illustration of Mito-Killer-Red (MKR) experimental. setup, used. for specifically targeting oxidative stress to NMJ mitochondria.; b Images of MKR in NMJ pre-synapse before and after bleach (white line=bleached region); c, d Representative OGB time-trace of OGB indicating muscle contraction (c) and quantification (d) of contraction ratio before and after MKR bleaching; e Schematic illustration of experimental procedure for puromycin local protein synthesis inhibition in NMJ pre-synapse using puromycin resistant muscles; f Left panel: Time-series images of OGB-labeled co-cultures treated, or not with puromycin in NMJ compartment. Right panel; Demonstration of paired axon-muscle calcium activity only in control NMJs and its absence upon puromycin application; g Time traces of OGB in pre-synaptic neurons and post-synaptic muscles in control. (upper plot), and in puromycin-treated (lower plot) cultures; h Quantification of the percent of innervated and. contracting muscles after puromycin application; i Images and j quantification of the percent of degenerating axons in TDPΔNLS and control MN cultures following 16 and 24 h of puromycin treatment.

FIG. 23 shows at a Western-blot and quantification of TDP-43, hTDP-43 levels in sciatic axoplasm of control, TDPΔNLS, and recovered.-TDPΔNLS (Rec.); b Images and c quantification of TDP-43 intensity within ChAT-positive axons in sciatic nerve sections of control, TDPΔNLS, and recovered-TDPΔNLS mice.; d Images and e quantification of the percent of NMJs with apparent TDP-43 condensates in control, TDPΔNLS, and recovered-TDPΔNLS mice; f Images and g analysis of OPP puncta density in in vitro NMJs of control, TDPΔNLS and Recovered-TDPΔNLS co-cultures; h Images and i analysis of the OPP labeling intensity in pre-synaptic NMJs of control, TDPΔNLS and recovered-TDPΔNLS mice. n=32,30,39 NMJs, from 3,3,3 mice; j Images and k representative channel histograms of Cox4i and OPP intensities within pre-synaptic axon (ChAT) in NMJs of control TDPΔNLS mice; l-m quantification of Cox4i area and of Cox4i-OPP color-area within pre-synaptic axons (ChAT) in NMJs of control, TDPΔNLS, and recovered-TDPΔNLS mice.

FIG. 24 shows at: a images and b Quantification of NMJs from control, TDPΔNLS, and recovered-TDPΔNLS mice; c Images and d quantification of NMJ innervation in vitro, assessed by co-expression of pre- and post-synaptic elements (percent of ChAT-BTX clusters) in control, TDPΔNLS and. recovered-TDPΔNLS co-cultures; e, f Quantification of the percent of contracting muscles in control., TDPΔNLS and. recovered-TDPΔNLS co-cultures (e) or in control, TDPΔNLS and TDPΔNLS co-cultures treated with G3BP1 peptides in NMJ compartment (f). n=7,6,7 (e), 10,9,9 (f) MFCs; g Model: axonal mislocalization of TDP-43 and. its phosphorylated form create G3BP1 positive RNP condensates, which decrease local. translation of nuclear-encoded mitochondrial proteins by sequestering their mRNA.

FIG. 25 shows Table 1, Patient Details.

FIG. 26 shows Table 2, Radial microfluidic chamber properties.

FIG. 27 shows Table 3, Standard PCR Primers.

FIG. 28 shows Table 4, qPCR primers.

FIG. 29 shows Table 5, shows smFISH probe sequences.

FIG. 30 shows at a) Representative immunofluorescent images of C9ORF72-mutated and isogenic control iPS-MN at 9-days in vitro (DIV) demonstrating Doxycyhn-induced differentiation into; b) Representative immunofluorescent images of TDP43 cytoplasmic mi.sl.ocalization C9ORF72 iPS-MN; c) Representative bright-field images; and quantification (d-e) demonstrating differences in neurite length between C9ORF72 iPS-MN and their isogenic controls.

FIG. 31 shows at : a) Representative images of in vivo SC MNs from TDPΔNLS-ChATtdTomato mouse and control stained with tTDP-43 antibody (green) and DAPI (blue); b) Representative images of TDPΔNLS primary cultured MNs cell bodies with (upper panel-control) or without dox (middle and lower panel), stained with TDP-43 antibody (green) and DAPI (blue); c) Western blot and d) PCR of axons extracted from radial MFCs; e) Western blot analysis of the normalized overall TDP-43 protein levels in control MNs or upon induction of TDPΔNLS expression; f) Western blots showing all three repeats of hTDP43 and TDP43 levels in distal axons of control. or TDPΔNLS MNs cultured in radial. MFC; g) Full-uncropped blot of pTDP43 levels in distal axons of control or TDPΔNLS MNs cultured in radial MK. Tubulin was used as loading control.

FIG. 32 shows at a) Representative images and b) quantification results from 3D-co: localization analysis examining the colocalization (yellow) of phosphorylated TDP-43 (pTDP; green) and G3BP1 (magenta) in cell bodies of cultured control or TDPΔNLS MNs; c-d) Quantification of G3BP1 (C) and pTDP43 (D) intensity in control or TDPΔNLS MNs. n=38,40 cells; e) Representative images and f) quantification of the pTDP43-G3BP1 colocalization in proximal axons of TDPΔNLS and control MNs; g-j) Representative images (g) and quantification (h-j) of TDP-43 (green) and G3BP1 (magenta) colocalization (h), average TDP-43 (i) and average G3BP1 (j) intensities in distal axons of ChAT; k) Representative western-blot and l) quantification of G3BP1 levels in distal axons of control or TDPΔNLS cultured MNs within radial MFCs. Tubulin was used for loading control. n=3 experiments. SE. Unpaired Mann-Whitney test. *p=0.05; in-n) Representative images (m) and quantification (n) results from 3D-colocalization analysis examining the colocalization (yellow) of FMRP (green) and G3BP1 (magenta) in axons of cultured control or TDPΔNLS MNs; o-p) Representative images (o) and quantification (p) results from 3D-colocalization analysis examining the colocalization (yellow) of Staufen1 (green) and G3BP1 (magenta) in axons of cultured control or TDPΔNLS MNs. White indicates Scale bar=5 μm. n=29,29 axons. SD. Unpaired t-test, two-sided. q-r) Representative images (q) and colocalization analysis (r) showing pTDP (green), G3BP1 (magenta), Syto RNA (cyan) and mitotra.cker (white) staining in axons of cultured control or TDPΔNLS MNs.

FIG. 33 shows at a) Representative images and b) quantification results from 3D-colocalization analysis examining the colocalization (yellow) of phosphorylated TDP-43 (pTDP; green) and G3BP1 (magenta) in cell bodies of cultured C9ORF72 or isogenic control iPS-MNs; c-d) Quantification of G3BP1 (C) and pTDP43 (D) intensity in C9ORF72 or control iPS-MNs; e) Representative images and f) quantification of the pTDP43-G3BP1 colocalization in proximal axons of C9ORF72 and control MNs iPS-MNs; g-i) Quantification of G3BP1 colocalization and intensity in C9ORF72 and control iPS-MN; h) The percent of TDP43-G3BP1 colocalized area out of the total axonal area in C9ORF72 and control iPS-MN; i) Average G3BP1 intensity in iPS-MN axons n=4 repeats SE.

FIG. 34 shows at a) Representative images and b) quantification of OPP density and c) OPP puncta fluorescence intensity in distal axons in MFC labeled with OPP (20 μM) for different time periods (1 min, 5 min, 30 min); d) Representative images and e) Quantification of OPP puncta in axons without (control) or with application of NaAsO₂ (250 μM) to axonal compartment of MFC; f) Representative images g) and quantification of OPP puncta in axons without (control) or with application of protein synthesis inhibitors Cycloheximide (CI-IX) and Anisomycin (Aniso).

FIG. 35 shows at a) Representative images and b) quantification of OPP signal intensity in C9ORF72 and control iPS-MNs cell-bodies; d) Representative images and c) quantification of OPP signal intensity in TDPΔNLS and control MNs cell-bodies.

FIG. 36 shows at a-b) Representative phase and fluorescent images demonstrating penetration of TAT-fused G3BP1 (190-208)-FITC peptides into primary MNs cell bodies (a) and axons (b); c) Quantification of G3BP1 particle size in C9ORF72 or control MN axons treated with G3BP1 peptides exclusively in the axonal compartment of the MFC; d) Quantification of pTDP-43 particle size in C9ORF72 or control. MN axons treated with G3BP1 peptides exclusively in the axonal. compartment of the MFC; and e) Quantification of pTDP-43 particle density in C9ORF72 or control MN axons treated with G3BP1 peptides exclusively in the axonal compartment of the MFC.

FIG. 37 shows at a) Representative images and b) quantification of puromycin labeling in control, versus puromycin resistant muscles (transfected with PQCXIP-mCherry backbone vector expressing Puromycin Acetyltransferase gene (PAC)) demonstrating the ability of PAC to prevent puromycin labeling of newly synthesized peptides; c) Representative images of puromycin-resistant muscles stable morphology following 16 h and 24 hr incubation with 100 μg/mL puromycin compared to muscles that were not exposed to puromycin. d) Representative images and e) quantification of OPP puncta density of in vitro NMJs in the presence of absence of distal NaAsO₂.

FIG. 38 shows at a) Representative images of OPP labeling versus only color labeling but no puromycin (no OPP) in HB9:GFP mouse SN sections; and b) .Additional unmasked. OPP images and overlay images of SN sections obtained from TDPΔNLS and control mice.

FIG. 39 shows at a) Uncropped western blot of Cox4i. protein levels in isolated axons of TDPΔNLS MNs cultured in radial MFC; b) RT-qPCR of Cox4i1. and ATP5A1 mRNA levels from somata of C9ORF72 and control iPS-MNs; c) quantification of Cox4i1 mRNA abundance in C9ORF72 and control iPS-MN axons; d) OPP pull-down controls in HEK293T cells validation for specificity and sensitivity of assay; and e) Streptavidin-HRP (top) and Coomassie stained (bottom) blots from OPP-labeled sciatic nerve axoplasms of Control. TDPΔNLS, Anisomycin-treated, and no-OPP controls.

FIG. 40 shows at a) Representative images and b) quantification of the percent of mitochondria (area; green)) colocalization with OPP (area; red) in MN axons treated exclusively with cycloheximide (CHX) versus control; c) Representative images; d) and. quantification of the TAME signal in TDPΔNLS and control MN axons; and e) Quantification of MIRE signal in TDPΔNLS and control MN axons, following transient 4-hour anisomycin treatment and. washout.

FIG. 41 shows at a) Representative images and b) quantification of the percent of degenerating HB9:GFP MN axons in the distal compartment of MFC following 24 h axonal incubation with protein synthesis inhibitor Puromycin.

FIG. 42 shows at a) Western-blots for total-TDP-43 and human-TDP-43 (hTDP-43) in GC muscles, spinal cords (SC) and sciatic nerves of control, TDPΔNLS, and recovery mice; b) Representative images and c) Quantitative analysis of the percent of spinal cord. MNs (ChAT-red) with nuclear-localized TDP-43 in recovered mice compared to TDPΔNLS mice with no recovery; d) Quantitative analysis of the percent of MNs (ChAT-red) with nuclear versus cytoplasmic localization of pTDP-43 in spinal cords of control, TDPΔNLS, and recovery mice; e) Representative images and f) quantification of OPP signal (green) within ChAT axons (red) in sciatic nerves from recovered mice compared to control and ANIS mice; g) Images and h) representative channel histograms of ATP5A1 (red) and. OPP (green) intensities within pre-synaptic axon (ChAT) in NMJs of control TDPΔNLS mice, and i-j) colocalization analysis of the percent of ATP5A1 area (i) and. ATP5A1 colocalization with OPP (j) within the pre-synapse area (ChAT) in TDPΔNLS mice compared with control and recovered mice; k) Representative images of NMJ innervation from TDPΔNLS mice compared with control and recovered mice; l) Representative images and in) quantitative analysis of BTX (green) post synaptic area cluster size in GC muscle NMJs in recovered mice as compared with ΔNLS and LM mice; and ii) TDPΔNLS, control and recovered mice weight measurements after dox retraction.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### DETAILED DESCRIPTION OF A PREFERRED EMBODIMENT

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, ‘less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, ‘greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammal organism, for example by puncture, or other collecting or sampling procedures.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “active agent” or “active ingredient” refers to a substance, compound, or molecule, which is biologically active or otherwise that induces a biological or physiological effect on a subject to which it is administered to. In other words, “active agent” or “active ingredient” refers to a component or components of a composition to which the whole or part of the effect of the composition is attributed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition to the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “control” can refer to an alternative subject or sample used in an experiment for comparison purpose and included to minimize or distinguish the effect of variables other than an independent variable.

The term “optional” or “optionally” means that the subsequent described event, circumstance or substituent may or may not occur, and that the description includes instances where the event or circumstance occurs and instances where it does not.

As used herein, “dose,” “unit dose,” or “dosage” can refer to physically discrete units suitable for use in a subject, each unit containing a predetermined quantity of a pharmaceutical formulation thereof calculated to produce the desired response or responses in association with its administration.

The term “molecular weight”, as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “polymer” refers to molecules made up of monomers repeat units linked together. “Polymers” are understood to include, but are not limited to, homopolymers, copolymers, such as for example, block, graft, random and alternating copolymers, terpolymers, etc. and blends and modifications thereof. “A polymer” can be can be a three-dimensional network (e.g. the repeat units are linked together left and right, front and back, up and down), a two-dimensional network (e.g. the repeat units are linked together left, right, up, and down in a sheet form), or a one-dimensional network (e.g. the repeat units are linked left and right to form a chain). “Polymers” can be composed, natural monomers or synthetic monomers and combinations thereof. The polymers can be biologic (e.g. the monomers are biologically important (e.g. an amino acid), natural, or synthetic.

As used herein, the term “radiation sensitizer” refers to agents that can selectively enhance the cell killing from irradiation in a desired cell population, such as tumor cells, while exhibiting no single agent toxicity on tumor or normal cells.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer and/or indirect radiation damage. The effect can be therapeutic in terms of a partial or complete cure of a disease, condition, symptom or adverse effect attributed to the disease, disorder, or condition. The term “treatment” as used herein covers any treatment of cancer and/or indirect radiation damage, in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “water-soluble”, generally means at least about 10 g of a substance is soluble in 1 L of water, i.e., at neutral pH, at 25° C.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

### Kits

Any of the methods, treatments, compounds and/or formulations described herein can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as an active ingredient, contained therein. Such additional components include, but are not limited to, packaging, syringes, blister packages, bottles, and the like. When one or more of the compounds, compositions, formulations, particles, cells, described herein or a combination thereof (e.g., agent(s)) contained in the kit are administered simultaneously, the combination kit can contain the active agent(s) in a single formulation, such as a pharmaceutical formulation, (e.g., a tablet, liquid preparation, dehydrated preparation, etc.) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds and/or formulations, safety information regarding the content of the compounds and formulations (e.g., pharmaceutical formulations), information regarding the dosages, indications for use, and/or recommended treatment regimen(s) for the compound(s) and/or pharmaceutical formulations contained therein. In some embodiments, the instructions can provide directions and protocols for administering the compounds and/or formulations described herein to a subject in need thereof. In some embodiments, the instructions can provide one or more embodiments of the methods and therapeutic treatments and/or a pharmaceutical formulation thereof such as any of the methods described in greater detail elsewhere herein.

The current disclosure demonstrates that disassembly of stress granules is neuro-protective across a number of neurodegenerative disorders. Thus, modalities which may foster the disassembly of stress granules would be deemed to have therapeutic potential and are considered as neuro-protective.

The inventors report the application of the cell permeable 190-208 G3BP1 peptide derived from a highly conserved region in G3BP1 and that has been shown to specifically target mRNA storage sites in neurons and increases rates of regeneration after traumatic injury, to neurons exposed to neurodegenerative insults or cells expressing neurodegeneration-associated mutant proteins. Previous studies from the inventors' laboratory have shown that the 190-208 G3BP1 peptide interferes with endogenous stress granule formation possibly by blocking the aggregation of the SC nucleator G3BP1. The 190-208 G3BP1 peptide-treated neurons show reduced degeneration of neurons after treatment with neurotoxins such as MPP+ and beta-amyloid peptide, see infra. In addition, 190-208 G3BP1 peptide treatment reverses the increased protein aggregations and elevated levels of stress granule observed in neurons expressing neurodegeneration-associated mutant alleles of TDP-43 and TIA-1, see infra. The neuroprotective effects of the cell-permeable 190-208 G3BP1 peptide may represent a novel therapeutic lead for ameliorating neurodegeneration.

The potential importance of stress granules in neurodegenerative diseases is highlighted by the number of stress-granule associated-RBPs implicated in neurodegenerative and neurological disorders such as Ataxin-2 (in spinocerebellar ataxia), survival motor neuron (SMN) (in spino-muscular atrophy), fragile X mental retardation protein (FMRP) (in fragile X syndrome), Tar-DNA binding protein 43 (TDP-43) (in amyotrophic lateral sclerosis (ALS) and fronto-temporal lobar degeneration (FTLD)), fused in sarcoma (FUS) (in ALS) and TIA-1 (in ALS and FTD).

Neurodegeneration-associated mutations in the above-mentioned proteins show increased protein aggregation and elevated levels of stress granules with reduced stress granule dynamics. Therefore, suggesting that the formation of these pathologically persistence granules contribute significantly to the disease development in neurodegenerative diseases. Thus, modalities which may foster the disassembly of stress granules would be deemed to have therapeutic potential and are considered as neuro-protective.

Accumulating evidence from neurodegeneration-associated mutations in ribonucleoproteins (RNPs) show that aberrant stress granule (SG) dynamics contribute to initiation and progression of diseases such as ALS, FTD and AD. Stress granules are RNP complexes formed during stress to transiently sequester mRNAs whose protein products are not immediately needed, thus tailoring neuronal protein production to best respond to and survive the insult. Mutations that enhance aggregation of RNA binding proteins or cells exposed to chronic stress and disease lead to formation of atypical SGs, with altered SG dynamics that sequester RNA binding proteins and mRNAs. Moreover, insults that model neurodegeneration in Alzheimer's and Parkinson's diseases result in formation of SGs. These atypical SGs likely contribute to disease pathogenesis and/or progression. The inventors have developed a cell-permeable G3BP1-derived peptide that can block SG protein aggregation and facilitates SG disassembly. Treatment of neurons with this peptide prevents neurotoxin-induced neurodegeneration.

Currently, approximately 20 genes encoding RNA binding proteins have been shown to be mutated in neurodegenerative diseases. An increasing number of these proteins form aberrant protein aggregates and also effect stress granule dynamics, thereby contributing to disease initiation and progression. Thus, alleviating the protein aggregation and restoring homeostatic SG dynamics may prove to be remedial. The currently available strategies focus on enhancing chaperone-mediated refolding of aggregates or improving clearance of the aggregates and SG via the proteasomal and autophagic pathways. However, there is a lack of modalities that directly target stress granule assembly and disassembly. The inventors propose that a cell permeable 190-208 G3BP1 derived peptide can prevent aberrant stress granule aggregation, dissassemble aggregates, and restore SG dynamics so as to prevent the onset and progression of neurodegeneration.

Several studies have shown that aberrant stress granules aggregates are present in neurons of a number of neurodegenerative, thus suggesting a role for these RNA-protein complexes in disease initiation and progression. However, no reagents have yet been reported that can directly modulate stress granule assembly or disassembly. The inventors herein show that cell permeable 190-208 G3BP1 peptide is able to trigger dissociation of aberrant stress granules formed by expression of ALS and FTD associated mutant proteins and prevent neurotoxin (MPP+and amyloid)-induced cell death in neurons. The SG disassembly and the neuroprotective effects of the cell-permeable 190-208 G3BP1 peptide represent a novel therapeutic lead for preventing neurodegeneration. The cell permeable G3BP1 190-208 peptide has been shown to trigger endogenous SG disassembly in naïve neurons. Interestingly, treatment of neurons expressing disease-associated mutant proteins with the cell permeable G3BP1 190-208 peptide reduces the aberrant cytoplasmic aggregation observed in neurons and could be a significant target molecule for future drug development.

The inventors have shown that the treatment of neurons with cell permeable G3BP1 190-208 peptide leads to disassembly of stress granules and an increase in axonal protein synthesis. Based on these results, the inventors propose that cell permeable G3BP1 190-208 peptide-mediated disassembly of stress granules is neuroprotective against a number of neurodegenerative insults and may potentially be a novel drug target.

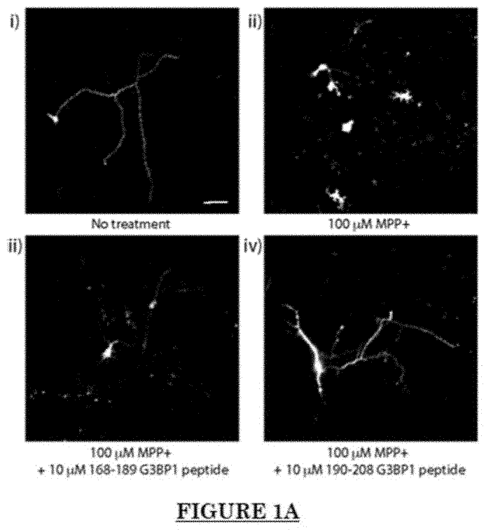

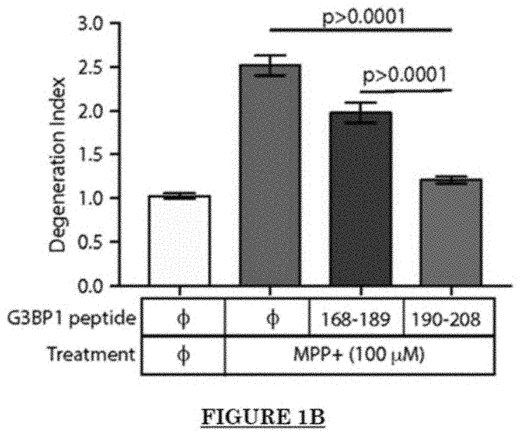

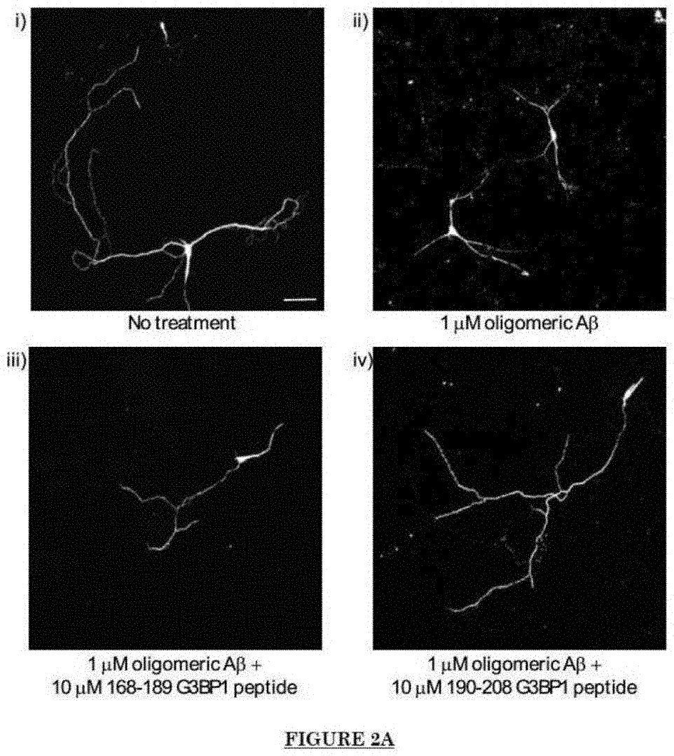

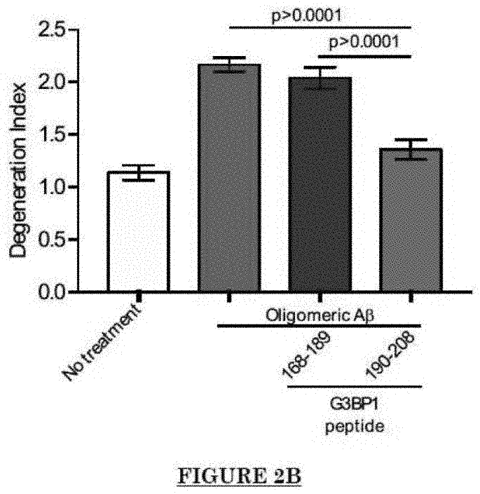

To test whether the triggering disassembly of stress granules would be protective in Parkinson's and Alzheimer's disease, the inventors employed 1-methyl-4-phenylpyridinium (MPP+)-induced and amyloid β (Aβ)-mediated neurotoxicity models. Rat embryonic day 18 (E18) midbrain neurons cultured for 7 days were treated for 16-18 hours with 100 μM MPP+, see FIGS. 1A and B, while E18 cortical neurons were treated with 1 μM Aβ oligomer, see FIGS. 2A-2B, with or without 10 μM G3BP1 190-208 or 168-189 peptide. As a control, the inventors used non-treated neurons.

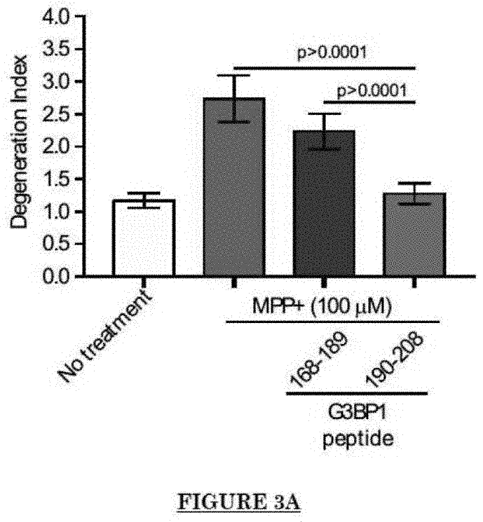

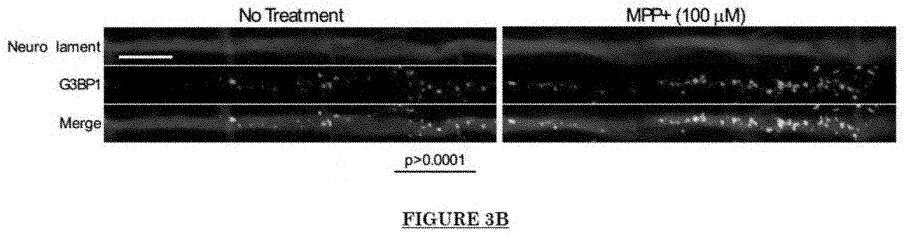

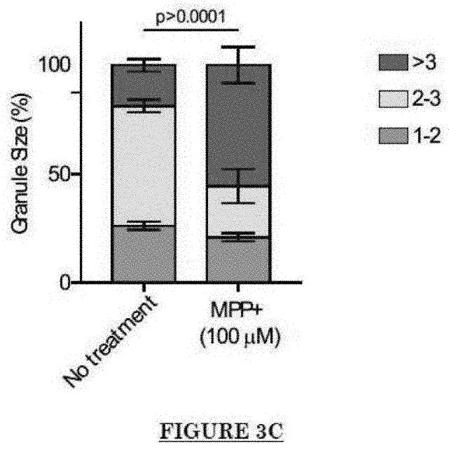

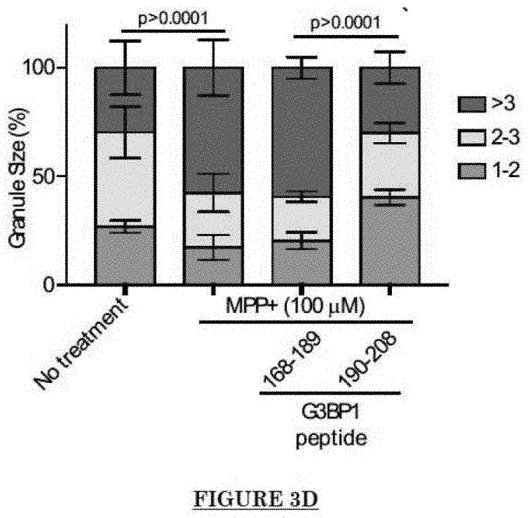

The results show that treatment of neurons with G3BP1 190-208 prevented neurite degeneration observed in neurons exposed to neurotoxins MPP+, see FIGS. 1A and B, and Aβ, see FIGS. 2A and B. Next, the inventors tested the efficacy of the G3BP1 190-208 peptides on an in vitro amyotrophic lateral sclerosis (ALS) model, mouse embryonic day 12.5 motor neurons were cultured for 7 days and then treated for 16-18 hours with mitotoxin, 100 μM MPP+ with or without 10 μM G3BP1 190-208 or 168-189 peptide. Non-treated cultures were used as controls (FIG. 3A). Treatment with G3BP1 190-208 protected against MPP+-induced degeneration as compared to neurons treated with MPP+ alone or those treated with control G3BP1 168-189. Taken together, these results show that G3BP1 190-208 peptide is protective against neurodegeneration induced by treatment number of neurotoxic insults.

Previous studies have shown that, exposure of midbrain neurons results in formation of stress granules. To test whether treatment of motor neurons with mitotoxin MPP+ results in the formation of stress granules the inventors stained the MPP+-treated neurons for G3BP1 protein. The inventors results show that presence of G3BP1 aggregates in non-treated motor neurons and treatment of the neurons with MPP+ results in an increase in G3BP1 aggregates, see FIGS. 3B and 3C. Moreover, treatment of MPP+-treated neurons with G3BP1 190-208 lead to a decrease in the size of aggregates with higher number smaller sized aggregates as compared to control 168-189 peptide, see FIG. 3D.

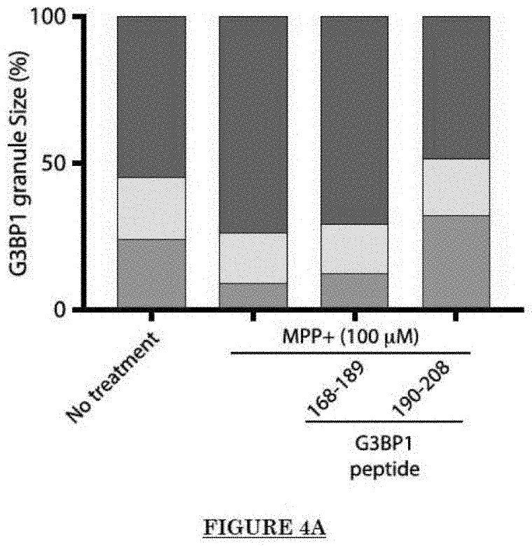

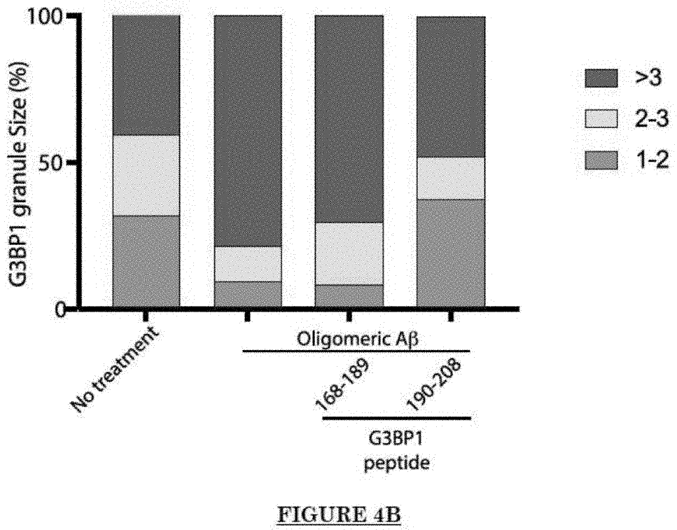

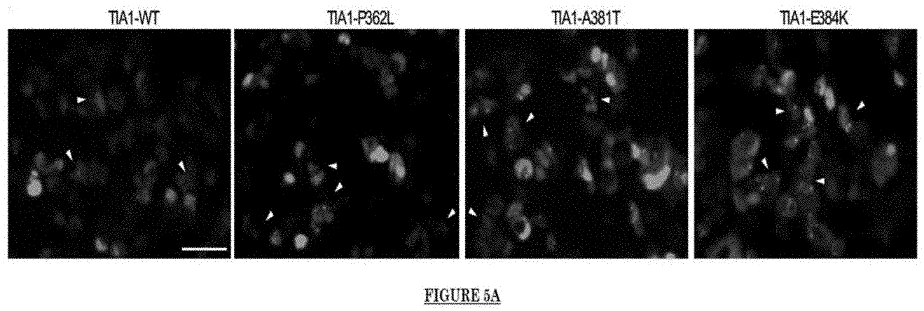

Similar increase in endogenous G3BP1 aggregates were observed after treatment of midbrain neurons with MPP+ and cortical neurons with Aβ oligomers, see FIGS. 4A and 4B. Consistent to the inventors results from motor neurons treated with MPP+, treatment with G3BP1 190-208 peptide leads to a decrease in the size of G3BP1 aggregates in midbrain neurons exposed to MPP+ or cortical neurons treated with Aβ oligomers. Together, these data indicate that treatment of neurons with neurotoxins results in formation of larger G3BP1 aggregates and that treatment with the G3BP1 190-208 peptide results in decrease in the size of the G3BP1 aggregates. Given that the G3BP1 190-208 peptide prevents degeneration induced by treatment with neurotoxins such as MPP+ and Aβ oligomer and decreases G3BP1 aggregates formed due MPP+ and Aβ treatment.

Currently, approximately 20 genes encoding RNA binding proteins have been shown to be mutated in neurodegenerative diseases. A number of stress-granule associated-RBPs have been implicated in the neurodegenerative and neurological disorders such as Ataxin-2 (in spinocerebellar ataxia), survival motor neuron (SMN) (in spino muscular atrophy), fragile X mental retardation protein (FMRP) (in fragile X syndrome), TDP-43 (in amyotrophic lateral sclerosis (ALS) and fronto-temporal lobar degeneration (FTLD)), FUS (in ALS) and TIA-1 (in ALS and FTD). An increasing number of these proteins form aberrant protein aggregates and also effect stress granule dynamics, thereby contributing to disease initiation and progression. Thus, alleviating the protein aggregation and restoring homeostatic SG dynamics may prove to be remedial.

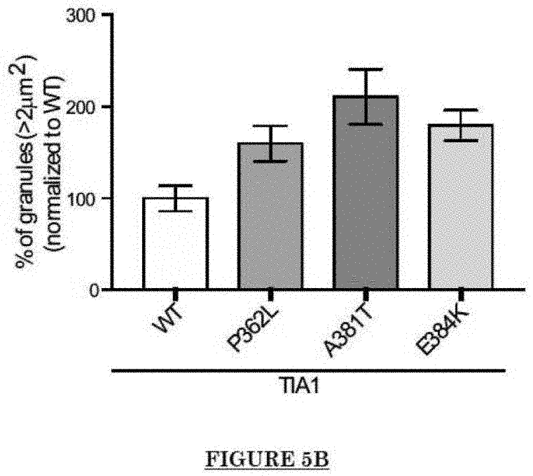

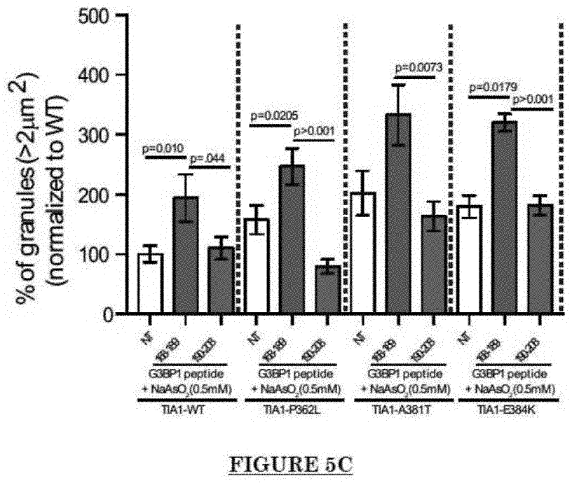

The inventors tested whether the G3BP1 190-208 peptide would prevent the aggregation of granules formed by TIA1 and TDP43 mutant proteins associated with ALS and FTD. The inventors expressed GFP-tagged wild-type or ALS/FTD-associated mutants(P362L, A381T, or E384K) TIA1 in HEK cells and the inventors imaging results show that consistent with previous studies, the ALS/FTD-associated mutants form larger TIA1 granules (>2 μm²), see FIGS. 5A and 5B. Next, we treated HEK cells expressing wild-type and or ALS/FTD-associated mutants (P362L, A381T, or E384K)TIA1, with arsenite a potent inducer of SG aggregation, in addition to either G3BP1 190-208 peptide or control G3BP1 168-189 peptide. Treatment with arsenite increases size of TIA1 aggregates for wild-type and ALS/FTD-associated mutants, however, exposure to G3BP1 190-208 peptide was able to decrease the size of the TIA1 aggregates, while no such change in aggregate size was observed upon treatment with the control peptide, see FIG. 5C. These data suggest that G3BP1 190-208 peptide can affects the aggregation of both wild-type and ALS/FTD-associated TIA1.

To further evaluate whether mutant TIA1 and TDP43 protein aggregates are affected by G3BP1 190-208 peptide, the inventors expressed the mutant proteins in embryonic motor neurons and assessed granules formed in neurites with or without arsenite treatment either in the presence of G3BP1 190-208 or control 168-189 peptide. Arsenite-treatment of motor neurons results in an increase in the size of endogenous TIA1 and G3BP1 aggregates, and addition of G3BP1 190-208 peptide reverses the increase number of these larger granules. Consistent with results observed in HEK cells, arsenite treatment leads to an increase in the number of larger granules (>2 μm²) for both wild-type and mutant TIA1-GFP proteins and the decrease in the number of large granules were observed upon addition of G3BP1 190-208 peptide, while no such effect was observed due to exposure to the control 168-189 peptide. Similar results were observed in neurons expressing flag-tagged wild-type and ALS-associated mutant TDP43, i.e., increased number of large flag-TDP43 aggregates (>2 μm²) were observed in neurites treated with arsenite and this effect was reversed by addition of the G3BP1 190-208 peptide. Taken together, these results suggest that the G3BP1 190-208 not only effects the dynamics G3BP1 granules, but also other stress granule proteins like TIA1 and TDP43 as well as the disease-associated mutant alleles. Therefore, the data suggests that the G3BP1 190-208 peptide may provide an efficacious means to treat patients suffering for protein aggregation related neurodegenerative disorders.

FIG. 1: Cell permeable G3BP1 190-208 peptide rescues MPP+-induced degeneration of midbrain neurons. (A) Representative images for NF-labeled (white) DIV7 E18 midbrain neurons. DIV7 midbrain cultures were treated with 100 μM MPP+ (ii) or control (i) and 100 μM 190-208 G3BP1 peptide (iv) or 168-189 control peptide (iii) [scale bar=100 μm]. (B) Quantification of neurite degeneration in midbrain neurons in control or 100 μM MPP+-treated neurons after addition of 190-208 G3BP1 or 168-189 control peptide. Results show that significant decrease in MPP+-induced degeneration for 190-208 peptide-treated cultures compared to 168-189 peptide exposure or MPP+-treated neurons. (N≥95 over 3 cultures; by one-way ANOVA with Tukey HSD post-hoc).

FIG. 2: Cell permeable G3BP1 190-208 peptide blocks pathological degeneration beta-amyloid-induced neurotoxicity in cortical neurons. (A) Representative images of NF-labeled (white) E18 cortical neurons. DIV7 cortical neurons were treated with 10 μM Aβpeptide (ii) or control (i) and 10 μM 190-208 G3BP1 peptide (iv) or 10 μM 168-189 control peptide (iii) [scale bar=100 μm]. (B) Quantitation of degeneration in cortical neurons treated with 1 μM Aβpeptide-treated neurons after addition of 190-208 G3BP1 or 168-189 control peptide. Solvent treated cells were used as control. A significant rescue from Aβ-induced neurite degeneration is observed in 190-208 peptide-treated neurons as compared to treatment with either 168-189 peptide or Aβ-treated controls. (N≥95 over 3 cultures; by one-way ANOVA with Tukey HSD post-hoc).

FIG. 3: Cell permeable G3BP1 190-208 peptide prevents mitotoxin-induced degeneration in motor neurons and axonal mRNA translation and causes disassembly of stress granules. (A) Quantification of mitotoxin/neurotoxin MPP+-induced degeneration of DIV7 E12.5 motor neuron neurites after exposure of cultures to 100 μM MPP+ and either 10 μM 190-208 G3BP1 or 168-189 control peptide. As controls, neurons treated with solvents were used. (B) Representative images of G3BP1 (green) and NF-labeled (red) E12.5 motor neurons under control or after treatment with 100 μM MPP+ [scale bar=10 μm]. (C) distribution of sizes of endogenous G3BP1 aggregate per 100 μm neurite is shown as indicated bins from motor neurons cultures as treated in (B). (N≥80 neurites over three repetitions; by one-way ANOVA with Tukey HSD post-hoc). Results show a shift towards larger G3BP1 granule sizes in neurons treated with MPP+ as compared to controls. (D) Endogenous G3BP1 aggregate sizes per 100 μm neurite indicated as bins for motor neurons either untreated or treated with MPP+ alone or with addition of 10 μM 190-208 G3BP1 or 168-189 control peptide. Treatment with MPP+ results in formation of larger G3BP1 granules as compared to no treatment control. Addition of 190-208 G3BP1 peptide in the presence of MPP+ results in a decrease in the number of larger G3BP1 granules and reverses the G3BP1 granule distribution to those observed in the non-treated neurons. (N≥90 over 3 cultures; by one-way ANOVA with Tukey HSD post-hoc)

FIG. 4: Treatment of neurons with cell permeable G3BP1 190-208 peptide leads to disassembly of G3BP1 granules formed due to exposure of neurons to neurotoxins MPP+ and Aβpeptide. (A) Size of endogenous G3BP1 aggregates per 100 μm of neurite is shown as indicated bins from midbrain cultures under control conditions and after treatment with MPP+alone or with 190-208 G3BP1 or 168-189 peptides. (B) Size distribution of endogenous G3BP1 aggregates per 100 μm of neurite is shown from cortical neurons treated with 1 μM Aβpeptide or with 100 μM 190-208 G3BP1 or 168-189 control peptide. Non-treated neurons were used as a control. The results show that treatment with 190-208 G3BP1 peptide leads to disassembly of G3BP1 granules. (N≥90 over 3 cultures; by two-way ANOVA with Tukey HSD post-hoc).

FIG. 5: Cell permeable G3BP1 190-208 peptide decreases TIA1-GFP puncta formed due to expression of disease causing TIA1 mutants. (A) Representative images of HEK cells transfected with GFP-tagged wild-type or ALS/FTD-associated mutants (P362L, A381T, or E384K) TIA1. Arrows show TIA1-GFP puncta. (B) Quantification of the percentage of cells with TIA1-GFP puncta greater than 2 μm² area in transfected cells relative TIA1-WT-GFP expressing cells. Consistent with previous studies, ALS/FTD- associated TIA1 mutants show increased number of TIA1-GFP puncta as compared to WT-TIA1. (C) HEK cells expressing TIA1-GFP or ALS/FTD-associated mutants TIA1-P362L, TIA1-A381T, TIA1-E384K were treated with sodium arsenite (0.5 mM) for 30 minutes followed by treatment with either 10 μM 190-208 G3BP1 or 168-189 control peptide. The number of cells containing TIA1-GFP puncta were measured and results are reported relative to non-treated controls. Number of HEK cells with TIA1-GFP granules both for WT and ALS/FTD mutants decreased after treatment with 10 μM 190-208 G3BP1 as compared to the 168-189 control peptide treated cells. (N≥90 over 3 cultures; by two-way ANOVA with Tukey HSD post-hoc). Scale bar: 20 μm.

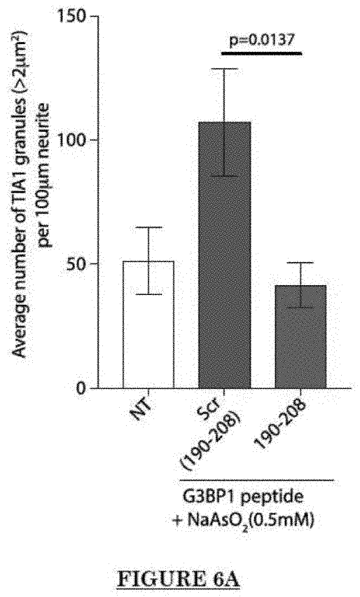

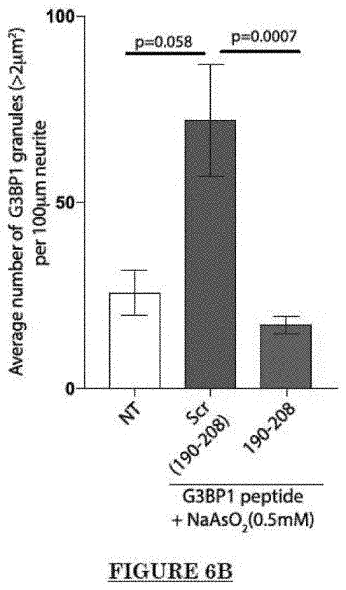

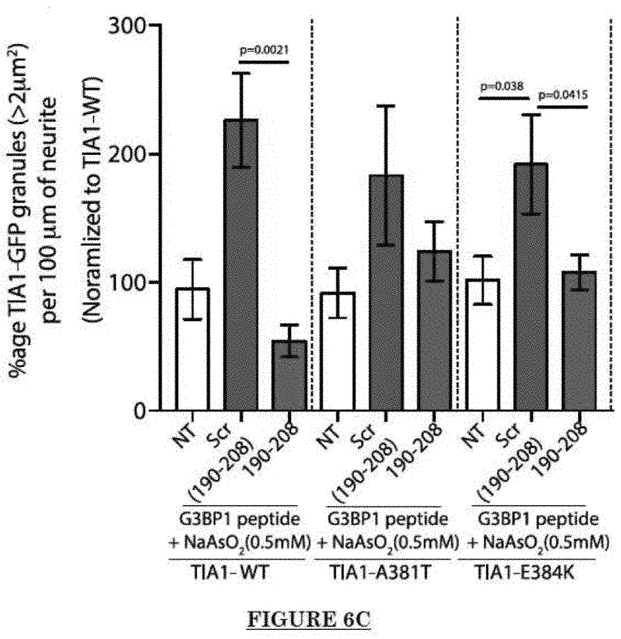

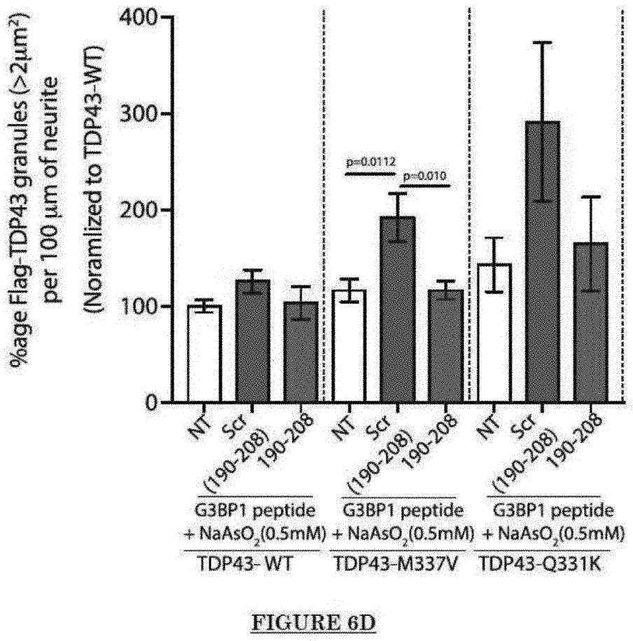

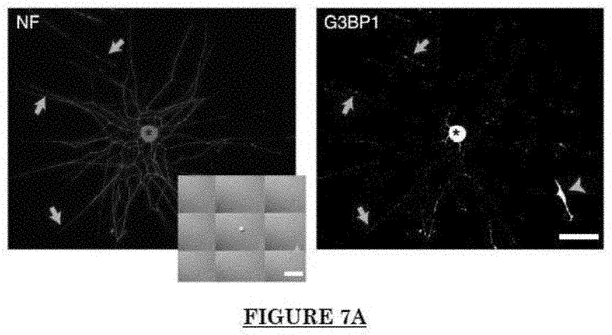

FIG. 6: Cell permeable G3BP1 190-208 peptide decreases granules formed by endogenous TIA1 and TDP43 and exogenous TDP43 and TIA1 mutants in motor neurons after sodium arsenite exposure. (A) Embryonic motor neurons isolated from E12.5 mouse embryos were exposed to sodium arsenite (0.5 mM) for 30 minutes and then treated with 10 μM 190- 208 or 168-198 peptide. After fixation endogenous TIA1 was visualized by immunostaining. The number of TIA1 granules greater than 2 μm² per 100 μm of neurite were measured. (B) E12.5 motor neurons were treated as described in (A) and immunostaining was used to detect endogenous G3BP1 protein. The number of G3BP1 granules greater than 2 μm² per 100 μm of neurite were measured. The results show significantly decreased levels of TIA1 and G3BP1 granules in neurons treated with 190-208 treated neurons compared to 160-189 peptide treated neurons. (C) E12.5 motor neurons expressing TIA1-GFT or ALS/FTD-associated mutants TIA1-A381T, TIA1-E384K were treated with sodium arsenite (0.5 mM) for 30 minutes followed by treatment with either 10 μM 190-208 G3BP1 or 168-189 control peptide. The number of cells containing TIA1-GFP puncta were measured 2 μm² per 100 μm of neurite and results are reported relative to non-treated controls. The number of TIA1-GFP granules in neurites decreased for WT and ALS/FTD mutants after treatment with 190-208 G3BP1 as compared to the 168-189 control peptide treated cells. (I)) E12.5 motor neurons expressing flag-tagged wildtype TDP43 or ALS-associated mutants TDP43-M337V and TDP43-Q331.K were treated with sodium arsenite (0.5 mM) for 30 minutes followed by treatment with either 10 μM 190-208 G3BP1 or 168189 control peptide. Flag-TDP43 was detected by immunostaining using the anti-flag-antibody. The number of flag-TDP43 granules greater than 2 μm² were measured per 100 μm of neurite and the results are reported relative to non-treated controls. The number of Flag-TDP43 granules in neurites were decreased after treatment with 190-208 G3BP1 peptide as compared to the 168-189 control peptide in both WT and ALS/FTD mutants expressing cells. (N≤50 over 3 cultures; by two-way ANOVA with Tukey HSD post-hoc),

Critical functions of intra-axonally synthesized proteins are thought to depend on regulated recruitment of mRNA from storage depots in axons. Here the inventors show that axotomy of mammalian neurons induces translation of stored axonal mRNAs via regulation of the stress granule protein G3BP 1, to support regeneration of peripheral nerves. G3BP1 aggregates within peripheral nerve axons in stress granule-like structures that decrease during regeneration, with a commensurate increase in phosphorylated G3BP1. Colocalization of G3BP1 with axonal mRNAs is also correlated with the growth state of the neuron. Disrupting G3BP functions by overexpressing a dominant-negative protein activates intra-axonal mRNA translation, increases axon growth in cultured neurons, disassembles axonal stress granule-like structures, and accelerates rat nerve regeneration in vivo.

Injured axons in the peripheral nervous system (PNS) use locally translated proteins for retrograde injury-signaling and regenerative growth. Translation of axonal mRNAs can be activated by different stimuli including axotomy in mature neurons and in response to guidance cues in developing neurons, indicating that a significant fraction of axonal mRNAs are stored until a particular stimulus activates their translation. Stress granules (SG) serve as storage depots for mRNAs in nonneuronal systems, providing a mechanism to respond to cellular stress by sequestering unneeded mRNAs from translation. Aggregation-prone mutations of the SG protein TIA1 and the RNA-binding protein TDP-43 have been shown to cause SG aggregation in neurons, but it is not known if SGs have roles in the normal function of neurons. Further, although SGs have been detected in dendrites, it is not clear if functional SGs are assembled in axons. The Ras GAP SH3 domain binding protein 1(G3BP1) interacts with the 48S pre-initiation complex when translation is stalled, and it assembles SGs by virtue of its NTF2-like domain. Murine G3BP1 knockout is embryonic lethal in 129/Sv mouse strain with CNS apoptosis, but not a mixed Balb/c/129/Sv background where altered synaptic plasticity and neuronal calcium homeostasis were seen. This emphasizes roles for G3BP1 protein in the nervous system. Proteomics analyses recently reported G3BP1 interactomes from neurites of cultured motor neurons, where a core of SG-associated proteins was detected in the absence of stress. Thus, G3BP1 aggregates may have functions in axons.

Here the inventors show that translation of specific axonal mRNAs is negatively regulated in intact axons by G3BP1, and that this negative regulation is removed by dispersion of aggregated G3BP1 in regenerating peripheral nerves post injury to support accelerated axon growth. When phosphorylated on serine 149 (G3BP 1PS¹⁴⁹), G3BP1′s oligomerization is blocked and SGs disassemble, presumably releasing bound mRNAs for translation. Loss of G3BP1 aggregation in SG-like structures in regenerating axons is accompanied by an increase in phosphorylated G3BP 1.

Disrupting G3BP1 function with a dominant-negative approach activates intra-axonal mRNA translation, increases axon growth in cultured neurons and accelerates nerve regeneration in vivo, and therefore represents a new pro-regenerative therapeutic approach.

Results

Axonal G3BP1 aggregates decrease during nerve regeneration. The inventors initially asked if axons of cultured primary sensory neurons contain stress granule-associated protein G3BP1. Sensory neurons in dissociated cultures from adult rat dorsal root ganglia (DRG) show strong immunoreactivity for G3BP1 in cell bodies and focally along their axons (FIG. 6A). By confocal microscopy, axonal G3BP1 signals appeared to show higher colocalization with other SG components compared with components of processing bodies (PB) that are linked to RNA degradation (FIG. 6B).

Comparing Pearson's coefficients from these colabelings showed a significantly higher colocalization of axonal G3BP1 with SG markers than with PB markers (FIG. 6C). Colocalization of axonal G3BP1 with the SG protein HuR but not the PB protein DCP1a was further confirmed by proximity ligation assay (PLA; FIG. 6D). Overall, the axonal G3BP1 aggregates appeared smaller than those described for SGs in non-neuronal cells (diameter ˜0.2-0.8 μm versus≥1 μm for stress-induced aggregates), so the axonal SG-like entities approximate the ˜250 nm diameter described for SG core structure.

Confocal microscopy of sciatic nerve sections showed robust, granular G3BP1 signals that overlapped with neurofilament (NF) across optical planes of the Z stacks (FIG. 6E). Signals for the SG protein TIA1 focally overlapped with G3BP1, including the granular intra-axonal G3BP1 signals, and the axonal signals for both G3BP1 and TIA1 appeared to decrease in 7 d post-crush sciatic nerve just proximal to the crush site (FIG. 6E). The imaging parameters for these analyses were selected to visualize only the granular G3BP1 signals; on highly over-exposing the granular

G3BP1 signals, more diffuse signals were noted for G3BP1 along the axon (data not shown), suggesting that the granular signals represent aggregates of G3BP1 in axons. The inventors quantified the granular signals for G3BP1 and TIA1 in axons proximal to crush site at intervals over 3 h to 7 d after axotomy. Both proteins showed a striking increase in the intra-axonal signals at 3 h postcrush which fell to below the levels of the naive axons by 5 d postcrush; notably, the fold change for G3BP1 and TIA1 near perfectly overlapped across this time course (FIG. 6F). Axons are actively regenerating at 7 d after nerve crush (see below), and granular G3BP1 signals were largely excluded from the thin axons at the regenerating front of the injured nerve.

Immunoblotting of DRG neurons transfected with control versus G3BP1 targeting siRNAs confirmed the specificity of the anti-G3BP1 antibody used here. To gain a more quantitative assessment of G3BP1 protein levels in axons, the inventors used targeted mass spectrometry (MS) of sciatic nerve axoplasm taken over 3-28 d post injury. The MS analyses further confirmed presence of G3BP1 in axons and showed modest, but highly variable, declines in G3BP1 levels after an injury. Approximately 3 cm of nerve proximal to the crush site was used for axoplasm preparations in these MS studies. Immunoblotting axoplasm from shorter segments of injured sciatic nerve (0 to _1 cm and _1 to _2 cm proximal to the crush site) showed a clear reduction in G3BP1 signals in 7 d injured compared to naive sciatic nerves.

Taken together, these data indicate that axonal SG-like structures and G3BP1 protein levels change after axonal injury and subsequent regeneration of PNS nerves. Thus, the inventors wondered if the decrease in axonal SG-like aggregation might be a feature of growing axons. So the inventors asked if axonal SG-like structures show alterations in vitro in DRG neurons with different axon growth capacity. DRG neurons that are conditioned by an in vivo crush injury 7 d prior to culture show more rapid axonal outgrowth over 18-48 h in vitro compared to uninjured (naive) DRGs 13 , and the rapidly growing axons of those injury-conditioned neurons showed a decrease in G3BP1 aggregates compared to those of naive DRG cultures (FIGS. 6G and 6H). Together, these data raise the possibility that aggregation of axonal G3BP 1 in PNS axons is associated with a lower axon growth activity.

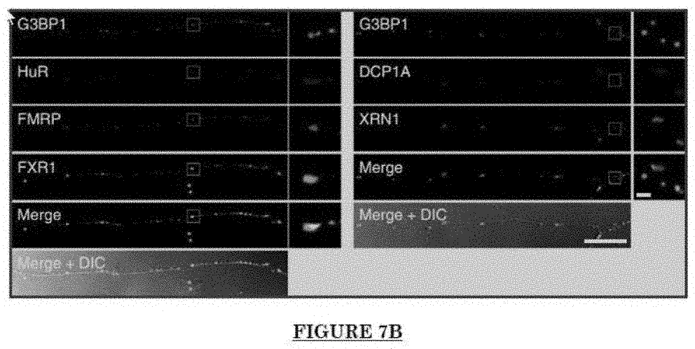

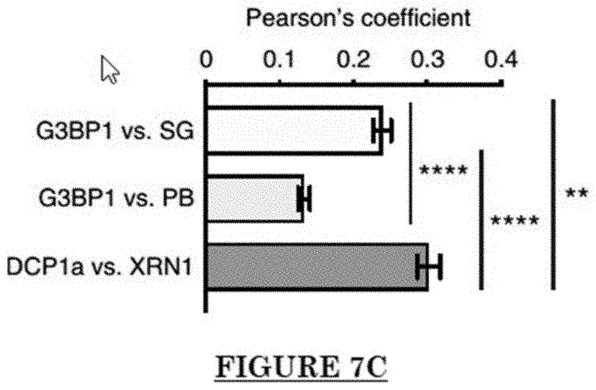

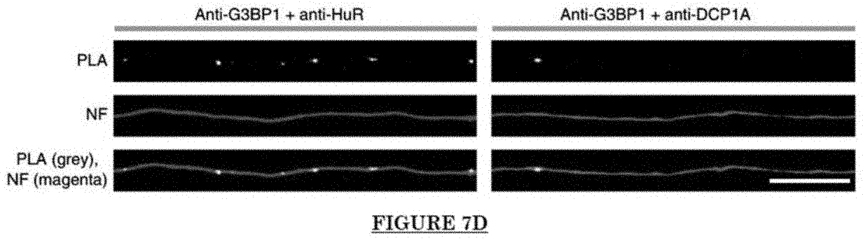

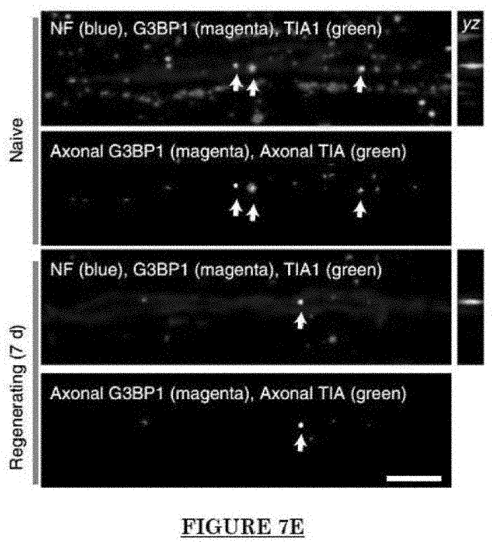

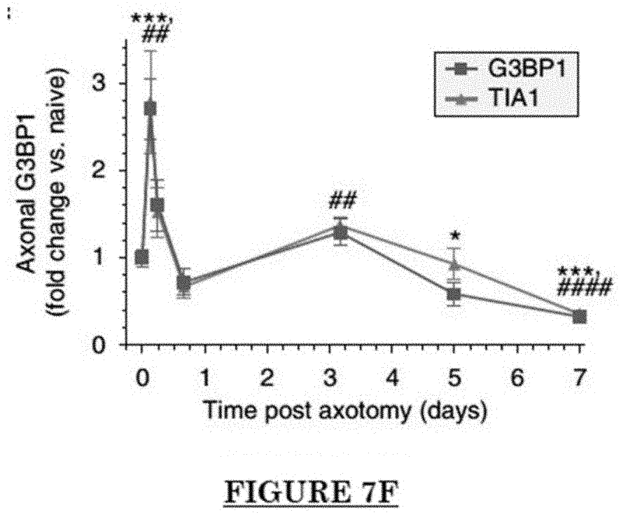

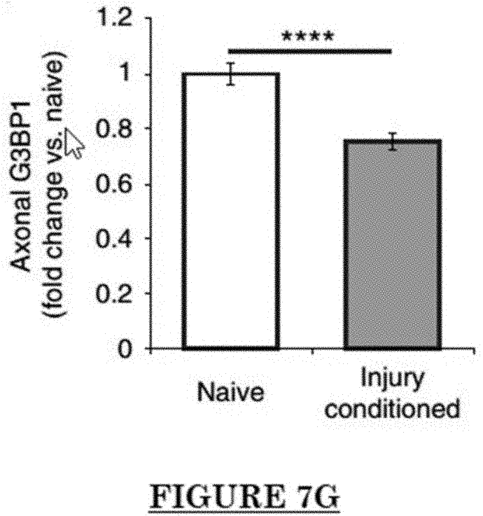

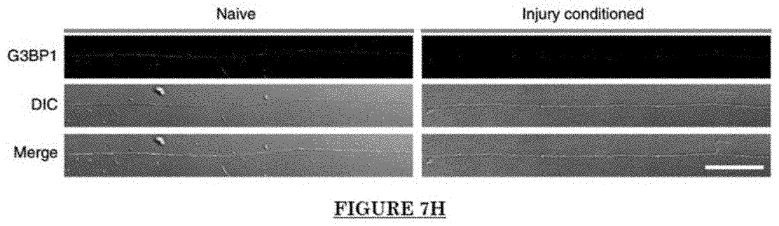

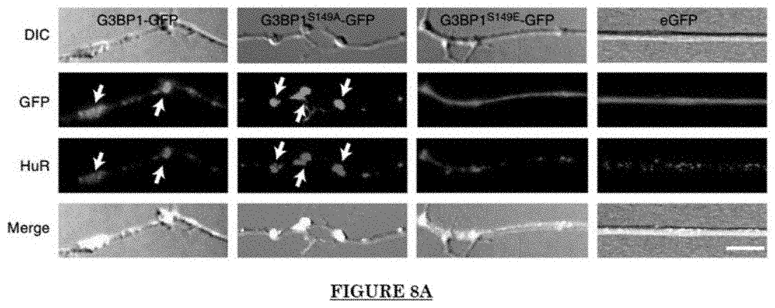

G3BP1 is phosphorylated in regenerating axons. Phosphorylation of G3BP1 on Serine 149 has been shown to trigger disassembly of SGs. To determine if phosphorylation alters aggregation of axonal G3BP1, the inventors expressed nonphosphorylatable and phosphomimetic G3BP1 mutants (G3BP1^{S149A}-GFP and G3BP1^{S149E}-GFP, respectively) in cultured DRGs. Axonal G3BP1^{S149A}-GFP showed aggregated signals that overlapped with the SG-associated protein HuR, while axonal G3BP1^{S149E}-GFP appeared diffuse (FIGS. 7A and 7B). G3BP1^{S149E}-GFP also showed significantly higher mobility in axons than G3BP1^{S149A}-GFP, and G3BP1-GFP showed mobility intermediate between G3BP1^{S149E}-GFP and G3BP1^{S149A}-GFP (FIG. 7C). This is consistent with G3BP1^{S149A}-GFP aggregating into SG-like structures in axons.

The inventors next asked whether endogenous G3BP 1 is phosphorylated in axons using phospho-specific G3BP1P^{S149} antibodies.

FIGS. 6A-6H show G3BP1 localizes to axons in stress granule-like aggregates. Immunofluorescence for G3BP1 shows signals in the cell body (asterisk) and axons (arrows) of a cultured DRG neuron; arrowheads indicate Schwann cell with prominent G3BP1 immunoreactivity visible in the inset DIC image. Previous work has shown that neurites of these adult DRG neurons have axonal features and lack dendritic features; the inventors will use ‘axon’ for describing these hereafter [scale bar=50 μm]. b, c Single planes for axons of naive DRG cultures co-labeled for indicated proteins are shown; box represents the area for high magnification insets to right (b). Axonal G3BP1 shows higher colocalization coefficients for SG than PB proteins by Fisher's Z transformation (c; N≥30 axons over 3 repetitions; **p≤0.01, ****p≤0.001 by one-way ANOVA with Tukey HSD post-hoc) [scale bar=10 μm for large panels, 1 μm for insets]. d PLA shows higher colocalization for G3BP1 and HuR than G3BP1 and DCP1A (G3BP1+HuR PLA=0.038±0.003 and G3BP1+DCP1A PLA=0.027±0.002 signals/gm; N≥40 neurons over 6 repetitions, p=0.016 by Student's t-test) [scale bar=20 μm]. e, f Confocal images for G3BP1 and TIA1 in naive and 7 d post-injured (‘regenerating’) sciatic nerve are shown (e). Upper image panels of each pair show G3BP1 and TIA1 merged with NF signals in single plane. Lower panels of each pair show XYZ for G3BP1 and TIA1 signals that overlap with NF across the Z stack Quantitation of axonal G3BP1 and TIA1 signals are shown (f) as mean±SEM (N=6 animals; *p≤0.05, ***p≤0.001 for G3BP1 and p≤0.01, **** p≤0.0001 for TIA1 by Student's t-test for versus naive) [scale bar=5 μm]. g, h Quantification of G3BP1 levels (g) and G3BP1 immunofluorescence (h) in axons of DRGs cultured from naive versus 7 d injury-conditioned animals are shown (mean±SEM for N≥66 neurons over 3 repetitions; ***p≤0.001 by Student's t-test) [scale bar 20 μm]

Immunoblotting with lysates from control versus G3BP1 siRNA transfected DRGs showed a single band for anti-G3BP1P1^{PS149}. Treating DRG cultures with arsenic, a known inducer of SG aggregation, also decreased levels of G3BP1PS¹⁴⁹ without affecting overall G3BP1 levels by immunoblotting (data not shown). By immunofluorescence, intra-axonal signals for anti-G3BP1PS¹⁴⁹increased in proximal sciatic nerves 7 d post-crush injury (FIGS. 7D and 7E). Thus, as the prevalence of axonal SG like structures decreased in regenerating axons, there was a corresponding increase in axonal G3BP1^{PS149}. Moreover, in cultured DRG neurons, the ratio of axonal G3BP1^{PS149} to axonal G3BP1 aggregates increases in distal axons and growth cones (FIGS. 7F and 7G), suggesting that the axonal G3BP1 aggregation and phosphorylation are dynamically regulated along the growing axon.

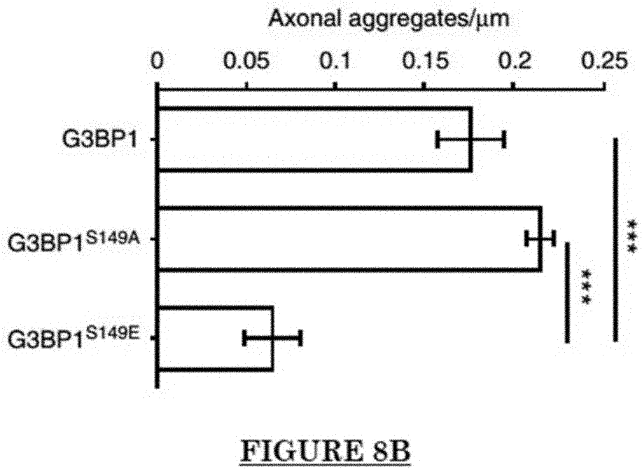

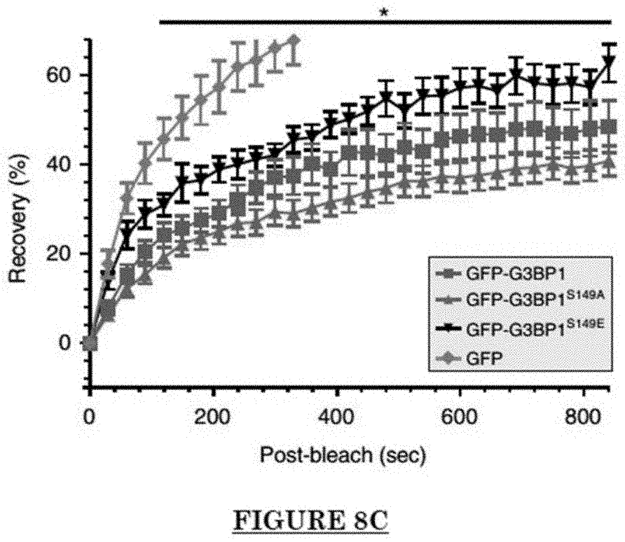

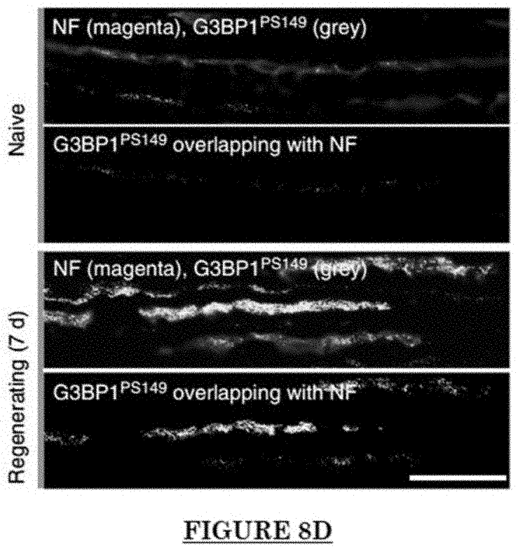

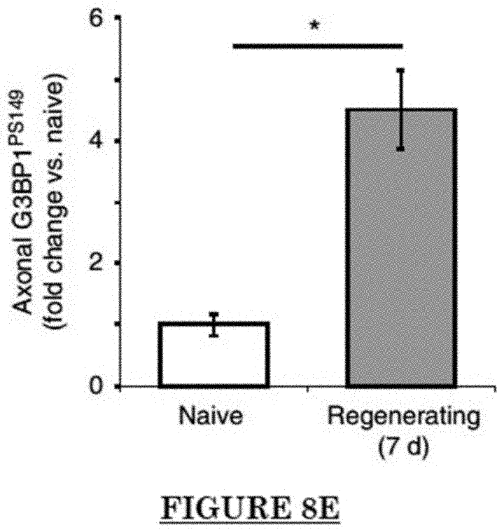

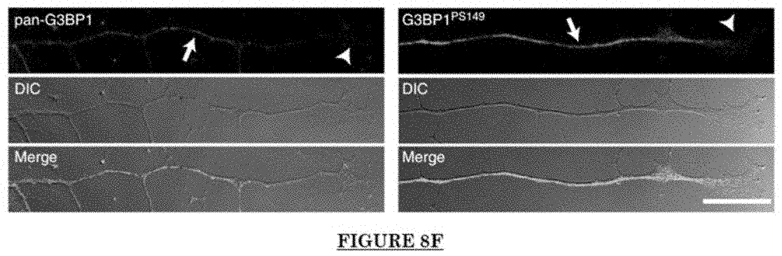

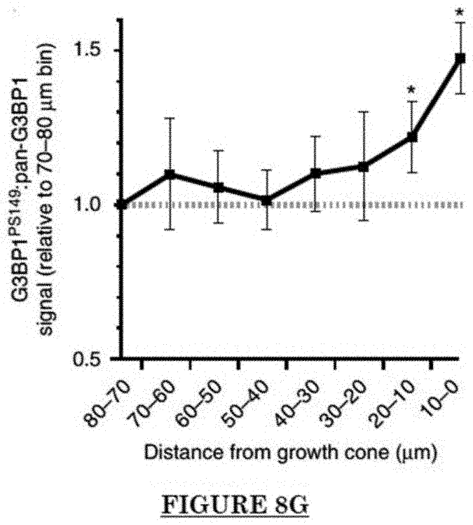

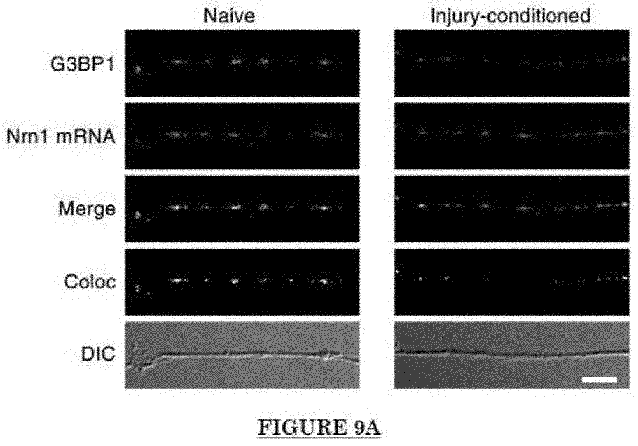

Axonal G3BP1 modulates axonal mRNA translation. Previous studies detected ribosomes and translation factors in regenerating PNS axons in vivo, so the decrease in SG-like structures in distal axons could reflect increased protein synthesis in those axons. Thus, the inventors asked if axonal mRNAs colocalize with G3BP1 in cultured neurons. Endogenous Neuritin1 (Nrn1) and Importin β1 (Impβ1) mRNAs showed clear colocalization with axonal G3BP1, but the mRNA encoding Growth-associated protein 43 (Gap43) did not (FIG. 8A). The more rapidly growing axons of injury-conditioned DRG neurons showed higher colocalization of Impβ1 with G3BP1 than those of naive DRGs, while axonal Nrnl showed the opposite (FIG. 8B). Axonal Gap43 showed overall lower G3BP1 colocalization coefficients that did not change with injury conditioning (FIG. 8B). IMPβ1 protein is used for injury response after axotomy and negatively regulates axon growth under basal conditions, while NRN1 protein supports regenerative growth of axons. Thus, these distinct colocalizations of axonal Impβ1 and Nrn1 mRNAs with G3BP1 protein in naive versus injury-conditioned neurons may reflect different functions of the encoded proteins in these different growth states.

The inventors next used fluorescent reporters to determine if axonal SG like structures contribute to translation. For this, the inventors generated axonally targeted GFP^{MYR} and mCherry^{MYR} containing the 5′ and 3′ untranslated regions (UTR) of Impβ1, Nrn1, and Gap43 mRNAs (GFP^{MYR} 5′/3′impβ1, GFP^{MYR} 5′/3′nrn1, and mCh^{MYR} 5′/3′gap43, respectively; FIG. 8C). The membrane localizing myristoylation (MYR) of the fluorescent reporter proteins dramatically limits their diffusion from sites of translation, so GFP^{MYR} and mCherry^{MYR} proteins provide versatile reporters for localized protein synthesis in dendrites and axons using fluorescence recovery after photobleaching (FRAP). The 3′ (Impβ1 and Gap43) and 5′ (Nrnl) UTRs provide axonal targeting for reporter mRNAs, and with both 5′ and 3′UTRs, the reporters approximate the translational regulation of the endogenous mRNAs. Recovery of axonal GFP^{MYR} 5′/3′nrn1 and GFP^{MYR} 5′/3′impβ1 fluorescence was decreased in DRGs expressing G3BP1-BFP compared to the BFP control, but mCh^{MYR} 5′/3′gap43 recovery was not significantly affected by G3BP1-BFP expression (FIGS. 8D-8G). Treatment with translation inhibitors confirmed that the fluorescence recovery in axons after photo bleaching represents new protein synthesis, and, interestingly, overexpression of G3BP1-BFP approximated the effect of protein synthesis inhibition for GFP^{MYR} 5′/3′nrn1 and GFP^{MYR} 5′/3′impβ1 fluorescence recovery (FIGS. 8E-8G). Additionally, RNA immunoprecipitation (RIP) analyses showed enrichment of GFP^{MYR} 5′/3′impβ1 and GFP^{MYR} 5′/3′nrn1, but not mCh^{MYR} 5′/3′gap43, in G3BP1 immunoprecipitates (FIG. 8H). Taken together, these data suggest that G3BP1 binds to Nrn1 and Impf31 mRNAs and attenuates their translation in axons.

FIGS. 7A-7G show G3BP1 is phosphorylated in regenerating axons. a Representative images for axons of DRG neurons transfected with indicated G3BP1 constructs versus eGFP are shown. G3BP1-GFP and G3BP1^{S149A}-GFP show prominent aggregates in axons that colocalize with HuR (arrows). In contrast, axonal signals for G3BP1^{S149E}-GFP and eGFP appear diffuse [scale bar=5 μm]. b Quantification of axonal aggregates for G3BP1-GFP, G3BP1^{S149A}-GFP, and G3BPP1^{S149E}-GFP is shown as average±SEM (N≥10 neurons over 3 repetitions; ***p≤0.005 by one-way ANOVA with Tukey HSD post-hoc). c FRAP analyses for neurons transfected with constructs as in A are shown as average normalized % recovery±SEM. G3BP1^{S149A}-GFP shows much lower recovery than G3BP1^{S149E}-GFP; G3BP1-GFP is intermediate between G3BP1^{S149A}-GFP and G3BP1^{S149E}-GFP (N≥13 axons over 3 repetitions; *p≤0.05 between G3BP1^{S149A}-GFP versus G3BP1^{S149E}-GFP by one-way ANOVA with Tukey HSD post-hoc). Only the 0-320s. recovery signals for GFP are shown (at 840 s. GFP showed 85.5±4.7% recovery with p≤0.0001 versus G3BP1^{S149E}-GFP by one-way ANOVA with Tukey HSD post-hoc). d-e Exposure-matched confocal images for G3BP1^{PS149} and NF are shown for sciatic nerve (d) as in FIG. 6E. There is a striking increase in G3BP1^{PS149} immunoreactivity in the regenerating axons. Quantifications of these signals are shown as mean±SEM (e; N=3; *p≤0.05 by one-way ANOVA with Tukey HSD post-hoc) [scale bar=20 μm]. f-g Distal axons of cultured DRGs immunostained with pan-G3BP1 versus G3BP1^{PS149} antibodies are shown as indicated (f). Aggregates of G3BP1 are visible in the axon shaft (arrow), but decrease moving distally towards the growth cone (arrowhead).

G3BP1^{PS149}signals are fairly consistent and extend into the growth cone (arrowhead). Quantification of signals (g) shows significant increase in ratio of G3BP1^{PS149}immunoreactivity to G3BP1 aggregates moving distally to the growth cone (N≥9 neurons each over 3 repetitions; *p≤0.05 versus 70-80 μm bin by one-way ANOVA with Tukey HSD post-hoc) [scale bar=20 μm]

FIGS. 8A-8H show 3 G3BP1 regulates translation of axonal mRNAs. a Images of FISH/IF for Nrn1 mRNA and G3BP1 protein are shown for axons of naive and 7 d injuryconditioned DRG neurons. Colocalization panel (Coloc) represents the mRNA:G3BP1 colocalization in a single optical plane [scale bar=5 μm]. b Quantification of colocalizations for Nrn1, Impβ1, and Gap43 mRNAs with G3BP1 in axons of neurons cultured from naive or 7 d injury-conditioned animals shown as average Pearson's coefficient±SEM (N≥21 neurons over 3 repetitions; **p≤0.01 and ***p≤0.005 by one-way ANOVA with Tukey HSD post-hoc). c Schematics of translation reporter constructs used in panels d-h. d Representative FRAP image sequences for DRG neurons co-transfected with GFP^{MYR} 5′/3′nrn1 plus BFP or G3BP1-BFP. Boxed regions represent the photobleached ROIs. e-g Quantifications of FRAP assays from DRGs expressing GFP^{MYR} 5′/3′nrn1 (e) or GFP^{MYR} 5′/3′impβ1 (f) or mCh^{MYR} 5′/3′gap43 translation reporters along with G3BP1-BFP or control BFP are shown as normalized, average % recovery±SEM (N≥11 neurons over 3 repetitions; *p≤0.05, and **p≤0.01 for BFP versus G3BP1-BFP, p≤0.05, p≤0.01, and p≤0.0001 for BFP versus translation inhibitors by one-way ANOVA with Tukey HSD post-hoc).

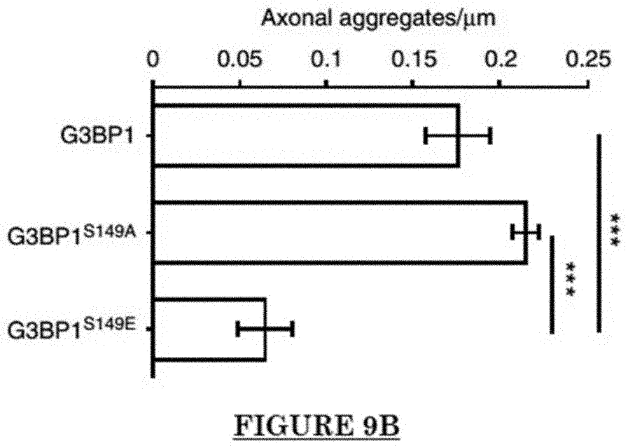

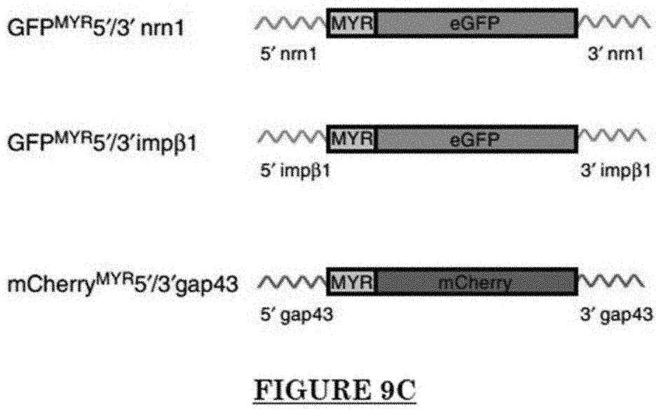

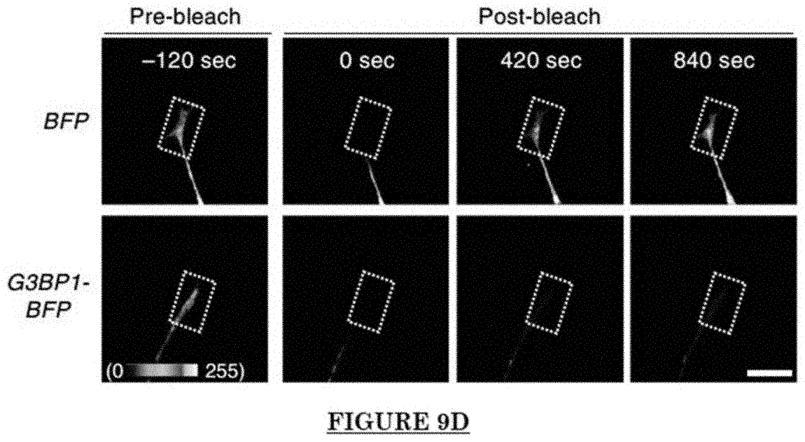

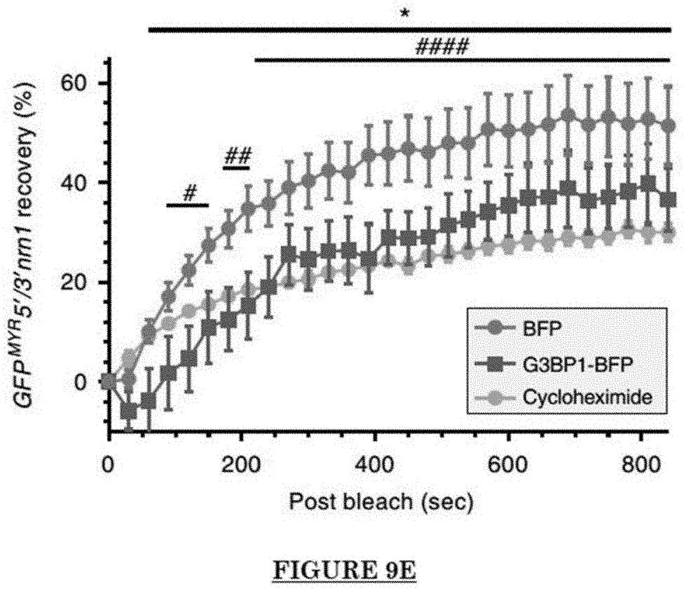

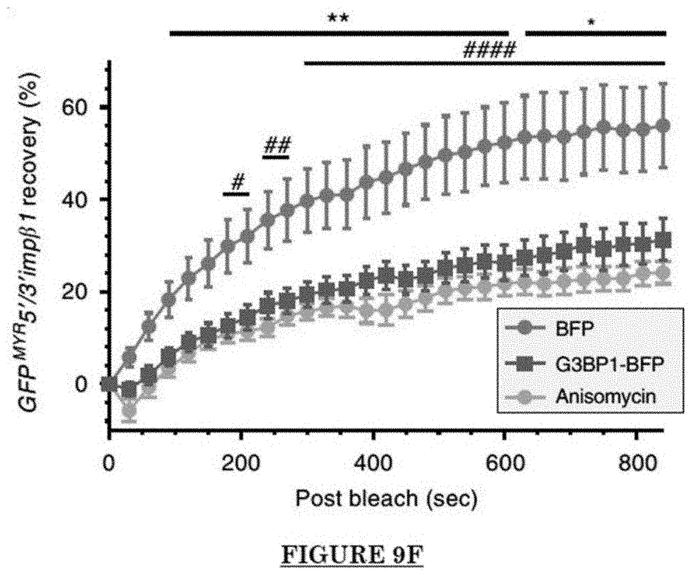

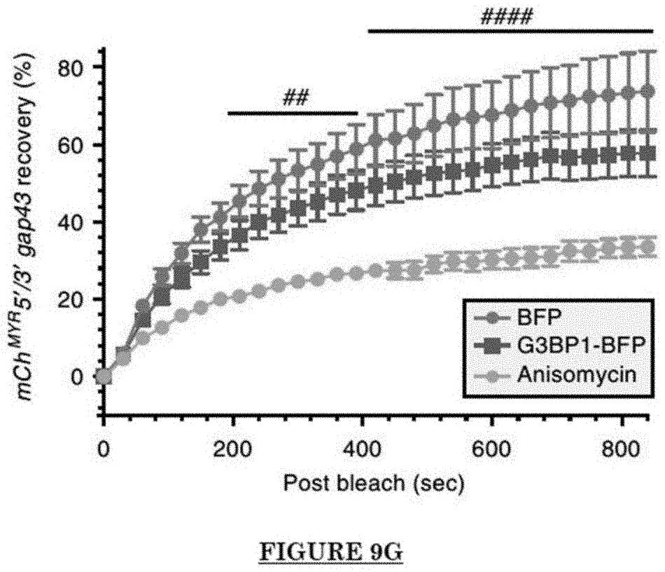

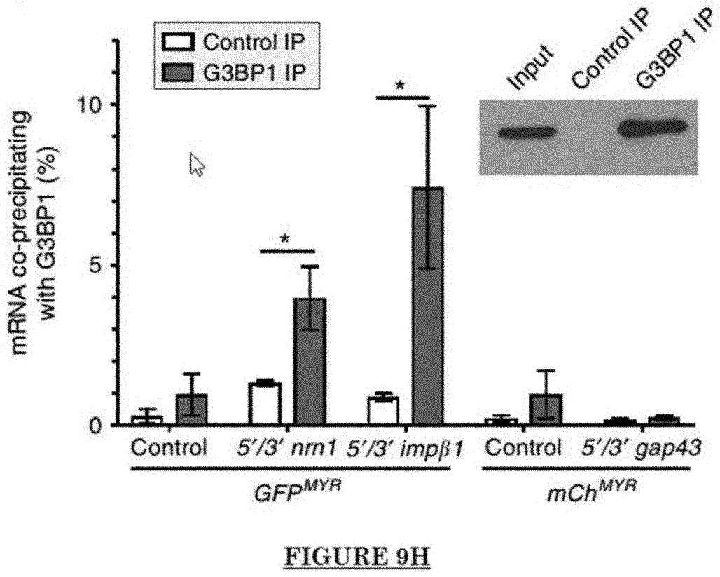

HEK293T cells transfected with GFP^{MYR} 5′/3′nrn1, GFP^{MYR} 5′/3′impβ1, and mCh^{MYR} 5′/3′gap43 show significant enrichment of GFP^{MYR} 5′/3′nrn1 and GFP^{MYR} 5′/3′impβ1 mRNAs coimmunoprecipitating with G3BP1 versus control (N=4 culture preparations; *p≤0.05 by Student's t-test). Western blot validating G3BP1 immunoprecipitation shown as inset. Values shown as average percent bound mRNA relative to input±SEM. The acidic domain of G3BP1 increases axonal growth. Four domains have been defined for G3BP1 protein: an N-terminal NTF2-like ‘A domain’, a highly acidic ‘B domain’, a PxxP motif containing ‘C domain’, and a C-terminal RNA-binding motif containing ‘D domain’ (FIG. 9A).

The inventors acquired expression constructs for the B, C, and D domains and combinations of these to determine if they might affect the function of endogenous G3BP1 in the DRG neurons. Expression of these G3BP1 deletion constructs in naive DRG cultures showed that G3BP1 B, CD, BCD, and D domain proteins all localized to axons. Neurons expressing the G3BP1 B domain showed significantly longer axons, while those expressing the D or CD domains showed shorter axons (FIG. 9B).

The G3BP1 D domain was previously shown to reduce protein synthesis in non-neuronal cells by triggering phosphorylation of the translation initiation factor eIF2α. Interestingly, a combined construct of the B domain with the CD domain significantly increased axon outgrowth, pointing to a dominant-negative effect of the B domain in absence of G3BP1's aggregating NTF-2 like region. Though the G3BP1 B domain contains Ser 149 whose phosphorylation causes SG disassembly, neither G3BP1^{S149E}-GFP nor G3BP1^{S149A}-GFP altered axon growth in the DRGs compared to GFP. DRGs expressing the B domain- and CD domain-GFP showed modest decline in neurites per neuron, as did the expression of full-length G3BP1-GFP. However, overexpression of full length G3BP1 had no significant effect on axon growth, perhaps indicating that G3BP1 is at saturating levels in DRG neurons. Consistent with this, siRNA-mediated G3BP1 depletion significantly increased axon growth and this was completely reversed by co-transfection with a siRNA-resistant G3BP1-GFP. Co-transfecting with the G3BP1 B domain did not further increase axon length in the G3BP1 depleted neurons, suggesting that the B domain inhibits function of endogenous G3BP1.

In light of the axon growth-promoting effect of the G3BP1 B domain, the inventors asked if introducing the G3BP1 B domain might alter axon regeneration in vivo. For this, adult rats were transduced with adeno-associated virus (AAV) expressing B domain, D domain, or full length G3BP1 and then subjected to sciatic nerve crush 7 d later. At 7 d after crush injury (14 d post-transduction), G3BP1-BFP, G3BP1 B domain-BFP, and G3BP1 D domain-BFP were visible in the regenerating sciatic nerve axons. The G3BP1 B domain-BFP transduced animals showed significantly increased axon regeneration compared to G3BP1-BFP, and G3BP1 D domain-BFP, and GFP transduced animals (FIG. 9C). To test for the possibility of accelerated regeneration, the inventors measured compound muscle action potentials (CMAP) in lateral gastrocnemius (LG) and tibialis anterior (TA) muscles to assess functional reinnervation after axotomy in control versus B domain-transduced animals.

Significantly accelerated recovery of CMAPs was seen with G3BP1 B domain expression in the LG at 4 and 6 wks and the TA at 4 wk after sciatic nerve crush, with control catching up by 8 wk in LG and 6 wk in TA (FIG. 9D). The apparent faster recovery in the TA likely relates to the shorter regeneration distance and smaller muscle mass compared to the LG. Taken together, these data indicate that expression of the G3BP1 B domain accelerates peripheral nerve regeneration.

To determine if a smaller region of the G3BP1 B domain is sufficient to increase axon growth, the inventors generated fluorescently labeled, cell-permeable Tat fusion peptides corresponding to residues 147-166,168-189, and 190-208 of rat G3BP1. These peptides each penetrated the neurons in DRG cultures by 30 min. after application. When added to DRG cultures immediately after plating, both the 147-166 and 190-208 peptides increased axon length; the 190-208 peptide also increased the number of neurites per neuron. Since the 190-208 peptide showed the longest axons and increased the overall number of neurites extended from each neuron, the inventors focused their efforts on this peptide, in comparison to the 168-189 peptide that lacked activity.

To discriminate between increased axon extensions versus earlier initiation of axon growth, the inventors exposed DRG cultures to peptides after the neurons had fully initiated axonal growth. With delayed application, the 190-208 peptide significantly increased axon length in both naive and preinjured DRG neurons (FIG. 9E). E18 cortical neuron cultures also showed a significant increase in axon growth when the 190-208 peptide was applied to the axonal compartment of microfluidlic culture devices (FIG. 9E). Finally, the 190-208 peptide significantly increased neurite length in cultures of motor neurons generated from human induced pluripotent stem cells. These data indicate that introducing amino acids 190-208 of rat G3BP1 increases axon growth in rodent and human neurons, and likely does so through an axon intrinsic mechanism(s).

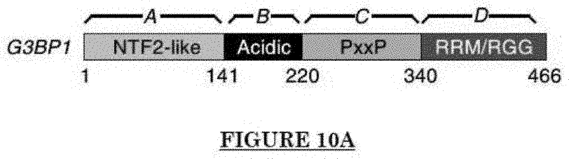

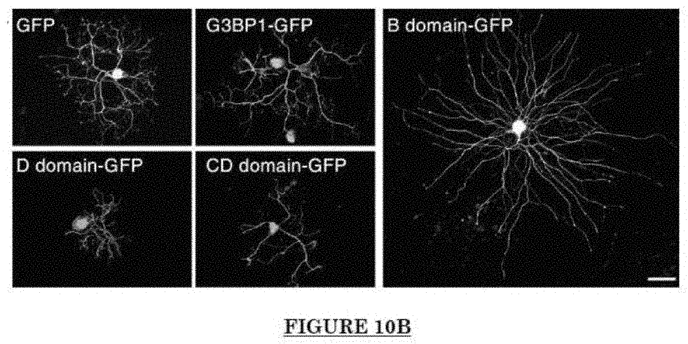

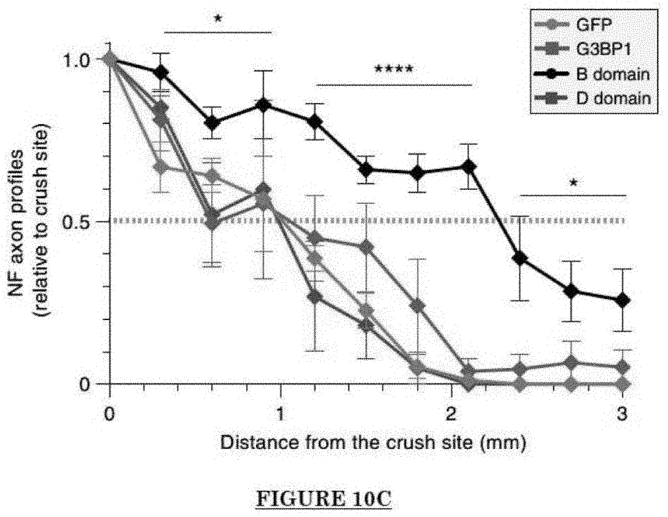

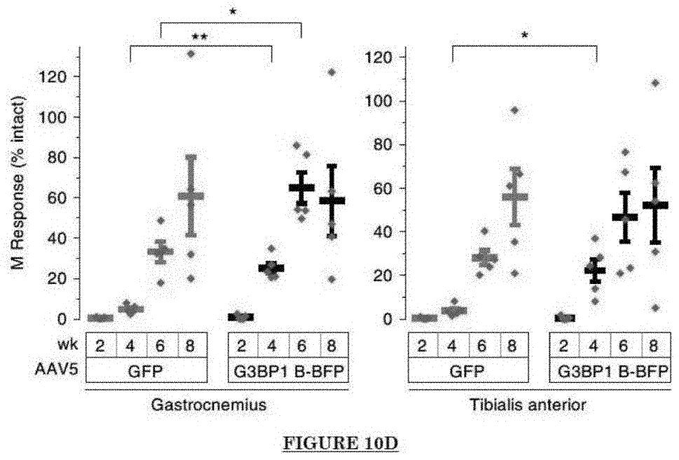

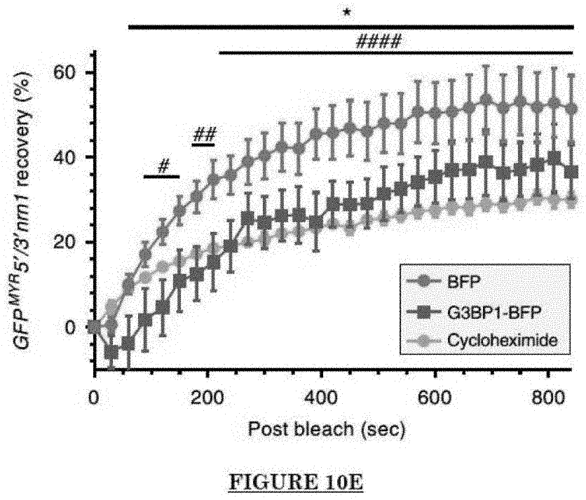

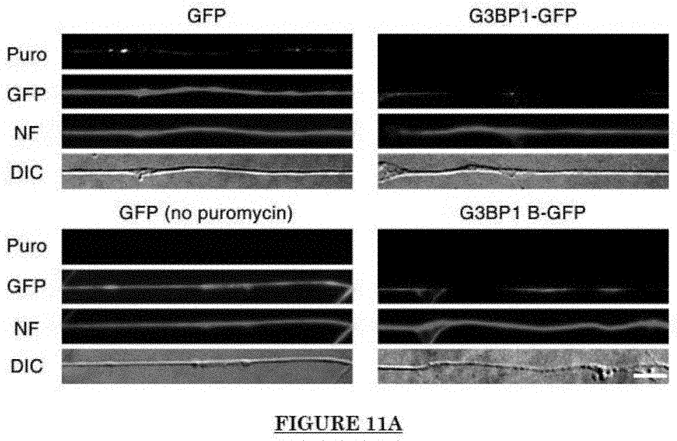

The G3BP1 acidic domain disassembles stress granule protein aggregates. To determine if expression of the G3BP1 B domain interrupts the function of endogenous G3BP1, the inventors asked if expressing the B domains alters axonal mRNA translation. Using a puromycinylation assay to test for translation of endogenous mRNAs, G3BP1 B domain expression led to significantly higher protein synthesis in axons but not cell bodies of cultured DRGs (FIGS. 10A and 10B). Depletion of G3BP1 similarly increased protein synthesis in the DRG axons with no significant effect on protein synthesis in the cell bodies (FIG. 10C).

Expression of the G3BP1 B domain also leads to increases in axonal but not in cell body levels of Nrn1 protein, without affecting axonal or cell body levels of Impβ1 or Gap43 proteins (FIG. 10D). Overexpression of full-length G3BP1 caused a decrease in axonal levels of both Nrn1 and Impβ1 proteins. Since both Nrn1 and Imp@1 mRNAs colocalized with G3BP1 and the GFP^{MYR} 5′/3′nrn1 and GFP^{MYR} 5′/3′impb1 reporter mRNAs coprecipitated with G3BP1, the inventors asked if endogenous Nrn1 and Impβ1 mRNA binding to G3BP1 might be affected by the introduction of the B domain. Co-precipitation of these mRNAs with G3BP1-BFP was significantly reduced in neurons co-transfected with B domain-BFP construct (FIG. 10E).

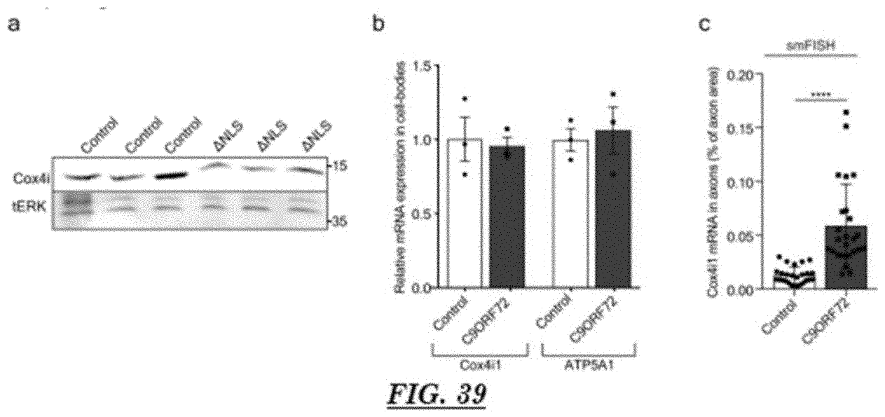

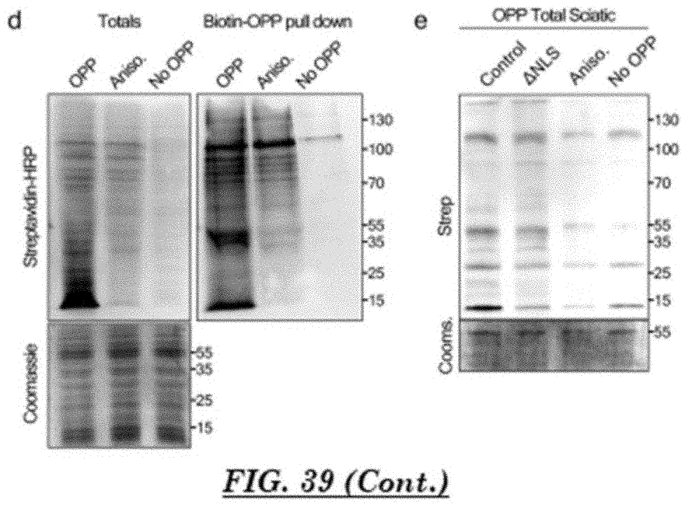

GAP43 mRNA did show some binding to G3BP1-BFP, but this was not affected by the B domain expression, and none of these mRNAs precipitated with G3BP1 B domain-GFP or the control GFP (FIG. 10E). These data suggest that the G3BP1 B domain increases axonal protein synthesis by causing release of mRNAs from axonal G3BP1 aggregates. FIG. 39, TDP-43 RNP-condensates limit the axonal synthesis of nuclear-encoded mitochondrial proteins, shows at a) Uncropped western blot of Cox4i protein levels in isolated axons of TDPΔNLS MNs cultured in radial MFC. n=3 experiments. b) RT-qPCR of Cox4i1 and ATP5A1 mRNA levels from somata of C9ORF72 and control iPS-MNs. n=3 independent repeats. SE. Unpaired-t-test, two-sided. c) quantification of Cox4i1 mRNA abundance in C9ORF72 and control iPS-MN axons. n=24,25 images from 3 repeats. SD. Unpaired-t-test, two-sided ****p<0.0001. d) OPP pull-down controls in HEK293T cells validation for specificity and sensitivity of assay. Left panel: Streptavidin-HRP (top) and Coomassie stained blots (bottom) of input lysates (Totals) from OPP-treated cultures compared to Anisomycin (Aniso.) and NoOPP controls. Right panel: Streptavidin-HRP blot of same samples following Biotin-OPP streptavidin pulldown. n=1 experiment per condition e) Streptavidin-HRP (top) and Coomassie stained (bottom) blots from OPP-labeled sciatic nerve axoplasms of Control, TDPΔNLS, Anisomycin-treated, and no-OPP controls. n=6 sciatic nerves (from 3 mice) per lane.

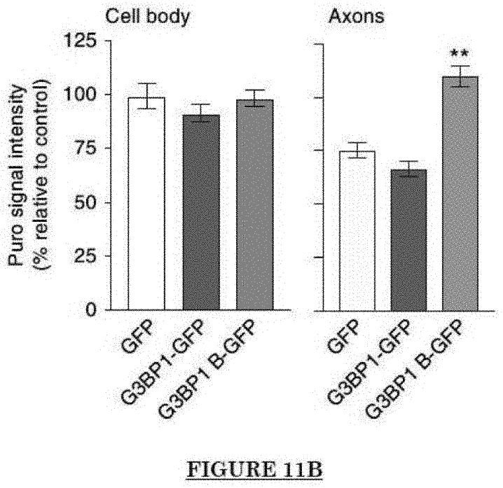

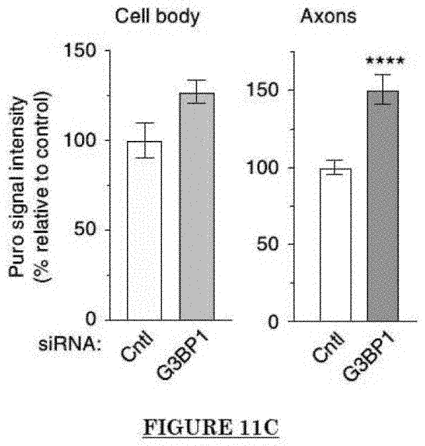

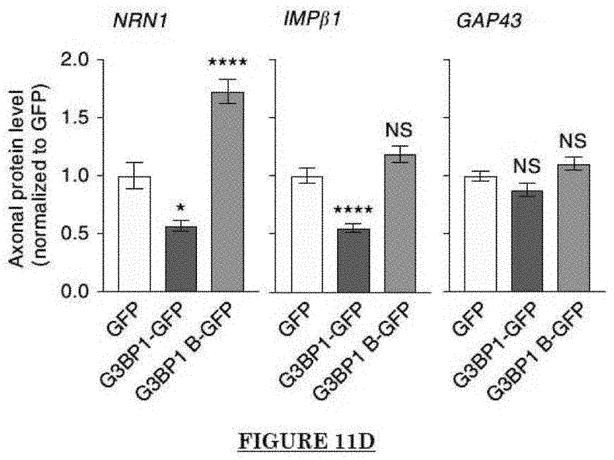

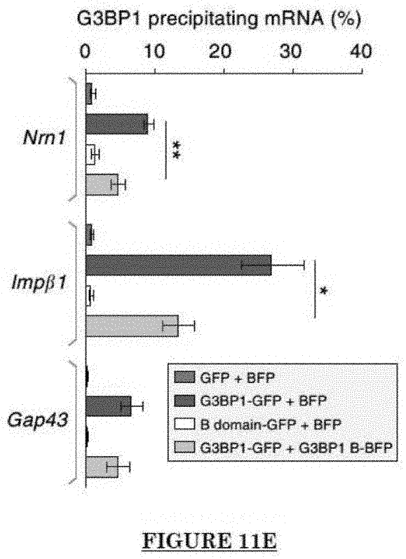

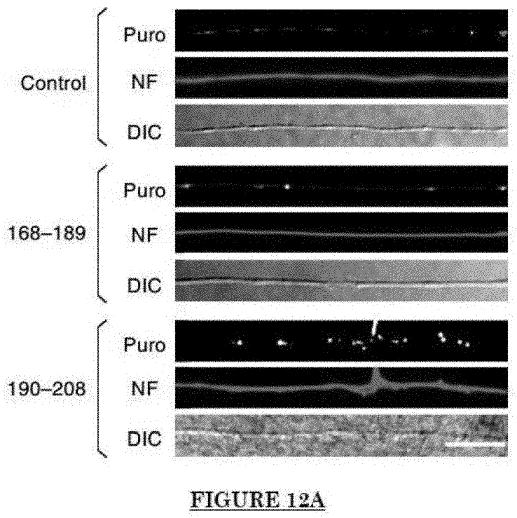

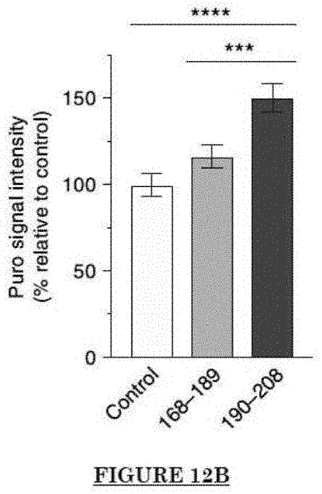

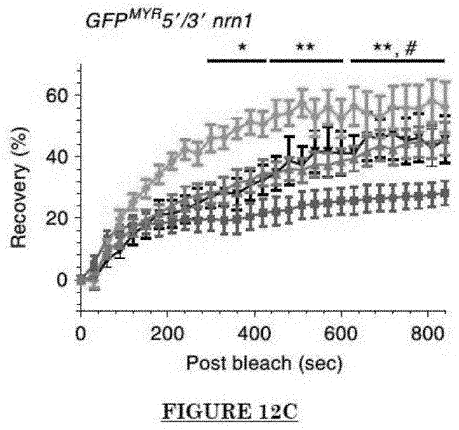

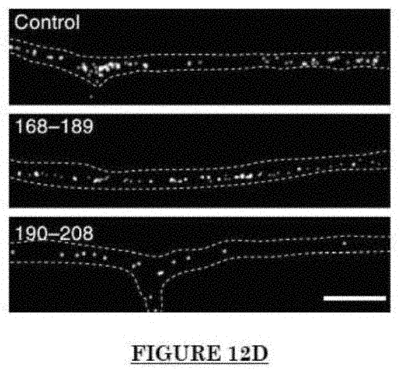

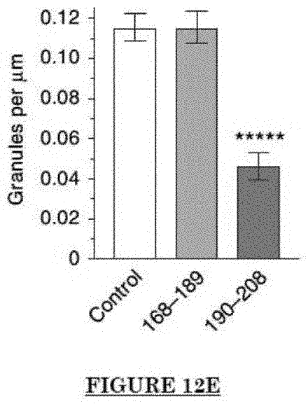

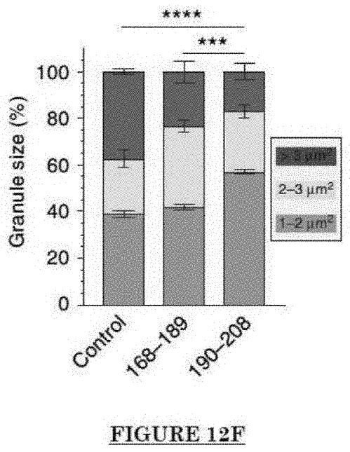

In light of the changes in translation upon expression of the G3BP1 B domain, the inventors asked if the growth-promoting 190-208 peptide might also affect axonal protein synthesis. Treatment with the cell-permeable G3BP1 190-208 peptide significantly increased puromycin incorporation in DRG axons compared to untreated and G3BP1 168-189 peptide-treated cultures (FIGS. 11A and 11B). The 190-208 peptide also significantly increased axonal recovery of GFP^{MYR} 5′/3′nrn1 fluorescence after photobleaching under control conditions and reversed the decrease in axonal translation seen with G3BP1 overexpression (FIG. 11C). The lack of effect previously observed for B domain expression on endogenous Impβ1 protein levels in axons was mirrored by the 190-208 peptide effects on GFP^{MYR} 5′/3′impβ1 and mCh^{MYR} 5′/3′gap43 translation in axons under control conditions. Moreover, the 190-208 peptide did not rescue the decline in axonal translation of GFP^{MYR} 5′/3′impβ1 seen with G3BP1 overexpression (FIG. 11C). Together, these data indicate that disrupting G3BP1 function with overexpression of the B domain or the 190-208 G3BP1 peptide can specifically increase intra-axonal translation of some mRNAs. Considering the effects of the G3BP1 B domain and 190-208 peptide on axonal mRNA translation, the inventors reasoned that these agents might disrupt SGs. Indeed, G3BP1 B domain expression attenuated SG aggregation in NIH 3T3 cells exposed to sodium arsenite.

As noted above, arsenite is a potent inducer of SG aggregation, so the inventors performed time-lapse imaging on DRG cultures to determine if the G3BP1 B domain affects the axonal SG-like structures. To this end, the inventors treated DRG cultures with the cell-permeable 190-208 G3BP1 peptide, and monitored G3BP1-mCherry aggregates. The 190-208 peptide caused a striking decrease in axonal G3BP1-mCherry aggregates after 15 min. (FIG. 11D and 11E). Moreover, the remaining SG-like structures in axons were significantly smaller after 190-208 peptide treatment (FIG. 11F), and the remaining aggregates showed greater motility than in control conditions. In contrast, the 168-189 G3BP1 peptide caused no change in SG density along the axons (FIGS. 11D and 11E), and only modest but insignificant decrease in SG size (FIG. 11F). Together, these data indicate that the B domain expression and treatment with the 190-208 G3BP1 peptide disrupt aggregation of SG-like structures.

FIGS. 9A-9E show G3BP1 acidic domain expression accelerates nerve regeneration. a Schematic of G3BP1 domains as defined by Tourriere et al. (2003). b Representative images for NF-labeled DRG neurons transfected with indicated constructs are shown. Images were acquired at 60 h post-transfection [scale bar=100 p.m]. c Extent of axon regeneration at 7 d post sciatic nerve crush in adult rats transduced with AAVS encoding G3BP1-BFP, G3BP1 B domain-BFP, G3BP1 D domain-BFP, or GFP control is shown as mean axonal profiles relative to crush site (0 mm)±SEM. *=p≤0.05 and ****=p≤0.0001 between B domain-BFP versus GFP transduced animals by one-way ANOVA with Tukey HSD post-hoc). d Animals transduced with AAV5 encoding G3BP1 B domain-BFP versus GFP were subjected to sciatic nerve crush and regeneration was assessed by muscle M response in tibialis anterior and gastrocnemius. Values are shown as average % intact M responses ±SEM (lines) with data points for individual animals plotted (*p≤0.05 and **p≤0.01 for B domain versus GFP by Student's t-test for indicated data pairs). e Quantitation of axon growth from DRGs (left) and cortical neurons (right) treated with cell-permeable 168-189 or 190-208 G3BP1 peptides is shown. For DRGs, peptides were added to dissociated naive or 7 d injury-conditioned DRGs at 12 h and axon growth was assessed at 36 h in vitro. For cortical neurons, peptides were added to the axonal compartment of microfluidic devices at 3 d in vitro (DIV), and axon growth was assessed at 6 DIV. ***p≤0.005 by one-way ANOVA with Tukey HSD post-hoc)

FIGS. 10A-10E show G3BP1 acidic domain increases axonal mRNA translation and disassembles stress granules. a, b Representative images for puromycin (Puro) incorporation in DRG neurons transfected with the indicated constructs are shown (a). Significant increase in axonal puromycin signals in the G3BP1 B domain-expressing neurons is seen, with no significant change in the cell body puromycin incorporation (b; N≥23 axons over three repetitions; **p≤0.01, ****p≤0.0001 by one-way ANOVA with Tukey HSD post hoc) [scale bar=5 μm]. c G3BP1 depleted DRG cultures similarly show increased puromycin incorporation in axons with no significant change in cell body puromycin incorporation (N≥23 axons over three repetitions; **p≤0.01, ****p≤0.0001 by one-way ANOVA with Tukey HSD post hoc). d Quantitation of endogenous axonal NRN1, IMP@1, and GAP43 protein levels in DRG cultures transfected with GFP, G3BP1-GFP, and G3BP1 B domain-GFP is shown.

Axonal NRN1 and IMP@ 1 but not GAP43 levels are significantly reduced in G3BP1 overexpression. G3BP1 B domain-expressing neurons show significantly higher axonal NRN1, but no change in axonal IMPβ1 and GAP43 levels (N≥33 axons over three repetitions; *p≤0.05, ****p≤0.0001 by one-way ANOVA with Tukey HSD post hoc). e RTddPCR for axonal mRNAs co-precipitating with G3BP1-GFP in DRG neurons are shown as average % mRNA associated with G3BP1-GFP±SEM. Nrnl and Impβ1 mRNAs association with G3BP1-GFP significantly reduced by cotransfection with the G3BP1 B domain, but neither RNA coprecipitates with the B domain (N=4 culture preparations; * p≤0.05, **p≤0.01 by Student's t-test for the indicated data pairs)

Studies have now documented mRNA translation in axons, and this is particularly prominent in the PNS where intra-axonal protein synthesis contributes to axon regeneration after injury. Some SG proteins have been detected in axons of PNS nerves, but intra-axonal functions for these proteins were only inferred from known functions of these proteins in other cellular systems. The inventors' data indicate that blocking G3BP1′s function in the assembly of axonal S and accelerates PNS axon regeneration. Thus, axonal G3BP1 is a negative modulator of intra-axonal protein synthesis and axon growth. Since several thousand mRNAs have been identified in axons of cultured neurons, it is likely that translation of many axonal mRNAs could be regulated by G3BP1, as the inventors show here for Impβ1 and Nrn1 mRNAs. The colocalization of different mRNAs G-like structures increases intra-axonal protein synthesis with these G3BP1 aggregates correlates with the growth status of neurons, and blocking G3BP1 aggregation provides a novel strategy to accelerate regeneration.

Assembly of SGs has been well characterized in response to different metabolic and oxidative stressors in non-neural systems. The rapid increase in SG-like structures seen here by 3 h after axotomy could reflect a stress response by the PNS axons, with a decrease in SG-like aggregates during axon growth at later time points. The decrease in G3BP1 aggregation was accompanied by an increase in phospho-G3BP1. Casein kinase 2 and AKT have recently been reported to phosphorylate G3BP1 on Ser 149 in other cellular systems. Both of these proteins are present in axons, and it will be of interest for future work to determine their roles in intra-axonal signaling cascades regulating G3BP1 phosphorylation. Notably, the inventors see aggregates of G3BP1 and TIA1 in uninjured PNS axons and G3BP1 aggregates in axon shafts of cultured neurons with growing axons. The inventors suspect that these structures correspond to the ‘core SGs’ that have been defined in other cell types. Recent proteomics analyses for SG protein interactomes, including the G3BP1 protein analyzed herein, point to pre-assembly of some SG proteins under non-stress conditions. These interactomes also included HuR (also known as ELAVL1), FXR1, FMRP, and TIA1 proteins that the inventors show colocalize with axonal G3BP1 protein. Consistent with the possibility of a core SG present in uninjured and growing axons, as the inventors' work suggests, core SG components were recently shown to interact in neurites of human IPSC-derived motor neurons before application of arsenic.

The inventors study notably shows that axonal G3BP1 and TIA1 do not always colocalize. Likewise, the Pearson's coefficient for colocalization of G3BP1 with SG components is low despite being statistically higher than the coefficients for G3BP1 with PB proteins. Overexpression of G3BP1 was shown to precipitate SG assembly in the absence of any stress in non-neuronal cells. Only some of the aggregates seen with overexpressed G3BP1 colocalize with TIA1, but G3BP1-associating mRNAs were found in both TIA1-positive and TIA1-negative G3BP1 aggregates. Thus, it is likely that the axonal G3BP1 aggregates seen here that are separate from TIA1 can also interact with mRNAs.

Regardless of whether the axonal G3BP1 aggregates are classic SGs or even core SG aggregates, the inventors' data clearly show these axonal aggregates attenuate axonal protein synthesis and limit rates of axon growth, so the axonal G3BP1 aggregates are biologically significant. Future studies will be needed to compare and contrast the constituents of these axonal G3BP1 aggregates to those of classic SGs.

The translation of different axonal mRNAs will undoubtedly show a high degree of regulatory complexity, and it is likely that numerous axonal mRNAs could be regulated by G3BP1 interactions.

Interestingly, all axonal mRNAs were not regulated by the G3BP1 aggregates, since Gap43 showed comparatively low interaction with G3BP1 and its translation was not affected by G3BP1 overexpression or B domain manipulations. This may reflect differences in post-transcriptional regulation between Gap43, Nrn1 and Impβ1 mRNAs in axons. Impβ1 mRNA is constitutively transported into axons, where its localized translation is activated through Ca²⁺-dependent pathways after axotomy. In contrast, Nrn1's transport into axons is increased after axotomy, with the mRNA shifting from soma-predominant to axon-predominant during regeneration. On the other hand, Gap43's transcription is increased approximately 5 fold after axotomy, with increased axonal localization commensurate with an overall increase in Gap43 levels. Based on the low colocalization of Gap43 with G3BP1 and the lack of effect of G3BP1 on its translation, Gap43 mRNA does not seem to be regulated by the axonal SG-like structures. It is intriguing to speculate that differences in transcriptional versus post-transcriptional regulation contribute to whether individual mRNAs are regulated by the axonal SG-like structures. Such distinctions would segregate the axonal transcriptome into mRNA cohorts based on the mechanisms of their transcriptional and/or translational regulation and axonal transport.

The difference between Impβ1 and Nrn1 colocalization with G3BP1 in naive versus injury-conditioned neurons likely reflects different needs for the corresponding proteins in different growth states. DRG neurons that are pre-injured by an in vivo axotomy days prior to culturing show rapid elongation of relatively unbranched axons that is transcription independent. This rapid axonal growth occurs through translational control of existing mRNAs, and the injury-conditioned neurons show higher intra-axonal protein synthesis than naive DRG neurons. Nrnl protein promotes neurite growth, and increasing axonal targeting of Nrnl mRNA increases axon growth. Hence, the decrease in Nrnl mRNA associated with SG-like aggregates in axons of injury-conditioned neurons would free the mRNA for translation to promote axon growth. On the other hand, Impβ1 mRNA translation is induced by axotomy, with its protein product providing a retrograde signal to activate regeneration-associated gene expression in the soma. Continued translation of Impβ1 mRNA likely decreases axon elongation due to its role in axon length sensing. Consequently, rapid axon growth after injury conditioning could also be facilitated by sequestering Impβ1 mRNA from translation.

Both Nrn1 and Impβ1 mRNAs were released from interaction with G3BP1 when the G3BP1 B domain was introduced. Since the B domain did not co-precipitate these mRNAs, the release of mRNAs from G3BP1 interaction is not via a competitive interaction for mRNA binding by the B domain with full length G3BP1. Though axonal translation of both Nrnl and Impβ1 was decreased by overexpression of G3BP1, only Nrnl showed increased translation in response to B domain expression and 190-208 peptide treatment. This indicates that the release of stored mRNAs in axons is not sufficient for their translation.

Additional stimuli are undoubtedly needed to translate Impβ1 mRNA compared to Nrn1 mRNA. Impβ1 mRNA was initially shown to be translated in axons after injury and this requires an increase in axoplasmic Ca₂₊ levels. Increased Ca₂₊ is known to trigger phosphorylation of eIF2α, and phosphomimetic eIF2α was shown to increase the translation of axonal Calr and Hspa5 mRNAs in cultured DRG neurons. Thus, axoplasmic Ca₂₊ levels may provide one regulatory mechanism for determining which mRNAs are translated upon release from the axonal SG-like structures. Differential susceptibility to mTOR regulation may provide an additional layer of regulation, as frequently reported for survival promoting retrograde injury signals in peripheral nerve.

In summary, the inventors study points to axonal G3BP1 as a specific modulator of intra-axonal protein synthesis and axon growth. Since G3BP1 is aggregated in uninjured PNS axons, the inventors' data point to unrealized functions for SG-like aggregates in axons under non-stress conditions. Preventing this SG-like aggregation of axonal proteins during regeneration increases the rate of axon regrowth. Considering that Tat fusion peptides for NR2B9c have been used in a clinical trial for ischemic protection during endovascular repair for intracranial aneurysms, the growth promoting effects of the cell-permeable 190-208 G3BP1 peptide may represent a novel therapeutic lead for accelerating nerve regeneration. Since peripheral nerves typically regenerate at only 1-2 mm per day, accelerating axon growth rates by interfering with axonal G3BP1 function could significantly shorten recovery times and allow axons to reach a more receptive environment to reinnervate target tissues.

FIGS. 11A-11F show Cell permeable G3BP1 190-208 peptide increases axonal mRNA translation and disassembles stress granules. a, b, Representative images for puromycin incorporation in axons of control, 168-189 peptide and 190-208 peptide-treated DRG cultures are shown (a). Quantitation of puromycin incorporation into distal DRG axons under these conditions shows a significant increase in axonal protein synthesis for the 190-208 peptide-treated cultures compared to control and 168-189 peptide exposure (b; N≥83 axons over 3 DRG cultures; ***p≤0.005, ****p≤0.0001 by one-way ANOVA with Tukey HSD post-hoc). c FRAP analyses for DRGs for GFP^{MYR} 5′/3′nrn1, GFP^{MYR} 5′/3′impβ1 and GFP^{MYR} 5′/3′gap43 in axons of DRGs expressing BFP or G3BP1-BFP±10 μM 190-208 G3BP1 peptide (30 min. treatment). Only translation of GFP^{MYR} 5′/3′nrn1 is increased by the 190-208 peptide with G3BP1 overexpression (N≥11 axons over three culture repetitions; all statistics were done by one-way ANOVA with Tukey HSD post-hoc: *p≤0.05, **p≤0.01 for BFP versus BFP+190-208 peptide; #p≤0.05 for G3BP1-BFP versus G3BP1-BFP+190-208; p≤0.05 for BFP versus G3BP1; and, †p≤0.05 for BFP versus G3BP1-BFP+190-208 peptide; no values for GFP^{MYR} 5′/3′gap43 were statistically significant). d Representative images of G3BP1-mCh in DRG axons under control conditions and after treatment with 190-208 G3BP1 or 168-189 peptides for 15 min. are shown. Axon tracing was generated from DIC images [scale bar=10 μm]. e Density of G3BP1-mCh aggregates along 100 μm length axons from DRG cultures treated as in d is shown (N≥38 axons over three repetitions; ****p≤0.0001 by ANOVA with Tukey HSD post-hoc). f Size of G3BP1-mCh aggregate is shown as indicated bins for from DRG cultures treated as in d (N≥221 aggregates over three repetitions; ****p≤0.0005, *****p≤0.0001 for entire population distributions by Kolmogorov—Smirnov test)

Methods

Animal use and survival surgery. Institutional Animal Care and Use Committees of University of South Carolina, Emory University, and Weizmann Institute of Science approved all animal procedures. Male Sprague Dawley rats (175-250 g) were used for all sciatic nerve injury and DRG culture experiments. Embryonic day 18 (E18; male and female) rat pups were used for cortical neuron culture experiments.

Isofluorane was used for anesthesia for AAV transduction and peripheral nerve injuries, and ketamine plus xylazine was used for electrophysiology studies (see below).

For peripheral nerve injury, anesthetized rats were subjected to a sciatic nerve crush at mid-thigh as described. In cases where animals were transduced with virus prior to injury, AAV5 was injected into the proximal sciatic nerve 7 d prior to crush injury (at sciatic notch level; 9-14×10¹⁰particles in 0.6 M NaCl).

Axoplasm was obtained from sciatic nerve at 3-28 d after crush injury at mid-thigh level. Approximately 3 cm segments of nerve proximal to the injury site (or equivalent level on contralateral [naive] side) were dissected and axoplasm extruded into 20 mM HEPES [pH 7.3], 110 mM potassium acetate, and 5 mM magnesium acetate (nuclear transport buffer) supplemented with protease/phosphatase inhibitor cocktail (Roche) and RNasin Plus (Promega). After clearing by centrifugation at 20,000×g, 4° C. for 30 min., supernatants were mixed with 3 volumes of Trizol LS (Invitrogen) and processed for mass spectrometry (see below). Three animals were used for each time point.

Cell culture. For primary neuronal cultures, L4-5 DRG were harvested in Hybernate-A medium (BrainBits) and then dissociated as described. After centrifugation and washing in DMEM/F12 (Life Technologies), cells were resuspended in DMEM/F12, 1×N1 supplement (Sigma), 10% fetal bovine serum (Hyclone), and 10 μM cytosine arabinoside (Sigma). Dissociated DRGs were plated immediately on laminin/poly-L-lysine-coated coverslips or transfected (see below) and then plated on coated coverslips.

For cortical neuron cultures, E18 cortices were dissected in Hibernate E (BrainBits) and dissociated using the Neural Tissue Dissociation kit (Miltenyi Biotec). For this, minced cortices were incubated in a pre-warmed enzyme mix at 37° C. for 15 min; tissues were then triturated and applied to a 40 μm cell strainer.

After washing and centrifugation, neurons were seeded at a density of 1×10⁵ cells per poly-D-lysine-coated microfluidic device (Xona Microfluidics). NbActive-1 medium (BrainBits) supplemented with 100 U/ml of Penicillin-Streptomycin (Life Technologies), 2 mM L-glutamine (Life Technologies), and 1×N21 supplement (R&D Systems) was used as culture medium.

Human induced pluripotent stem cells (hiPSCs) were maintained in dishes coated with Matrigel (Corning) in Flex8 media (ThermoFisher). hiPSCs were differentiated into human motor neurons using a directed differentiation protocol optimized by Kevin Eggan. 7000 neurons/well were plated on laminin—or CSPG coated 96 well plates. 100 μl at 10 μg/ml Laminin (ThermoFisher) and 25 ng CSPGs (Millipore) were used per well.

NIH-3T3 and HEK293T cells were maintained in DMEM (Life Technologies) supplemented with 10% FBS (Gibco) and 100 U/ml of Penicillin-Streptomycin (Life Technologies).

For DRG neuron transfection, dissociated ganglia were pelleted by centrifugation at 100×g for 5 min and resuspended in ‘nucleofector solution’ (Rat Neuron Nucleofector kit; Lonza). 5-7 μg plasmid was electroporated using an AMAXA Nucleofector apparatus (program SCN-8; Lonza). For siRNA transfection, 100 nM siRNAs (Dharmacon) were used with DharmaFECT 3 reagent and incubated for 36 h. A 3′UTR targeted siRNA (5′CCACAUAGGAGCUGGGAAUUU 3′) was used for depleting G3BP1 for experiments assessing axon growth where siRNA-resistant G3BP1 constructs were used for rescue. Dharmacon On-target plus-SMART pool siRNA (Cat no. L101659-02-0005) against G3bp1 was used in antibody specificity testing and Puromycinylation assays. Non-targeting siRNAs were as control. RTddPCR and immunoblotting was used to test the efficiency of G3BP1 depletion (see below).

HEK293T cells were transfected using Lipofectamine® 2000 per manufacturer's instructions (Invitrogen). AAV5 preparations were titrated in DRG cultures by incubating with 1.8-2.8×10¹⁰ particles of AAV5 overnight.

For arsenic treatment to induce SG aggregation, transfected NIH3T3 cells were grown to 60-80% confluence and were then treated with 0.5 mM sodium arsenite (Sigma) for 30 min.

For peptide treatments, 10 μM Tat-fused peptides were added to dissociated DRG cultures at 2 or 12 h after plating. Neurite outgrowth was assessed 24 h after addition of peptides. For the cortical cultures, 10 μM peptide was applied to the axonal compartment at 3 d in vitro (DIV) and axonal growth was assessed at 6 DIV. For iMotor neurons, 20 μM peptides were immediately added and neurite growth was assessed 24 h later.

Plasmid and viral expression constructs. All fluorescent reporter constructs for analyses of RNA translation were based on eGFP with myristoylation element (GFP^{MYR}; originally provided by Dr. Erin Schuman, Max-Plank Inst., Frankfurt) or mCherry plasmid with myristoylation element (mCh^{MYR}). Reporter constructs containing 5′ and 3′UTRs of rat Nrn1 and Gap43 mRNAs have been published. For Impβ1, the rat 5′UTR was cloned by PCR and inserted directly upstream of the initiation codon in GFP^{MYR} 5′/3′impβ1.

Human G3BP1 wild type, S149A, S149E and deletion constructs as GFP-tagged proteins were generously provided by Dr. Jamal Tazi, Institut de Génétique Moléculaire de Montpellier. The G3BP1-mCherry construct was generated by PCR, amplifying G3BP1 coding sequence with 5′ NheI and 3′ HindIII restriction sites. After NheI+HindIII digestion, G3BP1 CDS was subcloned into NheI+HindIII-digested pmCherry-N1 vector (Clontech).

AAV5 preparations were generated in UNC Chapel Hill Viral Vector Core. All plasmid inserts were fully sequenced prior to generating AAV. BglII+XhoI digested human G3BP1 cDNA (from pGFP-G3BP1) was subcloned into BamHI+XhoI digested pAAV-cDNA6-V5His vector (Vector Biolabs). G3BP1 deletion constructs were amplified by PCR with terminal HindIII and XhoI restriction sites (primer sequences available on request). After digestion with HindIII and XhoI, products were cloned into HindIII+XhoI-digested pAAV-cDNA6-V5His vector.

BFP was excised from the pTagBFP-N vector (Evrogen) using EcoRI+NotI and ligated in-frame directly 3′ to the G3BP1 sequences in pAAV-cDNA6-V5His.

Generation of Tat-tagged G3BP1 B domain peptides. Three peptides were generated from the rat G3BP1 B domain sequence (amino acids 140-220; UniProt ID #D3ZYS7_RAT) by Bachem Americas, Inc. Peptides were synthesized with Nterminal dansyl chloride or FITC and N- or C-terminal HIV Tat peptide for cell permeability; the Tat sequence was placed at the least conserved end of the sequence based on P-BLAST of vertebrate G3BP1 sequences available in UniProt database. Peptide sequences were: 147-166, EESEEEVEEPEENQQSPEVV-YGNKKNNQNNN; 168-189, DDSGTFYDQTVSNDLEEHLEEP-YGNKKNNQNNN; and 190-208, YGNKKNNNQNNN-VVEPEPEPEPEPEPEPVSE. Meanvwhile, human G3BP1 peptide, NCBI Reference Sequence: NP_005745.1, is 189-209, EPVAEPEPDPEPEPEEEPVSE and may be applied as a treatment method for patients as described herein.

Immunofluorescent staining. All procedures were performed at room temperature (RT) unless specified otherwise. Cultured neurons were fixed in 4% paraformaldehyde (PFA) in phosphate-buffered saline (PBS) and processed as described. Primary antibodies consisted of: rabbit anti-G3BP1 (1:200, Sigma), RT97 mouse anti-neurofilament (NF; 1:500, Devel. Studies Hybridoma Bank), goat anti-NRN1 (1:100, Novartis), rabbit anti-IMM (1:100, My Biosciences), rabbit anti-GAP43 (1:5000, Novus), and rabbit anti-G3BP1^{S149}(1:300, Sigma). FITC conjugated donkey anti-rabbit and Cy3-conjugated donkey anti-mouse (both at 1:200, Jackson ImmunoRes.) were used as secondary antibodies.

For G3BP1 colocalization with SG and PB proteins, Zenon antibody labeling kit (Life Technologies) was used to directly label antibodies with fluorophores.

Combinations of rabbit anti-G3BP1 (Sigma)+Alexa-488, rabbit anti-HuR (Millipore)+Alexa-405, rabbit anti-FMRP (Cell Signaling Tech)+Alexa-555, and rabbit anti-FXR1 (a kind gift from Dr. Khandiah, Institut Universitaire en Santé Mentale de Québec)+Alexa-633 or rabbit anti-G3BP1+Alexa-488, rabbit anti-DCP1A (Abcam)+Alexa-405, and rabbit anti-XRN1 (Bethyl Lab)+Alexa-633 were used at 1:50 dilution for each antibody. Equal amounts of rabbit-IgG labeled with Alexa-405, -488, -555 and -633 were used as control.

For quantifying axonal content of G3BP1, TIA1, and G3BP1^{PS149} in peripheral nerve, sciatic nerve segments were fixed for 4 h in 4% PFA and then cryoprotected overnight in 30% sucrose, PBS at 4° C. 10 μm cryostat sections were processed for immunostaining as previously described. Primary antibodies consisted of rabbit anti-G3BP1 (1:100), rabbit anti-phospho-G3BP1^{PS149} (1:100), and RT97 mouse anti-NF (1:300). Secondary antibodies were FITC-conjugated donkey anti-rabbit and Cy3-conjugated donkey anti-mouse (both at 1:200, Jackson ImmunoRes.).

Immunoblotting confirmed the specificity of the anti-G3BP1 and -G3BP1^{PS149} antibodies and by immunofluorescence signals for both antibodies were decreased in DRGs transfected with siRNAs to G3BP1.

Paraffin sections were used for analyses of nerve regeneration. For this, 10 μm thick paraffin sections of sciatic nerve were deparaffinized in 100% xylene (2×10 min) followed by 100% ethanol (2×10 min). Sections were rehydrated by sequential incubations in 95, 75 and 50% ethanol for 5 min each, and then rinsed in deionized water. Sections were permeabilized in 0.3% Triton X-100 in PBS, and then rinsed in PBS for 20 min and equilibrated in 50 mM Tris [pH 7.4], 150 mM NaCl, 1% heat-shock bovine serum albumin (BSA), and 1% protease-free BSA (Roche) (‘IF buffer’). Sections were then blocked in IF buffer plus 2% heat-shock BSA, and 2% fetal bovine serum for 1.5-2 h. After blocking, samples were incubated overnight at 4° C. in a humidified chamber with the primary antibodies in IF buffer. Samples were washed in IF buffer three times and then incubated with secondary antibodies diluted in IF buffer for 45 min. Samples were washed in IF buffer three times followed by a rinse in PBS and deionized water. Primary antibodies consisted of: RT97 mouse anti-NF (1:300) and rabbit anti-RFP (1:100, Rockland Immun. Chem.). The RFP antibody was confirmed to detect BFP by immunoblotting (see below) and immunolabeling of transfected DRG neurons (data not shown). Secondary antibodies were used as above.

All samples were mounted with Prolong Gold Antifade (Invitrogen) and analyzed by epifluorescent or confocal microscopy. Leica DMI6000 epifluorescent microscope with ORCA Flash ER CCD camera (Hamamatsu) or Leica SP8X confocal microscope with HyD detectors was used for imaging unless specified otherwise. For quantitation between samples, imaging parameters were matched for exposure, gain, offset and post-processing. For protein—protein colocalizations, HyVolution (Leica/Huygens) deconvolution was used to optimize optical resolution in confocal image stacks acquired with parameters optimized for this post-processing.

Fluorescence in situ hybridization (FISH). For FISH, DRG cultures were fixed for 15 min in 2% PFA in PBS. RNA-protein colocalization was performed using custom 5′ Cy3-labeled ‘Stellaris’ probes (probe sequences available upon request; BioSearch Tech.). Scrambled probes were used as control for specificity; samples processed without the addition of primary antibody were used as control for antibody specificity. Primary antibodies consisted of rabbit anti-G3BP1 (1:100) and RT97 mouse anti-NF (1:200). FITC-conjugated donkey anti-rabbit and Cy5-conjugated donkey anti-mouse (both at 1:200) were used as secondaries. Samples were mounted as above and analyzed using a Leica SP8X confocal microscope.

Samples were post-processed with HyVolution integrated into the Leica LAX software and analyzed as outlined below for RNA-protein colocalization.

Proximity ligation assay (PLA). PLA has been used to show protein colocalization within a range of approximately 40 nm. For this, the inventors used Duolink kit per the manufacturer's instructions (Sigma). Briefly, dissociated DRGs were cultured for 48 h, fixed with 4% PFA in PBS. Samples were blocked and permeabilized in PBS plus 0.1% Triton X-100, 5% donkey serum, 1% BSA for 30 min. Samples were incubated with the following primary antibodies overnight at 4° C. in PBS plus 1% donkey serum: rabbit anti-G3BP (1:100), mouse anti-HuR (1:100), and mouse anti-DCP1a (1:100). After washing in PBS, samples were incubated with PLA reagent±probes for 1 h at 37° C. Following three washes in 0.01 M Tris [pH 7.4], 0.15 M NaCl and 0.05% Tween 20 (‘buffer A’), ligation-ligase mix was applied and samples were incubated for 30 min at 37° C. Subsequently, samples were washed 2× in buffer A, then the amplification-polymerase mix was added and samples were incubated for 110 min at 37° C. Finally, coverslips were washed three times in 0.2 M

Tris-Cl [pH 7.5], 0.1 M NaCl (‘buffer B’) and then incubated with chicken anti-NF H antibody (1:2000; Abcam) for 45 min at RT. Coverslips were washed in buffer B three times, incubated for 45 min with Alexa 488-conjugated donkey anti-chicken (Jackson ImmunoRes., 1:1000), washed and mounted with Mowiol. PLA with only one of the two primary antibodies (but adding both PLA probes) was used as a technical control.

Imaging was performed using an Olympus FV1000 confocal microscope (60×/NA 1.35 UPLSAPO oil immersion objective). Only NFH positive neurites at >200 μm distances from the cell body were analyzed using Fiji software. Ostu thresholding was applied to generate a binary mask of the NFH signal, and PLA signal was then detected using the “Find Maxima . . . ” function.

Fluorescence recovery after photobleaching (FRAP). FRAP was used to test for axonal mRNA protein synthesis using diffusion-limited GFP^{MYR} and mCherry^{MYR} reporters as described with minor modifications. In each case, DRG neurons were co-transfected with GFP^{MYR} 5′/3′nrn1+mCherry^{MYR} 5′/3′gap43 or GFP^{MYR} 5′/3′impβ1+mCherry^{MYR}5′/3′gap43 so that recovery of both reporters could be analyzed simultaneously. Cells were maintained at 37° C., 5% CO₂ during imaging sequences. 488 nm and 514 nm laser lines on Leica SP8X confocal microscope were used to bleach GFP and mCherry signals, respectively (Argon laser at 70% power, pulsed every 0.82 s for 80 frames). Pinhole was set to 3 Airy units to ensure full thickness bleaching and acquisition (63×/1.4 NA oil immersion objective). Prior to photobleaching, neurons were imaged every 60 s for 2 min to acquire baseline fluorescence the region of interest (ROI; 15% laser power, 498-530 nm for GFP and 565-597 nm for mCherry emissions, respectively). The same excitation and emission parameters were used to assess recovery over 15 min post-bleach with images acquired at 30 s intervals. To determine if fluorescence recovery in axons was from translation, cultures were treated with 150 μg/ml cycloheximide (Sigma) or 100 μm anisomycin (Sigma) for 30 min prior to photobleaching for GFP^{MYR} 5′/3′nrn1+mCherry^{MYR} 5′/3′gap43 and GFP^{MYR} 5′/3′impβ1+mCh^{MYR} 5′/3′gap43 transfected DRGs, respectively. For peptide treatments, G3BP1-mCh transfected DRG neurons were treated with 10 μM G3BP1 peptides after acquiring the baseline expression values. Photobleaching followed by analyses of recovery was performed after 30 min of peptide exposure.

For testing G3BP1 protein mobility in axons, DRG neurons were transfected with G3BP1^{S149A}-GFP or G3BP1^{S149E}-GFP and imaged as above but only the 488 nm laser was used for photobleaching (Argon laser at 70% power, pulsed every 0.82 s for 80 frames). Fluorescent intensities in the ROIs were calculated by the Leica LASX software.

For normalizing across experiments, fluorescence intensity value at t=0 min postbleach from each image sequence was set as 0%. The percentage of fluorescence recovery at each time point after photobleaching was then calculated by normalizing relative to the pre-bleach fluorescence intensity (set at 100%).

Live cell imaging for G3BP1-mCherry granules. DRG neurons were transfected with G3BP1-mCherry, and 36 h later distal axons were imaged using Leica SP8X confocal microscope with environmental chamber maintained at 37° C., 5% CO₂ (with 63×/1.4 NA oil immersion objective). G3BP1-mCherry signals were imaged as single optical planes in the axon shaft every 2 s for 100 frames (at 540 nm excitation and 23% white light laser power; 565-597 nm emission). To study the effect of the G3BP1 190-208 or 168-189 peptide, 10 μM FITC-conjugated peptide was added to the media and 15 min later imaging was continued.

For quantitation of G3BP1-mCherry aggregates density and size, a 100 μm of the axon shaft was considered (≥200 μm from cell body). Thresholding was applied to acquired image sequences using ImageJ to generate binary masks.

ImageJ particle analyzer was used for analysis. G3BP1 aggregates with area≥1 μm² were considered as SG-like structures. For analyzing the G3BP1 aggregate velocity, ImageJ Trackmate plug-in was used.

Puromycinylation assay. To visualize newly synthesized proteins in cultured neurons, the inventors used the Click-iT® Plus OPP Protein Synthesis Assay Kit per manufacturer's instructions (Invitrogen/Life Technologies). Briefly, 3 DIV cultures were incubated with 20 μM₀-propargyl-puromycin (OPP) for 30 min at 37° C. OPP labeled proteins were detected by crosslinking with Alexa Fluor-594 picolyl azide molecule. Coverslips were then mounted with Prolong Gold Antifade (Invitrogen) and imaged with Leica DMI6000 epifluorescent microscope as above. ImageJ was used to quantify the Puromycinylation signals in distal axons and cell bodies.

Immunoblotting. For immunoblotting, protein lysates or immunoprecipitates were denatured by boiling in Laemmle sample buffer, fractionated by SDS-PAGE, and transferred to nitrocellulose membranes. Blots were blocked for 1 h at room temperature with 5% non-fat dry milk in Tris-buffered saline with 0.1% Tween 20 (TBST) for anti-tagBFP, -GAPDH and -G3BP1 antibodies; 5% BSA in TBST was used for blocking anti-G3BP1^{PS149} antibody. Primary antibodies diluted in appropriate blocking buffer were added to the membranes and incubated overnight incubation at 4° C. with rocking. Primary antibodies consisted of: rabbit anti-G3BP1 (1:2000; Sigma), rabbit anti-G3BP1^{PS149} (1:1000; Sigma), rabbit anti-TagBFP (1:2000; Evrogen), and rabbit anti-GAPDH (1;2,000; CST). After washing in TBST, blots were incubated HRP-conjugated anti-rabbit IgG antibodies (1:5000; Jackson lab) diluted in blocking buffer for 1 h at room temperature. After washing signals were detected using ECL PrimeTM (GE Healthcare).

Mass spectrometry by parallel reaction monitoring (PRM). Protein extraction was carried out according to the standard manufacturer's protocol using axoplasm samples suspended in 0.5 ml of TrIzol LS. Protein pellets were then reconstituted in urea, reduced, alkylated, digested with trypsin and desalted as previously described. PRM was performed using nano-Acquity UPLC system (Waters) online with Q Exactive Plus mass spectrometer (Thermo-Fisher). Digested peptides were loaded at 0.5 μg per sample and separated by low pH, two-buffer reverse phase chromatography on a 200 cm monolithic silica-C18 column (GL Sciences, Japan) over a 6 h gradient as previously described. Q Exactive Plus instrument was used in PRM mode with the following parameters: positive polarity, R=17,500 at 200 m/z, AGC target 1e6, maximum IT 190 ms, MSX count 1, isolation window 3.0 m/z, NCE 35%. Unique previously detected tryptic peptide DFFQSYGNVVELR from rat G3BP1 (Uniprot ID D3ZYS7) was targeted (as part of a set of 184 target peptides from 84 proteins with possible roles in axonal mRNA transport; subject of a separate study). PRM data analysis was performed using Skyline v. 3.5.

RNA immunoprecipitation (RIP). HEK293T cells or DRG neurons were lysed in 100 mM KCl, 5 mM MgCl₂, 10 mM HEPES [pH 7.4], 1 mM DTT, and 0.5% NP-40 (RIP buffer) supplemented with 1× protease inhibitor cocktail (Roche) and RNasin Plus (Invitrogen). Cells were passed through 25 Ga needle 5-7 times and cleared by centrifugation at 12,000×g for 20 min. Cleared lysates were pre-absorbed with Protein A-Dynabeads (Invitrogen) for 30 min. Supernatants were then incubated with primary antibodies for 3 h and then immunocomplexes precipitated with Protein G-Dynabeads (Invitrogen) for additional 2 h at 4° C. with rotation. Mouse anti-G3BP1 (5 μg, BD Biosciences) and rabbit anti-GFP (5 μg, Abcam) antibodies were used for immunoprecipitation. Beads were washed six times with cold RIP buffer. Bound RNAs were purified and analyzed by RTddPCR (see below).

RNA isolation and PCR analyses. RNA was isolated from immunoprecipitates and cultures using the RNeasy Microisolation kit (Qiagen). Fluorimetry with Ribogreen (Invitrogen) was used for RNA quantification. For analyses of total RNA levels and inputs for RIP analyses, RNA yields were normalized across samples prior to reverse transcription using Sensifast (Bioline). For RIP assays, an equal proportion of each RIP was used for reverse transcription with Sensifast. ddPCR products were detected using Evagreen or Taqman primer and probe sets (Biorad or Integrated DNA Tech; sequences available on request) and QX200™ droplet reader (Biorad). In GFP RIP experiment, B domain-BFP expression consistently increased G3BP1-GFP levels in the DRG neurons. So the level of mRNA precipitating with G3BP1-GFP was normalized to the G3BP1-GFP signals from immunoblotting across each sample in each experiment.

Assessment of muscle reinnervation. AAV5 encoding GFP or B domain-BFP was injected into the sciatic nerve near the sciatic nerve notch (left and right nerves, respectively). 7 d post-virus injections, bilateral sciatic nerve crushes were performed at mid-thigh level. The extent of innervation of the LG and TA muscles was evaluated using in vivo electromyography (EMG). For this, animals were anesthetized by IP injection with Ketamine HCl (80 mg/kg, Hospira) and AnaSed (10 mg/kg, LLOYD Laboratories) to achieve a surgical plane of general anesthesia; additional IP injections of Ketamine HCl (40 mg/kg) were given to maintain this plane throughout the experiment.

Bipolar fine wire EMG electrodes were constructed from insulated nickel alloy wire (California Fine Wire, Stablohm 800). The insulation over the distal 1 mm of the tips was removed by scraping with a scalpel blade and the tips of the two wires were staggered by 1 mm. Electrodes were then placed into the lateral head of LG and the mid-belly of the TA muscles using a 25 Ga hypodermic needle. Once in place, the needle was removed and the wires were connected to the differential amplifiers. To stimulate the sciatic nerve, a small skin incision was made just inferior to the ischial tuberosity, exposing the sciatic nerve as it coursed between the gluteal and hamstring muscles, proximal to the crush injury site. A small rectangle of Parafilm® was wrapped loosely around the nerve and pierced by two unipolar needle electrodes (Neuroline monopolar, 28 G, Ambu/AS). The tips of the two needle electrodes were separated from each other by approximately 1 mm.

Lead wires from the needles were connected to an optically isolated constant voltage stimulator under computer control.

Evoked EMG activity from LG and TA was then recorded after sciatic nerve stimulation. Stimulation and recording were controlled by a laboratory computer system running custom software written in Labview®. Ongoing EMG activity in the LG was sampled at 10 kHz; when the rectified and integrated voltage over a 20 ms period fell within a user-defined range, a 0.3 ms duration stimulus pulse was delivered to the nerve via the needle electrodes. Muscle activity was sampled from 20 ms prior to the stimulus until 100 ms after the stimulus and recorded to disc.

Stimuli were delivered no more frequently than once every 3 s to avoid fatigue. A range of stimulus intensities was applied in each experiment to sample evoked muscle activity from sub-threshold to supramaximal. In a typical experiment approximately 200 stimulus presentations were studied. At the end of each experiment, all electrodes were removed and the skin incisions closed with sutures.

The recorded compound muscle action potentials (M waves) in LG and TA evoked by sciatic nerve stimulation were analyzed off-line. The amplitude of the evoked M waves was measured as the average rectified voltage within a defined time window after the stimulus application. In intact anesthetized animals, this window is 0.5-2.0 ms, as described. After nerve crush, M waves evoked from sciatic nerve stimulation are, by definition, generated by reinnervated muscle fibers.

The latency and duration of these potentials are longer than those found in intact animals. Thus, the time window used to measure the amplitude of the M waves was adjusted to accommodate this change. Recordings were made from intact animals, immediately following and 1, 2, 4, 6, and 8 wk after nerve crush. At each time point, the amplitude of the largest evoked M wave (Mmax) was determined and scaled to Mmaxrecorded from that animal prior to nerve crush. Means of these scaled responses recorded from muscles in which motor neurons were induced to express B domain-BFP and those in which motor neurons expressed only GFP were compared at each time studied.

Image analyses and processing. For protein—protein and protein—mRNA colocalization, xyz image sequences captured 100 μm segments of the axon shaft (separated from the cell body and growth cone by ≥200 μm) were deconvolved using Huygens HyVolution software. Colocalization was analyzed using ImageJ JACoP plug-in (nih.gov/ij/plugins/track/jacop.html) to calculate Pearson's coefficient. These coefficient calculations were independently validated with Volocity software (Perkin Elmer).

For analyses of protein levels in tissues, z planes of the xyz tile scans from 3-5 locations along each nerve section were analyzed using ImageJ. Colocalization plug-in was used to extract protein signals that overlap with axonal marker (NF) in each plane, with the extracted ‘axon-only’ signal projected as a separate channel.

For calculating axonal G3BP1 aggregate and G3BP1^{PS149} signal intensities, absolute signal intensity was quantified in each xy plane of the ‘Colocalization’ extracted images for axonal only G3BP1 and G3BP1^{PS149} using ImageJ. Protein signal intensities across the individual xy planes were then normalized to NF immunoreactivity area. The relative protein signal intensity was averaged for all image locations in each biological replicate.

For neurite outgrowth, images from 60 h DRG cultures were analyzed for neurite outgrowth using WIS-Neuromath. Axon morphology was visualized using GFP and/or NF immunofluorescence as described. Differentiated hiPSC neuron image acquisition and neurite length quantification was performed using Arrayscan XTI (Thermo Fisher).

To assess regeneration in vivo, tile scans of NF-stained nerve sections were post-processed by Straighten plug-in for ImageJ (imagej.nih.gov/ij/). NF positive axon profiles were then counted in 30 μm bins at 0.3 mm intervals distal from crush site. Number of axon profiles present in the proximal crush site was treated as the baseline, and values from the distal bins were normalized to this to calculate the percentage of regenerating axons.

Statistical analyses. Kaleidagraph (Synergy), Prism (GraphPad), and Excel (Microsoft) software packages were used for statistical analyses. One-way ANOVA was used to compare means of independent groups and Student's t-test was used to compare smaller sample sizes of the in vivo analyses. p values of ≤0.05 were considered as statistically significant. For statistical analyses of Pearson's coefficients, Fishers Z-transformation was used to compare: G3BP1+ HuR, FXR1, and FMRP colocalization versus DCP1a+XRN1 coefficients and G3BP1+DCP1a and XRN1 versus DCP1a+XRN1 coefficients.

The current disclosure provides evidence that blocking stress granule aggregation by treatment with cell permeable G3BP1 190-208 peptide effectively prevents MPP+-induced (PD model) and Aβ-mediated neurotoxicity (AD model), as well as prevents protein aggregation associated with expression of mutant TIA1 and TDP43 proteins that cause ALS. Here, we provide data indicating that the cell permeable G3BP1 190-208 peptide not only triggers disassembly of pre-existing stress granule protein aggregates in these neurodegenerative disease models, but also effectively blocks neurodegenerative disease associated axon degeneration.

Cell Permeable G3BP1 190-208 Peptide Prevents Neurodegeneration-Associated Aggregation of Endogenous Stress Granule Protein in Axons.

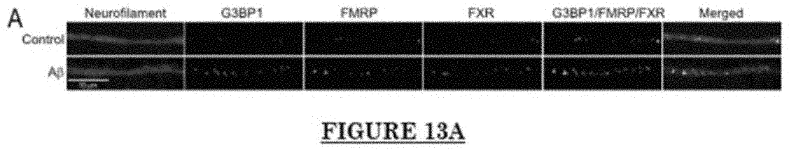

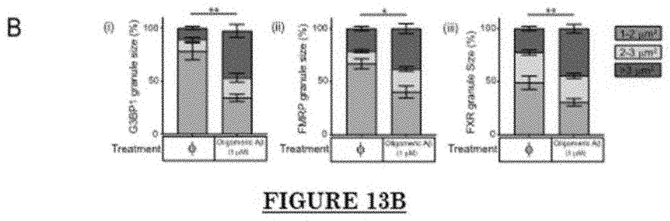

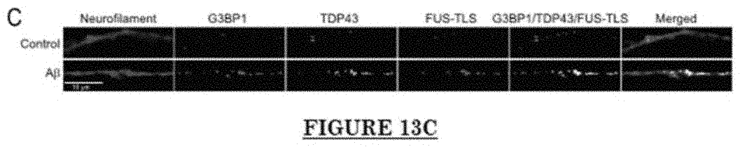

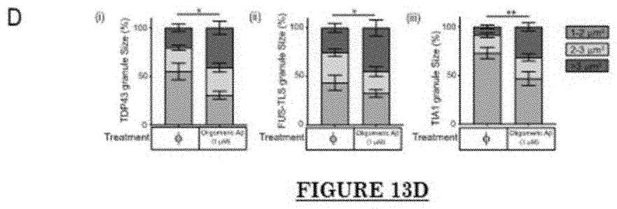

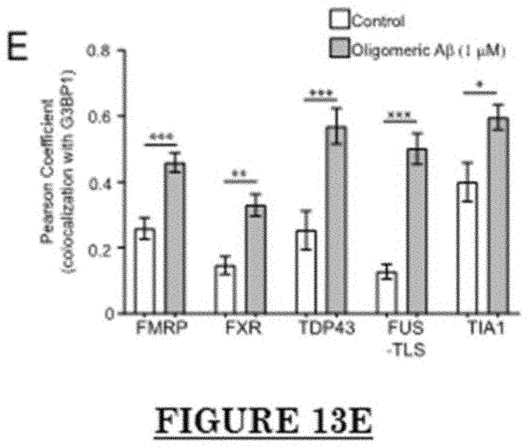

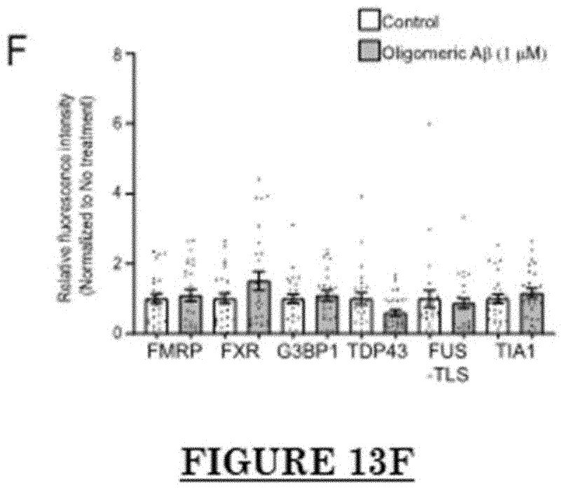

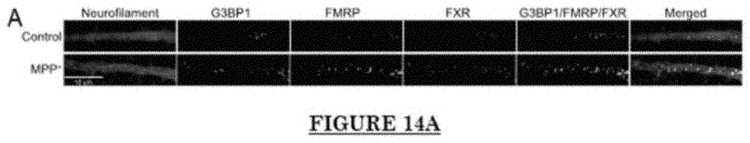

The current disclosure first tested whether treatment of E 18 cortical or midbrain neurons with neurotoxins Aβ and MPP+, respectively, induces protein aggregations of endogenous stress granule and neurodegeneration-associated proteins preceding axon degeneration and cell death. For cortical neurons, a 6-hour exposure to 1 μM Aβ oligomer significantly increased aggregate size for the stress granule proteins G3BP1, TIA1, FMRP and FXR as well as the ALS- and FTD-associated proteins TDP43 and FUS-TLS along axons (see FIGS. 13A, 13B, 13C, and 13D).

FIGS. 13A-13F show Amyloid beta oligomer treatment increases in RNA binding protein aggregation. FIGS. 13A and 13B show representative confocal. images for G3BP1 (magenta), FMRP (green), FXR (red) and neurofliament (blue) immunoreactivity along axons for E18 cortical neuron cultures (7 DIV)±1 μM Aβ oligomer for 6 hours are shown in FIG. 13A. FIG. 13B shows the size distribution for aggregates of G3BP1 (1), FMRP (ii) and FXR (iii) along control vs. AP oligomer treated axons as in 13A. FIGS. 13C and 13D show representative confocal images for G3BP1 (magenta), FUS-TLS (green), TDP43 (red) and neurofilament (blue) immunoreactivity along axons for cortical neurons treated as in A are shown in FIG. 13C. FIG. 13D shows the size distribution for TDP43 (i), FUS-TLS (ii), TIA1 (iii) aggregates along control vs. Aβ oligomer-treated axons as in 13C. FIGS. 13E and 13F show quantification for colocalization of FMRP, FXR, TDP43, FUS-TLS and TIA1 with G3BP1 in axons of E18 cortical neurons treated as in. A shown as average Pearson's coefficient±SEM shown in FIG. 13E. FIG. 13F shows the overall levels of these proteins based in exposure matched images (N≤100 aggregates over three repetitions and *p≤0.01, **p≤0.005, ***p≤0.001 for entire population distributions by Fishers exact test for B and D; N≥25 neurons over 3 repetitions and *p≤0.01, **p≤0.005, ***p≤0.001 by one-way ANOVA with Tukey HSD post-hoc for FIG. 13E and FIG. 13F).

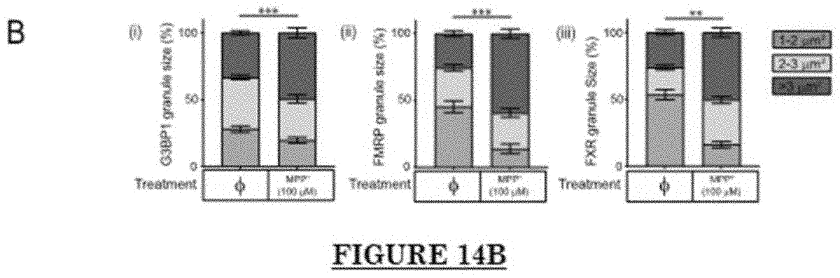

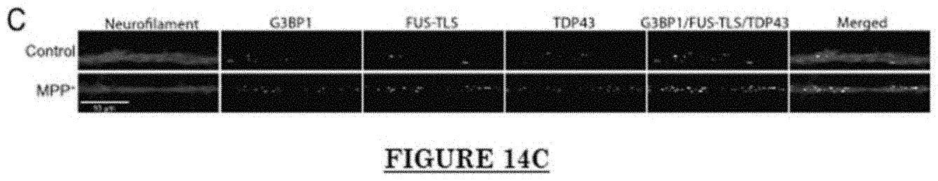

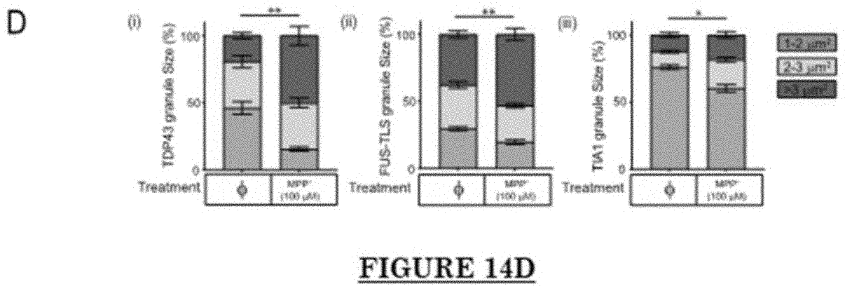

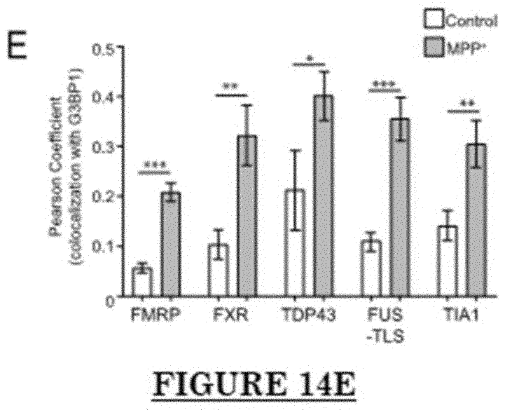

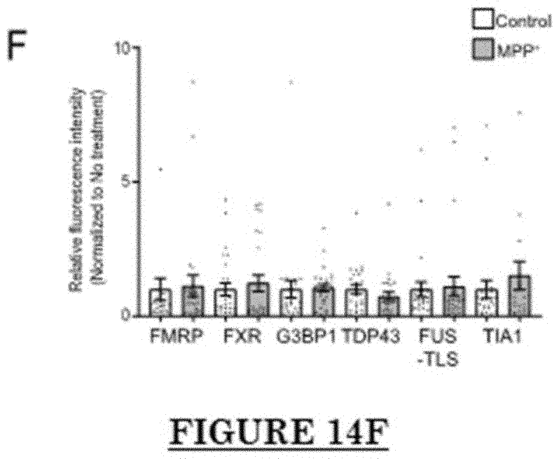

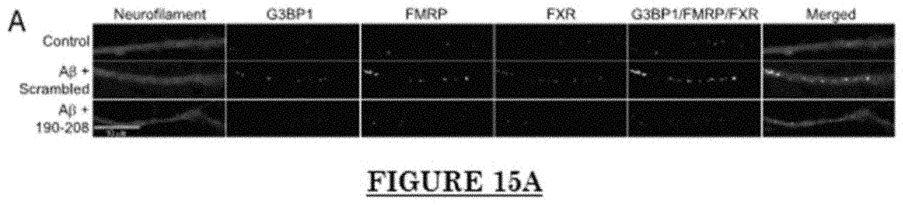

This treatment duration is well before we observe axon degeneration and cell death, so this is a ‘pre-neurodegeneration’ response to Aβ peptide. The Aβ peptide treatment also significantly increased colocalization of TIA1, FMRP, FXR, TDP43 and FUS-TLS with G3BP1 aggregates with no overall change in the levels of these proteins (FIGS. 13E-13F). In midbrain neurons, MPP⁺ treatment for 6 hours also significantly increases the aggregate sizes for G3BP1, TIA1, FMRP, FXR, TDP43 and FUS-TLS aggregates along axons (FIGS. 14A-14D). Similar to Aβ results above, this MPP⁺ treatment duration is well before we observe axon degeneration and cell death, so this is a ‘pre-neurodegeneration’ response to the neurotoxin MPP⁺. Also, MPP⁺ treatment causes a significant increase in TIA1, FMRP, FXR, TDP43 and FUS-TLS colocalization with G3BP1 without any change in overall levels for any of these proteins (FIG. 14E-14F). These data point to aggregation of stress granule and neurodegeneration-associated RNA binding proteins as a pathophysiological event shared between different neurodegeneration-associated stressors.

FIGS. 14A-14F show PD-causing MPP⁺ increases RNA binding protein aggregation. Representative confocal images for G3BP1 (magenta), FMRP (green), FXR (red) and neurofilament immunoreactivity along axons for E18 midbrain neuron cultures (7 DIV)±100 μM MPP⁺ for 6 hours are shown in FIG. 14A. FIG. 14B shows the size distribution for aggregates of G3BP1 (i), FMRP (ii) and FXR (iii) along control vs. MPP⁺-treated axons as in A. Representative confocal images for G3BP1 (magenta), FUS-TLS (green), TDP43 (red) and neurofilament (blue) immunoreactivity along axons for midbrain neurons treated as in A are shown in FIG. 14C. FIG. 14D shows the size distribution for TDP43 (i), FUS-TLS (ii), TIA1 (iii) aggregates along control vs. MPP⁺-treated axons as in FIG. 14C. Quantifications for colocalization of FMRP, FXR, TDP43, FUS-TLS and TIA1 with G3BP1 in axons of E18 midbrain neurons treated. as in FIG. 14A are shown as average Pearson's coefficient±SEM shown in FIG. 14E. FIG. 14F shows the overall levels of these proteins based in exposure matched images (N≥100 aggregates over three repetitions and *p≤0.01, **p≤0.005, ***p≤0.001 for entire population distributions by Fishers exact test for FIG. 14B and FIG. 14D; N≥25 neurons over 3 repetitions and *p≤0.01, **p≤0.005, ***p≤0.001 by one-way ANOVA with Tukey HSD post-hoc for FIGS. 14E-14F).

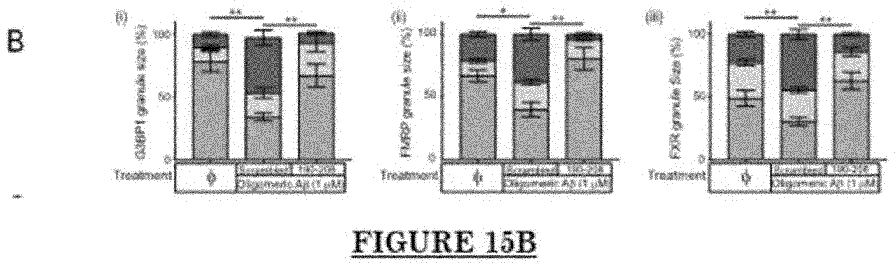

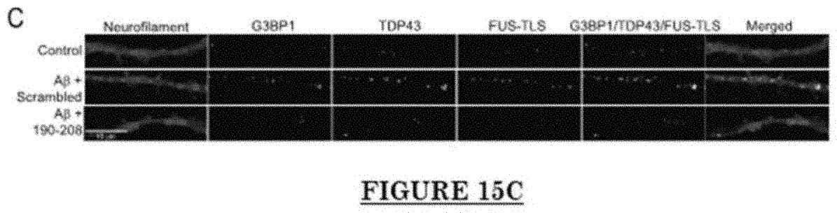

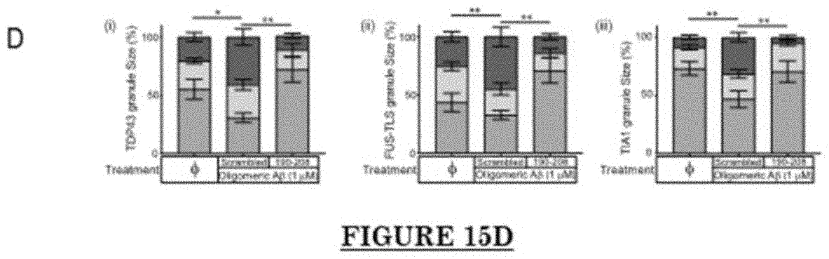

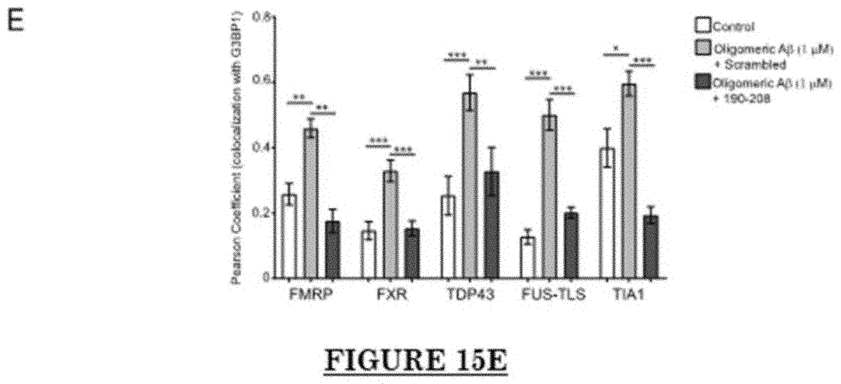

The data in FIGS. 13A-F and 14A-F raise the possibility that stress granule targeting therapies like the cell permeable G3BP1 190-208 peptide may decrease or even prevent loss of neurons across different types of neurodegenerative diseases. To address this possibility, the current disclosure asked if the cell permeable G3BP1 190-208 peptide could disassemble pathological protein aggregates in neurons. For this, 7 day cortical neuron cultures were treated with 1 μM A□ oligomer for 2 hours and then treated with cell permeable G3BP1 190-208 peptide or a scramble sequence cell permeable peptide for an additional 4 hours. As above, the Aβ oligomer treated cultures showed a significantly increased size of G3BP1, TIA1, FMRP, FXR, TDP43 and FUS-TLS aggregates along axons (FIGS. 15A-15D). Cell permeable G3BP1 190-208 peptide significantly decreased size of these aggregates (FIGS. 15A-15D) and TIA1, FMRP, FXR, TDP43 and FUS-TLS colocalization with G3BP1 compared to Aβ without peptide treatment as well as Aβ plus the scrambled peptide control (see FIG. 15E). Thus, the cell permeable G3BP1 190-208 peptide can not only prevent RNA binding protein aggregation in axons after exposure to neurotoxins associated AD and PD, but it can also trigger disassembly of these pathological aggregates after they begin to form.

FIGS. 15A-15E show reduction in amyloid beta treatment dependent G3BP1-associated protein aggregates by addition of G3BP1 190-208 peptide. Representative confocal images for G3BP1. (magenta), FMRP (green), FXR (red) and. neurofilament (blue) immunoreactivity along axons for control and 1 μM A oligomer-treated E18 cortical neurons (7 DIV)±cell permeable G3BP1 190-208 peptide are shown in FIG. 15A, FIG. 15B shows the size distribution for aggregates of G3BP1 (i), FMRP (ii) and FXR (iii) along control vs. Aβ-treated axons and Aβ treated+G3BP1 190-208 or cell permeable peptide with scrambled sequence. Representative confocal images for G3BP1 (magenta), FUS-TLS (green), TDP43 (red) and neurofilament (blue) immunoreactivity along axons for cortical neurons treated as in FIG. 15A are shown in FIG. 15C, FIG. 15 D shows the size distribution for TDP43 (i), FUS-TLS (ii), TIA1 (iii) aggregates along axons of control vs. Aβ-treated and Aβ treated+G3BP1 190-208 or cell permeable peptide with scrambled sequence. Quantifications for co:localization of FMRP, FXR, TDP43, FUS-TLS and TIA1 with G3BP1 in axons of E18 cortical neurons treated as in C are shown as average Pearson's coefficient±SEM (N≥120 aggregates over three repetitions and *p≤0.01, **p≤0.005, ***p≤0.001 for entire population distributions by Fishers exact test for FIG. 15B and FIG. 15D; N≤25 neurons over 3 repetitions and *p≤0.01 **p≤0.005, ***p≤0.001 by one-way ANOVA with Tukey HSD post-hoc for FIG. 15E).

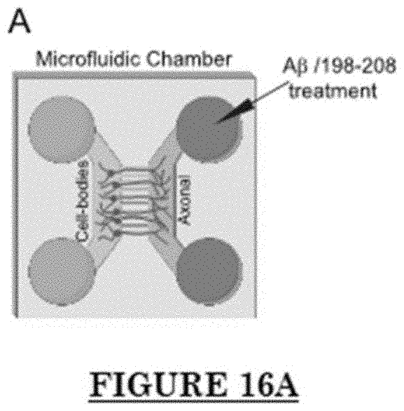

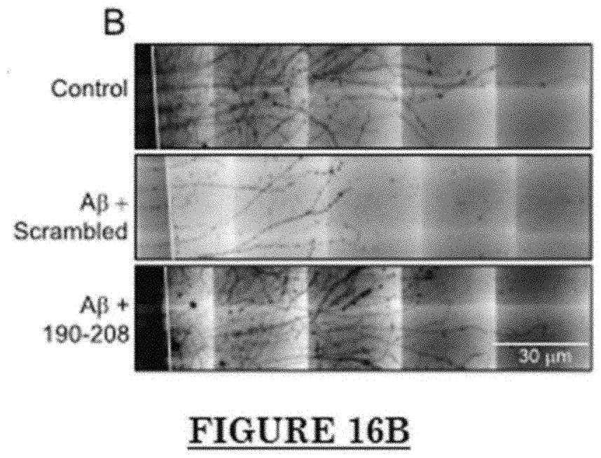

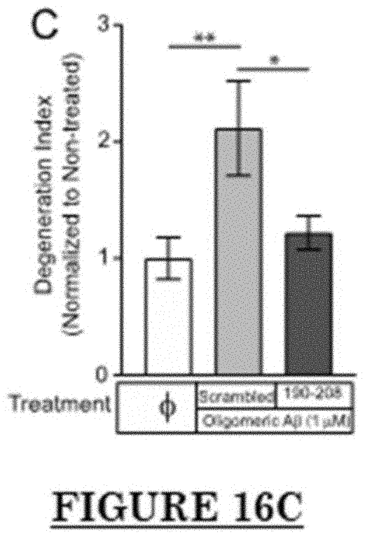

Cell permeable G3BP1 190-208 peptide prevents neurotoxin-induced axon degeneration through localized mechanisms. Axonal degeneration is thought to precede neuron death (and hence, neurodegeneration) in several neurodegenerative diseases including AD, PD and ALS. To test whether the cell permeable G3BP1 190-208 peptide protects against this early axonal degeneration, the current disclosure exposed only the axons of E18 cortical neuron cultures to Aβ peptide using microfluidic cultures (FIG. 16A). Axons exposed to A@ oligomer showed axon degeneration at 16 hours (FIG. 16B-16C). Treating the axons with the cell permeable G3BP1 190-208 peptide near completely prevented this axon degeneration (FIG. 4B-C). Thus, the cell permeable G3BP1 190-208 peptide protects axons from the neurotoxic effects of Aβ oligomers through axon intrinsic mechanisms.

FIGS. 16A-16C show cell permeable G3BP1 190-208 peptide rescues amyloid beta oligomer-mediated axonal degeneration. FIG. 16A shows a schematic of the microfluidic culture set up. E18 cortical neurons were plated into the cell-body compartment (blue); after 7 DIV axons from these neurons extend through the microchannels into the axon compartment (pink). FIG. 16B shows representative montage images of axonal compartment of DIV7 E18 cortical neurons stained with neurofilament for control and Aβ oligomer (1 μM for 16 hours)±190-208 G3BP1 peptide (1 μM). Yellow line indicates the exit of the axons from the microchannels. FIG. 16C shows quantitation of degeneration indices for cultures from B (N=3 repetitions and *p≤0.01, **p≤0.005 by one-way ANOVA with Tukey HSD post-hoe).

Together, these data indicate that exposure of neurons to toxins known to be causative for AD and symptoms associated with PD trigger aggregation of RNA binding proteins associated with stress granules (G3BP1, TIA1, FMRP, and FXR) and are mutated in ALS (TDP43 and FUS-TLS). The cell permeable G3BP1 190-208 peptide that is the subject of this application disassembles these protein aggregates along axons and prevents axonal degeneration. These findings support our application for use of G3BP1, of between 19-21 peptides, such as rattus 190-208 or homo sapiens 189-209 peptide, as a prophylactic and/or treatment method for neurodegenerative disorders.

Neurodegenerative disease symptoms typically manifest from loss of synaptic connections between neruons or neurons and their target tissues. To determine if targeting G3BP1 granule aggregation could prevent synapse loss, we focused on neuromuscular junctions in ALS. Mislocalization of the predominantly nuclear RNA/DNA binding protein, TDP-43, occurs in motor neurons of —95% of amyotrophic lateral sclerosis (ALS) patients, but the contribution of axonal TDP-43 to this neurodegenerative disease is unclear. Here, we show TDP-43 accumulation in intra-muscular nerves from ALS patients and in axons of human iPSC-derived motor neurons of ALS patient, as well as in motor neurons and neuromuscular junctions (NMJs) of a TDP-43 mislocalization mouse model. In axons, TDP-43 is hyper-phosphorylated and promotes G3BP1-positive ribonucleoprotein (RNP) condensate assembly, consequently inhibiting local protein synthesis in distal axons and NMJs. Specifically, the axonal and synaptic levels of nuclear-encoded mitochondrial proteins are reduced. Clearance of axonal TDP-43 or dissociation of G3BP1 condensates restored local translation and resolved TDP-43-derived toxicity in both axons and NMJs. These findings support an axonal gain of function of TDP-43 in ALS, which can be targeted for therapeutic development.

Amyotrophic lateral sclerosis (ALS) is an adult-onset neurological disease characterized by neuromuscular junction (NMJ) disruption and motor neuron (MN) degeneration. See, Taylor, J. P., Brown, R. H. & Cleveland, D. W. Decoding ALS: from genes to mechanism. Nature 539, 197-206 (2016) and Cook, C. & Petrucelli, L. Genetic convergence brings clarity to the enigmatic red line in ALS. Neuron 101, 1057-1069 (2019). An important pathological hallmark in ALS patients is mislocalization of the primarily nuclear RNA and DNA binding protein TAR-DNA-binding-protein 43 (TDP-43) to the cytoplasm of MNs. See, Neumann, M. et al. Ubiquitinated TDP-43 in frontotemporal lobar degeneration and amyotrophic lateral sclerosis. Science 314, 130-133 (2006), Melamed, Z. et al. Premature polyadenylation-mediated loss of stathmin-2 is a hallmark of TDP-43-dependent neurodegeneration. Nat. Neurosci. 22, 180-190 (2019), Tsuji, H. et al. Molecular analysis and biochemical classification of TDP-43 proteinopathy. Brain 135, 3380-3391 (2012), Barmada, S. J. et al. Cytoplasmic mislocalization of TDP-43 is toxic to neurons and enhanced by a mutation associated with familial amyotrophic lateral sclerosis. J. Neurosci. 30, 639-649 (2010), Cook, C. N. et al. C9orf72 poly(GR) aggregation induces TDP-43 proteinopathy. Sci. Transl. Med. 12, eabb3774 (2020). TDP-43 participates in transcription, RNA splicing, processing, and nucleocytoplasmic transport. See, Klim, J. R. et al. ALS-implicated protein TDP-43 sustains levels of STMN2, a mediator of motor neuron growth and repair. Nat. Neurosci. 22, 167-179 (2019), Chou, C. C. et al. TDP-43 pathology disrupts nuclear pore complexes and nucleocytoplasmic transport in ALS/FTD. Nat. Neurosci. 21, 228-239 (2018), and Ling, J. P., Pletnikova, O., Troncoso, J. C. & Wong, P. C. TDP-43 repression of nonconserved cryptic exons is compromised in ALS-FTD. Science 349, 650-655 (2015). Additionally, mutations in TDP-43 were identified in a subset of ALS patients. See, Kabashi, E. et al. TARDBP mutations in individuals with sporadic and familial amyotrophic lateral sclerosis. Nat. Genet. 40, 572-574 (2008). Cytoplasmic accumulation of TDP-43 has been implicated in ALS via several pathways, see Suk, T. R. & Rousseaux, M. W. C. The role of TDP-43 mislocalization in amyotrophic lateral sclerosis. Mol. Neurodegener. 15, 1-16 (2020), some are related to the loss of TDP-43 nuclear function. See, Melamed, Klim and Ling, supra. However, the mechanism sensitizing MNs and NMJs to TDP-43 cytoplasmic condensation remains unclear.

One key event which develops due to TDP-43 mislocalization is the formation of phase separated cytoplasmic condensates that alter RNA localization and translation. See, Chen, Y. & Cohen, T. J. Aggregation of the nucleic acid— binding protein TDP-43 occurs via distinct routes that are coordinated with stress granule formation. J. Biol. Chem. 294, 3696-3706 (2019), Russo, A. et al. Increased cytoplasmic TDP-43 reduces global protein synthesis by interacting with Rackl on polyribosomes. Hum. Mol. Genet 26, 1407-1418 (2017), Gasset-Rosa, F. et al. Cytoplasmic TDP-43 de-mixing independent of stress granules drives inhibition of nuclear import, loss of nuclear TDP-43, and cell death article. Neuron 102, 339-357 (2019), Mann, J. R. et al. RNA binding antagonizes neurotoxic phase transitions of TDP-43. Neuron 102, 321-338.e8 (2019), and Wang, I.-F., Wu, L.-S., Chang, H.-Y. & Shen, C.-K. J. TDP-43, the signature protein of FTLD-U, is a neuronal activity-responsive factor. J. Neurochem. 105, 797-806 (2008). Additionally, ALS-associated mutations in TDP-43 directly interfere with mRNA transport. See, Alami, N. H. et al. Axonal transport of TDP-43 mRNA granules is impaired by ALS-causing mutations. Neuron 81, 536-543 (2014) and Gopal, P. P., Nirschl, J. J., Klinman, E. & Holzbaur, E. L. F. Amyotrophic lateral sclerosis-linked mutations increase the viscosity of liquid-like TDP-43 RNP granules in neurons. Proc. Natl Acad. Sci. USA 114, E2466—E2475 (2017). This process is associated with development of pathological ribonucleoprotein (RNP) condensates, which deregulate mRNA localization and translation. Liao, Y. C. et al. RNA granules hitchhike on lysosomes for long-distance transport, using Annexin All as a molecular tether. Cell 179, 147-164.e20 (2019) and Sahoo, P. K. et al. Axonal G3BP1 stress granule protein limits axonal mRNA translation and nerve regeneration. Nat. Commun. 9, 1-14 (2018).

Formation of RNP condensates, see Liao, Y. C. et al. RNA granules hitchhike on lysosomes for long-distance transport, using Annexin All as a molecular tether. Cell 179, 147-164.e20 (2019) and Sahoo, P. K. et al. Axonal G3BP1 stress granule protein limits axonal mRNA translation and nerve regeneration. Nat. Commun. 9, 1-14 (2018), and mRNA transport defects, see Costa, C. J. & Willis, D. E. To the end of the line: axonal mRNA transport and local translation health and neurodegenerative disease, Dev. Neurobiol. 7 8, 209-220 (2018), affect localized protein synthesis, an important regulator of axonal and synaptic health. See, Terenzio, M. et al. Locally translated mTOR controls axonal local translation in nerve injury. Science 359, 1416-1421 (2018), Cioni, J.-M. et al. Late endosomes act as mRNA translation platforms and sustain mitochondria in axons article late endosomes act as mRNA translation platforms and sustain mitochondria in axons. Cell 176, 56-72 (2019), Hafner, A. S., Donlin-Asp, P. G., Leitch, B., Herzog, E. & Schuman, E. M. Local protein synthesis is a ubiquitous feature of neuronal pre- and postsynaptic compartments. Science. 364, eaau3644, (2019), and Shigeoka, T. et al. Dynamic axonal translation in developing and mature visual circuits. Cell 166, 181-192 (2016). Several studies demonstrated altered protein synthesis in ALS models. See, Russo, A. et al. Increased cytoplasmic TDP-43 reduces global protein synthesis by interacting with Rack1 on polyribosomes. Hum. Mol. Genet 26, 1407-1418 (2017), López-Erauskin, J. et al. ALS/FTD-linked mutation in FUS suppresses intra-axonal protein synthesis and drives disease without nuclear loss-of-function of FUS. Neuron 100, 816-830.e7 (2018), and Zhang, Y. J. et al. Poly(GR) impairs protein translation and stress granule dynamics in C9orf72-associated frontotemporal dementia and amyotrophic lateral sclerosis. Nat. Med. 24, 1136-1142 (2018). However, most of those observations were made in non-neuronal cells or within neuronal cell body, not in the NMJ. Given that NMJs and MN axons are highly vulnerable in ALS, see, Dadon-Nachum, M., Melamed, E. & Offen, D. The “Dying-Back” phenomenon of motor neurons in ALS. J. Mol. Neurosci. 43, 470-477 (2011) and So, E. et al. Mitochondrial abnormalities and disruption of the neuromuscular junction precede the clinical phenotype and motor neuron loss in hFUSWT transgenic mice. Hum. Mol. Genet 27, 463-474 (2018), the consequences of TDP-43 mislocalization in these compartments are key to understanding disease pathology.

To study the effect of TDP-43 mislocalization on the NMJ in a precise and controlled environment, we used a neuromuscular co-culture setup in microfluidic chambers (MFCs) we developed, see, Zahavi, E. E. et al. A compartmentalized microfluidic neuromuscular co-culture system reveals spatial aspects of GDNF functions. J. Cell Sci. 128, 1241-1252 (2015), Maimon, R. et al. Mir126-5p downregulation facilitates axon degeneration and nmj disruption via a non-cell-autonomous mechanism in ALS. J. Neurosci. 38, 5478-5494 (2018), Altman, T., Geller, D., Kleeblatt, E., Gradus-Perry, T. & Perlson, E. An in vitro compartmental system underlines the contribution of mitochondrial immobility to the ATP supply in the NMJ. J. Cell Sci. 132, 23 (2019), Ionescu, A. et al. Targeting the sigma-1 receptor via pridopidine ameliorates central features of ALS pathology in a SOD1 G93A model. Cell Death Dis. 10, 210 (2019), and Ionescu, A., Zahavi, E. E., Gradus, T., Ben-Yaakov, K. & Perlson, E. Compartmental microfluidic system for studying muscle-neuron communication and neuromuscular junction maintenance. Eur. J. Cell Biol. 95, 69-88 (2016). This platform models pathological features of ALS, such as axon degeneration, NMJ dysfunction and MN death. See, Maimon, R. et al. Mir126-5p downregulation facilitates axon degeneration and nmj disruption via a non-cell-autonomous mechanism in ALS. J. Neurosci. 38, 5478-5494 (2018), Ionescu, A. et al. Targeting the sigma-1 receptor via pridopidine ameliorates central features of ALS pathology in a SOD1 G93A model. Cell Death Dis. 10, 210 (2019), and Osaki, T., Uzel, S. G. M. & Kamm, R. D. Microphysiological 3D model of amyotrophic lateral sclerosis (ALS) from human iPS-derived muscle cells and optogenetic motor neurons. Sci. Adv. 4, eaat5847 (2018). The fluidic separation between MN cell-body and axon allows formation of functional NMJs exclusively at distal compartments, see, Zahavi, E. E. et al. A compartmentalized microfluidic neuromuscular co-culture system reveals spatial aspects of GDNF functions. J. Cell Sci. 128, 1241-1252 (2015), Maimon, R. et al. Mir126-5p downregulation facilitates axon degeneration and nmj disruption via a non-cell-autonomous mechanism in ALS. J. Neurosci. 38, 5478-5494 (2018), Altman, T., Geller, D., Kleeblatt, E., Gradus-Perry, T. & Perlson, E. An in vitro compartmental system underlines the contribution of mitochondrial immobility to the ATP supply in the NMJ. J. Cell Sci. 132, 23 (2019), Ionescu, A. et al. Targeting the sigma-1 receptor via pridopidine ameliorates central features of ALS pathology in a SOD1 G93A model. Cell Death Dis. 10, 210 (2019), http://scholar.goggle.com/scholar_lookup?&title=Targeting%20the%20sigma-1%20receptor%20via%20pridopidine%20ameliorates%20central%20features%20ALS%20pathology%20in%20a%20SOD1%20G93A%20model&journal=Cell%20Death20Dis&volumn=10&publication_year=2019&author=Ionescu%2CA Ionescu, A., Zahavi, E. E., Gradus, T., Ben-Yaakov, K. & Perlson, E. Compartmental microfluidic system for studying muscle-neuron communication and neuromuscular junction maintenance. Eur. J. Cell Biol. 95, 69-88 (2016), Osaki, T., Uzel, S. G. M. & Kamm, R. D. Microphysiological 3D model of amyotrophic lateral sclerosis (ALS) from human iPS-derived muscle cells and optogenetic motor neurons. Sci. Adv. 4, eaat5847 (2018), and Ionescu, A. & Perlson, E. Patient-derived co-cultures for studying ALS. Nat. Biomed. Eng. (2018). doi.org-10.1038/s41551-018-0333-8, enabling to study local protein synthesis events at the subcellular level.

Here, we describe a toxic gain-of-function of TDP-43 accumulation in axons and NMJs, which impacts synaptic protein synthesis and provokes neurodegeneration. We demonstrate that TDP-43 mislocalization leads to formation of axonal RNP-complexes that interfere with local protein synthesis in axons and NMJs. This process reduces the levels of nuclear-encoded mitochondrial proteins, consequently leading to NMJ dysfunction. Finally, clearance of TDP-43 or breakdown of G3BP1 condensates in axons reverses the pathological events, signifying a possible pathway for MN recovery in ALS.

Results

Phosphorylated TDP-43 Accumulates in Motor Axons in ALS or Upon Induced TDP-43 Mislocalization

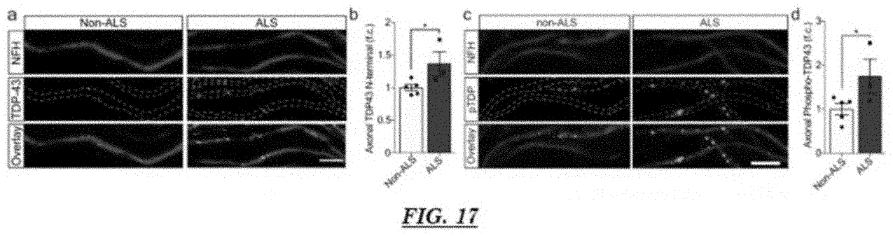

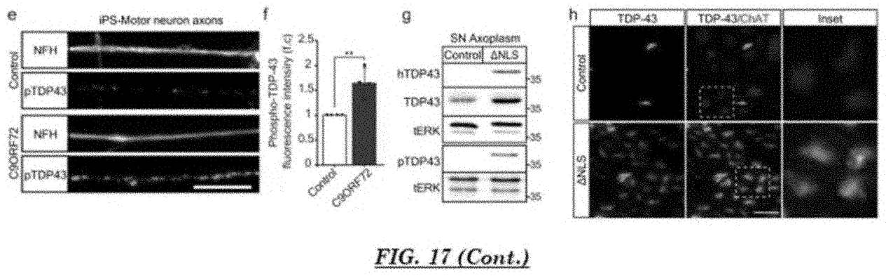

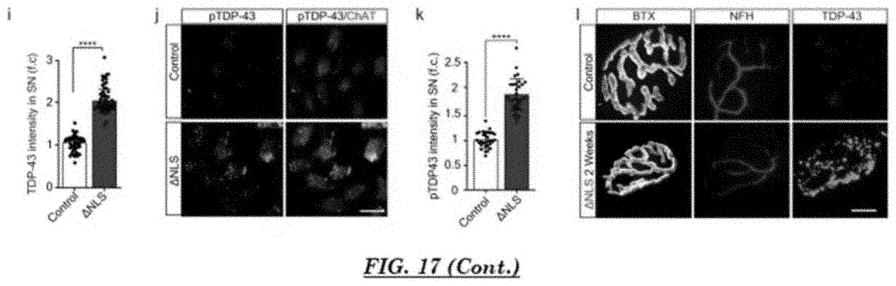

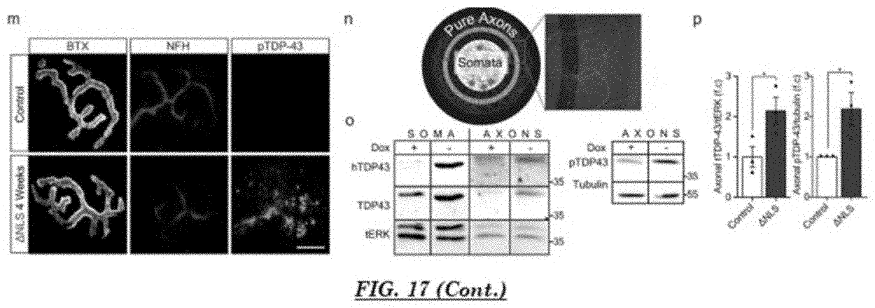

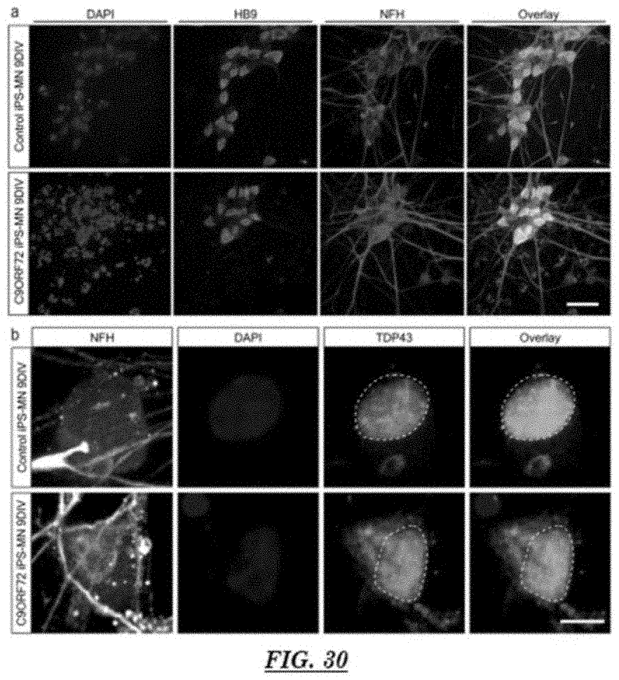

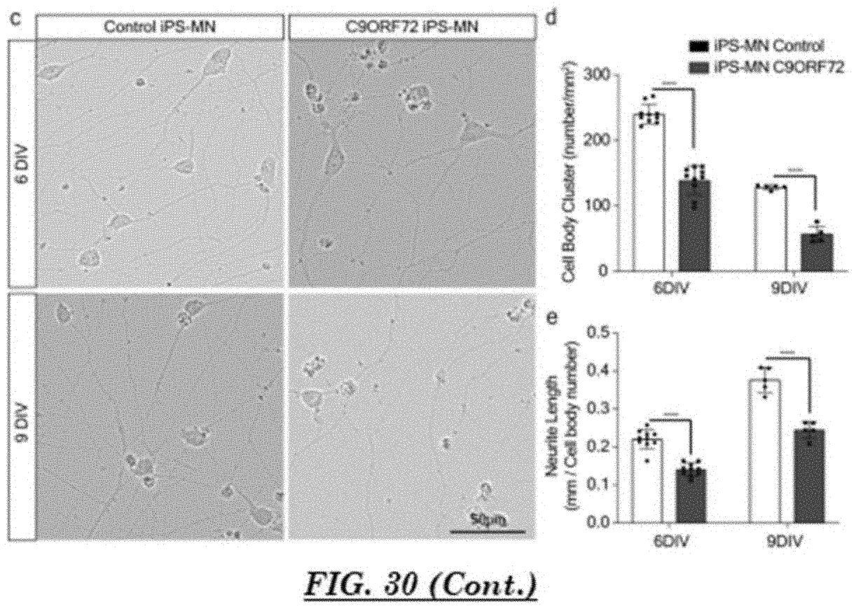

TDP-43 mislocalizes to the cytoplasm in ALS patient spinal cord MNs, where it is observed in highly ubiquitinated and phosphorylated insoluble aggregate-like structures. See, Neumann, M. et al. Ubiquitinated TDP-43 in frontotemporal lobar degeneration and amyotrophic lateral sclerosis. Science 314, 130-133 (2006) and Melamed, Z. et al. Premature polyadenylation-mediated loss of stathmin-2 is a hallmark of TDP-43-dependent neurodegeneration. Nat. Neurosci. 22, 180-190 (2019). However, the spread of TDP-43 pathology to MN axons and NMJs was not thoroughly investigated. To determine whether TDP-43 axonal mislocalization occurs in ALS patients, we immuno-stained TDP-43 in muscle biopsies from sporadic ALS and non-ALS patients. This revealed that the levels of both TDP-43 and its pathological phosphorylated form (pTDP-43), seeNeumann, M. et al. Ubiquitinated TDP-43 in frontotemporal lobar degeneration and amyotrophic lateral sclerosis. Science 314, 130-133 (2006) and Mann, J. R. et al. RNA binding antagonizes neurotoxic phase transitions of TDP-43. Neuron 102, 321-338.e8 (2019), are elevated in intra-muscular nerves of ALS patients (FIG. 17 at a-d). Next, we tested the existence of axonal TDP-43 pathology in C9ORF72 ALS patient iPS-MNs, see Ababneh, N. A. et al. Correction of amyotrophic lateral sclerosis related phenotypes in induced pluripotent stem cell-derived motor neurons carrying a hexanucleotide expansion mutation in C9orf72 by CRISPR/Cas9 genome editing using homology-directed repair. Hum. Mol. Genet. 29, 2200-2217 (2020) and ernandopulle, M. S. et al. Transcription factor-mediated differentiation of human iPSCs into neurons. Curr. Protoc. Cell Biol. 79, e51 (2018), as previously reported for cell-bodies7. Evidently, C9ORF72 iPS-MNs had a significant increase in axonal levels of pTDP-43 compared to its isogenic control line (FIG. 17 at e-f, FIG. 30). Thus, pTDP-43 accumulates in MN axons of ALS patients. FIG. 17, TDP-43 mislocalizes to MN axons in ALS patients and TDPΔNLS mice, shows at a, c Immunofluorescence images and b, d Quantification of non-ALS and ALS patient intra-muscular nerves TDP-43 (b) or phosphorylated TDP-43 (pTDP-43) (d) signal within NFH-positive axons, normalized to NFH intensity. Scale bar=10 μm n=5, 3 patients. Data are presented as mean values±SD. Unpaired-t-test, one-sided, *P=0.026 (b), 0.031 (d). e, f Images (e) and quantification (f) of pTDP-43 in axons (NFH) of C9ORF72 and control iPS-MN. Scale bar=10 μm. n=210,181 axons. Data are presented as mean values±SEM. Unpaired-t-test, two-sided. **P=0.0042. g Western-blots for TDP-43, human-specific-TDP-43 (hTDP-43) and pTDP-43 in TDPΔNLS and control mice sciatic nerve axoplasm (SN axoplasm). ERK1/2 (tERK) used as loading control. n=3, 3 mice. h-k Images (h, j) and quantification (i, k) of TDP-43 (h) or pTDP-43 (j) intensity in ChAT-positive sciatic nerve axons of TDPΔNL^{ChAT::tdTomato} and control^{ChAT::tdTomato} mice. Scale bar=20 μm (h), 10 μm (j). n (h-i)=50,48, n (j-k)=31,31 images from 3,3 mice. Data are presented as mean values±SD. Unpaired-t-test, two-sided. ****P<0.0001. l-m Images of TDP-43 (l) or pTDP-43 (m) in NMJs before, two-weeks (l) and four-weeks (m) after dox retraction from TDPΔNLS mice diet. Bungarotoxin (BTX) and NFH mark the pre- and postsynaptic compartments respectively. Scale bar=10 μm. n (l, m)=3,3 mice. n Radial MFCs structure. MNs axons (HB9::GFP) grow radially-outwards through microgrooves for large-scale protein/RNA purification. o, p Western-blots (o) and quantification (p) of hTDP-43, TDP-43 and tERK (left), and pTDP-43 and tubulin (right) in protein lysates of TDPΔNLS or control MNs/axons isolated from radial MFCs. n=3 repeats. Data are presented as mean values±SEM. Unaired-t-test, one-sided, *P=0.028 (left panel), Unpaired-t-test, two-sided, *P=0.043 (right panel). f.c stands for Fold Change. FIG. 30, Differentiation and characterization on C9ORF72 ALS patient-derived iPSMN and corrected isogenic control, shows a) Representative immunofluorescent images of C9ORF72-mutated and isogenic control iPS-MN at 9- days in vitro (DIV) demonstrating Doxycylin-induced differentiation into MN. DAPI (Blue). HB9 (Green). NFH (Red). Scale bar=50 μm. n=3 independent experiments. b) Representative immunofluorescent images of TDP43 cytoplasmic mislocalization in C9ORF72 iPS-MN. Grey indicates NFH, blue indicates nuclei (DAPI), green indicates TDP-43. Scale bar=10 μm. n=3 independent experiments. c) Representative bright-field images and quantification (d-e) demonstrating differences in neurite length between C9ORF72 iPS-MN and their isogenic controls. Quantification of the d) number of cell-body cluster per mm2 and e) the neurite length in mm per cell body cluster in C9ORF72 iPS-MN and isogenic control. Scale bar=50 μm. n=10 wells for 6-DIV, and n=5 for 9-DIV wells. SD. Unpaired t-test, two-sided. ****p<0.0001.

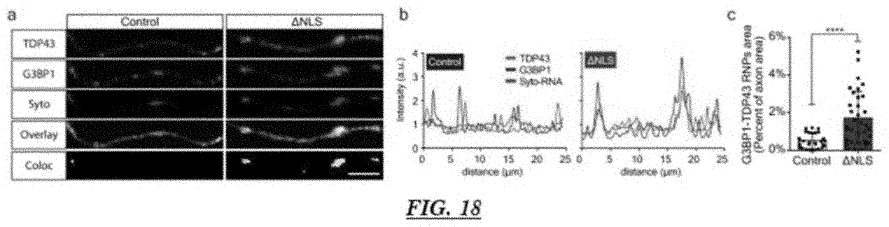

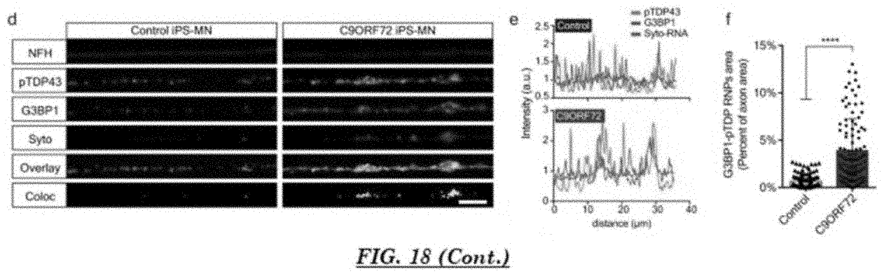

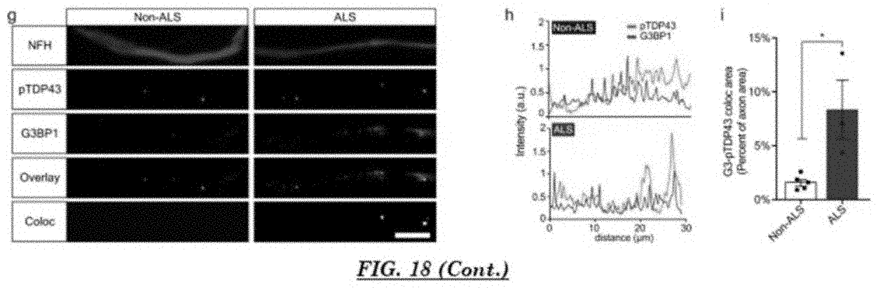

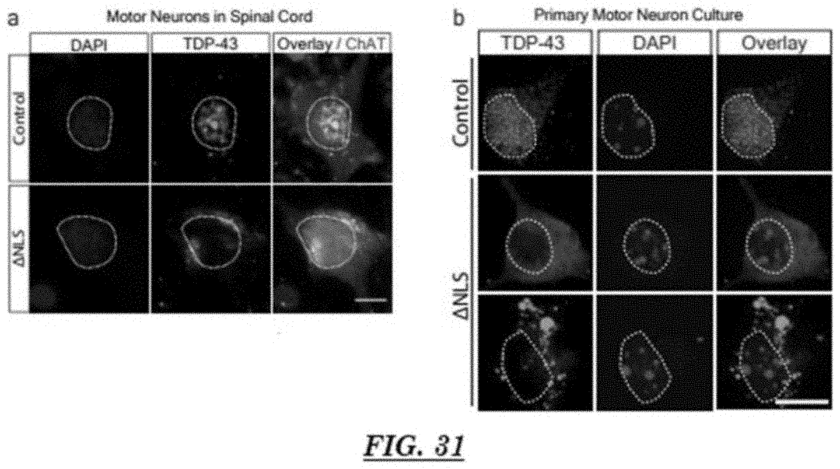

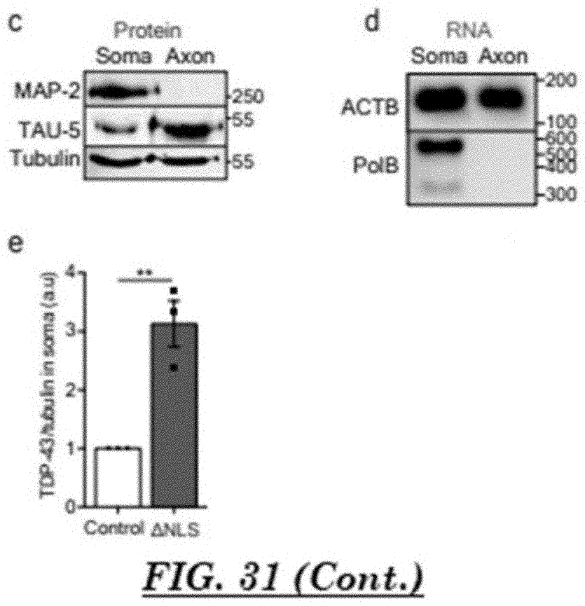

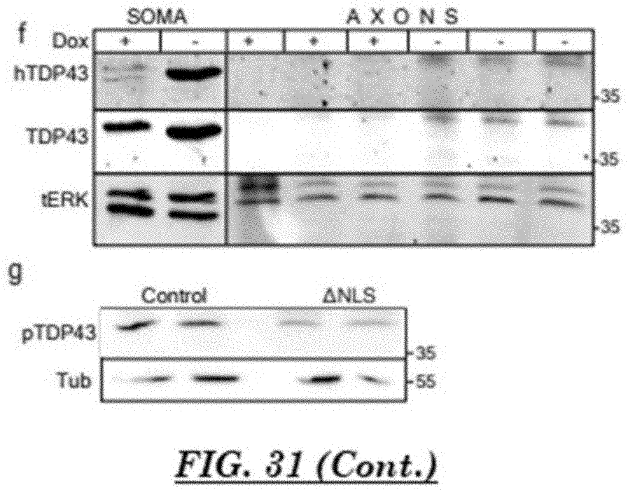

To further study the role of TDP-43 axonal mislocalization, we utilized inducible transgenic mice expressing the human TDP-43 lacking the nuclear-localization-signal (ANLS) through the doxycycline (dox) TET-off system. This TDPΔNLS mouse model recapitulates ALS-like MN disease pathologies, see Walker, A. K. et al. Functional recovery in new mouse models of ALS/FTLD after clearance of pathological cytoplasmic TDP-43. Acta Neuropathol. 130, 643-660 (2015), Spiller, K. J. et al. Selective motor neuron resistance and recovery in a new inducible mouse model of TDP-43 proteinopathy. J. Neurosci. 36, 7707-7717 (2016), Spiller, K. J. et al. Microglia-mediated recovery from ALS-relevant motor neuron degeneration in a mouse model of TDP-43 proteinopathy. Nat. Neurosci. 21, 329-340 (2018), including NMJ disruption, in a mechanism that is not fully understood. To precisely monitor MNs, TDPΔNLS mice were crossbred to Choline-Acetyltransferase (ChAT)cre-tdTomatolox mice (hereafter ChATtdTomato). Retraction of dox from adult animals or from primary MN culture, resulted in TDP-43 cytoplasmic mislocalization, and to elevated TDP-43 and pTDP-43 levels in sciatic nerve motor axons and even remote NMJs (FIG. 17 at g-m, FIG. 31 at a-b). To confirm TDP-43 mislocalization into axons, we developed a radial MFC that allows collection of pure axonal protein and RNA in large quantities. Using this system, we found an increase in axonal TDP-43 and pTDP-43 levels upon dox-retraction from MNs (FIG. 17 at n-p and 31 at c-g). Thus, inducing TDP-43 mislocalization in TDPΔNLS mouse MNs, mimics our observations from ALS patients MN axons. FIG. 18, Axonal TDP-43 accumulation leads to formation of pathological RNP condensates, shows at a-c Images (a), colocalization profiles (b) and quantification (c) of TDP-43-G3BP1-SytoRNA colocalization in control or ΔNLS axons. Scale bar=10 μm. rt=28,35 axons. Data are presented as mean values±SD. Unpaired-t-test, two-sided. ****p<0.0001. d-f Images (d), colocalization profiles (e) and quantification (f) of pTDP-43-G3BP1-SytoRNA colocalization in control or C9ORF72 iPS-MN. Scale bar=10 μm. n=108,104 axons. Data are presented as mean values±SD. Unpaired-t-test, two-sided, ****p<0.0001. g-i Images (g), colocalization profiles (h) and quantification (i) of pTDP-43-G3BP1 colocalization in non-ALS or ALS patient intra-muscular nerves. Scale bar=10 μm. n=5,3 patients. Data are presented as mean values±SEM. Unpaired-t-test, two-sided. *P=0.0163. a.u stands for arbitrary units. FIG. 31, TDP-43 mis-localizes in TDPΔNLS mice in vivo and in vitro, shows at a) Representative images of in vivo SC MNs from TDPΔNLS-ChATtdTomato mouse and control stained with tTDP-43 antibody (green) and DAPI (blue). ChAT (red). Scale bar=10 μm. n=3,3 mice. b) Representative images of TDPΔNLS primary cultured MNs cell bodies with (upper panel-control) or without dox (middle and lower panel), stained with TDP-43 antibody (green) and DAPI (blue). Scale bar=10 μm. n=3 independent experiments. c) Western blot and d) PCR of axons extracted from radial MFCs. In c, MN axons and soma were blotted for MAP-2 (upper panel) to mark dendrites and TAU-5 (middle panel) to mark axons. Tubulin (lower panel) was used as a loading control. In d, Qualitative RT-PCR was performed for cell body-specific Polymerase B (Pol-B) as a control for fraction purity, together with BetaActin as a positive control. n=3 independent experiments. e) Western blot analysis of the normalized overall TDP-43 protein levels in control MNs or upon induction of TDPΔNLS expression. SE. n=3 repeats. Unpaired t-test, two-sided. **p=0.0056. f) Western blots showing all three repeats of hTDP43 and TDP43 levels in distal axons of control or TDPΔNLS MNs cultured in radial MFC. Total ERK (ERK1/2) was used for loading control. n=3 experiments. g) Full-uncropped blot of pTDP43 levels in distal axons of control or TDPΔNLS MNs cultured in radial MFC. Tubulin was used as loading control. n=3 experiments. a.u stands for arbitrary units.

Axonal TDP-43 Colocalizes with G3BP1 and Forms RNP Condensates

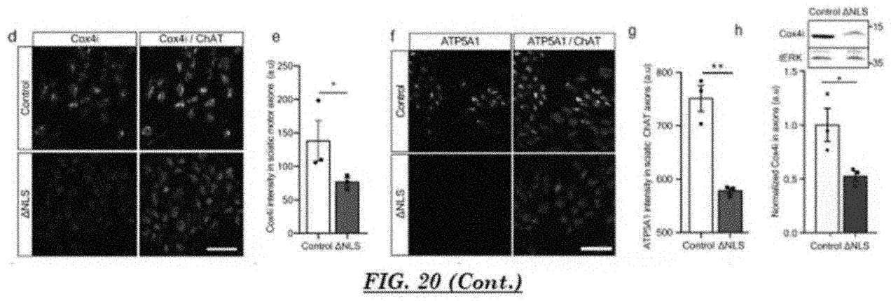

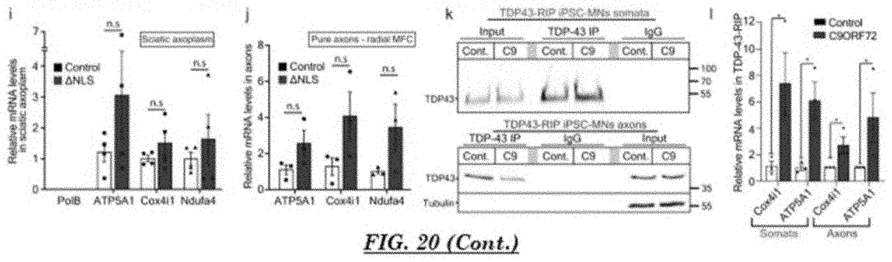

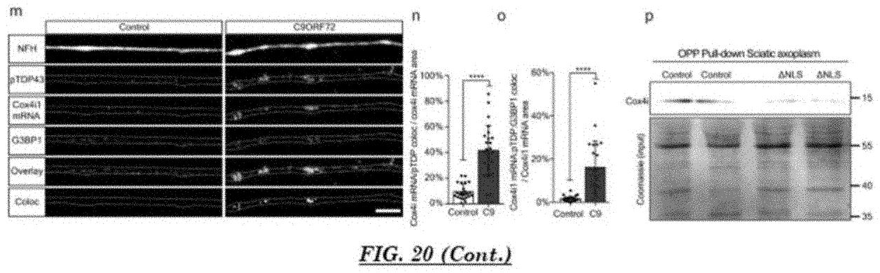

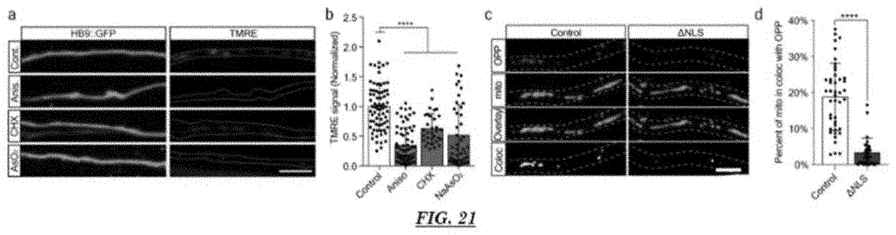

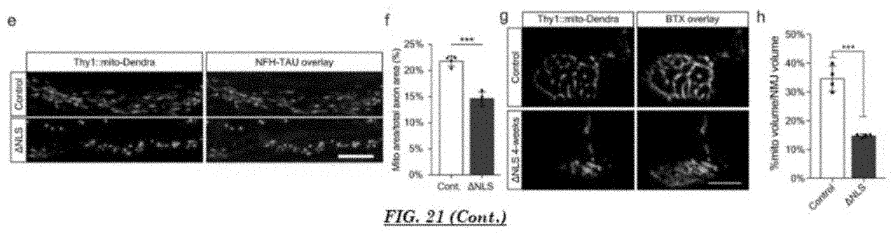

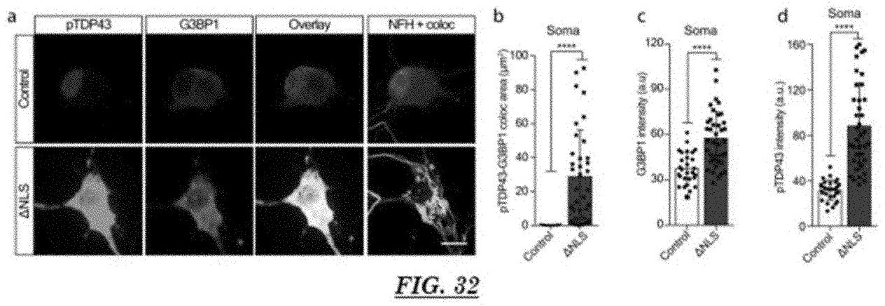

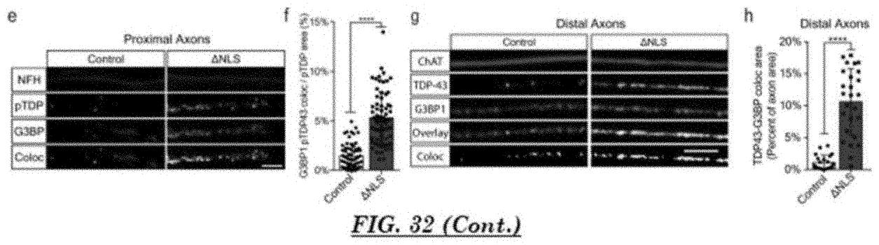

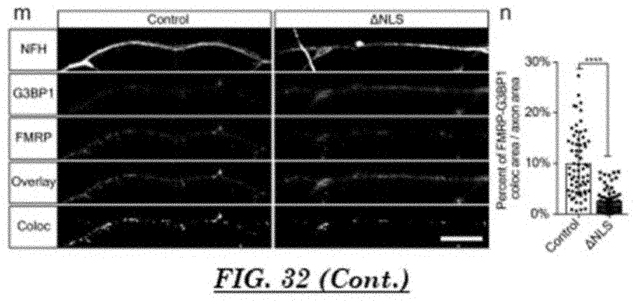

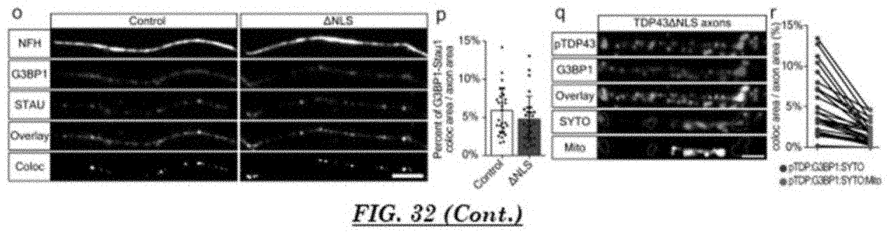

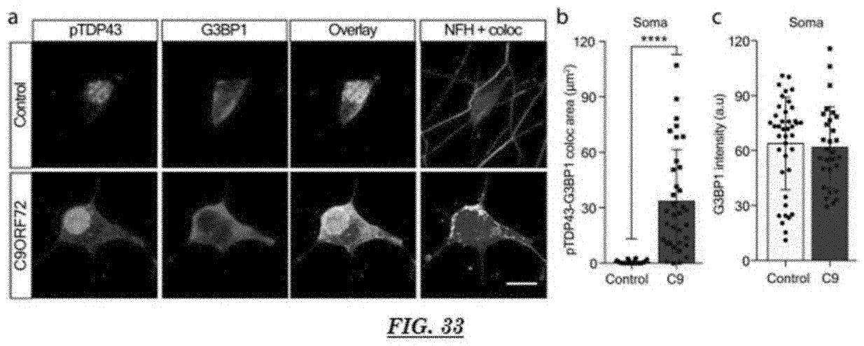

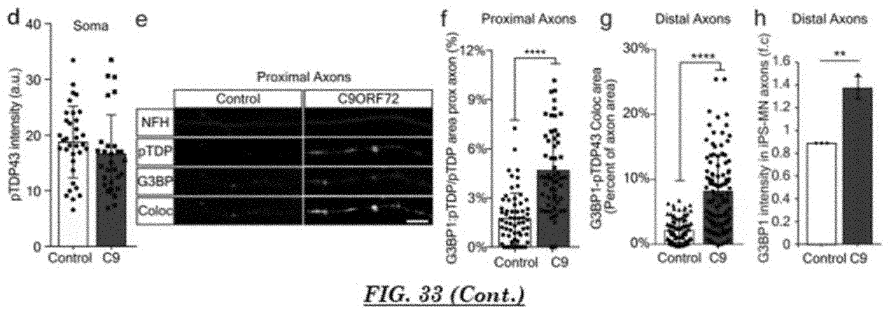

Upon its perinuclear mislocalization in ALS MNs, TDP-43 forms insoluble aggregate-like structures, often in its phosphorylated form. See, Chen, Y. & Cohen, T. J. Aggregation of the nucleic acid—binding protein TDP-43 occurs via distinct routes that are coordinated with stress granule formation. J. Biol. Chem. 294, 3696-3706 (2019), Russo, A. et al. Increased cytoplasmic TDP-43 reduces global protein synthesis by interacting with Rack1 on polyribosomes. Hum. Mol. Genet 26, 1407-1418 (2017), Gasset-Rosa, F. et al. Cytoplasmic TDP-43 de-mixing independent of stress granules drives inhibition of nuclear import, loss of nuclear TDP-43, and cell death article. Neuron 102, 339-357 (2019), and Mann, J. R. et al. RNA binding antagonizes neurotoxic phase transitions of TDP-43. Neuron 102, 321-338.e8 (2019). To determine whether TDP-43 creates similar condensates in axons, we grew primary TDPΔNLS MN in compartmentalized cultures, and tested TDP-43 colocalization with the RNP component, Ras GTPase-activating protein-binding-protein1 (G3BP1). See, Liao, Y. C. et al. RNA granules hitchhike on lysosomes for long-distance transport, using Annexin All as a molecular tether. Cell 179, 147-164.e20 (2019), and Sahoo, P. K. et al. Axonal G3BP1 stress granule protein limits axonal mRNA translation and nerve regeneration. Nat. Commun. 9, 1-14 (2018). Evidently, TDP-43 extensively colocalizes with G3BP1 upon dox retraction in all neuronal compartments of TDPΔNLS MNs (FIG. 32 at a-l). Other RNA binding proteins, such as FMRP and FUS, did not show similar colocalization with G3BP1 in TDPΔNLS MN axons (FIG. 32 at m-p). Notably, using co-labeling with SYTO RNA-select dye, see Alami, N. H. et al. Axonal transport of TDP-43 mRNA granules is impaired by ALS-causing mutations. Neuron 81, 536-543 (2014), we found that RNA, a critical component of cytoplasmic RNP condensates, is enriched in the axonal TDP-43-G3BP1 complexes (FIG. 18 at a-c, FIG. 32 at q-r). Furthermore, we observed formation of similar axonal RNP complexes between pTDP-43, G3BP1, and RNA in C9ORF72 iPS-MNs (FIG. 18 at d-f, FIG. 33), and between pTDP-43 and G3BP1 in patient intra-muscular nerves (FIG. 18 at g-i). Thus, mislocalized TDP-43 associates with G3BP1 to form RNP condensates along MN axons. FIG. 20 shows a Volcano-plot of sciatic nerve axoplasm proteome analysis from TDPΔNLS and control mice. Data is shown as Log₂FC of TDPΔNLS over control proteome. n=4,4 mice. Nuclear-encoded mitochondrial proteins (Mitocarta) in blue. b Venn-diagram of all Mitocarta proteins that were up-regulated (upper panel) or down-regulated (lower panel). Log₂FC>1 for up-regulated and <−1 for down-regulated. c GO analyses categories. Blue=down-regulated, red=up-regulated. Center of the box plots shows median values, boxes extent from 25% to the 75% percentile, whiskers show 5% and 95% percentile. n is the number of proteins as specified in the figure per category. d-g Images and quantification of Cox4i (d, e) and ATP5A1 (f, g) levels in ChAT-positive axons within TDPΔNLS^{ChAT::tdTomato} and control sciatic nerve cross-sections. Scale bar=20 μm. n=3,3 mice. Data are presented as mean values±SEM. Mann-Whitney test, one-tailed, *P=0.05, Unpaired-t-test, one-sided, **p<0.0011. h Western-blot and quantification of Cox4i in isolated axons from TDPΔNLS or control MNs cultures. tERK used as loading control. n=3,3 repeats. Unpaired-t-test, two-sided, *p=0.0413. i-j RT-qPCR of Cox4i1, ATP5A1 and Ndufa4 mRNA in TDPΔNLS and control sciatic axoplasm (i) or pure axons of TDPΔNLS or control MNs cultured in radial MFC (j). mRNA levels normalized to mitochondria content (MT-RNR1 levels). PolB mRNA indicates no nuclear-RNA contamination. n(i)=3,3 mice, n(j)=3,3 repeats. One-way-ANOVA with Holm-Sidak correction. Data are presented as mean values±SEM. k, l TDP-43-RIP of somata (upper panel) and pure axons from radial MFC (lower panel) in C9ORF72 and control iPS-MNs, immunoblotted for TDP-43 and tubulin as a loading control. l RT-qPCR of Cox4i1 and ATP5A1 mRNA levels following TDP-43-RIP from somata and axons. in C9ORF72 and control iPS-MNs. n=3 independent repeats. Data are presented as mean values±SEM. Mann-Whitney test, one-sided *P=0.05 (somata Cox4i1), Unpaired-t-test, two-sided *P=0.0261(somata ATP5A1), Unpaired t-test, one-sided, P=0.0275 (axons Cox4i1), Mann-Whitney test, one-sided, P=0.05. m-o Images (m) and quantification (n, o) of Cox4i1 mRNA-pTDP-43 colocalization (n) and Cox4i1 mRNA-pTDP-43-G3BP1 colocalization (o) in C9ORF72 and control iPS-MN axons. Scale bar=5 μm. n=24,25 images from 3 repeats. Data are presented as mean values±SD. Unpaired-t-test, two-sided ****p<0.0001. p Immunoblot for Cox4 following OPP pull-down (upper panel) and Coomassie staining (total protein) of input lysates (lower panel) from TDPΔNLS and control sciatic axoplasms labeled with OPP. n=10 mice (20 sciatic nerves) per each lane. a.0 stands for arbitrary units. FIG. 32, TDP-43 cytoplasmic mislocalization leads to co-localization with RNP granule marker G3BP1 in Cell bodies, proximal and distal axons of TDPΔNLS MNs, shows a) Representative images and b) quantification results from 3D-colocalization analysis examining the colocalization (yellow) of phosphorylated TDP-43 (pTDP; green) and G3BP1 (magenta) in cell bodies of cultured control or TDPΔNLS MNs. Cyan indicates NFH. Scale bar=10 μm. n=38,40 cells. SD. Unpaired t-test, two-sided. ****p<0.0001. c-d) Quantification of G3BP1 (C) and pTDP43 (D) intensity in control or TDPΔNLS MNs. n=38,40 cells. Data is shown as the mean ±SD. Unpaired t-test, two-sided. ****p<0.0001. e) Representative images and f) quantification of the pTDP43-G3BP1 colocalization in proximal axons of TDPΔNLS and control MNs. Blue indicates NFH, Green indicates pTDP43, Magenta indicates G3BP1. Scale bar=5 μm. n=66,70 axons. SD. Unpaired t-test, two-sided. ****p<0.0001 g-j) Representative images (g) and quantification (h-j) of TDP-43 (green) and G3BP1 (magenta) colocalization (h), average TDP-43 (i) and average G3BP1 (j) intensities in distal axons of ChAT::Rosa (red) expressing TDPΔNLS or control MNs. Scale bar=10 μm. n=22,26 axons. SD. Unpaired t-test, two-sided. ****p<0.00001, ***p=0.0008. k) Representative western-blot and 1) quantification of G3BP1 levels in distal axons of control or TDPΔNLS cultured MNs within radial MFCs. Tubulin was used for loading control. n=3 experiments. SE. Unpaired Mann-Whitney test. *p=0.05. m-n) Representative images (m) and quantification (n) results from 3D-colocalization analysis examining the colocalization (yellow) of FMRP (green) and G3BP1 (magenta) in axons of cultured control or TDPΔNLS MNs. White indicates NFH. Scale bar=10 μm. n=68,83 axons. SD. Unpaired t-test, two-sided. ****p<0.0001. o-p) Representative images (o) and quantification (p) results from 3D-colocalization analysis examining the colocalization (yellow) of Staufenl (green) and G3BP1 (magenta) in axons of cultured control or TDPΔNLS MNs. White indicates NFH. Scale bar=5 μm. n=29,29 axons. SD. Unpaired t-test, two-sided. q-r) Representative images (q) and colocalization analysis (r) showing pTDP (green), G3BP1 (magenta), Syto RNA (cyan) and mitotracker (white) staining in axons of cultured control or TDPΔNLS MNs. Scale bar=3 μm. n=28,28 axons. a.0 stands for arbitrary units. f.c stands for Fold Change.

FIG. 33, Cytoplasmic mislocalization of phosphorylated-TDP-43 leads to co-localization with RNP granule marker G3BP1 in cell bodies, proximal and distal axons of ALS C9ORF72 iPS-MN, shows at a) Representative images and b) quantification results from 3D-colocalization analysis examining the colocalization (yellow) of phosphorylated TDP-43 (pTDP; green) and G3BP1 (magenta) in cell bodies of cultured C9ORF72 or isogenic control iPS-MNs. Cyan indicates NFH. Scale bar=10 μm. n=46,35 cells. SD. Unpaired t-test, two-sided. ****p<0.0001. c-d) Quantification of G3BP1 (C) and pTDP43 (D) intensity in C9ORF72 or control iPS-MNs. n=46,35 cells. SD. e) Representative images and f) quantification of the pTDP43-G3BP1 colocalization in proximal axons of C9ORF72 and control MNs iPS-MNs. Blue indicates NFH, Green indicates pTDP43, Magenta indicates G3BP1. Scale bar=5 μm. n=64,55 axons. SD. Unpaired t-test, two-sided. ****p<0.0001. g-i) Quantification of G3BP1 colocalization and intensity in C9ORF72 and control iPS-MN h) The percent of TDP43-G3BP1 colocalized area out of the total axonal area in C9ORF72 and control iPS-MN. n=108,104 axons. SD. Unpaired t-test, two-sided. ****p<0.0001. i) Average G3BP1 intensity in iPS-MN axons n=4 repeats. SE. Unpaired t-test, two-sided. *p=0.028. a.0 stands for arbitrary units. f.c stands for Fold Change.

Axonal TDP-43 RNP Assembly Reduces Local Protein Synthesis in MN Axons and NMJs

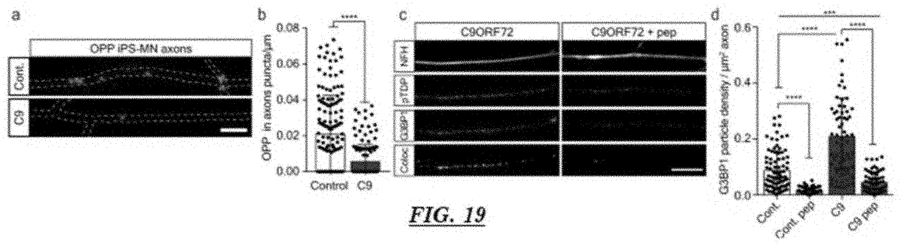

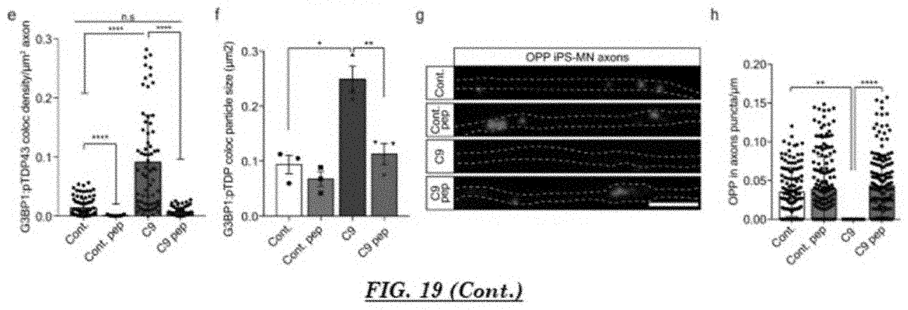

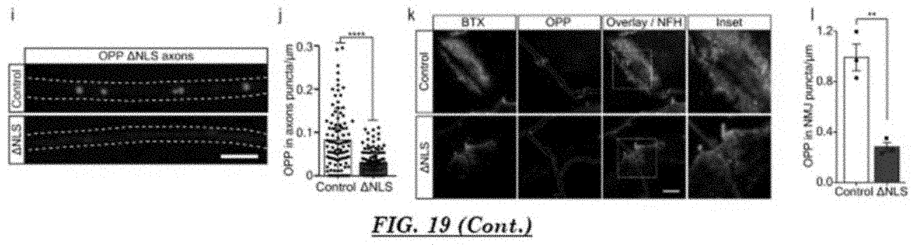

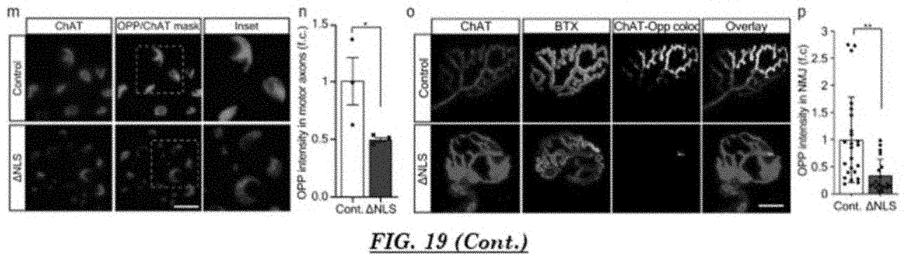

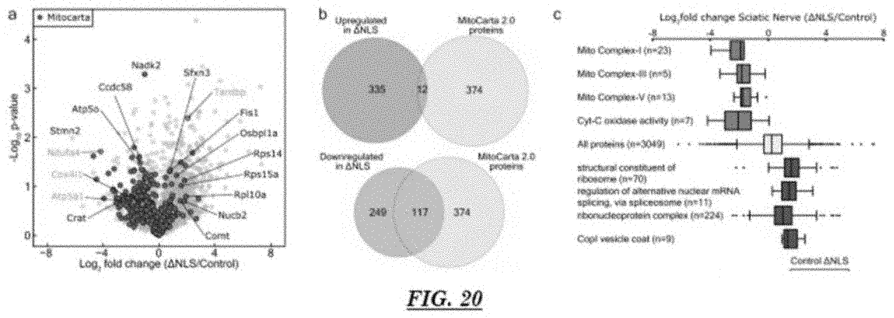

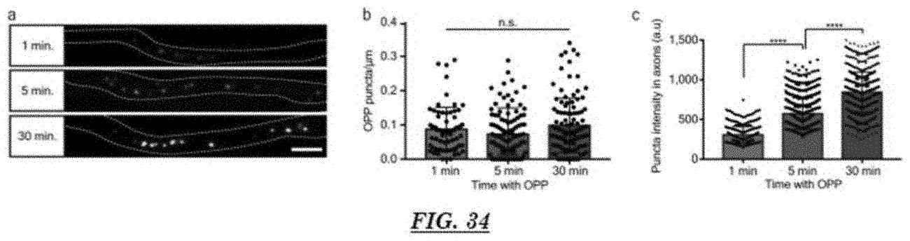

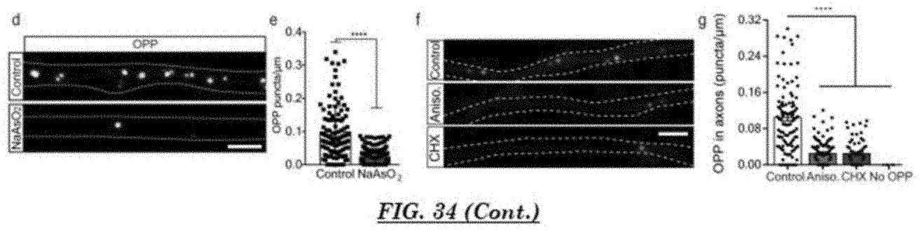

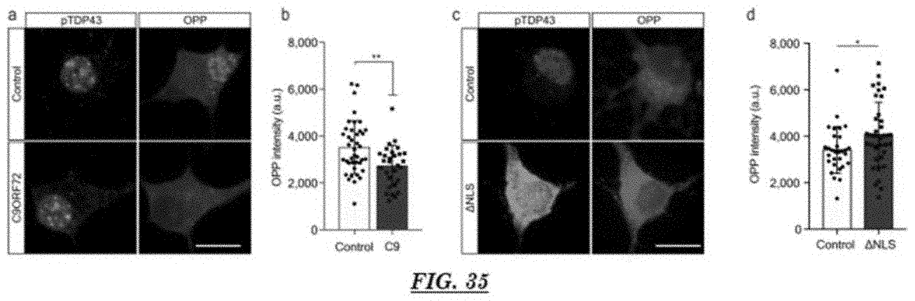

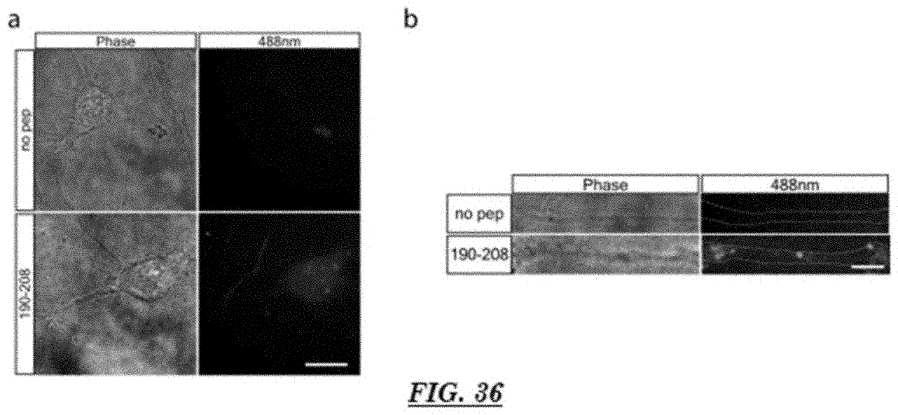

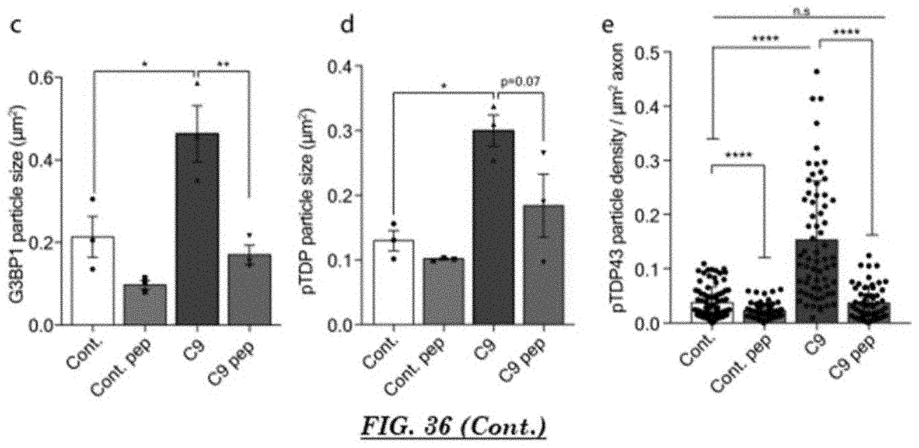

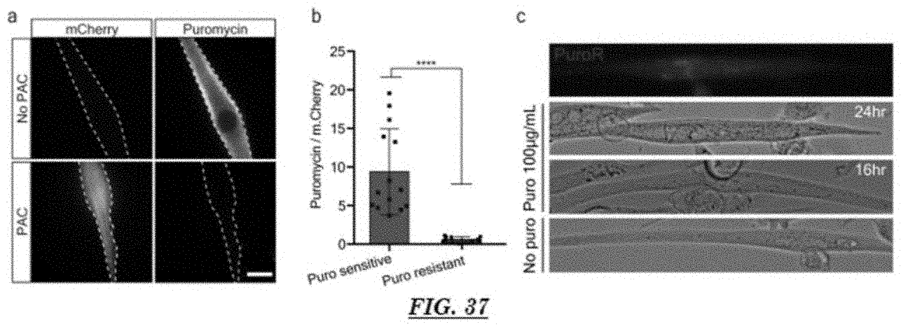

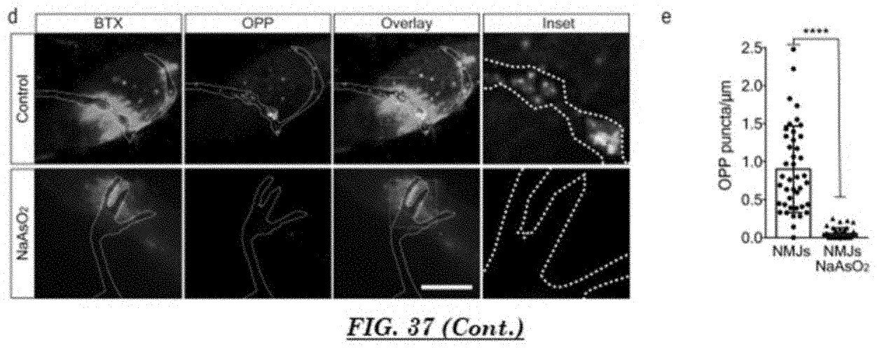

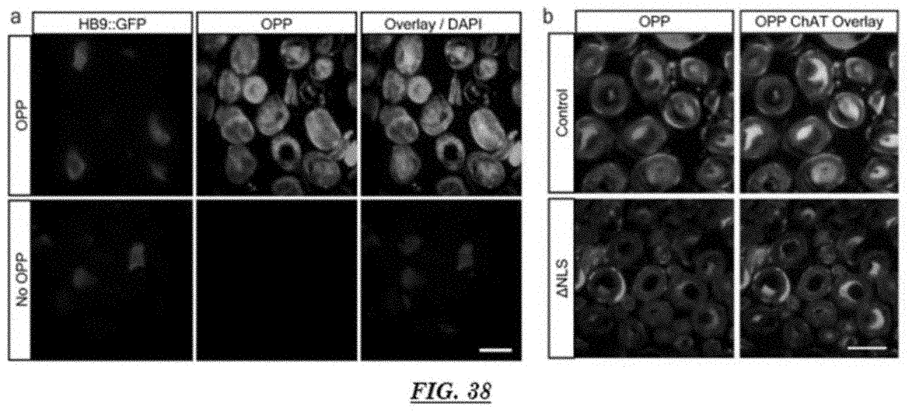

Formation of G3BP1-positive RNP condensates is strongly associated with repressed RNA translation, see Sahoo, P. K. et al. A Ca²⁺ dependent switch activates axonal casein kinase 2α translation and drives G3BP1 granule disassembly for axon regeneration. Curr. Biol. 30, 4882-4895.e6 (2020), and altered mRNA transport, see Liao, Y. C. et al. RNA granules hitchhike on lysosomes for long-distance transport, using Annexin All as a molecular tether. Cell 179, 147-164.e20 (2019), leading to reduced protein synthesis, see Sahoo, P. K. et al. Axonal G3BP1 stress granule protein limits axonal mRNA translation and nerve regeneration. Nat. Commun. 9, 1-14 (2018). We therefore sought to determine whether TDP-43 axonal accumulation, and subsequent RNP condensate formation, impair local protein synthesis in MN axons and NMJs. To test this, we cultured C9ORF72 iPS-MNs in MFCs and applied 0-Propargyl-Puromycin (OPP) to the axonal compartment to label newly synthesized proteins. We found a substantial decrease in density of the OPP puncta in C9ORF72 axons (FIG. 19 a-b, FIG. 34 at a-c), which was less apparent in cell-bodies (FIG. 35). This decrease in translation was reversed by axonal-exclusive application of TAT-fused peptide corresponding to residues 190-208 of G3BP1 (G3BP1 peptide), which was recently reported to dissociate G3BP1 condensates, see Id., FIG. 19 c-h, FIG. 36). Thus, axonal RNP condensate assembly drives suppression of local protein synthesis. To ensure that local protein synthesis inhibition occurs due to axonal TDP-43, we cultured primary TDPΔNLS MNs in MFCs, and applied OPP to the axonal compartment. Again, we observed a major decrease in axonal translation (FIG. 19 i-j), similar to that seen by inducing RNP-condensates with sodium-arsenite (NaAsO2), see Mark Fang, A. Y. et al. Small-molecule modulation of TDP-43 recruitment to stress granules prevents persistent TDP-43 accumulation in ALS/FTD. (2019). doi.org-10.1016/j.neuron.2019.05.048, or by inhibiting protein synthesis using Anisomycin and Cycloheximide, all exclusively in axons (FIG. 33 at d-g). Next, we aimed to evaluate local protein synthesis also in the most remote point of the MN axon, the NMJ. To that end, we performed co-cultures of TDPΔNLS MNs with healthy puromycin-resistant muscles to avoid post-synaptic staining (FIG. 37 at a-c). By quantifying OPP puncta density within in vitro NMJs, we identified robust protein synthesis within control co-cultures, while protein synthesis in TDPΔNLS NMJs was severely impaired (FIG. 19 3k-l). A similar decrease in OPP density was observed after NaAsO₂ application (FIG. 37 at d-e). Following these findings, we examined the extent of protein synthesis interference in adult TDPΔNLS mice. Labeling and quantification of the OPP signal in sciatic nerves and hind-limb muscles revealed a reduction in the amount of newly synthesized proteins both in sciatic MN axons and in NMJ pre-synapse of TDPΔNLS mice (FIG. 19 m-p, FIG. 38), see, Terenzio, M. et al. Locally translated mTOR controls axonal local translation in nerve injury. Science 359, 1416-1421 (2018). Taken together, we demonstrate that TDP induced axonal assembly of RNP condensates disrupts protein synthesis in axons and in the NMJ pre-synapse. FIG. 19, TDP-43 mediated formation of axonal condensates reduces Local Synthesis in MN axons and NMJs, shows a Images and b quantification of OPP puncta density in C9ORF72 or control iPS-MN axons. Scale bar=5 μm. n=159,144 axons. Data are presented as mean values±SD. Unpaired-t-test, two-sided ****p<0.0001. c Images and d-f quantification of NFH, pTDP-43 and G3BP1 and their colocalization in C9ORF72 iPS-MN axons either treated or not with G3BP1 peptide. Quantifications of G3BP1 particle density (d), G3BP1-pTDP-43 coloc-particle density (e) and G3BP1-pTDP-43 coloc-particle size (f) from C9ORF72 and control iPS-MN axons, either treated or not with G3BP1 peptide. Scale bar=10 μm. n (d and e)=91,71,65,68 axons, n(f)=3 repeats, each over 1000 particles. Data are presented as mean values ±SD. One-way-ANOVA with Holm-Sidak correction. *P=0.0151,'P=0.0074, ***P=0.0005, ****p<0.0001. g Images and h quantification of OPP puncta density from C9ORF72 and control iPS-MN axons either treated or not with G3BP1 peptide. Scale bar=5 μm. n=130,71,193,173 axons. Data are presented as mean values ±SD. One-way-ANOVA with Holm-Sidak correction. **P=0.094, ****p<0.0001. i Images and j quantification of OPP puncta density in control or ΔNLS MN axons. Scale bar=5 μm. n=100,112 axons. Data are presented as mean values±SD. Unpaired-t-test, two-sided. ****p<0.0001. k Images and 1 quantification of OPP puncta density in in vitro NMJs from control or ΔNLS co-cultures. BTX indicates post-synapse, NFH indicates pre-synapse. Scale bar=5 μm. n=3 independent repeats. Data are presented as mean values±SEM. Unpaired-t-test, two-sided. **P=0.0031. m Images and n quantification of OPP-labeled sciatic nerve sections from TDPΔNLS^{ChAT::tdTomato} or control^{ChAT::tdTomato} mice. Scale bar=10 μm. Data are presented as mean values±SEM. n=3,3 mice. Unp aired-t-test, one-sided. *P=0.0364. o Images and p quantification of pre-synaptic OPP in NMJs from TDPΔNLS^{ChAT::tdTomato} or control^{ChAT::tdTomato} mice. Scale bar=10 μm. Data are presented as mean values±SD. n=24,19 NMJs, from 3,3 mice. Unpaired-t-test, two-sided. **P<0.0044. f.c stands for Fold Change.

FIG. 34, OPP Labeling in MFCs expose local protein synthesis in MN distal axons, shows at a) Representative images and b) quantification of OPP density and c) OPP puncta fluorescence intensity in distal axons in MFC labeled with OPP (2001) for different time periods (1 min, 5 min, 30 min). Scale bar=10 μm. SD. b) n=70, 92, 110 axons. c) n=472, 741, 819 puncta. One-way ANOVA with Holm-Sidak correction. ****p<0.0001. d) Representative images and e) Quantification of OPP puncta in axons without (control) or with application of NaAsO₂ (25001) to axonal compartment of MFC. Scale bar=10 μm. n=110,137 axons. SD. One-way ANOVA with Holm-Sidak correction. ****p<0.0001. f) Representative images g) and quantification of OPP puncta in axons without (control) or with application of protein synthesis inhibitors Cycloheximide (CHX) and Anisomycin (Aniso). An additional control, with only color labeling but no puromycin (no OPP) was included. Scale bar=10 μm. n=102,63,73,6 axons. One-way ANOVA with Holm-Sidak correction. ****pp<0.0001. a.u stands for arbitrary units.

FIG. 35, OPP Labeling of cell-bodies of C9ORF72 iPS-MNs and TDPΔNLS MNs, shows a) Representative images and b) quantification of OPP signal intensity in C9ORF72 and control iPS-MNs cell-bodies. Green indicates pTDP43, magenta indicates OPP. Scale bar=10 μm. n=40,29. SD. Unpaired t-test, two-sided. p=0.0025. d) Representative images and c) quantification of OPP signal intensity in TDPΔNLS and control MNs cell-bodies. Green indicates pTDP43, magenta indicates OPP. Scale bar=10 μm. n=30,38 cells. SD. Unpaired t-test, two-sided. *p=0.034. a.u stands for arbitrary units.

FIG. 36, G3BP1(190-208) Peptides Reduce G3BP1/pTDP43 particle size and pTDP43 RNP-condensate density in axons of C9ORF72 iPS-MNs, shows at a-b) Representative phase and fluorescent images demonstrating penetration of TAT-fused G3BP1 (190-208)-FITC peptides into primary MNs cell bodies (a) and axons (b). Scale bar=10 μm (a), 5 μm (b). n=3 independent experiments. c) Quantification of G3BP1 particle size in C9ORF72 or control MN axons treated with G3BP1 peptides exclusively in the axonal compartment of the MFC. n=3 experiments, with more than 1,000 G3BP1 particles analyzed in each. SE. One-way ANOVA with Holm-Sidak correction. *p=0.0151, **p=0.0074. d) Quantification of pTDP-43 particle size in C9ORF72 or control MN axons treated with G3BP1 peptides exclusively in the axonal compartment of the MFC. n=3 experiments, with more than 1,000 pTDP-43 particles analyzed in each. SE. One-way ANOVA with Holm-Sidak correction. *p<0.0139. e) Quantification of pTDP-43 particle density in C9ORF72 or control MN axons treated with G3BP1 peptides exclusively in the axonal compartment of the MFC. n=91,71,65,68 axons. SD. One-way ANOVA with Holm-Sidak correction. ****p<0.0001.

FIG. 37, Puromycin-resistant muscles enable visualization of pre-synaptic protein synthesis in in vitro NMJs, shows at a) Representative images and b) quantification of puromycin labeling in control, versus puromycin resistant muscles (transfected with PQCXIP-mCherry backbone vector expressing Puromycin Acetyltransferase gene (PAC)) demonstrating the ability of PAC to prevent puromycin labeling of newly synthesized peptides. Scale bar=20 μm. n=14,14 muscles. Unpaired t-test, two-sided. ****p<0.0001. c) Representative images of puromycin-resistant muscles stable morphology following 16 h and 24 hr incubation with 100 μg/mL puromycin compared to muscles that were not exposed to puromycin. d) Representative images and e) quantification of OPP puncta density of in vitro NMJs in the presence of absence of distal NaAsO₂. Scale bar=5 μm. n=42,44 NMJs. SD. Unpaired t-test, two-sided. ****p<0.0001.

FIG. 38, OPP labeling in sciatic nerves reveals reduced axonal protein synthesis in TDPΔNLS MN axons, shows at a) Representative images of OPP labeling versus only color labeling but no puromycin (no OPP) in HB9:GFP mouse SN sections. HB9 (red) indicates MN axons. Scale bar=10 μm. n=3,3 mice. b) Additional unmasked OPP images and overlay images of SN sections obtained from TDPΔNLS and control mice. Scale bar=10 μm. n=3,3 mice.

TDP-43 Axonal Accumulation Reduces the Levels of Nuclear-Encoded Mitochondrial Proteins by Suppressing Protein Translation

To determine which proteins are primarily affected by axonal TDP-43 accumulation and reduction in local protein synthesis, we performed proteome analysis of sciatic nerve axoplasm samples from TDPΔNLS and control mice. The analysis revealed a global reduction in nuclear-encoded mitochondrial proteins (Mitocarta 2.0, see Calvo, S. E., Clauser, K. R. & Mootha, V. K. MitoCarta2.0: An updated inventory of mammalian mitochondrial proteins. Nucleic Acids Res. 44, D1251—D1257 (2016)), including respiratory chain complex proteins (FIG. 20 at a-c). We specifically validated this reduction for two nuclear-encoded mitochondria proteins, Cox4i1 and ATP5A1 in TDPΔNLS MN axons (FIG. 20 at d-h, FIG. 39 at a).

Since mRNAs of nuclear-encoded mitochondrial genes are among the most abundant mRNAs in MN axons, see Farias, J., Holt, C. E., Sotelo, J. R. & Sotelo-Silveira, J. R. Axon micro-dissection and transcriptome profiling reveals the in vivo RNA content of fully differentiated myelinated motor axons. RNA (2020). doi.org-10.1261/rna.073700.119 and Maciel, R. et al. The human motor neuron axonal transcriptome is enriched for transcripts related to mitochondrial function and microtubule-based axonal transport. Exp. Neurol. 307, 155-163 (2018), we sought to determine whether the reduction in nuclear-encoded mitochondrial proteins occurs at the transcriptional or translational level. We conducted RT-qPCR analysis for three nuclear-encoded mitochondrial genes: ATP5A1, Cox4i1, and Ndufa4 in sciatic nerve axoplasm and cultured MN axons. This analysis revealed a slightly increased mRNA abundance, contradicting their reduced protein levels, suggesting that TDP-43 accumulation impairs nuclear-encoded mitochondrial proteins local translation (FIG. 20 at i-j, FIG. 39 at b).

Next, we hypothesized that the mRNAs of these proteins are directly bound and sequestered within TDP-43 axonal RNP condensates. To assess this, we performed TDP-43 RNA-immunoprecipitation (RIP) from the somata and axonal compartments of C9ORF72 iPS-MNs cultured in radial MFCs. RT-qPCR analysis of TDP-43-bound mRNAs detected a substantial increase in the binding of both Cox4i1 and ATP5A1 mRNAs to TDP-43 in diseased MN somata and axons (FIG. 20 at k-l). Additionally, smFISH for Cox4i1 mRNA in C9ORF72 iPS-MN axons combined with immunostaining for pTDP-43 and G3BP1 revealed extensive sequestration of Cox4i1 mRNA within TDP-43 positive axonal RNP condensates (FIG. 20 at m-o, FIG. 39 at c). Finally, to test if the localization of Cox4i1 mRNA into axonal RNP condensates inhibits its translation, we performed streptaviclin pull-downs of biotinylated-OPP from sciatic nerve axoplasm samples of TDPΔNLS mice. See, Terenzio, M. et al. Locally translated mTOR controls axonal local translation in nerve injury. Science 359, 1416-1421 (2018). Evidently, OPP bound Cox4i1 was decreased in TDPΔNLS sciatic axoplasm, demonstrating that reduction of Cox4i1 levels derives from impaired axonal translation (FIG. 20 at m, FIG. 39 at d-e). Thus, we show that TDP-43 mislocalization leads to deficient local protein synthesis of nuclear encoded mitochondrial genes such as Cox4i1, through sequestration of their mRNA within axonal RNP condensates.

TDP-43 Mislocalization Impairs Axonal and NMJ Mitochondria via Limitation of Mitochondria-Related Protein Synthesis

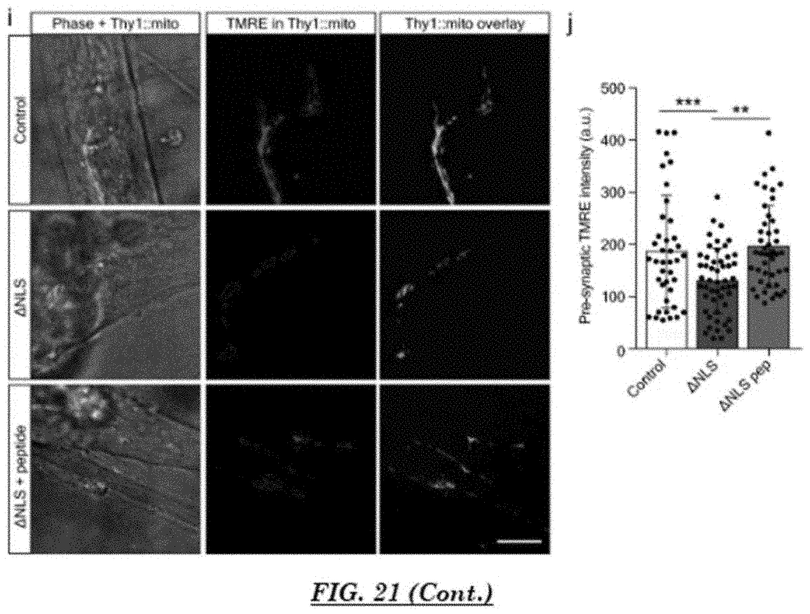

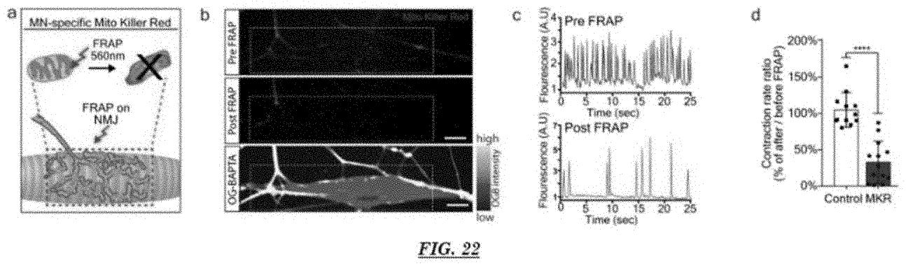

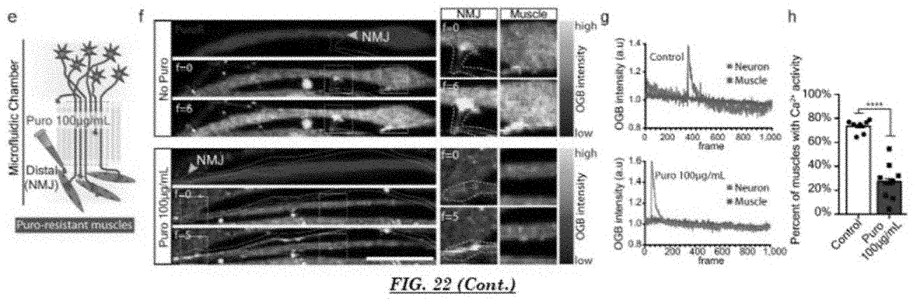

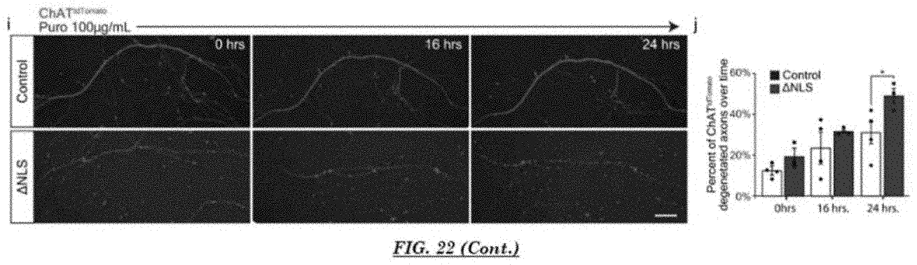

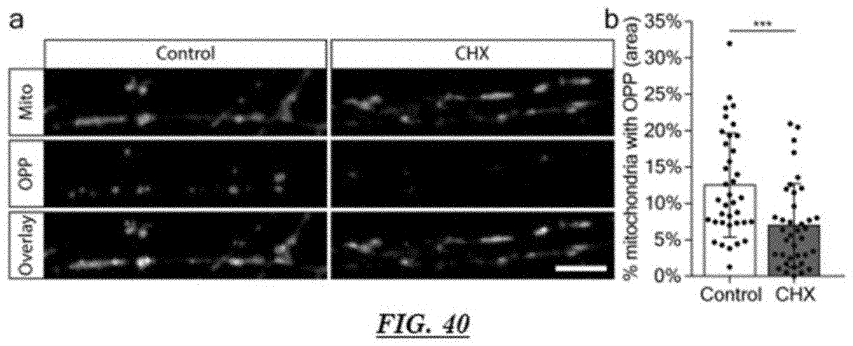

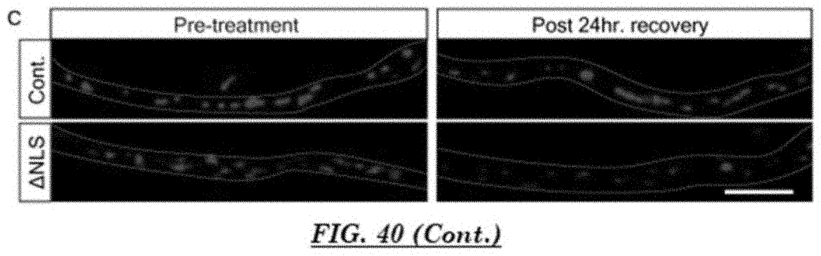

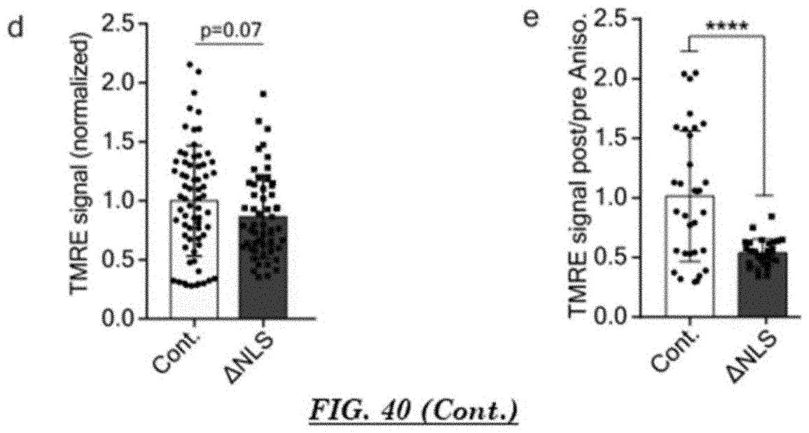

Our observations so far suggest TDP-43 mislocalization affects axonal synthesis of nuclear-encoded mitochondrial proteins through sequestration or their mRNAs in axonal RNP-condensates. Yet, the extent of damage this process projects on axonal and synaptic mitochondria remains elusive. We first assessed the effect of acute axonal protein synthesis inhibition over mitochondria activity using TMRE. Local axonal administration of both anisomycin and CHX, as well as NaAsO₂ led to a notable reduction in mitochondrial membrane potential (FIG. 21 at a-b). Thus, interfering with axonal protein synthesis impedes mitochondria function. As recent findings indicate that synthesis of nuclear-encoded mitochondrial proteins occurs in proximity to mitochondria, see Cioni, J.-M. et al. Late endosomes act as mRNA translation platforms and sustain mitochondria in axons article late endosomes act as mRNA translation platforms and sustain mitochondria in axons. Cell 176, 56-72 (2019), we further examined the association of OPP with mitochondria in TDPΔNLS axons. We identified a profound reduction in this colocalization, with a similar trend to that obtained with protein synthesis inhibition (FIG. 21 at c-d, FIG. 40 at a-b). Therefore, mitochondria associated protein synthesis in axons is decreased by TDP-43 mislocalization. FIG. 40, Mitochondria activity is dependent on local protein synthesis in MN axons, and is impaired in TDPΔNLS MNs, shows a) Representative images and b) quantification of the percent of mitochondria (area; green)) colocalization with OPP (area; red) in MN axons treated exclusively with cycloheximide (CHX) versus control (No CHX). Scale bar=5 μm. n=38,38 axons. SD. Unpaired t-test, two-sided. ***p=0.0005. c) Representative images d) and quantification of the TMRE signal in TDPΔNLS and control MN axons. Scale bar=10 μm. n=68,56 axons. SD. Unpaired t-test, two-sided. p=0.0791. e) Quantification of TMRE signal in TDPΔNLS and control MN axons, following transient 4-hour anisomycin treatment and washout. TMRE signal was collected from distal axons 24-hours after washout and compared to pre-treatment signal. n=32,29 axons. SD. Unpaired-t-test, two-sided. ****p<0.0001.

Next, we measured the direct effect of TDP-43 mislocalization on mitochondria integrity in distal axons by generating TDPΔNTLSThy1-mito-Dendra mice, which express mitochondria targeted florescent tag in neurons. This approach exposed an extensive reduction in axonal and pre-synaptic mitochondria upon induction of TDP-43 mislocalization (FIG. 21 at e-h). Furthermore, by co-culturing TDPΔNLSThy1-mito-Dendra primary MNs with healthy muscles we were capable of measuring TMRE signals in pre-synaptic mitochondria, that were significantly reduced in NMJs following TDP-43 mislocalization (FIG. 21 at 5i-j). Intriguingly, axonal TMRE signals in TDPΔNLS MNs cultured alone were only mildly affected by TDP-43 mislocalization and became more apparent when assessing mitochondrial recovery from acute anisomycin challenge (FIG. 40 at c-e). Critically, mitochondria activity in the NMJs of TDRΔNTLSThy1-mito-Dendra MNs was recovered following localized administration of G3BP1 peptides to the NMJ compartment (FIG. 21 at 5i-j). Hence, axonal TDP-43 RNP-condensates impair mitochondria activity and integrity in NMJs by limiting local translation of vital mitochondrial components. FIG. 21, TDP-43 mislocalization impairs axonal and NMJ mitochondria via limitation of mitochondria-related protein synthesis, shows a Images and b quantification of TMRE signal in HB9::GFP MN axons untreated (control) or treated with anisomycin (aniso), cycloheximide (CHX) or NaAsO₂. Scale bar=10 μm. rt=72,69,43,32 axons. Data are presented as mean values±SD. One-way-ANOVA with Holm-Sidak correction. ****p<0.0001. c Images and d quantification of OPP and mitochondria colocalization in TDPΔNLS or control MN axons. Scale bar=5 μm. n=43,36 axons. Data are presented as mean values±SD. Unpaired-t-test, two-sided ****p<0.0001. e images and f quantification of mitochondria density within sciatic nerve longitudinal sections of TDRΔNLS^{Thy1::MitoDendra} and control mice labeled also for NFH and Tau. Scale bar=10 μm. n=4,4 mice. Data are presented as mean values±SEM. Unpaired-t-test, two-sided. ***p=0.0001. g Images and h quantification of pre-synaptic mitochondrial volume within NMJs of TDPΔNLS^{Thy1::MitoDendra} and control^{Thy1::MitoDendra} mice. BTX marks post-synapse. Scale bar=10 μm. n=4,4 mice. Data are presented as mean values±SEM. Unpaired-t-test, two-sided. ***p=0.0001. i Images and j quantification of pre-synaptic TMRE signal intensity within in vitro NMJs from TDPΔNLS^{Thy1::MitoDendra}, control^{Thy1::MitoDendra} co-cultures and TDPΔNLS^{Thy1::MitoDendra} co-cultures treated with G3BP1 peptides in NMJ compartment. Scale bar=10 μm. n=40,51,45 NMJs. One-way-ANOVA with Holm-Sidak correction. Data are presented as mean values±SD. **p=0.0034, ***p=0.0005. a.u stands for arbitrary units.

Mitochondria Activity and Local Synthesis are Vital for NMJ Function and their Inhibition Leads to Neurodegeneration

Thus far, we demonstrated that mislocalized TDP-43 forms RNP-condensates along MN axons that interfere with protein synthesis, specifically of nuclear-encoded mitochondrial proteins. However, the functional outcome of this impairment remains unclear. We therefore tested whether NMJ activity depends on general mitochondrial health using MN infection with lentivirus encoding mitochondrial-targeted Killer-Red fusion protein (MKR). See, Rangaraju, V., Lauterbach, M. & Schuman, E. M. Spatially stable mitochondrial compartments fuel local translation during plasticity. Cell 176, 73-84.e15 (2019). Upon NMJ formation, pre-synaptic NMJ mitochondria were exclusively irradiated, followed by live imaging of calcium transients in post-synaptic muscles (FIG. 22 at a-b). In response to mitochondria irradiation, we observed a marked decrease in muscle activity (FIG. 22 at c-d. FIG. 22 shows a Schematic illustration of Mito-Killer-Red (MKR) experimental setup, used for specifically targeting oxidative stress to NMJ mitochondria. b Images of MKR in NMJ pre-synapse before and after bleach (white line=bleached region). Lower panel indicates post-synaptic muscle labeled with calcium indicator Oregon-Green-BAPTA (OGB). Scale bar=10 μm. n=13,12 NMJs from 3 independent experiments. c, d Representative OGB time-trace of OGB indicating muscle contraction (c) and quantification (d) of contraction ratio before and after MKR bleaching. As control, ChAT::tdTomato-expressing axons were bleached instead of MKR-expressing axons. n=13,12 NMJs from 3 independent experiments. Data are presented as mean values±SD. Unpaired-t-test, two-sided. ****p<0.0001. e Schematic illustration of experimental procedure for puromycin local protein synthesis inhibition in NMJ pre-synapse using puromycin resistant muscles. f Left panel: Time-series images of OGB-labeled co-cultures treated, or not with puromycin in NMJ compartment. Right panel: Demonstration of paired axon-muscle calcium activity only in control NMJs and its absence upon puromycin application. Scale bar=20 μm. n=7,9 MFCs from 3 independent experiments. g Time traces of OGB in pre-synaptic neurons and post-synaptic muscles in control (upper plot), and in puromycin-treated (lower plot) cultures. h Quantification of the percent of innervated and contracting muscles after puromycin application. n=7,9 MFCs from 3 independent experiments. Data are presented as mean values±SD. Unpaired-t-test, two-sided. ****p<0.0001. i Images and j quantification of the percent of degenerating axons in TDPΔNLS and control MN cultures following 16 and 24 h of puromycin treatment. Scale bar=50 μm. n=4,3 MFCs from 3 independent experiments. Data are presented as mean values±SD. Two-way-ANOVA with Holm-Sidak correction. *p=0.0407. a.u stands for arbitrary units.

Next, to determine if local protein synthesis has a similar contribution to NMJ function, we inhibited protein synthesis in pre-synaptic axons by applying puromycin exclusively to the NMJ compartment in co-cultures with puromycin-resistant muscles (FIG. 37 at a-c). Imaging calcium transients in post-synaptic muscles revealed a pronounced decrease in active muscles following axonal and synaptic protein synthesis inhibition (FIG. 22 at e-h). These results indicate that local protein synthesis is fundamental to maintain active NMJs.

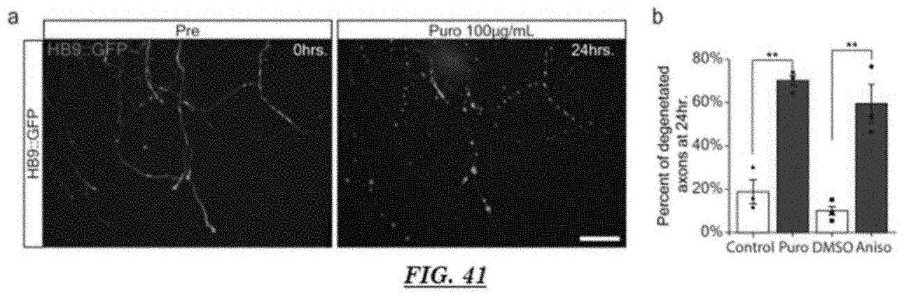

Finally, having found that both mitochondria and local protein synthesis are essential for NMJ activity, we aimed to determine if MN axons exhibit neurodegeneration in response to protein synthesis inhibition. An evaluation of axon health over time revealed that axonal application of protein synthesis inhibitors leads to extensive degeneration (FIG. 41). Furthermore, TDPΔNLS axons had increased sensitivity to protein synthesis inhibition (FIG. 22 at i-j). Altogether, we show that both mitochondrial function and local protein synthesis play crucial roles in maintaining axonal integrity and NMJ function. Both processes are consequently impaired following TDP-43 mislocalization, possibly leading to NMJ dysfunction and neurodegeneration. FIG. 41, local protein synthesis inhibition leads to axon degeneration, shows at a) Representative images and b) quantification of the percent of degenerating HB9:GFP MN axons in the distal compartment of MFC following 24 h axonal incubation with protein synthesis inhibitor Puromycin. DMSO application was used as a control for anisomycin treatment. Scale bar=100 μm. n=3,3,3,3 experiments. SE. Unpaired t-test, two-sided. **p=0.067(left), **p=0.0013(right).

Clearance of TDP-43 Condensates Recovers Local Translation of Nuclear-Encoded Mitochondrial Proteins and Enables NMJ Reinnervation

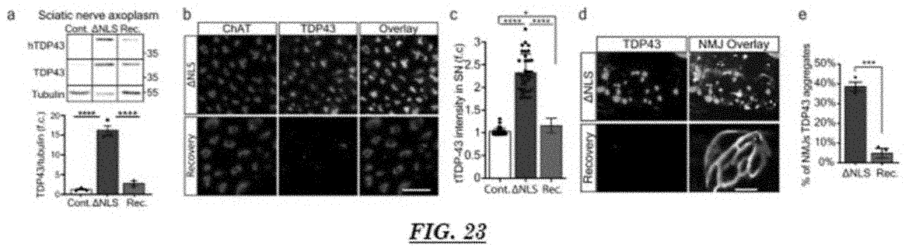

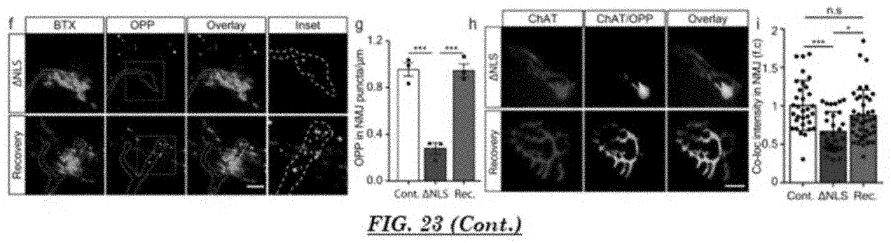

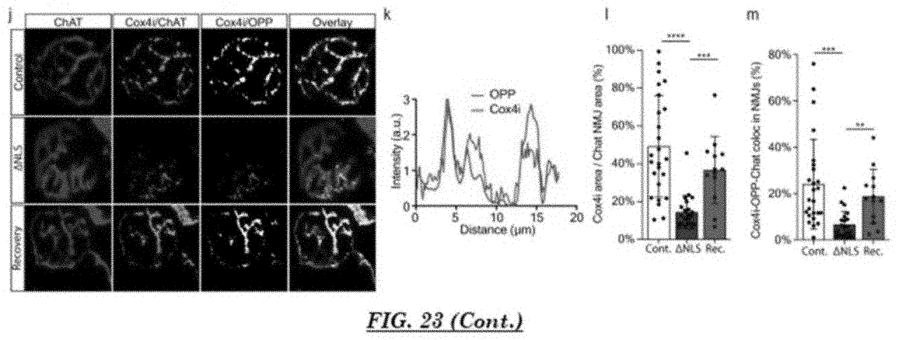

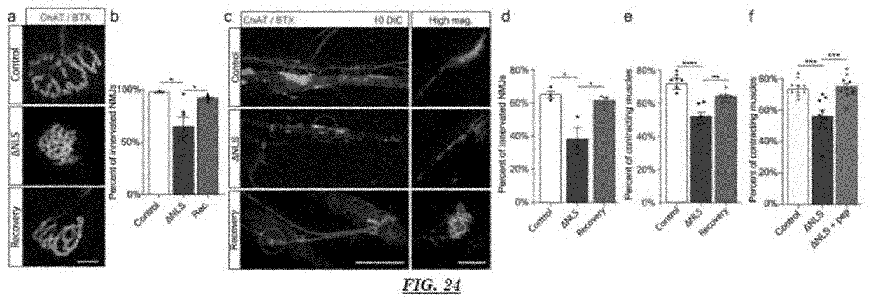

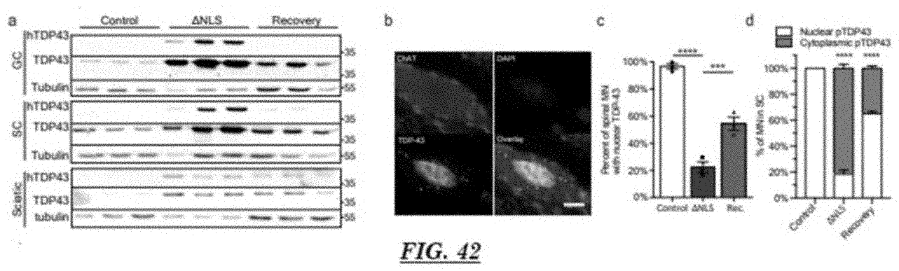

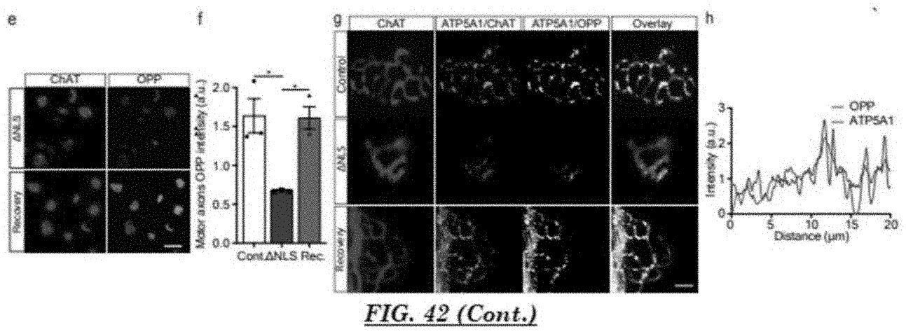

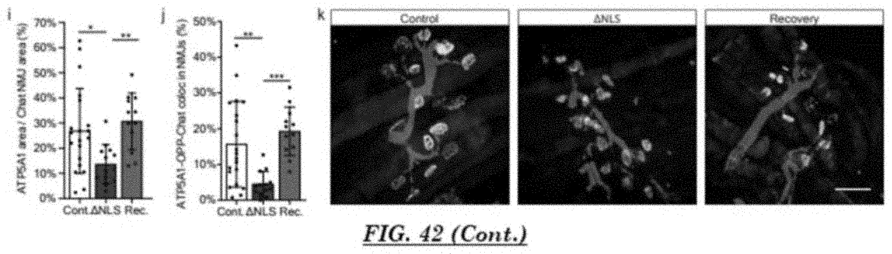

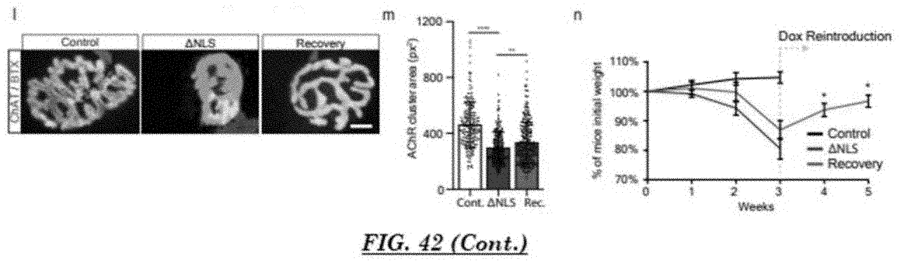

After gaining mechanistic understanding of the pathological outcome of axonal TDP-43 condensate formation on local translation and mitochondria health, we examined whether clearance of TDP-43 condensates can reverse this harmful process. To study the effect of axonal TDP-43 clearance, as previously performed in MNs cell bodies, see Spiller, K. J. et al. Microglia-mediated recovery from ALS-relevant motor neuron degeneration in a mouse model of TDP-43 proteinopathy. Nat. Neurosci. 21, 329-340 (2018), we investigated the ability of TDPΔNLS mice to recover after ceasing the expression of TDPΔNLS and allowing endogenous TDP-43 redistribution to normal localization. We employed a recovery paradigm in which doxycycline was reintroduced into the diet of TDPΔNLS mice after initial deprivation (same was done in vitro, see methods). Re-introduction of doxycycline lowered TDP-43 and pTDP-43 levels in the spinal cord and allowed partial re-distribution into nuclei (FIG. 42 at a-d). Evidently, recovery also had impact on the levels of hTDP-43 in sciatic axoplasm, that was accompanied by a reduction in the overall TDP-43 levels (FIG. 23 at a-c, FIG. 42 at a). Most importantly, TDP-43 was cleared from NMJs (FIG. 23 at d-e). Furthermore, quantification of the OPP signal in the pre-synaptic side of NMJs upon clearance of TDP-43 revealed that protein synthesis in MN axons returns to full capacity, both in vitro (FIG. 23 at f-g) and in vivo (FIG. 23 at h-i, FIG. 42 at e-f). We also performed colocalization analysis of OPP signal with mitochondrial proteins Cox4i and ATP5A1 in hind-limb muscles, which implied that both proteins undergo local synthesis in NMJs of control mice, but to a lesser extent in TDPΔNLS NMJs (FIG. 23 at j-m, FIG. 42 at g-j). Therefore, clearance of TDP-43 from NMJs lead to recovery of pre-synaptic Cox4i and ATP5A1. FIG. 23, Restoring TDP-43 localization recovers local translation of mitochondrial proteins in distal axons and the NMJs, shows a Western-blot and quantification of TDP-43, hTDP-43 levels in sciatic axoplasm of control, TDPΔNLS, and recovered-TDPΔNLS (Rec.). Tubulin was used as loading control. n=3,3,3 mice. Data are presented as mean values ±SEM. One-way-ANOVA with Holm-Sidak correction. ****p<0.0001. b Images and c quantification of TDP-43 intensity within ChAT-positive axons in sciatic nerve sections of control, TDPΔNLS, and recovered-TDPΔNLS mice. Scale bar=20 μm. n=34,34,33 images from 3 mice of each condition. Data are presented as mean values±SD. One-way-ANOVA with Holm-Sidak correction *p=0.0412, ****p<0.0001. d Images and e quantification of the percent of NMJs with apparent TDP-43 condensates in control, TDPΔNLS, and recovered-TDPΔNLS mice. Scale bar=10 μm. n=3,3,3 mice from each condition. Data are presented as mean values±SEM. Unpaired-t-test, two-sided ***p=0.0001. f Images and g analysis of OPP puncta density in in vitro NMJs of control, TDPΔNLS and Recovered-TDPΔNLS co-cultures. Scale bar=5 μm. n=3 independent repeats. Data are presented as mean values +SEM. One-way-ANOVA with Holm-Sidak correction. ***p=0.0004 (left), ***p=0.0004 (right). h Images and i analysis of the OPP labeling intensity in pre-synaptic NMJs of control, TDPΔNLS and recovered-TDPΔNLS mice. n=32,30,39 NMJs, from 3,3,3 mice. Scale bar=10 μm. Data are presented as mean values±SD. One-way-ANOVA with Holm-Sidak correction. *p=0.019, ***p=0.0001. j Images and k representative channel histograms of Cox4i and OPP intensities within pre-synaptic axon (ChAT) in NMJs of control TDPΔNLS mice, l-m quantification of Cox4i area (l), and of Cox4i-OPP coloc-area within pre-synaptic axons (ChAT) in NMJs of control, TDPΔNLS, and recovered-TDPΔNLS mice. Scale bar=10 μm. n=23,21,13 NMJs. 3,3,3 mice. Data are presented as mean values±SD. One-way-ANOVA with Holm-Sidak correction. ****p<0.0001, ***p=0.0007 (l), 0.0002 (m), **p=0.0027. a.u stands for arbitrary units. f.c stands for Fold Change.

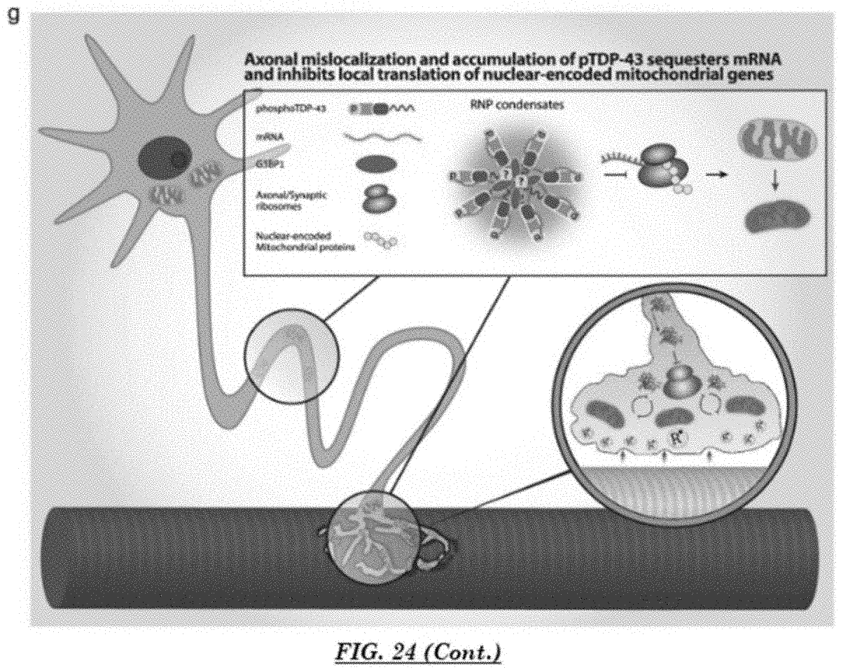

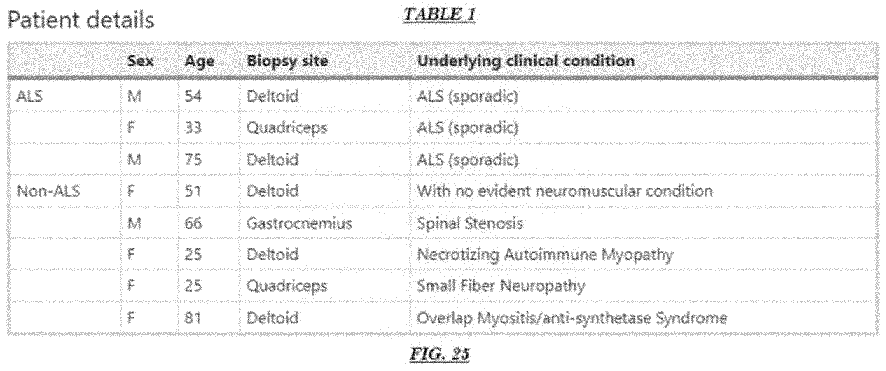

Finally, we investigated the functional impact of TDP-43 mislocalization on NMJ degeneration, and whether it could be reverted by applying the recovery paradigm. Strikingly, measurement of the innervation rate in TDPΔNLS NMJs in vivo and in vitro revealed that mislocalized TDP-43 facilitates NMJ dysfunction and disruption both in adult mice and in co-culture, a process which is reversed by restoring TDP-43 localization (FIG. 24 at a-d, FIG. 42 at k-n). Furthermore, aside of axonal regeneration and NMJ reinnervation, TDP-43 mislocalization also resulted in lack of functional NMJ activity, as measured by the percent of innervated muscles that contract. Importantly, this was reversed either by restoring TDP-43 localization with dox re-introduction (FIG. 25 at e) or by locally dissociating RNP-condensates with G3BP1 peptides (FIG. 25 at f). Thus, TDP-43 derived axon and NMJ degeneration can be reverted either by direct clearance of axonal TDP-43 or by breakdown of TDP-43 RNP-condensates. FIG. 24, Restoring TDP-43 Localization or Dissociation of TDP-43 Condensates Enables NMJ Functional Re-innervation, shows at a Images and b Quantification of NMJs from control, TDPΔNLS, and recovered-TDPΔNLS mice. Quantification is of the percentage of innervated (Chat-BTX positive) NMJs in control, TDPΔNLS and recovered-TDPΔNLS mice. Scale bar=10 μm. n=3,3,3 mice. Data are presented as mean values±SEM. One-way ANOVA with Holm-Sidak correction *p=0.291 (left), *p=0.291 (right). c Images and d quantification of NMJ innervation in vitro, assessed by co-expression of pre- and post-synaptic elements (percent of ChAT-BTX clusters) in control, TDPΔNLS and recovered-TDPΔNLS co-cultures. Scale bar=40 μm in main image, 5 μm in inset. n=3,3,3 MFCs. One-way-ANOVA with Holm-Sidak correction. *p=0.0174(left), 0.0214(right). e, f Quantification of the percent of contracting muscles in control, TDPΔNLS and recovered-TDPΔNLS co-cultures (e) or in control, TDPΔNLS and TDPΔNLS co-cultures treated with G3BP1 peptides in NMJ compartment (f). n=7,6,7 (e), 10,9,9 (f) MFCs. Data are presented as mean values±SEM. One-way-ANOVA with Holm-Sidak correction. **p<0.0039 (e), ***p=0.0004 (f-left), 0.0003 (f-right), ****p<0.0001. g Model: axonal mislocalization of TDP-43 and its phosphorylated form create G3BP1 positive RNP condensates, which decrease local translation of nuclear-encoded mitochondrial proteins by sequestering their mRNA. This results in lack of nuclear-encoded mitochondrial proteins which leads to mitochondria toxicity and NMJ degeneration.

FIG. 42, Dox re-application restores TDP-43 localization, reverses local synthesis inhibition and rescues TDP-43 mediated toxicity, shows at a) Western-blots for total-TDP-43 and human-TDP-43 (hTDP-43) in GC muscles, spinal cords (SC) and sciatic nerves of control, TDPΔNLS, and recovery mice. Tubulin was used as loading control. n=3 mice per condition (1 mouse per lane). b) Representative images and c) Quantitative analysis of the percent of spinal cord MNs (ChAT-red) with nuclear-localized TDP-43 in recovered mice compared to TDPΔNLS mice with no recovery. Nuclear localization was marked by colocalization of DAPI (blue) staining with TDP-43 (green). SE. Scale bar=5 μm. n=3,3,3 mice. one-way ANOVA with Holm-Sidak correction. p=0.0010, ****p<0.0001. d) Quantitative analysis of the percent of MNs (ChAT-red) with nuclear versus cytoplasmic localization of pTDP-43 in spinal cords of control, TDPΔNLS, and recovery mice. n=3,3,3 mice. SE. One-way ANOVA with Holm-Sidak correction. ****p<0.0001. e) Representative images and f) quantification of OPP signal (green) within ChAT axons (red) in sciatic nerves from recovered mice compared to control and ANLS mice. Scale bar=10 μm. SE. n=3,3,3 mice. One-way ANOVA with HolmSidak correction. *p=0.0166(left,right). g) Images and h) representative channel histograms of ATP5A1 (red) and OPP (green) intensities within pre-synaptic axon (ChAT) in NMJs of control TDPΔNLS mice, and i-j) colocalization analysis of the percent of ATP5A1 area (i) and ATP5A1 colocalization with OPP (j) within the pre-synapse area (ChAT) in TDPΔNLS mice compared with control and recovered mice. SD. n=20,13,12 NMJs. 3 mice from each group. One-way ANOVA with Holm-Sidak correction. ***p=0.0005, **p=0.0084(i), 0.0023(j), *p=0.0167. k) Representative images of NMJ innervation from TDPΔNLS mice compared with control and recovered mice. Scale bar=100 μm. n=3 mice per condition. l) Representative images and m) quantitative analysis of BTX (green) post synaptic area cluster size in GC muscle NMJs in recovered mice as compared with ANLS and LM mice. ChAT signal (red) marks pre-synaptic innervation. Sacle bar=10 μm. SD. n=202,263,318 NMJs. One-way ANOVA with Holm-Sidak correction. **p=0.0013, ****p<0.0001. n) TDPΔNLS, control and recovered mice weight measurements after dox retraction. Dox was introduced back to recovered mice at week 3. SE. n=18,15,9 mice for control, TDPΔNLS and recovery groups. One-way ANOVA with Holm-Sidak correction. *p=0.0194(left and right). a.u stands for arbitrary units.

Altogether, we show that the pathological mislocalization of TDP-43 in MN axons disrupts axonal and synaptic protein synthesis. This leads to altered mitochondrial protein turnover in axons and in the NMJ, and eventually sensitizes the entire synapse to degeneration (FIG. 24 at g), a process which is reversible upon clearance of TDP-43 RNP condensates.

Discussion

TDP-43 cytoplasmic mislocalization is a pathological hallmark of ALS, in both sporadic and familial cases, see Neumann, M. et al. Ubiquitinated TDP-43 in frontotemporal lobar degeneration and amyotrophic lateral sclerosis. Science 314, 130-133 (2006), Melamed, Z. et al. Premature polyadenylation-mediated loss of stathmin-2 is a hallmark of TDP-43-dependent neurodegeneration. Nat. Neurosci. 22, 180-190 (2019), Tsuji, H. et al. Molecular analysis and biochemical classification of TDP-43 proteinopathy. Brain 135, 3380-3391 (2012), including patients with C9ORF72 mutation. See, Cook, C. N. et al. C9orf72 poly(GR) aggregation induces TDP-43 proteinopathy. Sci. Transl. Med. 12, eabb3774 (2020). Previous research on TDP-43 focused on outcomes of cytoplasmic mislocalization in MN cell bodies, see, Suk, T. R. & Rousseaux, M. W. C. The role of TDP-43 mislocalization in amyotrophic lateral sclerosis. Mol. Neurodegener. 15, 1-16 (2020) and Gasset-Rosa, F. et al. Cytoplasmic TDP-43 de-mixing independent of stress granules drives inhibition of nuclear import, loss of nuclear TDP-43, and cell death article. Neuron 102, 339-357 (2019). Nonetheless, TDP-43 is regularly found in axons, see Fallini, C., Bassell, G. J. & Rossoll, W. The ALS disease protein TDP-43 is actively transported in motor neuron axons and regulates axon outgrowth. Hum. Mol. Genet 21, 3703-3718 (2012), where it serves a role in shuttling and localization of mRNAs18. Recent reports also revealed that TDP-43 is important for proper axonal protein synthesis. See, Briese, M. et al. Loss of Tdp-43 disrupts the axonal transcriptome of motoneurons accompanied by impaired axonal translation and mitochondria function. Acta Neuropathol. Commun. 8, 116 (2020) and http://scholar.google.com/scholar_lookup?title=Loss%20of%20Tdp-43%20disrupts%20the%20axonal%20transcriptome%20of%motoneurons%20accompanied% 20by%20impaired%20axonal%20translation%20and%20mitochondria%20function&journal=Acta% 20Neuropathol.%20Commun.&volume=&&publication_year=2020&author=Briese%2CMN agano, S. et al. TDP-43 transports ribosomal protein mRNA to regulate axonal local translation in neuronal axons. Acta Neuropathol. 1, 3 (2020). Here, we demonstrate that ALS patients display increased TDP-43 levels in intramuscular nerves, suggesting a forward propagation of TDP-43 to axons. We validate our findings using ALS patient-derived MNs from C9ORF72 mutated iPSCs, and an inducible mouse model that mimics cytoplasmic mislocalization of TDP-43. We show that TDP-43 axonal accumulation elicits the formation of RNA and G3BP1 containing RNP-condensates in axons, consequently interfering with axonal and pre-synaptic protein synthesis. Our observations indicate that TDP-43 specifically binds and sequesters nuclear-encoded mitochondrial mRNAs, thus depleting their protein levels in axons. As we show, mitochondria-related protein synthesis is essential to maintain the axon and the NMJ, and interference with protein synthesis leads to neurodegeneration. Finally, we demonstrate that inhibition of protein synthesis is reversible, even by local restriction with RNP granule assembly, underlining the origin of this pathology in axons and further providing findings regarding the mechanisms by which MN can cope with temporary insult to their local protein synthesis capacities and to mitochondrial alterations.

The concept of local protein synthesis in neuronal processes has mainly been studied in regenerative contexts, see Sahoo, P. K. et al. A Ca2+-dependent switch activates axonal casein kinase 2α translation and drives G3BP1 granule disassembly for axon regeneration. Curr. Biol. 30, 4882-4895.e6 (2020), Terenzio, M. et al. Locally translated mTOR controls axonal local translation in nerve injury. Science 359, 1416-1421 (2018), and Shigeoka, T. et al. Dynamic axonal translation in developing and mature visual circuits. Cell 166, 181-192 (2016), but recent data also suggests it is critical for understanding neurodegenerative disease mechanisms Cioni, J.-M. et al. Late endosomes act as mRNA translation platforms and sustain mitochondria in axons article late endosomes act as mRNA translation platforms and sustain mitochondria in axons. Cell 176, 56-72 (2019), Lopez-Erauskin, J. et al. ALS/FTD-linked mutation in FUS suppresses intra-axonal protein synthesis and drives disease without nuclear loss-of-function of FUS. Neuron 100, 816-830.e7 (2018), and Lehmkuhl, E. M. & Zarnescu, D. C. Lost in translation: evidence for protein synthesis deficits in ALS/FTD and related neurodegenerative diseases. Adv. Neurobiol. 20, 283-301 (2018). Our findings highlight how increased abundance of TDP-43, which is hypothesized to play an important role in local protein synthesis, see Briese, M. et al. Loss of TDP-43 disrupts the axonal transcriptome of motoneurons accompanied by impaired axonal translation and mitochondria function. Acta Neuropathol. Commun. 8, 116 (2020) and Nagano, S. et al. TDP-43 transports ribosomal protein mRNA to regulate axonal local translation in neuronal axons. Acta Neuropathol. 1, 3 (2020), can become harmful upon axonal accumulation. This is associated with TDP-43 induced formation of phase-separated cytoplasmic RNP accumulations, see Chen, Y. & Cohen, T. J. Aggregation of the nucleic acid— binding protein TDP-43 occurs via distinct routes that are coordinated with stress granule formation. J. Biol. Chem. 294, 3696-3706 (2019), Gasset-Rosa, F. et al. Cytoplasmic TDP-43 de-mixing independent of stress granules drives inhibition of nuclear import, loss of nuclear TDP-43, and cell death article. Neuron 102, 339-357 (2019), and Khalfallah, Y. et al. TDP-43 regulation of stress granule dynamics in neurodegenerative disease-relevant cell types /631/80/304 /631/378/87 /13/1 /13/31 /13/51 /13/109 /13/106 /13/89 /14/19 /14/32 /82/80 article. Sci. Rep. 8, 1-13 (2018). Recently, G3BP1 positive RNP granules were shown to inhibit translation. See, Sahoo, P. K. et al. Axonal G3BP1 stress granule protein limits axonal mRNA translation and nerve regeneration. Nat. Commun. 9, 1-14 (2018). We show that translation inhibition is strongly implicated upon accumulation of axonal pTDP-43 within G3BP1-positive RNP condensates (FIG. 18 and FIG. 19). Furthermore, locally dissolving G3BP1 axonal condensates restores local protein synthesis events (FIG. 19 c-h). Future work will be needed to further analyze the mechanisms through which protein synthesis is regulated by formation of axonal RNP condensates.

A fundamental finding in this research is that local translation in NMJs is performed to a greater extent than in axons (i.e OPP puncta density in FIG. 19 h vs. FIG. 19 j). As NMJs are enriched with mitochondria, Altman, T., Geller, D., Kleeblatt, E., Gradus-Perry, T. & Perlson, E. An in vitro compartmental system underlines the contribution of mitochondrial immobility to the ATP supply in the NMJ. J. Cell Sci. 132, 23 (2019), Lee, C. W. & Peng, H. B. Mitochondrial clustering at the vertebrate neuromuscular junction during presynaptic differentiation. J. Neurobiol. 66, 522-536 (2006), Misgeld, T., Kerschensteiner, M., Bareyre, F. M., Burgess, R. W. & Lichtman, J. W. Imaging axonal transport of mitochondria in vivo. Nat. Methods 4, 559-561 (2007), and possibly due to mitochondrial dependency on local protein synthesis, Cioni, J.-M. et al. Late endosomes act as mRNA translation platforms and sustain mitochondria in axons article late endosomes act as mRNA translation platforms and sustain mitochondria in axons. Cell 176, 56-72 (2019) and Kuzniewska, B. et al. Mitochondrial protein biogenesis in the synapse is supported by local translation. EMBO Rep. 21, (8): e48882 (FIG. 21 at 5a-d), the enhanced protein synthesis in NMJs can be attributed to its mitochondrial density. This suggests that the high polarization of MNs leads to higher dependency on local protein synthesis of mitochondrial proteins. As we show, NMJs rely on mitochondria activity (FIG. 22 at a-d), see Altman, T., Geller, D., Kleeblatt, E., Gradus-Perry, T. & Perlson, E. An in vitro compartmental system underlines the contribution of mitochondrial immobility to the ATP supply in the NMJ. J. Cell Sci. 132, 23 (2019), and on local protein synthesis (FIG. 22 at e-g). Therefore, interference of synaptic protein synthesis might initiate local energy deficiency that ultimately leads to NMJ degeneration. Our findings can supply an explanation for how TDP-43-mediated reduction in local protein synthesis specifically sensitizes the NMJs to rapid degeneration, and why NMJ degeneration is an early pathology in ALS, see Dadon-Nachum, M., Melamed, E. & Offen, D. The “Dying-Back” phenomenon of motor neurons in ALS. J. Mol. Neurosci. 43, 470-477 (2011) and Altman, T. & Perlson, E. Neuromuscular junction mitochondrial enrichment: a “double-edged sword” underlying the selective motor neuron vulnerability in amyotrophic lateral sclerosis. Neural Regen. Res. 16, 115 (2021). Further research will be needed to reveal the sequence of events, focusing on the initiation of mitochondrial toxicity in the NMJ.

Finally, we found that the TDP-43 cytoplasmic-related pathology could be reversed by ceasing TDP-434NLS expression, leading to reduction in axonal TDP-43 and clearance of synaptic condensates. Importantly, administration of peptides that interfere with G3BP1-TDP-43 RNP condensation seem to yield promising outcomes as well. This is a pivotal finding since in most ALS patients TDP-43 is not mutated, and yet is still mislocalized into the cytoplasm and forms aggregate-like structures. Hence, ceasing TDP-43 cytoplasmic mislocalization or dissociating TDP-43 RNP-condensates might reverse the disease outcome for a considerable number of patients, and become an important target for future drug development.

Methods

Transgenic Mice

NEFH-tTA line 8 (Jax Stock No: 025397) and B6;C3-Tg(tetO-TARDBP*)4Vle/J (Jax Stock No: 014650) were obtained from Jackson Laboratories and cross-bred to create NEFH-hTDP-43ΔNLS (TDP43ΔNLS) mice line. Those mice were constitutively fed with doxycycline containing diet (200 mg/kg Dox Diet #3888, Bio-Serv) as was done previously. See, Walker, A. K. et al. Functional recovery in new mouse models of ALS/FTLD after clearance of pathological cytoplasmic TDP-43. Acta Neuropathol. 130, 643-660 (2015), Spiller, K. J. et at Selective motor neuron resistance and recovery in a new inducible mouse model of TDP-43 proteinopathy. J. Neurosci. 36, 7707-7717 (2016), Spiller, K. J. et al. Microglia-mediated recovery from ALS-relevant motor neuron degeneration in a mouse model of TDP-43 proteinopathy. Nat. Neurosci. 21, 329-340 (2018). ChATcre-tdTomatolox-hTDP-43ANLS was obtained by crossing ChAT^{cre} and tdTomatolox mice. (Jax stock no. 006410 and 007908, respectively). Thy1-mito-Dendra-TDP-43ΔNLS was obtained by crossing the TDP-43ΔNLS with Thy1-COX8A/Dendra (Jax stock no. 025401).

After Dox retraction, all TDP-43ANLS mice were weighted weekly to track disease progression.

HB9-GFP (Jax stock no. 005029) mice were originally obtained from Jackson Laboratories. The colony was maintained by breeding with ICR mice (Institute of Animal Science, Harlan).

SOD1G93A (Jax stock No. 002726) mice were originally obtained from Jackson Laboratories and maintained by breeding with C57BL/6 J mice. Only non-transgenic (C57BL/6 J WT) females from this colony were used for the purpose of primary myocyte culture.

All animal experiments were approved and supervised by the Animal Ethics Committee of Tel-Aviv University.

ALS Patient-Derived Induced Pluripotent Stern-Cell MNs

Skin fibroblasts were obtained from an ALS patient carrying the (G4C2)n repeat expansion mutation in the C9ORF72 gene. Genetic confirmation, iPSC production, design of homology-directed repair templated and production of isogenic iPSC control line were performed by Prof. Kevin Talbot (University of Oxford). See, Ababneh, N. A. et al. Correction of amyotrophic lateral sclerosis related phenotypes in induced pluripotent stem cell-derived motor neurons carrying a hexanucleotide expansion mutation in C9orf72 by CRISPR/Cas9 genome editing using homology-directed repair. Hum. Mol. Genet. 29, 2200-2217 (2020).

MN transcription factor cassette including the transcription factors Islet-1 (ISL1) and LIM Homeobox 3 (LHX3) along with NGN2 were integrated into a safe-harbor locus in iPSCs under a doxycycline-inducible promoter by Prof. Michael Ward (NIH). See, Fernandopulle, M. S. et al. Transcription factor—mediated differentiation of human iPSCs into neurons. Curr. Protoc. Cell Biol. 79, e51 (2018).

iPSC clones were cultured in 6-well plates coated with Matrigel (Corning; 356234), grown in mTesrl medium (STEMCELL Technologies; 85850) and passaged with mTesrl medium containing 10 μM Rho-Kinase Inhibitor (RI) (Sigma-Aldrich; Y0503) for 1 day following passaging. Culture media was refreshed daily until colonies reached 80% confluence. Doxycyline-induced differentiation into lower MNs was performed according to the protocol in, see Fernandopulle, M. S. et al. Transcription factor—mediated differentiation of human iPSCs into neurons. Curr. Protoc. Cell Biol. 79, e51 (2018), with minor modifications. Briefly, the ˜300,000 iPSCs were plated in a 35 mm dish in mTesr1-RI medium. On the following day media was replaced with IM supplement containing DMEM/F12, (Gibco; 31330038), 1% N2-supplement (Gibco; 17502048), 1% NEAA (Biological Industries; #01-340-1B), 1% GlutaMAX (Gibco; 35050038) with 10 μM RI, 2 μg/mL doxycycline (Sigma, D9891), and 0.2 μM Compound E (Merck; 565790). After 48 h cells were resuspended with Accutase (Sigma-Aldrich; SCR005) and re-plated in the proximal compartment of MFCs at a concentration of 200,000 MN per MFC. Prior to plating, MFCs were coated overnight with 0.1 mg/mL PDL (Sigma-Aldrich; P6407) in PBS, and 15 μg/ml Laminin (Sigma-Aldrich; L2020) for 4 h on the following day. To prevent outgrowth of mitotically active cells 40 μM BrdU (Sigma, B9285) was added to the medium during the first 24 h after plating. At the 4^{th} day, cells were treated with MM medium containing: Neurobasal medium (Gibco; 21103049), 1% B27 (Gibco; 17504044), 1% N2, 1% NEAA, 1% Optimal-Culture-One supplement (Gibco; A3320201), 1 μg/mL Laminin, 20 ng/mL BDNF, 20 ng/mL GDNF, 10 ng/mL NT3 (Alomone labs; N-260). Medium was refreshed other day. For incucyte experiments 10,000 cells per well were plated in 96-well plate. Incucyte experiments were performed at 6DIV and 9DIV. Immunostaining, smFISH, OPP and RIP experiments were performed between 9DIV and 12DIV. The DIV count represents the number of days of Doxycycline-induced differentiation.

Human Muscle Biopsy for Intra-Muscular Nerve Staining

Intra-muscular nerve staining was performed on muscle biopsies from 3 ALS patients and 5 non-ALS patients (see table below). All clinical and muscle biopsy materials used in this study were obtained with written informed consent during 2016-2020 for diagnostic purposes followed by research application, approved by the Helsinki institutional review board of Sheba Medical Center, Ramat Gan, Israel. Deltoid, quadriceps or gastrocnemius skeletal muscle samples were excised via open biopsies and pathological analysis was performed at the neuromuscular pathology laboratory at Sheba Medical Center, Ramat-Gan, Israel. All 3 ALS patients were diagnosed with clinically definite or probable ALS according to El Escorial criteria. See, Brooks, B. R. El escorial World Federation of Neurology criteria for the diagnosis of amyotrophic lateral sclerosis. J. Neurol. Sci. 124, 96-107 (1994). Control muscles included a variation of findings, which were consistent with a diagnosis of normal muscle, severe, chronic ongoing denervation and reinnervation due to spinal stenosis, necrotic autoimmune myopathy, type 2 fiber atrophy due to disuse and overlap myositis syndrome.

Frozen muscle biopsies were cryo-sectioned to 10 μm thick slices, mounted onto slides and air dried for 30 min in room temperature (RT). Sections were washed in PBS, fixed in 4% PFA for 20 min, and permeabilized with 0.1% Triton, and blocked with 5% goat serum (Jackson Laboratories) and 1 mg/mL BSA (Amresco). Sections were then incubated with appropriate antibodies overnight at 4° C. in blocking solution [rabbit anti TDP43 or rabbit anti phosphorylated TDP-43 (both 1:1,000, Proteintech), Chicken anti NFH (Abcam, 1:1,000). Sections were washed again and incubated for 2 h with secondary antibodies (1:1000, goat anti chicken 488 and goat anti rabbit 640, Abcam. 1:1000, goat anti mouse 594, Invitrogen), washed and mounted with ProLong Gold (Life Technologies). See Table 1, FIG. 25 for Patient Details.

Micro Fluidic Chamber Preparation

The MFC design was established per Altman, T., Maimon, R., Ionescu, A., Pery, T. G. & Perlson, E. Axonal transport of organelles in motor neuron cultures using microfluidic chambers system. J. Vis. Exp. 2020, 60993 (2020). Briefly, PDMS (Dow Corning) was casted into custom-made epoxy replica molds, left to cure overnight at 70° C., punched (6 mm/7 mm punches), cleaned and positioned in 35 mm or 50 mm glass plates (WPI): All experiments were done in 6mm-well small MFCs, except for experiments in FIG. 18, where 7 mm-wells MFC were punched for spinal cord explant culture. See, Id.

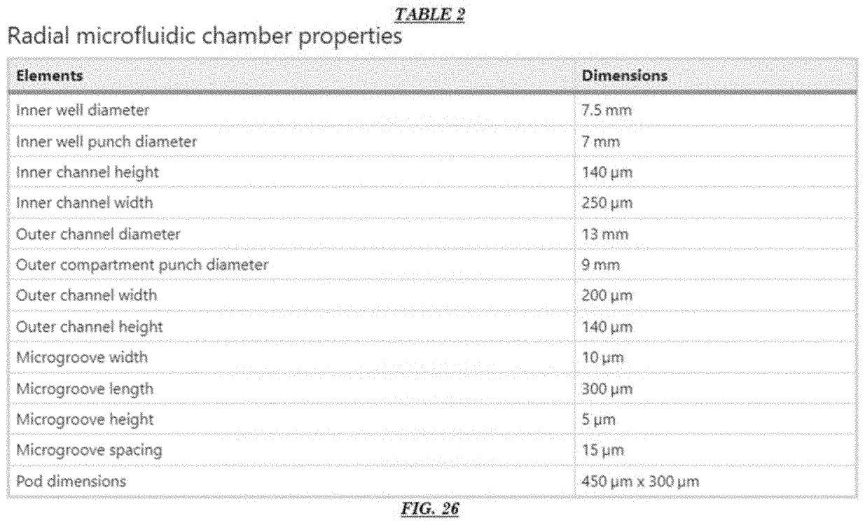

Radial PDMS molds (FIG. 17 at g-j) were designed and fabricated with SU-8 photoresist protocol, see Gluska, S., Chein, M., Rotem, N., Ionescu, A. & Perlson, E. Tracking quantum-dot labeled neurotropic factors transport along primary neuronal axons in compartmental microfluidic chambers. Methods Cell Biol. (2015). doi.org-10.1016/bs.mcb.2015.06.016, in the Tel-Aviv University Nano and Micro Fabrication Center, as described in Table 2, FIG. 26.

PDMS mold was pre-treated with Chlorotrimethylsilane (Sigma) prior to PDMS casting. PDMS casting was done in a similar manner as for regular MFC. Radial MFCs were then punched twice to form MFC rings. Inner well was punched with 7 mm biopsy punch, and outer well was punched with 9 mm punch. Cleaning procedure done in a similar manner as for regular MFC. Radial MFC rings were adhered to sterile 13 mm coverslips inside 24-well plates.

MN Culture and MN-Myocyte Co-Culture

E12.5 old embryos ventral spinal cord were dissected in HBSS prior to dissociation. For TDPΔNLS cultures, genotype of each embryo was determined at this phase by PCR. Meanwhile, spinal cords were kept in 37° C. 5% CO2 with Leibovitz L-15 medium (Biological industries) supplemented with 5% Fetal Calf serum and 1% Penicillin/Streptomycin (P/S-Biological Industries). Spinal cord explants were cut transversely to small pieces and plated in MFC proximal compartment in Neurobasal (Gibco), 2% B27 (Thermo Fisher), 1% Glutamax (Gibco),1% P/S, 25 ng/mL BDNF (Alomone labs).

Dissociated MN cultures were obtained by further, trypsinization and trituration of explants. Supernatant was collected and centrifuged through BSA (Sigma) cushion. The pellet was then resuspended and centrifuged through and Optiprep (Sigma) gradient (containing 10.4% Optiprep, 10 mM Tricine, 4% w/v glucose). MN-enriched fraction was collected from the interphase, resuspended and plated in the proximal MFC compartment at a concentration of 150,000 MN per regular MFC, 250,000 per radial MFC. MNs were maintained in complete neurobasal (CNB) medium containing Neurobasal, 4% B27, 2% horse serum (Biological Industries), 1% Glutamax, 1% P/S, 25 μM Beta-Mercapto ethanol, 25 ng/mL BDNF, 1 ng/mL GDNF (Alomone) and 0.5 ng/mL CNTF (Alomone). Glial cell proliferation was restricted by addition of 1 μM Cytosine Arabinoside (ARA-C; Sigma) to culture medium in 1-3DIV. At 3DIV BDNF concentration in proximal compartment was reduced (1 ng/mL), while medium in distal compartment was enriched with GDNF and BDNF (25 ng/mL) to direct axonal growth.

Myocyte culture was performed as previously described. See, Zahavi, E. E. et al. A compartmentalized microfluidic neuromuscular co-culture system reveals spatial aspects of GDNF functions. J. Cell Sci. 128, 1241-1252 (2015) and Ionescu, A., Zahavi, E. E., Gradus, T., Ben-Yaakov, K. & Perlson, E. Compartmental microfluidic system for studying muscle-neuron communication and neuromuscular junction maintenance. Eur. J. Cell Biol. 95, 69-88 (2016). Briefly, GC muscles from a P60 adult C57BL/6 J mouse were extracted into DMEM with 2.5% P/S/N (Biological Industries) with 2 mg/mL collagenase-I (Sigma) for 3 h, then dissociated and incubated with BioAmf 2.0 (BA; Biological Industries) in Matrigel (BD Corning) coated plates for 3 days. Myoblasts were purified by performing pre-plating for 3 consecutive days, and then plated at a density of 75,000 in small MFC, and 150,000 for large MFC (for SC explant experiments).

Muscles in co-culture were kept in BA medium for 7 days. To aid NMJ formation, media in all compartments was then replaced to poor neurobasal (PNB) medium, which contained only 1% P/S and 1% Glutamax. Doxycycline was applied to TDP-43ΔNLS cultures only at the proximal MN compartment, in a concentration of 0.1 μg/mL, immediately after plating and throughout the entire experimental timeline. For TDPΔNLS recovery experiments (FIGS. 7-8), Doxycline was applied at the proximal MN compartment after 5 DIV, and the cultures were grown until 11 DIV. For G3BP1 peptide treatment (FIG. 21 at i-j, FIG. 24 at f), 20 μM G3BP1 peptides were added exclusively to the distal compartment, starting after 7 DIV.

### Experiment

### Protein Extraction

Spinal cord and GC muscles were extracted from adult mice and homogenized in ice-cold PBS lysis buffer containing 1% Triton and protease inhibitors (Roche). Sciatic axoplasm was obtained from both sciatic nerves from every mouse. Sciatic nerves were sectioned and axoplasm was extracted into 100 μL PBS and protease inhibitors by gentle pressing the sections.

Extraction of axonal and somatic proteins from radial MFCs was performed as follows: Axons were extracted by first filling the inner well with high volume of PBS and applying 40 μL of RIPA lysis buffer (1% Triton, 0.1% SDS, 25 mM Tris-HCl (pH 8.0), 150 mM NaCl) to the outer compartment for 1 min. We used the same 40 μL to collect the axon lysate from two additional wells—in total, 3 wells per sample. Protein extraction from the inner (soma) compartment was then performed by replacing the PBS with 100 μL of RIPA buffer, 1-minute incubation, and then scraping of the cells. Tissue/culture lysates were centrifuged at 10,000 G for 10 min at 4° C. Protein concentration was determined using Bradford assay (BioRad).

Western Blotting

Protein samples were mixed with SDS sample buffer and boiled at 100° C. for 10 mM, and then loaded to 10% acrylamide gels for SDS-PAGE. Proteins were transferred to nitrocellulose membranes in buffer containing 20% MeOH. Membranes were blocked with 5% skim-milk (BD) or 5% BSA for 1 h, followed by overnight incubation at 4° C. with primary antibodies: mouse anti human TDP-43 (Proteintech, 1:4000), rabbit anti TDP-43 (Proteintech, 1:2000), rabbit anti ERK1/2 (tERK; Sigma, 1:10,000), mouse anti alpha-tubulin (Abcam, 1:5,000), rabbit anti MAP2 (Milipore, 1:1000), mouse anti TAU-5 (Abcam, 1:250). Membranes were then washed with TBST and incubated 2 h at RT with secondary HRP antibody (Donkey anti rabbit and donkey anti mouse, Jackson Laboratories, 1:10,000), or HRP-Goat-IgG2a-anti-mouse (Jackson, 1:20,000; for puromycin blots) washed with TBST and visualized in iBright 1500 ECL imager (Life Technologies) after 5 min incubation with ECL reagents. Quantification was performed using FIJI ImageJ V.2.0.0 software.

RNA Extraction and cDNA Synthesis

MN Axonal RNA was extracted from outer compartment of radial MFCs at 14DIV. Axonal RNA was extracted by removing the PBS (from prior wash) from the outer compartment and adding 100 μL TRI reagent lysis reagent (Sigma-Aldrich). Inner well was filled with higher volume of PBS to disable the inward flow of lysis reagent towards the inner (soma) compartment and prevent soma contamination. Axons washed off the plate by pipetting the TRI reagent around the outer well for 30 s. RNA from somata in the inner compartment was extracted with 100 μL TRI reagent, and lysate was collected in a similar manner. cDNA for axon and soma was prepared with High-Capacity Reverse Transcription Kit (Thermo; Cat. 4368814).

For sciatic nerve RNA extraction, sciatic axoplasm was obtained from 2 adult mice sciatic nerves in a tube containing 100 μL PBS and protease inhibitors, cut into small pieces and gently squeezed on ice. The axoplasm was then centrifuged at 10,000 G for 10 min at 4° C. RNA was extracted using the RNAeasy micro kit (Qiagen) according to manufacturer's protocols.

PCR and RT-qPCR

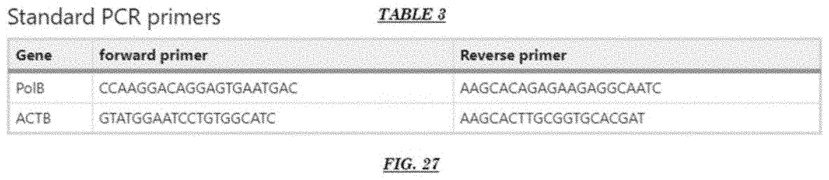

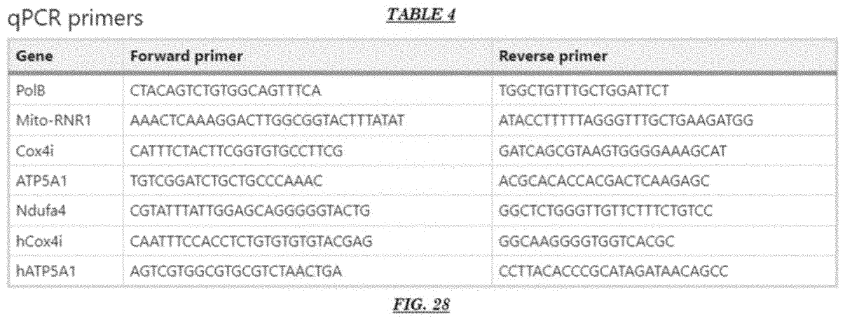

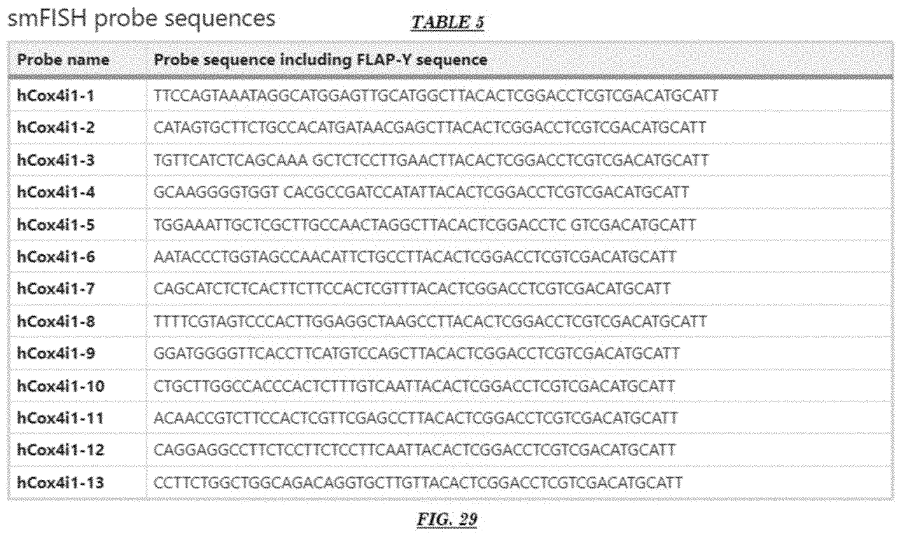

Reverse Transcription was performed with High-capacity Reverse Transcription cDNA kit using random primers (Thermo Fisher Scientific). Standard PCR was done to test radial chambers axonal purity using KAPA ReadyMix using the primers shown in Table 3, FIG. 27.

qRT-PCR of sciatic axoplasm was done for the following genes: PolB, mitochondrial-RNR1, Cox4i, ATP5A1 and NDUFA4. Mitochondrial-RNR1 gene was used as a reference gene when calculating ACT, as we aim to quantify relative mRNA levels of nuclear-encoded mitochondrial genes as a part of total axonal mitochondria.

qPCR Sybr-green reactions were performed with PerfeCTa SYBR green FastMix (QuanaBio) in a StepOne Real-Time PCR system (Thermo Fisher Scientific). See Table 4, FIG. 28 for qPCR primers.

Immunofluorescent Staining for Cryosections

Sciatic nerve and spinal cord sections were prepared from fixating respective tissues in 4% PFA for 16 h at 4° C., then incubation with 20% sucrose for 16 h at 4° C., and cryo-embedding in Tissue-Tek OCT compound (Scigen). Tissues were then cryo-sectioned to 10 μm thick slices, washed with PBS, followed by permeabilized and blocking in solution containing 10% goat serum, 1 mg/mL BSA and 0.1% Triton in PBS for 1 h. Later the sections were incubated overnight at 4° C. with primary antibody rabbit anti TDP-43 (Proteintech, 1:2,000), rabbit anti pTDP-43 (Proteintech, 1:2,000), chicken anti NFH (Abcam, 1:500), rabbit anti Cox4i1 (Abcam, 1:500), rabbit anti ATP5A1 (Abcam, 1:500). This was followed by 2 h incubation at RT with secondary antibody (Goat anti chicken 405, Abcam, 1:500. Goat anti chicken 488, Goat anti rabbit 640, Abcam, 1:1000. Goat anti rabbit 488, Invitrogen, 1:1000. Goat anti rabbit 594, Jackson laboratory, 1:1,000), wash with PBS and mounting with ProLong Gold (Life Technologies) containing DAPI nuclear staining.

Whole Mount NMJ Staining

Gastrocnemius (GC), Tibialis Anterior (TA) or Extensor Digitorum Longus (EDL) muscles were dissected from adult mice, cleared from connective tissue and kept in 4% PFA until use. Muscles were washed in PBS, stained for post synaptic AChR with αBTX-Atto-633 (Alomone labs) or aBTX-TMR-594 (Sigma) at 1 μg/mL for 15 min. Next, muscles were permeabilized with ice-cold MeOH at −20° C. for 5 min, blocked and further permeabilized with 20 mg/mL BSA and 0.4% Triton for 1 h. Muscle preparations were agitated overnight at RT with appropriate antibodies: chicken anti Neurofilament heavy-chain (NFH) (1:500, abcam), rabbit anti NFH (1:500, Sigma), rabbit anti TDP-43 (proteintech, 1:2,000), rabbit anti pTDP-43 (Proteintech, 1:2,000), rabbit anti Cox4i (1:500, Abcam), rabbit anti ATP5A1 (1:500, Abcam). Next, muscles were incubated with secondary antibodies (Goat anti chicken 405, Abcam, 1:500. Goat anti chicken 488, Abcam, 1:1000. Goat anti rabbit 488, Invitrogen, 1:1000. Goat anti rabbit 594, Jackson laboratory, 1:1,000). Finally, muscles were cut to small vertical pieces and mounted with VectaShield (Vector Laboratories). Cover slides were sealed until use with nail polish.

Immunofluorescent Staining for MNs

10DIV TDPΔNLS MNs cultures in MFCs were fixated for 20 min in 4% PFA. For NaAsO₂ experiment, NaAsO₂ (250 μM) was applied for 1 h prior to fixation. MNs were, permeabilized with 0.1% Triton for 30 min, and then blocked for 1 h with 10% goat serum, 1 mg/mL BSA and 0.1% Triton in PBS for 1 h. Primary antibodes rabbit anti TDP-43 (Proteintech, 1:2,000), mouse anti Puromycin (Millipore, 1:1,000). Antibodies were diluted in blocking solution, and incubated with samples overnight at 4° C. Secondary antibodies (Goat anti chicken 405, Abcam, 1:500. Goat anti chicken 488, goat anti rabbit 640, Abcam, 1:1000. Goat anti rabbit 488, Invitrogen, 1:1000. Goat anti rabbit 594, Jackson laboratory, 1:1,000) were diluted in blocking solution and incubated with samples for 2 h incubation at RT. MN Samples were mounted with ProLong Gold DAPI anti-fade reagent(Life Technologies).

Fluorescence Microscopy and Image Analysis

Confocal images were captured using Nikon Ti microscope equipped with a Yokogawa CSU X-1 spinning disc and an Andor iXon897 EMCCD camera controlled by Andor IQ3 software. Phase-contrast movies of muscle contraction were acquired using the same microscope in Epi-mode and images were captured with an Andor Neo sCMOS camera. All live imaging experiments were performed with 5% CO2 and 37° C. humidified using in situ microscope setup. Image analysis was performed using FIJI ImageJ V.2.0.0 and Bitplane Imaris 8.4.3 software.

Co-Culture Contraction Analysis

NMJ activity was assessed by quantifying the percent of innervated and contracting myocytes in co-culture as previously described36. Briefly, after 12 days in co-culture, phase-contrast time lapse image series were acquired at a frame rate of 25 frames-per-second (25 FPS) using a X20 air objective. During imaging, cultures were maintained in controlled temperature and CO₂ environment. For obtaining contraction time traces, we used the “Time Series Analyzer V3” plugin for FIJI ImageJ V.2.0.0, and marked a region of interest on a small, high contrast and mobile region on the muscle, and then obtained the mean values for each time point.

Turbofect Transfection of Puromycin Resistant Muscles

Primary muscle cells were transfected with either PLKO.1 or with PQCXIP-mCherry empty backbone vectors containing Puromycin-N-acetyltansferase (PAC) gene. 150,000 primary myoblasts plated per well were in a matrigel pre-coated 24-well plate. After 4 h, myoblasts in each well were transfected with 1 μg of DNA (PLKO.1/PQCXIP-mCherry) and 4 μL Turbofect transfection reagent (Thermo scientific) prepared in a serum-free medium. Cultures were incubated with transfection reagent for 12 h in an antibiotic-free BA medium, and then washed with fresh BA medium with 1% P/S. After 4 h, cultures lifted from the 24-well plated with trypsin-C and plated in the distal compartment MFCs.

OPP Labeling of MN Culture

OPP was used to label ribosome-nascent polypeptide chains in MN cell bodies, MN axons and in neuromuscular co-cultures. OPP stock (20 mM; Life Technologies) was diluted in the appropriate medium to a final concentration of 20 μM, and then applied to either proximal/distal compartments of the MFCs to label cell bodies or axons/NMJs, respectively. Cultures were incubated with OPP for 30 min, that was chosen as the preferred time point (FIG. 34), while the opposite compartment was maintained with higher medium volume to prevent OPP flow and unspecific labeling. Anisomycin (40 μM; Sigma), cycloheximide (150 μM; Sigma), or G3BP1-inhibiting 190-208 peptide (20 μM) were applied for 30 mM before OPP was added to cultures, and then together with OPP (total 1 h) for validating specific labeling of newly synthesized proteins. Cultures were then washed twice rapidly with cold PBS and fixed with 4% PFA for 15 min at RT. Co-cultures were labeled with 0.5 μg/mL aBTX-Atto-633 (alomone) for 15 mM. Cultures were permeabilized with 0.1% Triton in PBS for 30 mM at RT. ClickIT reaction with either Alexa-488 Picolyl-Azide or Alexa-594 Picolyl-Azide were performed following the protocols supplied by the manufacturer. Cultures were mounted with ProLong Gold Antifade Reagent. Tat-fused G3BP1-inhibiting peptide (190-208) was a kind gift from the laboratory of Prof. Jeffery L. Twiss.

OPP Labeling Ex-Vivo

OPP was used to label protein synthesis in freshly dissected TA/EDL muscles and sciatic nerves. Immediately after mice were euthanized, tissues were extracted into 95% 02 and 5% CO2 oxygenized-ringer solution containing OPP (20 μM). TA/EDL were separated and further dissected into thinner sections prior incubation with OPP. Tissues were incubated with OPP for 35 min in 37° C., then washed 3 times with PBS on an orbital shaker and fixed in 4% PFA for 12 h at 4° C. Sciatic nerves were further incubated with 20% sucrose for additional 12-24 h at 4° C., and then embedded in Tissue-Tek OCT compound. ClickIT procedure was performed only on 10 μm slices sectioned from the first/last 300 μm of sciatic nerves. Sciatic nerve sections were collected to Hitobond+slides (Marienfeld). Sciatic nerve sections were permeabilized with 0.1% triton for 30 min followed by 3 PBS washes.

TA/EDL muscles were labeled with 2 μg/mL aBTX-Atto-633 (alomone) for 15 min and permeabilized with ice-cold MeOH for 5 min at −20° C. Muscles were further permeabilized with 0.4% triton in PBS for 1 h at RT.

ClickIT reaction with either Alexa-488 Picolyl-Azide was performed following the protocols supplied by the manufacturer. Stained sciatic nerve sections were then mounted with ProLong Gold Antifade Reagent. Muscles were either mounted with VectaShield, or proceeded with to standard immunofluorescence labeling protocol.

OPP Biotinylation and Streptavidin Pull Down from Sciatic Nerves

OPP biotinylation and pull downs with streptaviding was performed as previously described by Terenzio et al., see Terenzio, M. et al. Locally translated mTOR controls axonal local translation in nerve injury. Science 359, 1416-1421 (2018), with mild modifications. Briefly, for the purpose of pull-down and immunoblot for Cox4i, sciatic nerves from 10 mice per condition in each repeat were extracted and cut into 3-4 mm fragments. OPP Labeling was performed in DMEM +10% FBS +1% penicillin/streptomycin with 100 μg/ml OPP for 1 h at 37° C. Axoplasm was extracted in PBS with 1× protease inhibitors mix and Samples were centrifuged for 10 min at 15,000×g at 4° C. to remove sciatic fragments and cell debris. SDS added to 1% and OPP-tagged proteins were conjugated to biotin by click chemistry with 100 μM biotin-PEG3-azide, 1 mM TCEP, 100 μM TBTA and 1 mM CuSO4, for 2 h at room temperature on a rotator. After click conjugation, proteins were precipitated using 5 vol ice-cold acetone overnight at −20° C. Pellets were washed twice in 1 mL methanol with sonication, resuspended in 1% SDS-PBS and desalted with Zeba-spin 0.5 mL 7 K cutoff columns. Protein concentrations were measured by BCA assay, 40 μg of each sample was taken for total coomassie staining, and equal amounts of protein (1.2 mg) were used for streptavidin pulldown which was carried out overnight at 4° C. in 1% NP40, 0.1% SDS in PBS and 1× protease inhibitor mix, with 80 μl of streptavidin magnetic beads (Pierce). After overnight incubation, beads were washed twice for 10 min with 1% NP40, 0.1% SDS and 1× protease inhibitor mix in PBS at room temperature, 3 times for 30 min with 6 M ice-cold urea in PBS with 0.1% NP-40 at 4° C., and once again for 10 min with 1% NP40, 0.1% SDS and 1× protease inhibitor mix in PBS at room temperature. Proteins were eluted by boiling the beads with 5× sample buffer. PVDF blots blocked with 5% BSA in TBS-T and were incubated overnight with Cox4i antibody (Rabbit; 1:1,000) in blocking solution at 4° C., and then for 2 h at room temperature with HRP-conjugated anti-rabbit antibody.

Specificity and sensitivity of OPP pull-down procedure also was validated in cultured HEK293T cells (CRL-3216, ATCC) similar to the procedure above, with the addition of the following controls: No OPP (1 hr at 37° C.) or anisomycin (200 μg/ml) for 2 h at 37° C., followed by co-incubation with OPP (100 μg/ml) for 1 h. Pull-down PVDF blots were blocked with 5% BSA in TBS-T, and then incubated for 1 h with HRP-conjugated streptavidin (1:10,000) in blocking solution. Total blots were either labeled with Coomassie or in a similar manner with HRP-streptavidin.

OPP labeling and biotinylation for sciatic nerve total controls was performed from sciatic nerves of 3 mice per condition, and similar to described above for HEK-293T cells.

Preparation of Mass Spectrometry Samples

For preparation of mass spectrometry samples, 30 μg axoplasmic protein lysates from WT and TDPΔNLS animals in 10% SDS buffer were precipitated in 80% acetone at −20° C. overnight. Samples were centrifuged at 18,000×g at 4° C. for 10 min. Protein pellets were washed twice with 80% acetone, air-dried for 10 min and reconstituted in 50 μL urea buffer (6 M urea, 2 M thiourea in 10 mM HEPES/KOH, pH 8.0). Samples were reduced and alkylated with 5 mM TCEP and 20 mM CAA at RT for 30 min, followed by digestion with 0.5 μg endoproteinase Lys-C (Wako) at RT for 3 h. Samples were diluted fourfold with 50 mM ammionium bicarbonate (ABC) buffer and digested with 0.5 μg trypsin (Sigma) at RT overnight. Digestion was stopped by adding 1% formic acid and peptides were desalted using Stop-and-Go extraction tips as previously described. See, Rappsilber, J., Ishihama, Y. & Mann, M. Stop and go extraction tips for matrix-assisted laser desorption/Ionization, nanoelectrospray, and LC/MS sample pretreatment in proteomics. Anal. Chem. 75, 663-670 (2003).

LC—MS/MS Analysis and Data Processing

Proteome analyses were performed using an Easy nLC 1000 ultra-high performance liquid chromatography (UHPLC) coupled to a QExactive Plus mass spectrometer (Thermo Fisher Scientific) with the same settings as described before. See, Nolte, H., Hölper, S., Selbach, M., Braun, T. & Krüger, M. Assessment of serum protein dynamics by native SILAC flooding (SILflood). Anal. Chem. 86, 11033-11037 (2014). Acquired MS spectra were correlated to the mouse FASTA databased using MaxQuant (v. 1.5.3.8) and its implemented Andromeda search engine, see Cox, J. et al. Andromeda: a peptide search engine integrated into the MaxQuant environment. J. Proteome Res. 10, 1794-1805 (2011), with all parameters set to default. N-terminal acetylation and methionine oxidation were set as variable modifications and cysteine carbamidomethylation was included as a fixed modification. Missing value imputation, statistical analyses and GO annotations were performed in Perseus (v. 1.6.2.3)58 and data were visualized in Instant Clue. See, Nolte, H., MacVicar, T. D., Tellkamp, F. & Kruger, M. Instant clue: a software suite for interactive data visualization and analysis. Sci. Rep. 8, 12648 (2018). Significance cutoff was set to a log2 fold change of at least ±0.58 and a −log10 p-value of 1.3.

Single-Molecule Fluorescent In Situ Hybridization Combined with Immunofluorescent Staining

Labeling of single mRNA molecules in iPS-MN was performed by smiFISH as previously described. See, Tsanov, N. et al. SmiFISH and FISH-quant—a flexible single RNA detection approach with super-resolution capability. Nucleic Acids Res. 44, e165 (2016). Briefly, ALS Patient-derived C9ORF72 and isogenic control iPS-MN were grown for 12 days in MFCs placed over 22 mm×22 mm coverslips. Labeling procedures were performed in RNase-free environment and using RNAse-free reagents. Cultures were fixed with 4% PFA and permeabilized overnight with 70% Ethanol. Samples were incubated with SSC (Sigma; 56639) based 15% Formamide (Thermo; 17899) buffer for 15 min. Samples were hybridized overnight at 37° C. with 13×FLAP-Y-Cy-3 tagged complementary oligonucleotide probes targeting regions in human Cox4i1 mRNA (IDT). Hybridization mix: 15% Formamide, 1.7% tRNA (Sigma; R1753), 2% FLAP:Probe mix, 1% VRC (Sigma; R3380), 1% BSA (Roche; 10711454001), 20% Dextran Sulfate (Sigma; D8906), 1×SSC. Samples were washed twice with warm 15% Formamide, X1 SSC buffer (1 h each), and then 30 min with 1×SSC buffer, and 30 min with 1×PBS. Prior to immunostaining samples were washed with TRIS-HCl (pH=7.5), 0.15 M NaCl buffer, and then permeabilized with same buffer with 0.1% triton. Samples blocked for 30 min with TRIS-HCl-NaCl buffer with 0.1% Triton and 2% BSA. Samples were incubated with primary antibodies in blocking buffer overnight at 4° C., and then with fluorescent secondary antibodies in blocking buffer for 2 h at room temperatures. Samples were mounted with Vectashield Antifade reagent. See Table 5, FIG. 29, for the smFISH probe sequences.

RNA Immunoprecipitation

RNA-IP was performed as previously described. See, Shigeoka, T. et al. Dynamic axonal translation in developing and mature visual circuits. Cell 166, 181-192 (2016).

Briefly, all solutions were prepared using ultra-pure RNase-free water and analytical grade reagents. Cells were Harvested using ice cold 1% NP-40 RIP buffer containing 20 mM HEPES-KOH, 5 mM MgCl₂, 150 mM KCl, 1 mM DTT, 1% NP-40, 200 U/mL RiboLock, 100 μg/mL Cycloheximide, 1× cOmplete EDTA-free protease Inhibitor. Lysate was centrifuged for 15 min at 15,000×g. Protein concentration was determined with Bradford protein assay.

For immunoprecipitation, 3 mg of protein were pre-cleared for 1 h and then divided into TDP43-RIP or IgG control tubes, each was incubated overnight at 4° C. with 2 μg of TDP-43 or IgG antibody respectively. Agarose Beads were cleared with RIP buffer at 4° C. for 2 h. TDP43-RIP and IgG samples was incubated with 2μg antibody. Samples were incubated with beads for 2 h at 4° C., and then centrifuges at 1,000×g for 1 min. Beads in pellet were washed 3 times with wash buffer containing 20 mM HEPES-KOH, 5 mM MgCl₂, 350 mM KCl, 1 mM DTT, 0.1% NP-40, 200 U/mL RiboLock, 100 μg/mL Cycloheximide and cOmplete EDTA-free protease inhibitor cocktail. RNA from IP fraction was extracted using miRNeasy micro kit (Qiagen). MT was calculated between the cT of each IP sample and its input sample.

Mitochondria Membrane Potential Measurement

Mitochondrial membrane potential was assessed with Tetramethylrhodamine Ethyl, Ester (TMRE; Thermo Fisher) dye. Cultures were incubated with 20 nM TMRE for 30 min in CO₂ incubator, then washed 3 times with culture medium. Images of TMRE labeled axons in the distal compartment of MFCs were acquired before and after cultures were treated with Anisomycin (40 μM) or NaAsO₂ (250 μM), Cycloheximide (150 μM) in ×60 magnification. The volume of medium was kept higher in the proximal compartment to prevent the flow of treatment and ensure cell bodies remain unaffected. The intensity of TMRE fluorescence in axonal mitochondria was measured using FIJI, and the fraction of change post/pretreatment was calculated for each image field.

Mito-KillerRed (MKR) Experiments

MKR construct was a kind gift from Prof. Thomas Schwartz (Boston Children's Hospital).

MKR experiments were performed by co-culturing WT MNs and muscles in MFCs. Immediately after their plating, MNs were infected with lentiviral particles containing the MKR transfer plasmid. WT ChAT MNs were used as a negative control in co-cultures without MKR expression. After 12 days in co-culture, upon NMJ formation, co-cultures were labeled with Oregon-Green BAPTA (OGB; life technologies) for 40 min. Axons expressing MKR or ChAT were tracked until their contact points with muscles in the distal compartment. An ROI was then marked around the axons that overlap with the muscles, which was later frapped with a 560 nm laser (100 repeats of 200 μS). High-speed image sequences of calcium transients were acquired before and after 560 nm laser irradiation, and the percent of change in contraction rate post/pre was calculated for each muscle.

Lentivirus Production and Infection

Lentivirus particles were used to infect MNs with the MKR gene. We used second generation packaging system. The helper pVSVG and pGag-Pol were gifts from Prof. Eran Bacharach (Tel-Aviv University). For lentiviral production, HEK293-T cells (CRL-3216, ATCC) grown on a 60-mm dish. Once 70 to 80% confluence was achieved, cells were transfected with 10 μg of transfer plasmid, 7.5 μg of pGag-Pol, and 2.5 μg of pVSVG. Plasmids were placed in a calcium-phosphate transfection mix (25 mM Hepes, 5 mM KCl, 140 mM NaCl, and 0.75 mM Na₂PO₄ with 125 mM CaCl2) immediately before their addition to cells, in a volume of 0.5 ml per plate. Culture supernatants were harvested 2 days after transfection and concentrated ×10 using PEG Virus Precipitation kit (Abcam). Final pellets were each resuspended in Neurobasal media, aliquoted, and kept in −80° C. until use. For transduction of MNs, 2 μL of concentrated lentiviral suspension was used per MFC containing 150,000 MNs. Lentiviral vectors were added 1 to 2 h after plating MNs and were washed out three times in CNB medium 24 h later.

Protein Synthesis Inhibition Functional Analysis

Analysis of axon degeneration following protein synthesis inhibition was performed by culturing either WT MNs from HB9::GFP embryos, or ANLS and control MNs from TDP43ΔNLS embryos in the proximal compartment of MFC. Once axons have extensively crossed to the distal compartment, or after 10 days (for TDP43ΔNLS cultures), protein synthesis inhibitors were added to the distal (axonal) compartment while maintaining a higher volume of medium in the proximal compartment to prevent exposure of the cell-bodies to inhibitors. Puromycin (100 μg/mL) or Anisomycin (40 μM; only for HB9::GFP MN) were applied exclusively to axons in CNB medium. Images of axons were acquired at low magnification before, and after 16 (TDP43ΔNLS) and 24 h to monitor the extent of axon degeneration.

Analysis of NMJ function following protein synthesis inhibition with puromycin was performed by co-culturing WT MNs with primary muscles transfected with empty PQCXIP-mCherry vectors expressing PAC gene for puromycin resistance. After 12 days in co-culture, once cultures matured and NMJ were formed, Puromycin (100 μg/mL) was added exclusively to the distal (NMJ) compartment for 16 h. The proximal compartment was kept with higher volume of medium for allowing puromycin to act only locally within the NMJ compartment. After 16 h, cultures were labeled with OGB, and the calcium activity of axons and muscles was recorded. Analysis of the percent of muscles with calcium transients in co-culture, was performed on muscles with at least one overlapping axon. Only muscles that expressed mCherry (as a reporter for the expression of PAC) were used for this analysis.

Co-Culture Calcium Imaging

OGB lyophilized stock (Life technologies) was resuspended with 20% (w/v) Pluronic acid for a stock concentration of 3 mM Stock was diluted 1:1,000 in the appropriate medium. OGB was incubated with cultures for 40 mM in 37° C., 5% CO2 incubator, and then washed 3 times with culture medium prior imaging. Calcium transients in axons and muscles were recorded in a spinning disk confocal microscope equipped with an EMCCD camera with ×40 oil objective using 488 nm laser. Image sequences of 1,000 frames were acquired at frame rate of 25 FPS. Image analysis was performed using the “Time Series Analyzer V3” plugin for FIJI. Briefly, the mean OGB values for a Region of Interest (ROI) were plotted over the complete movie length. This assisted us to determine whether or not a certain muscle was active, and whether the activity was paired with neuronal firing. For figure labeling, axon endings on muscles, which also had high basal OGB signal were considered as NMJs.

Statistical Analysis

Statistical parameters and test used are noted in figure legends. All experiments included at least 3 biological repeats. Images and micrographs are representative of all experimental repeats. Statistical significance was determined using student's-t-test or mann-whitney test when comparing between two groups, and multiple comparisons ANOVA test when comparing more than two groups. Multiple comparisons were corrected using Holm-Sidak correction. Threshold for determining statistical significance was P<0.05. All statistical analysis was performed with Graph-pad Prism 7.

Data Availability

The proteomics data (FIG. 20 at a-c) have been deposited in the ProteomeXchange Consortium via the PRIDE partner repository database under accession code PXDO21876. Acquired proteomics data was correlated to the mouse FASTA databased using MaxQuant (v. 1.5.3.8) and its implemented Andromeda search engine.

SEQUENCE LISTING
The following are included in the Sequence Listing provided herewith
and incorporated by reference.

| | |
|---|---|
| TDP-43 | SEQ ID NO: 1. |
| G3BP1 | SEQ ID NO: 2 |
| G3BP1 190-208 | SEQ ID NO: 3 |
| TIA-1 | SEQ ID NO: 4 |
| FMRP | SEQ ID NO: 5 |
| FXR1 | SEQ ID NO: 6 |
| FUS | SEQ ID NO: 7 |
| HuR | SEQ ID NO: 8 |
| DCP1A | SEQ ID NO: 9 |
| NRN1 | SEQ ID NO: 10 |
| Impβ1 | SEQ ID NO: 11 |
| Gap43 | SEQ ID NO: 12 |
| C9ORF72 | SEQ ID NO: 13 |
| COX4I | SEQ ID NO: 14 |
| ATP5A1 | SEQ ID NO: 15 |
| NDUFA4 | SEQ ID NO: 16 |
| POlB | SEQ ID NO: 17 Forward Primer |
| ACTB | SEQ ID NO: 18 Forward Primer |
| PolB | SEQ ID NO: 19 Forward Primer |
| Mito-RNR1 | SEQ ID NO: 20 Forward Primer |
| Cox4i | SEQ ID NO: 21 Forward Primer |
| ATP5A1 | SEQ ID NO: 22 Forward Primer |
| Ndufa4 | SEQ ID NO: 23 Forward Primer |
| hCox4i | SEQ ID NO: 24 Forward Primer |
| hATP5A1 | SEQ ID NO: 25 Forward Primer |
| G3BP1 147-166 | SEQ ID NO: 26 |
| G2BP1 168-189 | SEQ ID NO: 27 |
| G3BP1 190-208 | SEQ ID NO: 28 |
| G3BP1 189-209 | SEQ ID NO: 29 |
| hCox4i1-1 | SEQ ID NO: 30 |
| hCox4i1-2 | SEQ ID NO: 31 |
| hCox4i1-3 | SEQ ID NO: 32 |
| hCox4i1-4 | SEQ ID NO: 33 |
| hCox4i1-5 | SEQ ID NO: 34 |
| hCox4i1-6 | SEQ ID NO: 35 |
| hCox4i1-7 | SEQ ID NO: 36 |
| hCox4i1-8 | SEQ ID NO: 37 |
| hCox4i1-9 | SEQ ID NO: 38 |
| hCox4i1-10 | SEQ ID NO: 39 |
| hCox4i1-11 | SEQ ID NO: 40 |
| hCox4i1-12 | SEQ ID NO: 41 |
| hCox4i1-13 | SEQ ID NO: 42 |
| POlB | SEQ ID NO: 43 Reverse Primer |
| ACTB | SEQ ID NO: 44 Reverse Primer |
| PolB | SEQ ID NO: 45 Reverse Primer |
| Mito-RNR1 | SEQ ID NO: 46 Reverse Primer |
| Cox4i | SEQ ID NO: 47 Reverse Primer |
| ATP5A1 | SEQ ID NO: 48 Reverse Primer |
| Ndufa4 | SEQ ID NO: 49 Reverse Primer |
| hCox4i | SEQ ID NO: 50 Reverse Primer |
| hATP5A1 | SEQ ID NO: 51 Reverse Primer |

SEQUENCE LISTING-USC 2033101.000390

| | |
|---|---|
| <110> | University of South Carolina/ Tel-Aviv University Ltd. |
| <120> | TARGETING G3BP AGGREGATION TO PREVENT |
| | NEURODEGENERATION |
| <130> | 2033101.0000390 |
| <140> | Unknown |
| <141> | *** |
| <150> | U.S. Application No. 16/881,096 |
| <151> | 05/22/2020 |
| <150> | U.S. Application No. 62/876,852 |
| <151> | 07/22/2019 |
| <160> | 1 |
| <170> | PatentIn |
| <210> | 1 |
| <211> | 414 |
| <212> | PRT |
| <213> | Homo sapiens |
| <221> | CDS |
| <222> | 1...414 |
| <400> | 1 |

MetSerGluTyrIleArgValThrGluAspGluAsnAspGluProIleGluIleProSer
GluAspAspGlyThrValLeuLeuSerThrValThrAlaGlnPheProGlyAlaCysGlyLeuArgTyr
ArgAsnProValSerGlnCysMetArgGlyValArgLeuValGluGlyIleLeuHisAlaProAspAla
GlyTrpGlyAsnLeuValTyrValValAsnTyrProLysAspAsnLysArgLysMetAspGluThrAsp
AlaSerSerAlaValLysValLysArgAlaValGlnLysThrSerAspLeuIleValLeuGlyLeuPro
TrpLysThrThrGluGlnAspLeuLysGluTyrPheSerThrPheGlyGluValLeuMetValGlnVal
LysLysAspLeuLysThrGlyHisSerLysGlyPheGlyPheValArgPheThrGluTyrGluThrGln
ValLysValMetSerGlnArgHisMetIleAspGlyArgTrpCysAspCysLysLeuProAsnSerLys
GlnSerGlnAspGluProLeuArgSerArgLysValPheValGlyArgCysThrGluAspMetThrGlu
AspGluLeuArgGluPhePheSerGlnTyrGlyAspValMetAspValPheIleProLysProPheArg
AlaPheAlaPheValThrPheAlaAspAspGlnIleAlaGlnSerLeuCysGlyGluAspLeuIleIle
LysGlyIleSerValHisIleSerAsnAlaGluProLysHisAsnSerAsnArgGlnLeuGluArgSer
GlyArgPheGlyGlyAsnProGlyGlyPheGlyAsnGlnGlyGlyPheGlyAsnSerArgGlyGlyGly
AlaGlyLeuGlyAsnAsnGlnGlySerAsnMetGlyGlyGlyMetAsnPheGlyAlaPheSerIleAsn
ProAlaMetMetAlaAlaAlaGlnAlaAlaLeuGlnSerSerTrpGlyMetMetGlyMetLeuAlaSer
GlnGlnAsnGlnSerGlyProSerGlyAsnAsnGlnAsnGlnGlyAsnMetGlnArgGluProAsnGln
AlaPheGlySerGlyAsnAsnSerTyrSerGlySerAsnSerGlyAlaAlaIleGlyTrpGlySerAla
SerAsnAlaGlySerGlySerGlyPheAsnGlyGlyPheGlySerSerMetAspSerLysSerSerGly
TrpGlyMet

| | |
|---|---|
| <210> | 2 |
| <211> | 466 |
| <212> | PRTN |
| <213> | Homo sapiens |
| <221> | CDS |
| <222> | 1-466 |
| <400> | 2 |

MetValMetGluLysProSerProLeuLeuValGlyArgGluPheValArgGlnTyr
TyrThrLeuLeuAsnGlnAlaProAspMetLeuHisArgPheTyrGlyLysAsnSerSerTyrValHis
GlyGlyLeuAspSerAsnGlyLysProAlaAspAlaValTyrGlyGlnLysGluIleHisArgLysValMet
SerGlnAsnPheThrAsnCysHisThrLysIleArgHisValAspAlaHisAlaThrLeuAsnAspGly
ValValValGlnValMetGlyLeuLeuSerAsnAsnAsnGlnAlaLeuArgArgPheMetGlnThr
PheValLeuAlaProGluGlySerValAlaAsnLysPheTyrValHisAsnAspIlePheArgTyrGlnAsp
GluValPheGlyGlyPheValThrGluProGlnGluGluSerGluGluGluValGluGluProGluGlu
ArgGlnGlnThrProGluValValProAspAspSerGlyThrPheTyrAspGlnAlaValValSerAsn
AspMetGluGluHisLeuGluGluProValAlaGluProGluProAspProGluProGluProGluGln
GluProValSerGluIleGlnGluGluLysProGluProValLeuGluGluThrAlaProGluAspAla
GlnLysSerSerSerProAlaProAlaAspIleAlaGlnThrValGlnGluAspLeuArgThrPheSerTrp
AlaSerValThrSerLysAsnLeuProProSerGlyAlaValProValThrGlyIleProProHisValVal
LysValProAlaSerGlnProArgProGluSerLysProGluSerGlnIleProProGlnArgProGln
ArgAspGlnArgValArgGluGlnArgIleAsnIleProProGlnArgGlyProArgProIleArgGluAla
GlyGluGlnGlyAspIleGluProArgArgMetValArgHisProAspSerHisGlnLeuPheIleGlyAsn
LeuProHisGluValAspLysSerGluLeuLysAspPhePheGlnSerTyrGlyAsnValValGluLeu
ArgIleAsnSerGlyGlyLysLeuProAsnPheGlyPheValValPheAspAspSerGluProValGln
LysValLeuSerAsnArgProIleMetPheArgGlyGluValArgLeuAsnValGluGluLysLysThr
ArgAlaAlaArgGluGlyAspArgArgAspAsnArgLeuArgGlyProGlyGlyProArgGlyGlyLeu
GlyGlyGlyMetArgGlyProProArgGlyGlyMetValGlnLysProGlyPheGlyValGlyArgGly
LeuAlaProArgGln

| | |
|---|---|
| <210> | 3 |
| <211> | 31 |
| <212> | PRTN |
| <213> | Homo sapiens |
| <221> | CDS |
| <222> | 190-208 |
| <400> | 3 |

TyrGlyAsnLysLysAsnAsnAsnGlnAsnAsnAsnValValGluProGluProGlu
ProGluProGluProGluProGluProValSerGlu

| | |
|---|---|
| <210> | 4 |
| <211> | 386 |
| <212> | PRTN |
| <213> | Homo sapiens |
| <221> | CDS |
| <222> | Sequence Placement #s |
| <400> | 1-386 |

MetGluAspGluMetProLysThrLeuTyrValGlyAsnLeuSerArgAspValThr
GluAlaLeuIleLeuGlnLeuPheSerGlnIleGlyProCysLysAsnCysLysMetIleMetAspThr
AlaGlyAsnAspProTyrCysPheValGluPheHisGluHisArgHisAlaAlaAlaAlaLeuAlaAla
MetAsnGlyArgLysIleMetGlyLysGluValLysValAsnTrpAlaThrThrProSerSerGlnLysLys
AspThrSerSerSerThrValValSerThrGlnArgSerGlnAspHisPheHisValPheValGlyAsp
LeuSerProGluIleThrThrGluAspIleLysAlaAlaPheAlaProPheGlyArgIleSerAspAlaArg
ValValLysAspMetAlaThrGlyLysSerLysGlyTyrGlyPheValSerPhePheAsnLysTrpAsp
AlaGluAsnAlaIleGlnGlnMetGlyGlyGlnTrpLeuGlyGlyArgGlnIleArgThrAsnTrpAlaThr
ArgLysProProAlaProLysSerThrTyrGluSerAsnThrLysGlnLeuSerTyrAspGluValVal
AsnGlnSerSerProSerAsnCysThrValTyrCysGlyGlyValThrSerGlyLeuThrGluGlnLeu
MetArgGlnThrPheSerProPheGlyGlnIleMetGluIleArgValPheProAspLysGlyTyrSerPhe
ValArgPheAsnSerHisGluSerAlaAlaHisAlaIleValSerValAsnGlyThrThrIleGluGlyHis
ValValLysCysTyrTrpGlyLysGluThrLeuAspMetIleAsnProValGlnGlnGlnAsnGlnIle
GlyTyrProGlnProTyrGlyGlnTrpGlyGlnTrpTyrGlyAsnAlaGlnGlnIleGlyGlnTyrMetPro
AsnGlyTrpGlnValProAlaTyrGlyMetTyrGlyGlnAlaTrpAsnGlnGlnGlyPheAsnGlnThr
GlnSerSerAlaProTrpMetGlyProAsnTyrGlyValGlnProProGlnGlyGlnAsnGlySerMet
LeuProAsnGlnProSerGlyTyrArgValAlaGlyTyrGluThrGln

| | |
|---|---|
| <210> | 5 |
| <211> | 695 |
| <212> | PRTN |
| <213> | Homo sapiens |
| <221> | CDS |
| <222> | 1-695 |
| <400> | 5 |

MetGluGluLysProGlyGlnProGlnProGlnHisHisHisSerHisHisHisProHis
HisHisProGlnGlnGlnGlnGlnGlnProHisHisHisHisHisTyrTyrPheTyrAsnHisSerHis
AsnHisHisHisHisHisHisHisGlnGlnProHisGlnTyrLeuGlnHisGlyAlaGluGlySerProLys
AlaGlnProLysProLeuLysHisGluGlnLysHisThrLeuGlnGlnHisGlnGluThrProLysLys
LysThrGlyTyrGlyGluLeuAsnGlyAsnAlaGlyGluArgGluIleSerLeuLysAsnLeuSerSer
AspGluAlaThrAsnProIleSerArgValLeuAsnGlyAsnGlnGlnValValAspThrSerLeuLys
GlnThrValLysAlaAsnThrPheGlyLysAlaGlyIleLysThrLysAsnPheIleGlnLysAsnSer
MetAspLysLysAsnGlyLysSerTyrGluAsnLysSerGlyGluAsnGlnSerValAspLysSerAsp
ThrIleProIleProAsnGlyValValThrAsnAsnSerGlyTyrIleThrAsnGlyTyrMetGlyLysGly
AlaAspAsnAspGlySerGlySerGluSerGlyTyrThrThrProLysLysArgLysAlaArgArgAsn
SerAlaLysGlyCysGluAsnLeuAsnIleValGlnAspLysIleMetGlnGlnGluThrSerValProThr
LeuLysGlnGlyLeuGluThrPheLysProAspTyrSerGluGlnLysGlyAsnArgValAspGlySer
LysProIleTrpLysTyrGluThrGlyProGlyGlyThrSerArgGlyLysProAlaValGlyAspMet
LeuArgLysSerSerAspSerLysProGlyValSerSerLysLysPheAspAspArgProLysGlyLysHis
AlaSerAlaValAlaSerLysGluAspSerTrpThrLeuPheLysProProProValPheProValAsp
AsnSerSerAlaLysIleValProLysIleSerTyrAlaSerLysValLysGluAsnLeuAsnLysThrIle
GlnAsnSerSerValSerProThrSerSerSerSerSerSerSerSerThrGlyGluThrGlnThrGlnSer
SerSerArgLeuSerGlnValProMetSerAlaLeuLysSerValThrSerAlaAsnPheSerAsnGly
ProValLeuAlaGlyThrAspGlyAsnValTyrProProGlyGlyGlnProLeuLeuThrThrAlaAla
AsnThrLeuThrProIleSerSerGlyThrAspSerValLeuGlnAspMetSerLeuThrSerAlaAlaVal
GluGlnIleLysThrSerLeuPheIleTyrProSerAsnMetGlnThrMetLeuLeuSerThrAlaGln
ValAspLeuProSerGlnThrAspGlnGlnAsnLeuGlyAspIlePheGlnAsnGlnTrpGlyLeuSer
PheIleAsnGluProSerAlaGlyProGluThrValThrGlyLysSerSerGluHisLysValMetGluVal
ThrPheGlnGlyGluTyrProAlaThrLeuValSerGlnGlyAlaGluIleIleProSerGlyThrGluHis
ProValPheProLysAlaTyrGluLeuGluLysArgThrSerProGlnValLeuGlySerIleLeuLys
SerGlyThrThrSerGluSerGlyAlaLeuSerLeuGluProSerHisIleGlyAspLeuGlnLysAlaAsp
ThrSerSerGlnGlyAlaLeuValPheLeuSerLysAspTyrGluIleGluSerGlnAsnProLeuAla
SerProThrAsnThrLeuLeuGlySerAlaLysGluGlnArgTyrGlnArgGlyLeuGluArgAsnAsp
SerTrpGlySerPheAspLeuArgAlaAlaIleValTyrHisThrLysGluMetGluSerIleTrpAsnLeu
GlnLysGlnAspProLysArgIleIleThrTyrAsnGluAlaMetAspSerProAspGln

| | |
|---|---|
| <210> | 6 |
| <211> | 621 |
| <212> | PRTN |
| <213> | Homo sapiens |
| <221> | CDS |
| <222> | 1-621 |
| <400> | 6 |

MetAlaGluLeuThrValGluValArgGlySerAsnGlyAlaPheTyrLysGlyPheIle
LysAspValHisGluAspSerLeuThrValValPheGluAsnAsnTrpGlnProGluArgGlnValPro
PheAsnGluValArgLeuProProProProAspIleLysLysGluIleSerGluGlyAspGluValGlu
ValTyrSerArgAlaAsnAspGlnGluProCysGlyTrpTrpLeuAlaLysValArgMetMetLysGly
GluPheTyrValIleGluTyrAlaAlaCysAspAlaThrTyrAsnGluIleValThrPheGluArgLeuArg
ProValAsnGlnAsnLysThrValLysLysAsnThrPhePheLysCysThrValAspValProGlu
AspLeuArgGluAlaCysAlaAsnGluAsnAlaHisLysAspPheLysLysAlaValGlyAlaCysArgIle
PheTyrHisProGluThrThrGlnLeuMetIleLeuSerAlaSerGluAlaThrValLysArgValAsn
IleLeuSerAspMetHisLeuArgSerIleArgThrLysLeuMetLeuMetSerArgAsnGluGluAla
ThrLysHisLeuGluCysThrLysGlnLeuAlaAlaAlaPheHisGluGluPheValValArgGluAsp
LeuMetGlyLeuAlaIleGlyThrHisGlySerAsnIleGlnGlnAlaArgLysValProGlyValThrAla
IleGluLeuAspGluAspThrGlyThrPheArgIleTyrGlyGluSerAlaAspAlaValLysLysAlaArg
GlyPheLeuGluPheValGluAspPheIleGlnValProArgAsnLeuValGlyLysValIleGlyLys
AsnGlyLysValIleGlnGluIleValAspLysSerGlyValValArgValArgIleGluGlyAspAsnGlu
AsnLysLeuProArgGluAspGlyMetValProPheValPheValGlyThrLysGluSerIleGlyAsn
ValGlnValLeuLeuGluTyrHisIleAlaTyrLeuLysGluValGluGlnLeuArgMetGluArgLeuGln
IleAspGluGlnLeuArgGlnIleGlySerArgSerTyrSerGlyArgGlyArgGlyArgArgGlyPro
AsnTyrThrSerGlyTyrGlyThrAsnSerGluLeuSerAsnProSerGluThrGluSerGluArgLys
AspGluLeuSerAspTrpSerLeuAlaGlyGluAspAspArgAspSerArgHisGlnArgAspSerArg
ArgArgProGlyGlyArgGlyArgSerValSerGlyGlyArgGlyArgGlyGlyProArgGlyGlyLysSer
SerIleSerSerValLeuLysAspProAspSerAsnProTyrSerLeuLeuAspAsnThrGluSerAsp
GlnThrAlaAspThrAspAlaSerGluSerHisHisSerThrAsnArgArgArgArgSerArgArgArg
ArgThrAspGluAspAlaValLeuMetAspGlyMetThrGluSerAspThrAlaSerValAsnGluAsn
GlyLeuValThrValAlaAspTyrIleSerArgAlaGluSerGlnSerArgGlnArgAsnLeuProArgGlu
ThrLeuAlaLysAsnLysLysGluMetAlaLysAspValIleGluGluHisGlyProSerGluLysAla
IleAsnGlyProThrSerAlaSerGlyAspAspIleSerLysLeuGlnArgThrProGlyGluGluLysIle
AsnThrLeuLysGluGluAsnThrGlnGluAlaAlaValLeuAsnGlyValSer

| | |
|---|---|
| <210> | 7 |
| <211> | 526 |
| <212> | PRTN |
| <213> | Homo sapiens |
| <221> | CDS |
| <222> | 1-526 |
| <400> | 7 |

MetAlaSerAsnAspTyrThrGlnGlnAlaThrGlnSerTyrGlyAlaTyrProThrGln
ProGlyGlnGlyTyrSerGlnGlnSerSerGlnProTyrGlyGlnGlnSerTyrSerGlyTyrSerGln
SerThrAspThrSerGlyTyrGlyGlnSerSerTyrSerSerTyrGlyGlnSerGlnAsnThrGlyTyrGly
ThrGlnSerThrProGlnGlyTyrGlySerThrGlyGlyTyrGlySerSerGlnSerSerGlnSerSer
TyrGlyGlnGlnSerSerTyrProGlyTyrGlyGlnGlnProAlaProSerSerThrSerGlySerTyrGly
SerSerSerGlnSerSerSerTyrGlyGlnProGlnSerGlySerTyrSerGlnGlnProSerTyrGlyGly
GlnGlnGlnSerTyrGlyGlnGlnGlnSerTyrAsnProProGlnGlyTyrGlyGlnGlnAsnGlnTyr
AsnSerSerSerGlyGlyGlyGlyGlyGlyGlyGlyGlyGlyAsnTyrGlyGlnAspGlnSerSerMet
SerSerGlyGlyGlySerGlyGlyGlyTyrGlyAsnGlnAspGlnSerGlyGlyGlyGlySerGlyGlyTyr
GlyGlnGlnAspArgGlyGlyArgGlyArgGlyGlySerGlyGlyGlyGlyGlyGlyGlyGlyGlyGly
TyrAsnArgSerSerGlyGlyTyrGluProArgGlyArgGlyGlyGlyArgGlyGlyArgGlyGlyMet
GlyGlySerAspArgGlyGlyPheAsnLysPheGlyGlyProArgAspGlnGlySerArgHisAspSer
GluGlnAspAsnSerAspAsnAsnThrIlePheValGlnGlyLeuGlyGluAsnValThrIleGluSerVal
AlaAspTyrPheLysGlnIleGlyIleIleLysThrAsnLysLysThrGlyGlnProMetIleAsnLeuTyr
ThrAspArgGluThrGlyLysLeuLysGlyGluAlaThrValSerPheAspAspProProSerAlaLys
AlaAlaIleAspTrpPheAspGlyLysGluPheSerGlyAsnProIleLysValSerPheAlaThrArg
ArgAlaAspPheAsnArgGlyGlyGlyAsnGlyArgGlyGlyArgGlyArgGlyGlyProMetGlyArg
GlyGlyTyrGlyGlyGlyGlySerGlyGlyGlyGlyArgGlyGlyPheProSerGlyGlyGlyGlyGlyGly
GlyGlnGlnArgAlaGlyAspTrpLysCysProAsnProThrCysGluAsnMetAsnPheSerTrpArg
AsnGluCysAsnGlnCysLysAlaProLysProAspGlyProGlyGlyGlyProGlyGlySerHisMet
GlyGlyAsnTyrGlyAspAspArgArgGlyGlyArgGlyGlyTyrAspArgGlyGlyTyrArgGlyArg
GlyGlyAspArgGlyGlyPheArgGlyGlyArgGlyGlyGlyAspArgGlyGlyPheGlyProGlyLys
MetAspSerArgGlyGluHisArgGlnAspArgArgGluArgProTyr

| | |
|---|---|
| <210> | 8 |
| <211> | 326 |
| <212> | PRTN |
| <213> | Homo sapiens |
| <221> | CDS |
| <222> | 1-326 |
| <400> | 8 |

MetSerAsnGlyTyrGluAspHisMetAlaGluAspCysArgGlyAspIleGlyArgThr
AsnLeuIleValAsnTyrLeuProGlnAsnMetThrGlnAspGluLeuArgSerLeuPheSerSer
IleGlyGluValGluSerAlaLysLeuIleArgAspLysValAlaGlyHisSerLeuGlyTyrGlyPheVal
AsnTyrValThrAlaLysAspAlaGluArgAlaIleAsnThrLeuAsnGlyLeuArgLeuGlnSerLys
ThrIleLysValSerTyrAlaArgProSerSerGluValIleLysAspAlaAsnLeuTyrIleSerGlyLeu
ProArgThrMetThrGlnLysAspValGluAspMetPheSerArgPheGlyArgIleIleAsnSerArgVal
LeuValAspGlnThrThrGlyLeuSerArgGlyValAlaPheIleArgPheAspLysArgSerGluAla
GluGluAlaIleThrSerPheAsnGlyHisLysProProGlySerSerGluProIleThrValLysPheAla
AlaAsnProAsnGlnAsnLysAsnValAlaLeuLeuSerGlnLeuTyrHisSerProAlaArgArgPhe
GlyGlyProValHisHisGlnAlaGlnArgPheArgPheSerProMetGlyValAspHisMetSerGly
LeuSerGlyValAsnValProGlyAsnAlaSerSerGlyTrpCysIlePheIleTyrAsnLeuGlyGlnAsp
AlaAspGluGlyIleLeuTrpGlnMetPheGlyProPheGlyAlaValThrAsnValLysValIleArg
AspPheAsnThrAsnLysCysLysGlyPheGlyPheValThrMetThrAsnTyrGluGluAlaAlaMet
AlaIleAlaSerLeuAsnGlyTyrArgLeuGlyAspLysIleLeuGlnValSerPheLysThrAsnLys
SerHisLys

| | |
|---|---|
| <210> | 9 |
| <211> | 582 |
| <212> | PRTN |
| <213> | Homo sapiens |
| <221> | CDS |
| <222> | Sequence Placement #s |
| <400> | 1-582 |

MetGluAlaLeuSerArgAlaGlyGlnGluMetSerLeuAlaAlaLeuLysGlnHis
AspProTyrIleThrSerIleAlaAspLeuThrGlyGlnValAlaLeuTyrThrPheCysProLysAlaAsn
GlnTrpGluLysThrAspIleGluGlyThrLeuPheValTyrArgArgSerAlaSerProTyrHisGly
PheThrIleValAsnArgLeuAsnMetHisAsnLeuValGluProValAsnLysAspLeuGluPheGln
LeuHisGluProPheLeuLeuTyrArgAsnAlaSerLeuSerIleTyrSerIleTrpPheTyrAspLys
AsnAspCysHisArgIleAlaLysLeuMetAlaAspValValGluGluGluThrArgArgSerGlnGln
AlaAlaArgAspLysGlnSerProSerGlnAlaAsnGlyCysSerAspHisArgProIleAspIleLeuGlu
MetLeuSerArgAlaLysAspGluTyrGluArgAsnGlnMetGlyAspSerAsnIleSerSerProGly
LeuGlnProSerThrGlnLeuSerAsnLeuGlySerThrGluThrLeuGluGluMetProSerGlySer
GlnAspLysSerAlaProSerGlyHisLysHisLeuThrValGluGluLeuPheGlyThrSerLeuPro
LysGluGlnProAlaValValGlyLeuAspSerGluGluMetGluArgLeuProGlyAspAlaSerGln
LysGluProAsnSerPheLeuProPheProPheGluGlnLeuGlyGlyAlaProGlnSerGluThrLeu
GlyValProSerAlaAlaHisHisSerValGlnProGluIleThrThrProValLeuIleThrProAlaSer
IleThrGlnSerAsnGluLysHisAlaProThrTyrThrIleProLeuSerProValLeuSerProThrLeu
ProAlaGluAlaProThrAlaGlnValProProSerLeuProArgAsnSerThrMetMetGlnAlaVal
LysThrThrProArgGlnArgSerProLeuLeuAsnGlnProValProGluLeuSerHisAlaSerLeu
IleAlaAsnGlnSerProPheArgAlaProLeuAsnValThrAsnThrAlaGlyThrSerLeuProSerVal
AspLeuLeuGlnLysLeuArgLeuThrProGlnHisAspGlnIleGlnThrGlnProLeuGlyLysGly
AlaMetValAlaSerPheSerProAlaAlaGlyGlnLeuAlaThrProGluSerPheIleGluProPro
SerLysThrAlaAlaAlaArgValAlaAlaSerAlaSerLeuSerAsnMetValLeuAlaProLeuGlnSer
MetGlnGlnAsnGlnAspProGluValPheValGlnProLysValLeuSerSerAlaIleProValAla
GlyAlaProLeuValThrAlaThrThrThrAlaValSerSerValLeuLeuAlaProSerValPheGlnGln
ThrValThrArgSerSerAspLeuGluArgLysAlaSerSerProSerProLeuThrIleGlyThrPro
GluSerGlnArgLysProSerIleIleLeuSerLysSerGlnLeuGlnAspThrLeuIleHisLeuIleLys
AsnAspSerSerPheLeuSerThrLeuHisGluValTyrLeuGlnValLeuThrLysAsnLysAspAsn
HisAsnLeu

| | |
|---|---|
| <210> | 10 |
| <211> | 142 |
| <212> | PRTN |
| <213> | Homo sapiens |
| <221> | CDS |
| <222> | 1-142 |
| <400> | 10 |

MetGlyLeuLysLeuAsnGlyArgTyrIleSerLeuIleLeuAlaValGlnIleAlaTyr
LeuValGlnAlaValArgAlaAlaGlyLysCysAspAlaValPheLysGlyPheSerAspCysLeuLeu
LysLeuGlyAspSerMetAlaAsnTyrProGlnGlyLeuAspAspLysThrAsnIleLysThrValCys
ThrTyrTrpGluAspPheHisSerCysThrValThrAlaLeuThrAspCysGlnGluGlyAlaLysAsp
MetTrpAspLysLeuArgLysGluSerLysAsnLeuAsnileGlnGlySerLeuPheGluLeuOysGly
SerGlyAsnGlyAlaAlaGlySerLeuLeuProAlaPheProValLeuLeuValSerLeuSerAlaAla
LeuAlaThrTrpLeuSerPhe

| | |
|---|---|
| <210> | 11 |
| <211> | 419 |
| <212> | PRTN |
| <213> | Escherichia coli |
| <221> | CDS |
| <222> | 1-419 |
| <400> | 11 |

MetPheAlaLeuAlaAspIleAsnSerPheTyrAlaSerCysGluLysValPheArgPro
AspLeuArgAsnGluProValIleValLeuSerAsnAsnAspGlyCysValIleAlaArgSerProGlu
AlaLysAlaLeuGlyIleArgMetGlyGlnProTrpPheGlnValArgGlnMetArgLeuGluLysLys
IleHisValPheSerSerAsnTyrAlaLeuTyrHisSerMetSerGlnArgValMetAlaValLeuGlu
SerLeuSerProAlaValGluProTyrSerIleAspGluMetPheIleAspLeuArgGlyIleAsnHisCys
IleSerProGluPhePheGlyHisGlnLeuArgGluGlnValLysSerTrpThrGlyLeuThrMetGly
ValGlyIleAlaProThrLysThrLeuAlaLysSerAlaGlnTrpAlaThrLysGlnTrpProGlnPheSer
GlyValValAlaLeuThrAlaGluAsnArgAsnArgIleLeuLysLeuLeuGlyLeuGlnProValGly
GluValTrpGlyValGlyHisArgLeuThrGluLysLeuAsnAlaLeuGlyIleAsnThrAlaLeuGln
LeuAlaGlnAlaAsnThrAlaPheIleArgLysAsnPheSerValIleLeuGluArgThrValArgGluLeu
AsnGlyGluSerCysIleSerLeuGluGluAlaProProAlaLysGlnGlnIleValCysSerArgSer
PheGlyGluArgIleThrAspLysAspAlaMetHisGlnAlaValValGlnTyrAlaGluArgAlaAlaGlu
LysLeuArgGlyGluArgGlnTyrCysArgGlnValThrThrPheValArgThrSerProThrLeuLeu
GlnGlnCysAlaValGluLysLeuSerLeuProThrGlnAspSerArgAspIleleAlaAlaAlaCys
ArgAlaLeuAsnHisValTrpArgGluGlyTyrArgTyrMetLysAlaGlyValMetLeuAlaAspPhe
ThrProSerGlyIleAlaGlnProGlyLeuPheAspGluIleGlnProArgLysAsnSerGluLysLeuMet
LysThrLeuAspGluLeuAsnGlnSerGlyLysGlyLysValTrpPheAlaGlyArgGlyThrAlaPro
GluTrpGlnMetLysArgGluMetLeuSerGlnCysTyrThrThrLysTrpArgAspIleProLeuAla
ArgLeuGly

| | |
|---|---|
| <210> | 12 |
| <211> | 238 |
| <212> | PRTN |
| <213> | Homo sapiens |
| <221> | CDS |
| <222> | 1-238 |
| <400> | 12 |

MetLeuCysCysMetArgArgThrLysGlnValGluLysAsnAspAspAspGlnLys
IleGluGlnAspGlyIleLysProGluAspLysAlaHisLysAlaAlaThrLysIleGlnAlaSerPheArg
GlyHisIleThrArgLysLysLeuLysGlyGluLysLysAspAspValGlnAlaAlaGluAlaGluAla
AsnLysLysAspGluAlaProValAlaAspGlyValGluLysLysGlyGluGlyThrThrThrAlaGlu
AlaAlaProAlaThrGlySerLysProAspGluProGlyLysAlaGlyGluThrProSerGluGluLysLys
GlyGluGlyAspAlaAlaThrGluGlnAlaAlaProGlnAlaProAlaSerSerGluGluLysAlaGly
SerAlaGluThrGluSerAlaThrLysAlaSerThrAspAsnSerProSerSerLysAlaGluAspAlaPro
AlaLysGluGluProLysGlnAlaAspValProAlaAlaValThrAlaAlaAlaAlaThrThrProAla
AlaGluAspAlaAlaAlaLysAlaThrAlaGlnProProThrGluThrGlyGluSerSerGlnAlaGluGlu
AsnIleGluAlaValAspGluThrLysProLysGluSerAlaArgGlnAspGluGlyLysGluGluGlu
ProGluAlaAspGlnGluHisAla

| | |
|---|---|
| <210> | 13 |
| <211> | 480 |
| <212> | PRTN |
| <213> | Homo sapiens |
| <221> | CDS |
| <222> | 1-480 |
| <400> | 13 |

MetSerThrLeuCysProProProSerProAlaValAlaLysThrGluIleAlaLeuSer
GlyLysSerProLeuLeuAlaAlaThrPheAlaTyrTrpAspAsnIleLeuGlyProArgValArgHis
IleTrpAlaProLysThrGluGlnValLeuLeuSerAspGlyGluIleThrPheLeuAlaAsnHisThrLeu
AsnGlyGluIleLeuArgAsnAlaGluSerGlyAlaIleAspValLysPhePheValLeuSerGluLys
GlyValIleIleValSerLeuIlePheAspGlyAsnTrpAsnGlyAspArgSerThrTyrGlyLeuSerIleIle
LeuProGlnThrGluLeuSerPheTyrLeuProLeuHisArgValCysValAspArgLeuThrHisIle
IleArgLysGlyArgIleTrpMetHisLysGluArgGlnGluAsnValGlnLysIleIleLeuGluGlyThr
GluArgMetGluAspGlnGlyGlnSerIleIleProMetLeuThrGlyGluValIleProValMetGlu
LeuLeuSerSerMetLysSerHisSerValProGluGluIleAspIleAlaAspThrValLeuAsnAspAsp
AspIleGlyAspSerCysHisGluGlyPheLeuLeuAsnAlaIleSerSerHisLeuGlnThrCysGly
CysSerValValValGlySerSerAlaGluLysValAsnLysIleValArgThrLeuCysLeuPheLeuThr
ProAlaGluArgLysCysSerArgLeuCysGluAlaGluSerSerPheLysTyrGluSerGlyLeuPhe
ValGlnGlyLeuLeuLysAspSerThrGlySerPheValLeuProPheArgGlnValMetTyrAlaPro
TyrProThrThrHisIleAspValAspValAsnThrValLysGlnMetProProCysHisGluHisIle
TyrAsnGlnArgArgTyrMetArgSerGluLeuThrAlaPheTrpArgAlaThrSerGluGluAspMetAl
aGlnAspThrIleIleTyrThrAspGluSerPheThrProAspLeuAsnIlePheGlnAspValLeuHis
ArgAspThrLeuValLysAlaPheLeuAspGlnValPheGlnLeuLysProGlyLeuSerLeuArg
SerThrPheLeuAlaGlnPheLeuLeuValLeuHisArgLysAlaLeuThrLeuIleLysTyrIleGluAsp
AspThrGlnLysGlyLysLysProPheLysSerLeuArgAsnLeuLysIleAspLeuAspLeuThrAla
GluGlyAspLeuAsnIleIleMetAlaLeuAlaGluLysIleLysProGlyLeuHisSerPheIlePheGly
ArgProPheTyrThrSerValGlnGluArgAspValLeuMetThr

| | |
|---|---|
| <210> | 14 |
| <211> | 169 |
| <212> | PRTN |
| <213> | Mus musculus |
| <221> | CDS |
| <222> | 1-169 |
| <400> | 14 |

MetLeuAlaSerArgAlaLeuSerLeuIleGlyLysArgAlaIleSerThrSerValCys
LeuArgAlaHisGlySerValValLysSerGluAspTyrAlaPheProThrTyrAlaAspArgArgAsp
TyrProLeuProAspValAlaHisValThrMetLeuSerAlaSerGlnLysAlaLeuLysGluLysGlu
LysAlaAspTrpSerSerLeuSerArgAspGluLysValGlnLeuTyrArgIleGlnPheAsnGluSerPhe
AlaGluMetAsnArgGlyThrAsnGluTrpLysThrValValGlyMetAlaMetPhePheIleGlyPhe
ThrAlaLeuValLeuIleTrpGluLysSerTyrValTyrGlyProIleProHisThrPheAspArgAsp
TrpValAlaMetGlnThrLysArgMetLeuAspMetLysAlaAsnProIleGlnGlyPheSerAlaLys
TrpAspTyrAspLysAsnGluTrpLysLys

| | |
|---|---|
| <210> | 15 |
| <211> | 553 |
| <212> | PRTN |
| <213> | Homo sapiens |
| <221> | CDS |
| <222> | 1-553 |
| <400> | 15 |

MetLeuSerValArgValAlaAlaAlaValValArgAlaLeuProArgArgAlaGlyLeu
ValSerArgAsnAlaLeuGlySerSerPheIleAlaAlaArgAsnPheHisAlaSerAsnThrHisLeu
GlnLysThrGlyThrAlaGluMetSerSerIleLeuGluGluArgIleLeuGlyAlaAspThrSerVal
AspLeuGluGluThrGlyArgValLeuSerIleGlyAspGlyIleAlaArgValHisGlyLeuArgAsnVal
GlnAlaGluGluMetValGluPheSerSerGlyLeuLysGlyMetSerLeuAsnLeuGluProAsp
AsnValGlyValValValPheGlyAsnAspLysLeuIleLysGluGlyAspIleValLysArgThrGlyAla
IleValAspValProValGlyGluGluLeuLeuGlyArgValValAspAlaLeuGlyAsnAlaIleAspGly
LysGlyProIleGlySerLysThrArgArgArgValGlyLeuLysAlaProGlyIleIleProArgIleSer
ValArgGluProMetGlnThrGlyIleLysAlaValAspSerLeuValProIleGlyArgGlyGlnArgGlu
LeuIleIleGlyAspArgGlnThrGlyLysThrSerIleAlaIleAspThrIleIleAsnGlnLysArgPhe
AsnAspGlySerAspGluLysLysLysLeuTyrCysIleTyrValAlaIleGlyGlnLysArgSerThrVal
AlaGlnLeuValLysArgLeuThrAspAlaAspAlaMetLysTyrThrIleValValSerAlaThrAla
SerAspAlaAlaProLeuGlnTyrLeuAlaProTyrSerGlyCysSerMetGlyGluTyrPheArgAsp
AsnGlyLysHisAlaLeuIleIleTyrAspAspLeuSerLysGlnAlaValAlaTyrArgGlnMetSerLeu
LeuLeuArgArgProProGlyArgGluAlaTyrProGlyAspValPheTyrLeuHisSerArgLeuLeu
GluArgAlaAlaLysMetAsnAspAlaPheGlyGlyGlySerLeuThrAlaLeuProValIleGluThr
GlnAlaGlyAspValSerAlaTyrIleProThrAsnValIleSerIleThrAspGlyGlnIlePheLeuGluThr
GluLeuPheTyrLysGlyIleArgProAlaIleAsnValGlyLeuSerValSerArgValGlySerAlaAla
GlnThrArgAlaMetLysGlnValAlaGlyThrMetLysLeuGluLeuAlaGlnTyrArgGluValAla
AlaPheAlaGlnPheGlySerAspLeuAspAlaAlaThrGlnGlnLeuLeuSerArgGlyValArgLeu
ThrGluLeuLeuLysGlnGlyGlnTyrSerProMetAlaIleGluGluGlnValAlaValIleTyrAla
GlyValArgGlyTyrLeuAspLysLeuGluProSerLysIleThrLysPheGluAsnAlaPheLeuSerHis
ValValSerGlnHisGlnAlaLeuLeuGlyThrIleArgAlaAspGlyLysIleSerGluGlnSerAspAla
LysLeuLysGluIleValThrAsnPheLeuAlaGlyPheGluAla

| | |
|---|---|
| <210> | 16 |
| <211> | 81 |
| <212> | PRTN |
| <213> | Homo sapiens |
| <221> | CDS |
| <222> | 1-81 |
| <400> | 16 |

MetLeuArgGlnIleIleGlyGlnAlaLysLysHisProSerLeuIleProLeuPheVal
PheIleGlyThrGlyAlaThrGlyAlaThrLeuTyrLeuLeuArgLeuAlaLeuPheAsnProAspVal
CysTrpAspArgAsnAsnProGluProTrpAsnLysLeuGlyProAsnAspGlnTyrLysPheTyrSer
ValAsnValAspTyrSerLysLeuLysLysGluArgProAspPhe

| | |
|---|---|
| <210> | 17 |
| <211> | 21 |
| <212> | DNA |
| <213> | Homo sapiens |
| <221> | CDS |
| <222> | 1-21 |
| <400> | 17 |

CCAAGGACAGGAGTGAATGAC

| | |
|---|---|
| <210> | 18 |
| <211> | 20 |
| <212> | DNA |
| <213> | Homo sapiens |
| <221> | CDS |
| <222> | 1-20 |
| <400> | 18 |

GTATGGAATCCTGTGGCATC

| | |
|---|---|
| <210> | 19 |
| <211> | 21 |
| <212> | DNA |
| <213> | Homo sapiens |
| <221> | CDS |
| <222> | 1-21 |
| <400> | 19 |

CTACAGTCTGTGGCAGTTTCA

| | |
|---|---|
| <210> | 20 |
| <211> | 30 |
| <212> | DNA |
| <213> | Homo sapiens |
| <221> | CDS |
| <222> | 11-30 |
| <400> | 20 |

AAACTCAAAGGACTTGGCGGTACTTTATAT

| | |
|---|---|
| <210> | 21 |
| <211> | 24 |
| <212> | DNA |
| <213> | Homo sapiens |
| <221> | CDS |
| <222> | 1-24 |
| <400> | 21 |

CATTTCTACTTCGGTGTGCCTTCG

| | |
|---|---|
| <210> | 22 |
| <211> | 21 |
| <212> | DNA |
| <213> | Homo sapiens |
| <221> | CDS |
| <222> | 1-21 |
| <400> | 22 |

TGTCGGATCTGCTGCCCAAAC

| | |
|---|---|
| <210> | 23 |
| <211> | 26 |
| <212> | DNA |
| <213> | Homo sapiens |
| <221> | CDS |
| <222> | 1-26 |
| <400> | 23 |

CGTATTTATTGGAGCAGGGGGTACTG

| | |
|---|---|
| <210> | 24 |
| <211> | 27 |
| <212> | DNA |
| <213> | Homo sapiens |
| <221> | CDS |
| <222> | 1-27 |
| <400> | 24 |

CAATTTCCACCTCTGTGTGTGTACGAG

| | |
|---|---|
| <210> | 25 |
| <211> | Length |
| <212> | DNA |
| <213> | Homo sapiens |
| <221> | CDS |
| <222> | Sequence Placement #s |
| <400> | 25 |

AGTCGTGGCGTGCGTCTAACTGA

| | |
|---|---|
| <210> | 26 |
| <211> | 31 |
| <212> | PRTN |
| <213> | Rattus norvegicus |
| <221> | CDS |
| <222> | 147-166 |
| <400> | 26 |

GluGluSerGluGluGluValGluGluProGluGluAsnGlnGlnSerProGluVal
ValTyrGlyAsnLysLysAsnAsnGlnAsnAsnAsn

| | |
|---|---|
| <210> | 27 |
| <211> | 33 |
| <212> | PRTN |
| <213> | Rattus norvegicus |
| <221> | CDS |
| <222> | 168-189 |
| <400>27 | |

AspAspSerGlyThrPheTyrAspGlnThrValSerAsnAspLeuGluGluHisLeu
GluGluProTyrGlyAsnLysLysAsnAsnGlnAsnAsnAsn

| | |
|---|---|
| <210> | 28 |
| <211> | 31 |
| <212> | PRTN |
| <213> | Rattus norvegicus |
| <221> | CDS |
| <222> | 190-208 |
| <400> | 28 |

TyrGlyAsnLysLysAsnAsnAsnGlnAsnAsnAsnValValGluProGluProGlu
ProGluProGluProGluProGluProValSerGlu

| | |
|---|---|
| <210> | 29 |
| <211> | 21 |
| <212> | PRTN |
| <213> | Homo sapiens |
| <221> | CDS |
| <222> | 189-209 |
| <400> | 29 |

GluProValAlaGluProGluProAspProGluProGluProGluGluGluProVal
SerGlu

| | |
|---|---|
| <210> | 30 |
| <211> | 58 |
| <212> | DNA |
| <213> | Homo sapiens |
| <221> | CDS |
| <222> | 1-58 |
| <400> | 30 |

TTCCAGTAAATAGGCATGGAGTTGCATGGCTTACACTCGGACCTC
GTCGACATGCATT

| | |
|---|---|
| <210> | 31 |
| <211> | 57 |
| <212> | DNA |
| <213> | Homo sapiens |
| <221> | CDS |
| <222> | 1-57 |
| <400> | 31 |

CATAGTGCTTCTGCCACATGATAACGAGCTTACACTCGGACCTCG
TCGACATGCATT

| | |
|---|---|
| <210> | 32 |
| <211> | 57 |
| <212> | DNA |
| <213> | Homo sapiens |
| <221> | CDS |
| <222> | 1-57 |
| <400> | 32 |

TGTTCATCTCAGCAAAGCTCTCCTTGAACTTACACTCGGACCTCGT
CGACATGCATT

| | |
|---|---|
| <210> | 33 |
| <211> | 54 |
| <212> | DNA |
| <213> | Homo sapiens |
| <221> | CDS |
| <222> | 1-54 |
| <400> | 33 |

GCAAGGGGTGGTCACGCCGATCCATATTACACTCGGACCTCGTCG
ACATGCATT

| | |
|---|---|
| <210> | 34 |
| <211> | 55 |
| <212> | DNA |
| <213> | Homo sapiens |
| <221> | CDS |
| <222> | 1-55 |
| <400> | 34 |

TGGAAATTGCTCGCTTGCCAACTAGGCTTACACTCGGACCTC
GTCGACATGCATT

| | |
|---|---|
| <210> | 35 |
| <211> | 54 |
| <212> | DNA |
| <213> | Homo sapiens |
| <221> | CDS |
| <222> | 1-54 |
| <400> | 35 |

AATACCCTGGTAGCCAACATTCTGCCTTACACTCGGACCTCGTCG
ACATGCATT

| | |
|---|---|
| <210> | 36 |
| <211> | 54 |
| <212> | DNA |
| <213> | Homo sapiens |
| <221> | CDS |
| <222> | 1-54 |
| <400> | 36 |

CAGCATCTCTCACTTCTTCCACTCGTTTACACTCGGACCTCGTCGA
CATGCATT

| | |
|---|---|
| <210> | 37 |
| <211> | 57 |
| <212> | DNA |
| <213> | Homo sapiens |
| <221> | CDS |
| <222> | Sequence Placement #s |
| <400> | 1-57 |

TTTTCGTAGTCCCACTTGGAGGCTAAGCCTTACACTCGGACCTCG
TCGACATGCATT

| | |
|---|---|
| <210> | 38 |
| <211> | 54 |
| <212> | DNA |
| <213> | Homo sapiens |
| <221> | CDS |
| <222> | 1-54 |
| <400> | 38 |

GGATGGGGTTCACCTTCATGTCCAGCTTACACTCGGACCTCGTCG
ACATGCATT

| | |
|---|---|
| <210> | 39 |
| <211> | 54 |
| <212> | DNA |
| <213> | Homo sapiens |
| <221> | CDS |
| <222> | 1-54 |
| <400> | 39 |

CTGCTTGGCCACCCACTCTTTGTCAATTACACTCGGACCTCGTCGA
CATGCATT

| | |
|---|---|
| <210> | 40 |
| <211> | 54 |
| <212> | DNA |
| <213> | Homo sapiens |
| <221> | CDS |
| <222> | 1-54 |
| <400> | 40 |

ACAACCGTCTTCCACTCGTTCGAGCCTTACACTCGGACCTCGTCG
ACATGCATT

| | |
|---|---|
| <210> | 41 |
| <211> | 54 |
| <212> | DNA |
| <213> | Homo sapiens |
| <221> | CDS |
| <222> | 1-54 |
| <400> | 41 |

CAGGAGGCCTTCTCCTTCTCCTTCAATTACACTCGGACCTCGTCGA
CATGCATT

| | |
|---|---|
| <210> | 42 |
| <211> | 54 |
| <212> | DNA |
| <213> | Homo sapiens |
| <221> | CDS |
| <222> | 1-54 |
| <400> | 42 |

CCTTCTGGCTGGCAGACAGGTGCTTGTTACACTCGGACCTCGTCG
ACATGCATT

| | |
|---|---|
| <210> | 43 |
| <211> | 21 |
| <212> | PRTN |
| <213> | Homo sapiens |
| <221> | CDS |
| <222> | 1-21 |
| <400> | 43 |

AAGCACAGAGAAGAGGCAATC

| | |
|---|---|
| <210> | 44 |
| <211> | 20DNA |
| <212> | |
| <213> | Homo sapiens |
| <221> | CDS |
| <222> | 1-20 |
| <400> | 44 |

AAGCACTTGCGGTGCACGAT

| | |
|---|---|
| <210> | 45 |
| <211> | 19 |
| <212> | DNA |
| <213> | Homo sapiens |
| <221> | CDS |
| <222> | 1-19 |
| <400> | 45 |

TGGCTGTTTGCTGGATTCT

| | |
|---|---|
| <210> | 46 |
| <211> | 28 |
| <212> | DNA |
| <213> | Homo sapiens |
| <221> | CDS |
| <222> | 1-28 |
| <400> | 46 |

ATACCTTTTTAGGGTTTGCTGAAGATGG

| | |
|---|---|
| <210> | 47 |
| <211> | 24 |
| <212> | DNA |
| <213> | Homo sapiens |
| <221> | CDS |
| <222> | 1-24 |
| <400> | 47 |

GATCAGCGTAAGTGGGGAAAGCAT

| | |
|---|---|
| <210> | 48 |
| <211> | 22 |
| <212> | DNA |
| <213> | Homo sapiens |
| <221> | CDS |
| <222> | 1-22 |
| <400> | 48 |

ACGCACACCACGACTCAAGAGC

| | |
|---|---|
| <210> | 49 |
| <211> | 24 |
| <212> | DNA |
| <213> | Homo sapiens |
| <221> | CDS |
| <222> | 1-24 |
| <400> | 49 |

GGCTCTGGGTTGTTCTTTCTGTCC

| | |
|---|---|
| <210> | 50 |
| <211> | 18 |
| <212> | DNA |
| <213> | Homo sapiens |
| <221> | CDS |
| <222> | 1-18 |
| <400> | 50 |

GGCAAGGGGTGGTCACGC

| | |
|---|---|
| <210> | 51 |
| <211> | 25 |
| <212> | DNA |
| <213> | Homo sapiens |
| <221> | CDS |
| <222> | 1-25 |
| <400> | 51 |

CCTTACACCCGCATAGATAACAGCC

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A method for correcting disrupted axonal and synaptic protein synthesis comprising:
introducing an effective amount of at least one compound to a subject to restore an amount of at least one binding protein; or
disassociating at least one condensate in the subject comprising the binding protein via introduction of at least one peptide; and
to restore local protein synthesis events.

2. The method for correcting disrupted axonal and synaptic protein synthesis of claim 1, wherein the at least one binding protein comprises SEQ ID NO:

1.

3. The method for correcting disrupted axonal and synaptic protein synthesis of claim 1, wherein the at least one peptide comprises SEQ ID NO: 3.

4. The method for correcting disrupted axonal and synaptic protein synthesis of claim 1, wherein the subject has at least one nervouse system condition.

5. The method for correcting disrupted axonal and synaptic protein synthesis of claim 4, wherein the at least one nervouse system condition comprises amyotrophic lateral sclerosis.

6. The method for correcting disrupted axonal and synaptic protein synthesis of claim 5, wherein dissociating the at least one condensate comprising the binding protein reverses effects of amyotrophic lateral sclerosis.

7. The method for correcting disrupted axonal and synaptic protein synthesis of claim 1, wherein the at least one condensate comprises a pathological ribonucleoprotein.

8. The method for correcting disrupted axonal and synaptic protein synthesis of claim 6, wherein a presence of the pathological ribonucleoprotein interferes with axonal and pre-synaptic protein synthesis.

9. The method for correcting disrupted axonal and synaptic protein synthesis of claim 1, wherein mislocalization of the at least one binding protein binds and sequesters nuclear-encoded mitochondrial mRNAs and depletes a level of the nuclear-encoded mitochondrial mRNAs in at least one axon.

10. The method for correcting disrupted axonal and synaptic protein synthesis of claim 1, wherein dissociating condensates restores local translation and resolves binding protein derived toxicity in both axons and neuromuscular junctions.

11. A therapeutic treatment for at least one nervouse system condition comprising:
correcting disrupted axonal and synaptic protein synthesis in a subject via:
disassociating at least one condensate in the subject comprising the binding protein via introduction of at least one peptide; and
restoring local protein synthesis events in motor neuron axons and neuromuscular junction.

12. The therapeutic treatment for at least one nervouse system condition of claim 11, wherein the at least one binding protein comprises SEQ ID NO: 1.

13. The therapeutic treatment for at least one nervouse system condition of claim 11, wherein the at least one peptide comprises SEQ ID NO: 3.

14. The therapeutic treatment for at least one nervouse system condition of claim 11, wherein the at least one nervouse system condition comprises amyotrophic lateral sclerosis.

15. The therapeutic treatment for at least one nervouse system condition of claim 16, wherein dissociating the at least one condensate comprising the binding protein reverses effects of amyotrophic lateral sclerosis.

16. The therapeutic treatment for at least one nervouse system condition of claim 11, wherein the at least one condensate comprises a pathological ribonucleoprotein.

17. The therapeutic treatment for at least one nervouse system condition of claim 16, wherein a presence of the pathological ribonucleoprotein interferes with axonal and pre-synaptic protein synthesis.

18. The therapeutic treatment for at least one nervouse system condition of claim 11, wherein mislocalization of the at least one binding protein binds and sequesters nuclear-encoded mitochondrial mRNAs and depletes a level of the nuclear-encoded mitochondrial mRNAs in at least one axon.

19. The therapeutic treatment for at least one nervouse system condition of claim 11, wherein dissociating condensates restores local translation and resolves binding protein derived toxicity in both axons and neuromuscular junctions.
